(19) United States
(12) Patent Application Publication
(10) Pub. No.: US 2005/0048490 A1
(43) Pub. Date: Mar. 3, 2005

(54) Proteins associated with cell growth, differentiation, and death

(75) Inventors: Yalda Azimzai, Oakland, CA (US); Mariah R. Baughn, Los Angeles, CA (US); Shanya D. Becha, San Francisco, CA (US); Mark L. Borowsky, Needham, MA (US); Narinder K. Chawla, Union City, CA (US); Vicki S. Elliott, San Jose, CA (US); Brooke M. Emerling, Chicago, IL (US); Ameena R. Gandhi, San Francisco, CA (US); Kimberly J. Gietzen, San Jose, CA (US); Ann E Gorvad, Bellingham, WA (US); Jennifer A. Griffin, Fremont, CA (US); April JA Hafalia, Daly City, CA (US); Craig H. Ison, San Jose, CA (US); Amu E. Kable, Silver Spring, MD (US); Daniel P. Kalafus, San Francisco, CA (US); Patircia M. Lehr-Mason, Morgan Hill, CA (US); Dyung Aina M. Lu, San Jose, CA (US); Joseph P. Marquis, San Jose, CA (US); Danniel B. Nguyen, San Jose, CA (US); Jayalaxmi Ramkumar, Fremont, CA (US); Thomas W. Richardson, Redwood City, CA (US); Stephanie K. Kareht, Redwood City, CA (US); Anita Swarnakar, San Francisco, CA (US); Y Tom Tang, SAn Jose, CA (US); Uyen K. Tran, San Jose, CA (US); Bridget A. Warren, San Marcos, CA (US); Yuming Xu, Mountain View, CA (US); Monique G. Yao, Mountain View, CA (US); Huibin Yue, Cupertino, CA (US); Henry Yue, Sunnyvale, CA (US)

Correspondence Address: INCYTE CORPORATION;EXPERIMENTAL STATION, ROUTE 141 & HENRY CLAY ROAD, BLDG. E336, WILMINGTON, DE, 19880, US

(73) Assignee: incyte corp., palo alto, CA (US)

(21) Appl. No.: 10/491,213

(22) PCT Filed: Sep. 26, 2002
(86) PCT No.: PCT/US02/31095

(51) Int. Cl. C12Q 1/68; G01N 33/574; C07H 21/04; C12N 9/64

(52) U.S. Cl. 435/6; 536/23.2; 435/7.23; 435/226; 435/69.1; 435/325; 530/350

(57) ABSTRACT

Various embodiments of the invention provide human proteins associated with cell growth, differentiation, and death (CGDD) and polynucleotides whichidentify and encode CGDD. Embodiments of the invention also provide expression vectors, host cells, antibodies, agonists, and antagonists. Other embodiments provide methods for diagnosing, treating, or preventing disorders associated with aberrant expression of CGDD.

### TECHNICAL FIELD

The invention relates to novel nucleic acids, proteins associated with cell growth, differentiation, and death encoded by these nucleic acids, and to the use of these nucleic acids and proteins in the diagnosis, treatment, and prevention of cell proliferative disorders including cancer, developmental disorders, neurological disorders, autoimmune/inflammatory disorders, reproductive disorders, and disorders of the placenta. The invention also relates to the assessment of the effects of exogenous compounds on the expression of nucleic acids and proteins associated with cell growth, differentiation, and death.

### BACKGROUND OF THE INVENTION

Human growth and development requires the spatial and temporal regulation of cell differentiation, cell proliferation, and apoptosis. These processes coordinately control reproduction, aging, embryogenesis, morphogenesis, organogenesis, and tissue repair and maintenance. At the cellular level, growth and development is governed by the cell's decision to enter into or exit from the cell division cycle and by the cell's commitment to a terminally differentiated state. These decisions are made by the cell in response to extracellular signals and other environmental cues it receives. The following discussion focuses on the molecular mechanisms of cell division, embryogenesis, cell differentiation and proliferation, and apoptosis, as well as disease states such as cancer which can result from disruption of these mechanisms.

Cell Cycle

Cell division is the fundamental process by which all living things grow and reproduce. In unicellular organisms such as yeast and bacteria, each cell division doubles the number of organisms. In multicellular species many rounds of cell division are required to replace cells lost by wear or by programmed cell death, and for cell differentiation to produce a new tissue or organ. Progression through the cell cycle is governed by the intricate interactions of protein complexes. This regulation depends upon the appropriate expression of proteins which control cell cycle progression in response to extracellular signals, such as growth factors and other mitogens, and intracellular cues, such as DNA damage or nutrient starvation. Molecules which directly or indirectly modulate cell cycle progression fall into several categories, including cyclins, cyclin-dependent protein kinases, growth factors and their receptors, second messenger and signal transduction proteins, oncogene products, and tumor-suppressor proteins.

Details of the cell division cycle may vary, but the basic process consists of three principle events. The first event, interphase, involves preparations for cell division, replication of the DNA, and production of essential proteins. In the second event, mitosis, the nuclear material is divided and separates to opposite sides of the cell. The final event, cytokinesis, is division and fission of the cell cytoplasm. The sequence and timing of cell cycle transitions is under the control of the cell cycle regulation system which controls the process by positive or negative regulatory circuits at various check points.

Mitosis marks the end of interphase and concludes with the onset of cytokinesis. There are four stages in mitosis, occurring in the following order: prophase, metaphase, anaphase and telophase. Prophase includes the formation of bi-polar mitotic spindles, composed of microtubules and associated proteins such as dynein, which originate from polar mitotic centers. During metaphase, the nuclear material condenses and develops kinetochore fibers which aid in its physical attachment to the mitotic spindles. The ensuing movement of the nuclear material to opposite poles along the mitotic spindles occurs during anaphase. Telophase includes the disappearance of the mitotic spindles and kinetochore fibers from the nuclear material. Mitosis depends on the interaction of numerous proteins. For example, centromere-associated proteins such as CENP-A, -B, and -C, play structural roles in kinetochore formation and assembly (Saffery, R. et al. (2000) Human Mol. Gen. 9:175-185).

During the M phase of eukaryotic cell cycling, structural rearrangements occur ensuring appropriate distribution of cellular components between daughter cells. Breakdown of interphase structures into smaller subunits is common. The nuclear envelope breaks into vesicles, and nuclear lamins are disassembled. Subsequent phosphorylation of these lamins occurs and is maintained until telophase, at which time the nuclear lamina structure is reformed. cDNAs responsible for encoding M phase phosphorylation (MPPs) are components of U3 small nucleolar ribonucleoprotein (snoRNP), and relocalize to the nucleolus once mitosis is complete (Westendorf, J. M. et al. (1998) J. Biol. Chem. 9:437-449). U3 snoRNPs are essential mediators of RNA processing events.

Proteins involved in the regulation of cellular processes such as mitosis include the Ser/Thr-protein phosphatases type 1 (PP-1). PP-1s act by dephosphorylation of key proteins involved in the metaphase-anaphase transition. The gene PP1R7 encodes the regulatory polypeptide sds22, having at least six splice variants (Ceulemans, H. et al. (1999) Eur. J. Biochem. 262:36-42). Sds22 modulates the activity of the catalytic subunit of PP-1s, and enhances the PP-1-dependent dephosphorylation of mitotic substrates.

Cell cycle regulatory proteins play an important role in cell proliferation and cancer. For example, failures in the proper execution and timing of cell cycle events can lead to chromosome segregation defects resulting in aneuploidy or polyploidy. This genomic instability is characteristic of transformed cells (Luca, F. C. and M. Winey (1998) Mol. Biol. Cell. 9:29-46). A recently identified protein, mMOB1, is the mammalian homolog of yeast MOB1, an essential yeast gene required for completion of mitosis and maintenance of ploidy. The mammalian mMOB1 is a member of protein complexes including protein phosphatase 2A (PP2A), and its phosphorylation appears to be regulated by PP2A (Moreno, C. S. et al. (2001) J. Biol. Chem. 276:24253-24260). PP2A has been implicated in the development of human cancers, including lung and colon cancers and leukemias.

Cell cycle regulation involves numerous proteins interacting in a sequential manner. The eukaryotic cell cycle consists of several highly controlled events whose precise order ensures successful DNA replication and cell division. Cells maintain the order of these events by making later events dependent on the successful completion of earlier events. This dependency is enforced by cellular mechanisms called checkpoints. Examples of additional cell cycle regulatory proteins include the histone deacetylases (HDACs). HDACs are involved in cell cycle regulation, and modulate chromatin structure. Human HDAC1 has been found to interact in vitro with the human HusI gene product, whose Schizosaccharomyces pombe homolog has been implicated in G₂/M checkpoint control (Cai, R. L. et al. (2000) J. Biol. Chem. 275:27909-27916).

DNA damage (G₂) and DNA replication (S-phase) checkpoints arrest eukaryotic cells at the G₂/M transition. This arrest provides time for DNA repair or DNA replication to occur before entry into mitosis. Thus, the G₂/M checkpoint ensures that mitosis only occurs upon completion of DNA replication and in the absence of chromosomal damage. The Hus1 gene of Schizosaccharomyces pombe is a cell cycle checkpoint gene, as are the rad family of genes (e.g., rad1 and rad9) (Volkmer, E. and L. M. Karnitz (1999) J. Biol. Chem. 274:567-570; Kostrub C. F. et al. (1998) EMBO J. 17:2055-2066). These genes are involved in the mitotic checkpoint, and are induced by either DNA damage or blockage of replication. Induction of DNA damage or replication block leads to loss of function of the Hus1 gene and subsequent cell death. Human homologs have been identified for most of the rad genes, including ATM and ATR, the human homologs of rad3p. Mutations in the ATM gene are correlated with the severe congenital disease ataxia-telagiectasia (Savitsky, K. et al. (1995) Science 268:1749-1753). The human Hus1 protein has been shown to act in a complex with rad1 protein which interacts with rad9, making them central components of a DNA damage-responsive protein complex of human cells (Volkmer and Karnitz, supra).

The entry and exit of a cell from mitosis is regulated by the synthesis and destruction of a family of activating proteins called cyclins. Cyclins act by binding to and activating a group of cyclin-dependent protein kinases (Cdks) which then phosphorylate and activate selected proteins involved in the mitotic process. Cyclins are characterized by a large region of shared homology that is approximately 180 amino acids in length and referred to as the “cyclin box” (Chapman, D. L. and D. J. Wolgemuth (1993) Development 118:229-240). In addition, cyclins contain a conserved 9 amino acid sequence in the N-terminal region of the molecule called the “destruction box.” This sequence is believed to be a recognition code that triggers ubiquitin-mediated degradation of cyclin B (Hunt, T. (1991) Nature 349:100-101). Several types of cyclins exist (Ciechanover, A. (1994) Cell 79:13-21). Progression through G1 and S phase is driven by the G1 cyclins and their catalytic subunits, including Cdk2-cyclin A, Cdk2-cyclin E, Cdk4-cyclin D and Cdk6-cyclin D. Progression through the G2-M transition is driven by the activation of mitotic CDK-cyclin complexes such as Cdc2-cyclin A, Cdc2-cyclin B1 and Cdc2-cyclin B2 complexes (reviewed in Yang, J. and S. Kornbluth (1999) Trends Cell Biol. 9:207-210).

Cyclins are degraded through the ubiquitin conjugation system (UCS), a major pathway for the degradation of cellular proteins in eukaroytic cells and in some bacteria. The UCS mediates the elimination of abnormal proteins and regulates the half-lives of important regulatory proteins that control cellular processes such as gene transcription and cell cycle progression. The UCS is implicated in the degradation of mitotic cyclin kinases, oncoproteins, tumor suppressor genes such as p53, viral proteins, cell surface receptors associated with signal transduction, transcriptional regulators, and mutated or damaged proteins (Ciechanover, supra).

The process of ubiquitin conjugation and protein degradation occurs in five principle steps (Jentsch, S. (1992) Annu. Rev. Genet. 26:179-207). First ubiquitin (Ub), a small, heat stable protein is activated by a ubiquitin-activating enzyme (E1) in an ATP dependent reaction which binds the C-terminus of Ub to the thiol group of an internal cysteine residue in E1. Second, activated Ub is transferred to one of several Ub-conjugating enzymes (E2). Different ubiquitin-dependent proteolytic pathways employ structurally similar, but distinct ubiquitin-conjugating enzymes that are associated with recognition subunits which direct them to proteins carrying a particular degradation signal. Third, E2 transfers the Ub molecule through its C-terminal glycine to a member of the ubiquitin-protein ligase family, E3. Fourth, E3 transfers the Ub molecule to the target protein. Additional Ub molecules may be added to the target protein forming a multi-Ub chain structure. Fifth, the ubiquinated protein is then recognized and degraded by the proteasome, a large, multisubunit proteolytic enzyme complex, and Ub is released for re-utilization.

Prior to activation, Ub is usually expressed as a fusion protein composed of an N-terminal ubiquitin and a C-terminal extension protein (CEP) or as a polyubiquitin protein with Ub monomers attached head to tail. CEPs have characteristics of a variety of regulatory proteins; most are highly basic, contain up to 30% lysine and arginine residues, and have nucleic acid-binding domains (Monia, B. P. et al. (1989) J. Biol. Chem. 264:4093-4103). The fusion protein is an important intermediate which appears to mediate co-regulation of the cell's translational and protein degradation activities, as well as localization of the inactive enzyme to specific cellular sites. Once delivered, C-terminal hydrolases cleave the fusion protein to release a functional Ub (Monia et al., supra).

Ub-conjugating enzymes (E2s) are important for substrate specificity in different UCS pathways. All E2s have a conserved domain of approximately 16 kDa called the UBC domain that is at least 35% identical in all E2s and contains a centrally located cysteine residue required for ubiquitin-enzyme thiolester formation (Jentsch, supra). A well conserved proline-rich element is located N-terminal to the active cysteine residue. Structural variations beyond this conserved domain are used to classify the E2 enzymes. Class I E2s consist almost exclusively of the conserved UBC domain. Class II E2s have various unrelated C-terminal extensions that contribute to substrate specificity and cellular localization. Class III E2s have unique N-terminal extensions which are believed to be involved in enzyme regulation or substrate specificity.

A mitotic cyclin-specific E2 (E2-C) is characterized by the conserved UBC domain, an N-terminal extension of 30 amino acids not found in other E2s, and a 7 amino acid unique sequence adjacent to this extension. These characteristics together with the high affinity of E2-C for cyclin identify it as a new class of E2 (Aristarkhov, A. et al. (1996) Proc. Natl. Acad. Sci. 93:4294-99).

Ubiquitin-protein ligases (E3s) catalyze the last step in the ubiquitin conjugation process, covalent attachment of ubiquitin to the substrate. E3 plays a key role in determining the specificity of the process. Only a few E3s have been identified so far. One type of E3 ligases is the HECT (homologous to E6-AP C-terminus) domain protein family. One member of the family, E6-AP (E6-associated protein) is required, along with the human papillomavirus (HPV) E6 oncoprotein, for the ubiquitination and degradation of p53 (Scheffner, M. et al. (1993) Cell 75:495-505). The C-terminal domain of HECT proteins contains the highly conserved ubiquitin-binding cysteine residue. The N-terminal region of the various HECT proteins is variable and is believed to be involved in specific substrate recognition (Huibregtse, J.M. et al. (1997) Proc. Natl Acad. Sci. USA 94:3656-3661). The SCF (Skp1-Cdc53/Cullin-F box receptor) family of proteins comprise another group of ubiquitin ligases (Deshaies, R. (1999) Annu. Rev. Dev. Biol. 15:435-467). Multiple proteins are recruited into the SCF complex, including Skpl, cullin, and an F box domain containing protein. The F box protein binds the substrate for the ubiquitination reaction and may play roles in determining substrate specificity and orienting the substrate for reaction. Skp1 interacts with both the F box protein and cullin and may be involved in positioning the F box protein and cullin in the complex for transfer of ubiquitin from the E2 enzyme to the protein substrate. Substrates of SCF ligases include proteins involved in regulation of CDK activity, activation of transcription, signal transduction, assembly of kinetochores, and DNA replication.

Sgt1 was identified in a screen for genes in yeast that suppress defects in kinetochore function caused by mutations in Skp1 (Kitagawa, K. et al. (1999) Mol. Cell 4:21-33). Sgt1 interacts with Skp1 and associates with SCF ubiquitin ligase. Defects in Sgt1 cause arrest of cells at either G1 or G2 stages of the cell cycle. A yeast Sgt1 null mutant can be rescued by human Sgt1, an indication of the conservation of Sgt1 function across species. Sgt1 is required for assembly of kinetochore complexes in yeast.

Abnormal activities of the UCS are implicated in a number of diseases and disorders. These include, e.g., cachexia (Llovera, M. et al. (1995) Int. J. Cancer 61:138-141), degradation of the tumor-suppressor protein, p53 (Ciechanover, supra), and neurodegeneration such as observed in Alzheimer's disease (Gregori, L. et al. (1994) Biochem. Biophys. Res. Commun. 203:1731-1738). Since ubiquitin conjugation is a rate-limiting step in antigen presentation, the ubiquitin degradation pathway may also have a critical role in the immune response (Grant, E. P. et al. (1995) J. Immunol. 155:3750-3758).

Certain cell proliferation disorders can be identified by changes in the protein complexes that normally control progression through the cell cycle. A primary treatment strategy involves reestablishing control over cell cycle progression by manipulation of the proteins involved in cell cycle regulation (Nigg, E. A. (1995) BioEssays 17:471-480).

Embryogenesis

Mammalian embryogenesis is a process which encompasses the first few weeks of development following conception. During this period, embryogenesis proceeds from a single fertilized egg to the formation of the three embryonic tissues, then to an embryo which has most of its internal organs and all of its external features.

The normal course of mammalian embryogenesis depends on the correct temporal and spatial regulation of a large number of genes and tissues. These regulation processes have been intensely studied in mouse. An essential process that is still poorly understood is the activation of the embryonic genome after fertilization. As mouse oocytes grow, they accumulate transcripts that are either translated directly into proteins or stored for later activation by regulated polyadenylation. During subsequent meiotic maturation and ovulation, the maternal genome is transcriptionally inert, and most maternal transcripts are deadenylated and/or degraded prior to, or together with, the activation of the zygotic genes at the two-cell stage (Stutz, A. et al. (1998) Genes Dev. 12:2535-2548). The maternal to embryonic transition involves the degradation of oocyte, but not zygotic transcripts, the activation of the embryonic genome, and the induction of cell cycle progression to accommodate early development.

MATER (Maternal Antigen That Embryos Require) was initially identified as a target of antibodies from mice with ovarian immunity (Tong, Z-B. and L. M. Nelson (1999) Endocrinology 140:3720-3726). Expression of the gene encoding MATER is restricted to the oocyte, making it one of a limited number of known maternal-effect genes in mammals (Tong, Z-B. et al. (2000) Mamm. Genome 11:281-287). The MATER protein is required for embryonic development beyond two cells, based upon preliminary results from mice in which this gene has been inactivated. The 1111-amino acid MATER protein contains a hydrophilic repeat region in the amino terminus, and a region containing 14 leucine-rich repeats in the carboxyl terminus. These repeats resemble the sequence found in porcine ribonuclease inhibitor that is critical for protein-protein interactions.

The degradation of maternal transcripts during meiotic maturation and ovulation may involve the activation of a ribonuclease just prior to ovulation. Thus the function of MATER may be to bind to the maternal ribonuclease and prevent degradation of zygotic transcripts (Tong et al., supra). In addition to its role in oocyte development and embryogenesis, MATER may also be relevant to the pathogenesis of ovarian immunity, as it is a target of autoantibodies in mice with autoimmune oophoritis (Tong and Nelson, supra).

The maternal mRNA D7 is a moderately abundant transcript in Xenopus laevis whose expression is highest in, and perhaps restricted to, oogenesis and early embryogenesis. The D7 protein is absent from oocytes and first begins to accumulate during oocyte maturation. Its levels are highest during the first day of embryonic development and then they decrease. The loss of D7 protein affects the maturation process itself, significantly delaying the time course of germinal vesicle breakdown. Thus, D7 is a newly described protein involved in oocyte maturation (Smith, R. C. et al. (1988) Genes Dev. 2(10):1296-306.)

Many other genes are involved in subsequent stages of embryogenesis. After fertilization, the oocyte is guided by fimbria at the distal end of each fallopian tube into and through the fallopian tube and thence into the uterus. Changes in the uterine endometrium prepare the tissue to support the implantation and embryonic development of a fertilized ovum. Several stages of division have occurred before the dividing ovum, now a blastocyst with about 100 cells, enters the uterus. Upon reaching the uterus, the developing blastocyst usually remains in the uterine cavity an additional two to four days before implanting in the endometrium, the inner lining of the uterus. Implantation results from the action of trophoblast cells that develop over the surface of the blastocyst. These cells secrete proteolytic enzymes that digest and liquefy the cells of the endometrium. The invasive process is reviewed in Fisher, S. J. and C. H. Damsky (1993; Semin Cell Biol 4:183-188) and Graham, C. H. and P. K. Lala (1992; Biochem Cell Biol 70:867-874). Once implantation has taken place, the trophoblast and other sublying cells proliferate rapidly, forming the placenta and the various membranes of pregnancy. (See Guyton, A. C. (1991) Textbook of Medical Physiology, 8^{th} ed. W.B. Saunders Company, Philadelphia Pa., pp. 915-919.)

The placenta has an essential role in protecting and nourishing the developing fetus. In most species the syncytiotrophoblast layer is present on the outside of the placenta at the fetal-maternal interface. This is a continuous structure, one cell deep, formed by the fusion of the constituent trophoblast cells. The syncytiotrophoblast cells play important roles in maternal-fetal exchange, in tissue remodeling during fetal development, and in protecting the developing fetus from the maternal immune response (Stoye, J. P. and J. M. Coffin (2000) Nature 403:715-717).

A gene called syncytin is the envelope gene of a human endogenous defective provirus. Syncytin is expressed in high levels in placenta, and more weakly in testis, but is not detected in any other tissues (Mi, S. et al. (2000) Nature 403:785-789). Syncytin expression in the placenta is restricted to the syncytiotrophoblasts. Since retroviral env proteins are often involved in promoting cell fusion events, it was thought that syncytin might be involved in regulating the fusion of trophoblast cells into the syncytiotrophoblast layer. Experiments demonstrated that syncytin can mediate cell fusion in vitro, and that anti-syncytin antibodies can inhibit the fusion of placental cytotrophoblasts (Mi et al., supra). In addition, a conserved immunosuppressive domain present in retroviral envelope proteins, and found in syncytin at amino acid residues 373-397, might be involved in preventing maternal immune responses against the developing embryo.

Syncytin may also be involved in regulating trophoblast invasiveness by inducing trophoblast fusion and terminal differentiation (Mi et al., supra). Insufficient trophoblast infiltration of the uterine wall is associated with placental disorders such as preeclampsia, or pregnancy induced hypertension, while uncontrolled trophoblast invasion is observed in choriocarcinoma and other gestational trophoblastic diseases. Thus syncytin function may be involved in these diseases.

Cell Differentiation

Multicellular organisms are comprised of diverse cell types that differ dramatically both in structure and function, despite the fact that each cell is like the others in its hereditary endowment. Cell differentiation is the process by which cells come to differ in their structure and physiological function. The cells of a multicellular organism all arise from mitotic divisions of a single-celled zygote. The zygote is totipotent, meaning that it has the ability to give rise to every type of cell in the adult body. During development the cellular descendants of the zygote lose their totipotency and become determined. Once its prospective fate is achieved, a cell is said to have differentiated. All descendants of this cell will be of the same type.

Human growth and development requires the spatial and temporal regulation of cell differentiation, along with cell proliferation and regulated cell death. These processes coordinate to control reproduction, aging, embryogenesis, morphogenesis, organogenesis, and tissue repair and maintenance. The processes involved in cell differentiation are also relevant to disease states such as cancer, in which case the factors regulating normal cell differentiation have been altered, allowing the cancerous cells to proliferate in an anaplastic, or undifferentiated, state.

The mechanisms of differentiation involve cell-specific regulation of transcription and translation, so that different genes are selectively expressed at different times in different cells. Genetic experiments using the fruit fly Drosophila melanogaster have identified regulated cascades of transcription factors which control pattern formation during development and differentiation. These include the homeotic genes, which encode transcription factors containing homeobox motifs. The products of homeotic genes determine how the insect's imaginal discs develop from masses of undifferentiated cells to specific segments containing complex organs. Many genes found to be involved in cell differentiation and development in Drosophila have homologs in mammals. Some human genes have equivalent developmental roles to their Drosophila homologs. The human homolog of the Drosophila eyes absent gene (eya) underlies branchio-oto-renal syndrome, a developmental disorder affecting the ears and kidneys (Abdelhak, S. et al. (1997) Nat. Genet. 15:157-164). The Drosophila slit gene encodes a secreted leucine-rich repeat containing protein expressed by the midline glial cells and required for normal neural development.

At the cellular level, growth and development are governed by the cell's decision to enter into or exit from the cell cycle and by the cell's commitment to a terminally differentiated state. Differential gene expression within cells is triggered in response to extracellular signals and other environmental cues. Such signals include growth factors and other mitogens such as retinoic acid; cell-cell and cell-matrix contacts; and environmental factors such as nutritional signals, toxic substances, and heat shock. Candidate genes that may play a role in differentiation can be identified by altered expression patterns upon induction of cell differentiation in vitro.

The final step in cell differentiation results in a specialization that is characterized by the production of particular proteins, such as contractile proteins in muscle cells, serum proteins in liver cells and globins in red blood cell precursors. The expression of these specialized proteins depends at least in part on cell-specific transcription factors. For example, the homeobox-containing transcription factor PAX-6 is essential for early eye determination, specification of ocular tissues, and normal eye development in vertebrates.

In the case of epidermal differentiation, the induction of differentiation-specific genes occurs either together with or following growth arrest and is believed to be linked to the molecular events that control irreversible growth arrest. Irreversible growth arrest is an early event which occurs when cells transit from the basal to the innermost suprabasal layer of the skin and begin expressing squamous-specific genes. These genes include those involved in the formation of the cross-linked envelope, such as transglutaminase I and III, involucrin, loricin, and small proline-rich repeat (SPRR) proteins. The SPRR proteins are 8-10 kDa in molecular mass, rich in proline, glutamine, and cysteine, and contain similar repeating sequence elements. The SPRR proteins may be structural proteins with a strong secondary structure or metal-binding proteins such as metallothioneins. (Jetten, A. M. and B. L. Harvat (1997) J. Dermatol. 24:711-725; PRINTS Entry PR00021 PRORICH Small proline-rich protein signature.)

The Wnt gene family of secreted signaling molecules is highly conserved throughout eukaryotic cells. Members of the Wnt family are involved in regulating chondrocyte differentiation within the cartilage template. Wnt-5a, Wnt-5b and Wnt-4 genes are expressed in chondrogenic regions of the chicken limb, Wnt-5a being expressed in the perichondrium (mesenchymal cells immediately surrounding the early cartilage template). Wnt-Sa misexpression delays the maturation of chondrocytes and the onset of bone collar formation in chicken limb (Hartmann, C. and C. J. Tabin (2000) Development 127:3141-3159).

Glypicans are a family of cell surface heparan sulfate proteoglycans that play an important role in cellular growth control and differentiation. Cerebroglycan, a heparan sulfate proteoglycan expressed in the nervous system, is involved with the motile behavior of developing neurons (Stipp, C. S. et al. (1994) J. Cell Biol. 124:149-160).

Notch plays an active role in the differentiation of glial cells, and influences the length and organization of neuronal processes (for a review, see Frisen, J. and U. Lendahl (2001) Bioessays 23:3-7). The Notch receptor signaling pathway is important for morphogenesis and development of many organs and tissues in multicellular species. Drosophila fringe proteins modulate the activation of the Notch signal transduction pathway at the dorsal-ventral boundary of the wing imaginal disc. Mammalian fringe-related family members participate in boundary determination during segmentation (Johnston, S. H. et al. (1997) Development 124:2245-2254).

Recently a number of proteins have been found to contain a conserved cysteine-rich domain of about 60 amino-acid residues called the LIM domain (for Lin-11 Isl-1 Mec-3) (Freyd, G. et al. (1990) Nature 344:876-879; Baltz, R. et al. (1992) Plant Cell 4:1465-1466). In the LIM domain, there are seven conserved cysteine residues and a histidine. The LIM domain binds two zinc ions (Michelsen, J. W. et al. (1993) Proc. Natl. Acad. Sci. U.S.A. 90:4404-4408). LIM does not bind DNA; rather, it seems to act as an interface for protein-protein interaction.

Apoptosis

Apoptosis is the genetically controlled process by which unneeded or defective cells undergo programmed cell death. Selective elimination of cells is as important for morphogenesis and tissue remodeling as is cell proliferation and differentiation. Lack of apoptosis may result in hyperplasia and other disorders associated with increased cell proliferation. Apoptosis is also a critical component of the immune response. Immune cells such as cytotoxic T-cells and natural killer cells prevent the spread of disease by inducing apoptosis in tumor cells and virus-infected cells. In addition, immune cells that fail to distinguish self molecules from foreign molecules must be eliminated by apoptosis to avoid an autoimmune response.

Apoptotic cells undergo distinct morphological changes. Hallmarks of apoptosis include cell shrinkage, nuclear and cytoplasmic condensation, and alterations in plasma membrane topology. Biochemically, apoptotic cells are characterized by increased intracellular calcium concentration, fragmentation of chromosomal DNA, and expression of novel cell surface components.

The molecular mechanisms of apoptosis are highly conserved, and many of the key protein regulators and effectors of apoptosis have been identified. Apoptosis generally proceeds in response to a signal which is transduced intracellularly and results in altered patterns of gene expression and protein activity. Signaling molecules such as hormones and cytokines are known both to stimulate and to inhibit apoptosis through interactions with cell surface receptors. Transcription factors also play an important role in the onset of apoptosis. A number of downstream effector molecules, especially proteases, have been implicated in the degradation of cellular components and the proteolytic activation of other apoptotic effectors.

The Bcl-2 family of proteins, as well as other cytoplasmic proteins, are key regulators of apoptosis. There are at least 15 Bcl-2 family members within 3 subfamilies. These proteins have been identified in mammalian cells and in viruses, and each possesses at least one of four Bcl-2 homology domains (BH1 to BH4), which are highly conserved. Bcl-2 family proteins contain the BH1 and BH2 domains, which are found in members of the pro-survival subfamily, while those proteins which are most similar to Bcl-2 have all four conserved domains, enabling inhibition of apoptosis following encounters with a variety of cytotoxic challenges. Members of the pro-survival subfamily include Bcl-2, Bcl-x_{L}, Bcl-w, Mcl-1, and A1 in mammals; NF-13 (chicken); CED-9 (Caenorhabditis elegans); and viral proteins BHRF1, LMW5-HL, ORF16, KS-Bcl-2, and ElB-19K. The BH3 domain is essential for the function of pro-apoptosis subfamily proteins. The two pro-apoptosis subfamilies, Bax and BH3, include Bax, Bak, and Bok (also called Mtd); and Bik, Blk, Hrk, BNIP3, Bim_{L}, Bad, Bid, and Egl-1 (C. elegans); respectively. Members of the Bax subfamily contain the BH1, BH2, and BH3 domains, and resemble Bcl-2 rather closely. In contrast, members of the BH3 subfamily have only the 9-16 residue BH3 domain, being otherwise unrelated to any known protein, and only Bik and Blk share sequence similarity. The proteins of the two pro-apoptosis subfamilies may be the antagonists of pro-survival subfamily proteins. This is illustrated in C. elegans where Egl-1, which is required for apoptosis, binds to and acts via CED-9 (for review, see Adams, J. M. and S. Cory (1998) Science 281:1322-1326).

Heterodimerization between pro-apoptosis and anti-apoptosis subfamily proteins seems to have a titrating effect on the functions of these protein subfamilies, which suggests that relative concentrations of the members of each subfamily may act to regulate apoptosis. Heterodimerization is not required for a pro-survival protein; however, it is essential in the BH3 subfamily, and less so in the Bax subfamily.

The Bcl-2 protein has 2 isoforms, alpha and beta, which are formed by alternative splicing. It forms homodimers and heterodimers with Bax and Bak proteins and the Bcl-X isoform Bcl-x_{S}. Heterodimerization with Bax requires intact BH1 and BH2 domains, and is necessary for pro-survival activity. The BH4 domain seems to be involved in pro-survival activity as well. Bcl-2 is located within the inner and outer mitochondrial membranes, as well as within the nuclear envelope and endoplasmic reticulum, and is expressed in a variety of tissues. Its involvement in follicular lymphoma (type II chronic lymphatic leukemia) is seen in a chromosomal translocation T(14;18) (q32;q21) and involves immunoglobulin gene regions.

The Bcl-x protein is a dominant regulator of apoptotic cell death. Alternative splicing results in three isoforms, Bcl-xB, a long isoform, and a short isoform. The long isoform exhibits cell death repressor activity, while the short isoform promotes apoptosis. Bcl-xL forms heterodimers with Bax and Bak, although heterodimerization with Bax does not seem to be necessary for pro-survival (anti-apoptosis) activity. Bcl-xS forms heterodimers with Bcl-2. Bcl-x is found in mitochondrial membranes and the perinuclear envelope. Bcl-xS is expressed at high levels in developing lymphocytes and other cells undergoing a high rate of turnover. Bcl-xL is found in adult brain and in other tissues' long-lived post-mitotic cells. As with Bcl-2, the BH1, BH2, and BH4 domains are involved in pro-survival activity.

The Bcl-w protein is found within the cytoplasm of almost all myeloid cell lines and in numerous tissues, with the highest levels of expression in brain, colon, and salivary gland. This protein is expressed in low levels in testis, liver, heart, stomach, skeletal muscle, and placenta, and a few lymphoid cell lines. Bcl-w contains the BH1, BH2, and BH4 domains, all of which are needed for its cell survival promotion activity. Although mice in which Bcl-w gene function was disrupted by homologous recombination were viable, healthy, and normal in appearance, and adult females had normal reproductive function, the adult males were infertile. In these males, the initial, prepuberty stage of spermatogenesis was largely unaffected and the testes developed normally. However, the seminiferous tubules were disorganized, contained numerous apoptotic cells, and were incapable of producing mature sperm. This mouse model may be applicable to some cases of human male sterility and suggests that alteration of programmed cell death in the testes may be useful in modulating fertility (Print, C. G. et al. (1998) Proc. Natl. Acad. Sci. USA 95:12424-12431).

Studies in rat ischemic brain found Bcl-w to be overexpressed relative to its normal low constitutive level of expression in nonischemic brain. Furthermore, in vitro studies to examine the mechanism of action of Bcl-w revealed that isolated rat brain mitochondria were unable to respond to an addition of recombinant Bax or high concentrations of calcium when Bcl-w was also present. The normal response would be the release of cytochrome c from the mitochondria. Additionally, recombinant Bcl-w protein was found to inhibit calcium-induced loss of mitochondrial transmembrane potential, which is indicative of permeability transition. Together these findings suggest that Bcl-w may be a neuro-protectant against ischemic neuronal death and may achieve this protection via the mitochondrial death-regulatory pathway (Yan, C. et al. (2000) J. Cereb. Blood Flow Metab. 20:620-630).

The bfl-1 gene is an additional member of the Bcl-2 family, and is also a suppressor of apoptosis. The Bfl-1 protein has 175 amino acids, and contains the BH1, BH2, and BH3 conserved domains found in Bcl-2 family members. It also contains a Gln-rich NH2-terminal region and lacks an NH domain 1, unlike other Bcl-2 family members. The mouse Al protein shares high sequence homology with Bfl-1 and has the 3 conserved domains found in Bfl-1. Apoptosis induced by the p53 tumor suppressor protein is suppressed by Bfl-1, similar to the action of Bcl-2, Bcl-xL, and EBV-BHRF1 (D'Sa-Eipper, C. et al. (1996) Cancer Res. 56:3879-3882). Bfl-1 is found intracellularly, with the highest expression in the hematopoietic compartment, i.e. blood, spleen, and bone marrow; moderate expression in lung, small intestine, and testis; and minimal expression in other tissues. It is also found in vascular smooth muscle cells and hematopoietic malignancies. A correlation has been noted between the expression level of bfl-1 and the development of stomach cancer, suggesting that the Bfl-1 protein is involved in the development of stomach cancer, either in the promotion of cancerous cell survival or in cancer (Choi, S. S. et al. (1995) Oncogene 11:1693-1698).

Cancers are characterized by continuous or uncontrolled cell proliferation. Some cancers are associated with suppression of normal apoptotic cell death. Strategies for treatment may involve either reestablishing control over cell cycle progression, or selectively stimulating apoptosis in cancerous cells (Nigg, E. A. (1995) BioEssays 17:471-480). Immunological defenses against cancer include induction of apoptosis in mutant cells by tumor suppressors, and the recognition of tumor antigens by T lymphocytes. Response to mitogenic stresses is frequently controlled at the level of transcription and is coordinated by various transcription factors. For example, the Rel/NF-kappa B family of vertebrate transcription factors plays a pivotal role in inflammatory and immune responses to radiation. The NF-kappa B family includes p50, p52, RelA, RelB, cRel, and other DNA-binding proteins. The p52 protein induces apoptosis, upregulates the transcription factor c-Jun, and activates c-Jun N-terminal kinase 1 (JNK1) (Sun, L. et al. (1998) Gene 208:157-166). Most NF-kappa B proteins form DNA-binding homodimers or heterodimers. Dimerization of many transcription factors is mediated by a conserved sequence known as the bZIP domain, characterized by a basic region followed by a leucine zipper.

The Fas/Apo-1 receptor (FAS) is a member of the tumor necrosis factor (TNF) receptor family. Upon binding its ligand (Fas ligand), the membrane-spanning FAS induces apoptosis by recruiting several cytoplasmic proteins that transmit the death signal. One such protein, termed FAS-associated protein factor 1 (FAF1), was isolated from mice, and it was demonstrated that expression of FAF1 in L cells potentiated FAS-induced apoptosis (Chu, K. et al. (1995) Proc. Natl. Acad. Sci. USA 92:11894-11898). Subsequently, FAS-associated factors have been isolated from numerous other species, including fruit fly and quail (Frohlich, T. et al. (1998) J. Cell Sci. 111:2353-2363). Another cytoplasmic protein that functions in the transmittal of the death signal from Fas is the Fas-associated death domain protein, also known as FADD. FADD transmits the death signal in both FAS-mediated and TNF receptor-mediated apoptotic pathways by activating caspase-8 (Bang, S. et al. (2000) J. Biol. Chem. 275:36217-36222).

Fragmentation of chromosomal DNA is one of the hallmarks of apoptosis. DNA fragmentation factor (DFF) is a protein composed of two subunits, a 40-kDa caspase-activated nuclease termed DFF40/CAD, and its 45-kDa inhibitor DFF45/ICAD. Two mouse homologs of DFF45/ICAD, termed CIDE-A and CIDE-B, have recently been described (Inohara, N. et al. (1998) EMBO J. 17:2526-2533). CIDE-A and CIDE-B expression in mammalian cells activated apoptosis, while expression of CIDE-A alone induced DNA fragmentation. In addition, FAS-mediated apoptosis was enhanced by CIDE-A and CIDE-B, further implicating these proteins as effectors that mediate apoptosis.

Transcription factors play an important role in the onset of apoptosis. A number of downstream effector molecules, particularly proteases such as the cysteine proteases called caspases, are involved in the initiation and execution phases of apoptosis. The activation of the caspases results from the competitive action of the pro-survival and pro-apoptosis Bcl-2-related proteins (Print, C. G. et al. (1998) Proc. Natl. Acad. Sci. USA 95:12424-12431). A pro-apoptotic signal can activate initiator caspases that trigger a proteolytic caspase cascade, leading to the hydrolysis of target proteins and the classic apoptotic death of the cell. Two active site residues, a cysteine and a histidine, have been implicated in the catalytic mechanism. Caspases are among the most specific endopeptidases, cleaving after aspartate residues.

Caspases are synthesized as inactive zymogens consisting of one large (p20) and one small (p10) subunit separated by a small spacer region, and a variable N-terminal prodomain. This prodomain interacts with cofactors that can positively or negatively affect apoptosis. An activating signal causes autoproteolytic cleavage of a specific aspartate residue (D297 in the caspase-1 numbering convention) and removal of the spacer and prodomain, leaving a p10/p20 heterodimer. Two of these heterodimers interact via their small subunits to form the catalytically active tetramer. The long prodomains of some caspase family members have been shown to promote dimerization and auto-processing of procaspases. Some caspases contain a “death effector domain” in their prodomain by which they can be recruited into self-activating complexes with other caspases and FADD protein-associated death receptors or the TNF receptor complex. In addition, two dimers from different caspase family members can associate, changing the substrate specificity of the resultant tetramer.

Tumor necrosis factor (TNF) and related cytokines induce apoptosis in lymphoid cells. (Reviewed in Nagata, S. (1997) Cell 88:355-365.) Binding of TNF to its receptor triggers a signal transduction pathway that results in the activation of a proteolytic caspase cascade. One such caspase, ICE (Interleukin-1β converting enzyme), is a cysteine protease comprised of two large and two small subunits generated by ICE auto-cleavage (Dinarello, C. A. (1994) FASEB J. 8:1314-1325). ICE is expressed primarily in monocytes. ICE processes the cytokine precursor, interleukin-1β, into its active form, which plays a central role in acute and chronic inflammation, bone resorption, myelogenous leukemia, and other pathological processes. ICE and related caspases cause apoptosis when overexpressed in transfected cell lines.

A caspase recruitment domain (CARD) is found within the prodomain of several apical caspases and is conserved in several apoptosis regulatory molecules such as Apaf-2, RAIDD, and cellular inhibitors of apoptosis proteins (IAPs) (Hofmann, K. et al. (1997) Trends Biochem. Sci. 22:155-157). The regulatory role of CARD in apoptosis may be to allow proteins such as Apaf-1 to associate with caspase-9 (Li, P. et al. (1997) Cell 91:479-489). A human cDNA encoding an apoptosis repressor with a CARD (ARC) which is expressed in both skeletal and cardiac muscle has been identified and characterized. ARC functions as an inhibitor of apoptosis and interacts selectively with caspases (Koseki, T. et al. (1998) Proc. Natl. Acad. Sci. USA 95:5156-5160). All of these interactions have clear effects on the control of apoptosis (reviewed in Chan S. L. and M. P. Mattson (1999) J. Neurosci. Res. 58:167-190; Salveson, G. S. and V. M. Dixit (1999) Proc. Natl. Acad. Sci. USA 96:10964-10967).

ES18 was identified as a potential regulator of apoptosis in mouse T-cells (Park, E. J. et al. (1999) Nuc. Acid. Res. 27:1524-1530). ES18 is 428 amino acids in length, contains an N-terminal proline-rich region, an acidic glutamic acid-rich domain, and a putative LXXLL nuclear receptor binding motif. The protein is preferentially expressed in lymph nodes and thymus. The level of ES18 expression increases in T-cell thymoma S49.1 in response to treatment with dexamethasone, staurosporine, or C2-ceramide, which induce apoptosis. ES18 may play a role in stimulating apoptotic cell death in T-cells.

The rat ventral prostate (RVP) is a model system for the study of hormone-regulated apoptosis. RVP epithelial cells undergo apoptosis in response to androgen deprivation. Messenger RNA (mRNA) transcripts that are up-regulated in the apoptotic RVP have been identified (Briehl, M. M. and R. L. Miesfeld (1991) Mol. Endocrinol. 5:1381-1388). One such transcript encodes RVP.1, the precise role of which in apoptosis has not been determined. The human homolog of RVP.1, hRVP1, is 89% identical to the rat protein (Katahira, J. et al. (1997) J. Biol. Chem. 272:26652-26658). hRVP1 is 220 amino acids in length and contains four transmembrane domains. hRVP1 is highly expressed in the lung, intestine, and liver. Interestingly, hRVP1 functions as a low affinity receptor for the Clostridium perfringens enterotoxin, a causative agent of diarrhea in humans and other animals.

Cytokine-mediated apoptosis plays an important role in hematopoiesis and the immune response. Myeloid cells, which are the stem cell progenitors of macrophages, neutrophils, erythrocytes, and other blood cells, proliferate in response to specific cytokines such as granulocyte/macrophage-colony stimulating factor (GM-CSF) and interleukin-3 (IL-3). When deprived of GM-CSF or IL-3, myeloid cells undergo apoptosis. The murine requiem (req) gene encodes a putative transcription factor required for this apoptotic response in the myeloid cell line FDCP-1 (Gabig, T. G. et al. (1994) J. Biol. Chem. 269:29515-29519). The Req protein is 371 amino acids in length and contains a nuclear localization signal, a single Kruppel-type zinc finger, an acidic domain, and a cluster of four unique zinc-finger motifs enriched in cysteine and histidine residues involved in metal binding. Expression of req is not myeloid- or apoptosis-specific, suggesting that additional factors regulate Req activity in myeloid cell apoptosis.

Dysregulation of apoptosis has recently been recognized as a significant factor in the pathogenesis of many human diseases. For example, excessive cell survival caused by decreased apoptosis can contribute to disorders related to cell proliferation and the immune response. Such disorders include cancer, autoimmune diseases, viral infections, and inflammation. In contrast, excessive cell death caused by increased apoptosis can lead to degenerative and immunodeficiency disorders such as AIDS, neurodegenerative diseases, and myelodysplastic syndromes. (Thompson, C. B. (1995) Science 267:1456-1462.)

Impaired regulation of apoptosis is also associated with loss of neurons in Alzheimer's disease. Alzheimer's disease is a progressive neurodegenerative disorder that is characterized by the formation of senile plaques and neurofibrillary tangles containing amyloid beta peptide. These plaques are found in limbic and association cortices of the brain, including hippocampus, temporal cortices, cingulate cortex, amygdala, nucleus basalis and locus caeruleus. B-amyloid peptide participates in signaling pathways that induce apoptosis and lead to the death of neurons (Kajkowski, C. et al. (2001) J. Biol. Chem. 276:18748-18756). Early in Alzheimer's pathology, physiological changes are visible in the cingulate cortex (Minoshima, S. et al. (1997) Annals of Neurology 42:85-94). In subjects with advanced Alzheimer's disease, accumulating plaques damage the neuronal architecture in limbic areas and eventually cripple the memory process.

Signal transduction is the general process by which cells respond to extracellular signals. Signal transduction across the plasma membrane begins with the binding of a signal molecule, e.g., a hormone, neurotransmitter, or growth factor, to a cell membrane receptor. The receptor, thus activated, triggers an intracellular biochemical cascade that ends with the activation of an intracellular target molecule, such as a transcription factor. This process of signal transduction regulates all types of cell functions including cell proliferation, differentiation, and gene transcription.

Cancer

Cancers are characterized by continuous or uncontrolled cell proliferation. Some cancers are associated with suppression of normal apoptotic cell death. Understanding of the neoplastic process can be aided by the identification of molecular markers of prognostic and diagnostic importance. Cancers are associated with oncoproteins which are capable of transforming normal cells into malignant cells. Some oncoproteins are mutant isoforms of the normal protein while others are abnormally expressed with respect to location or level of expression. Normal cell proliferation begins with binding of a growth factor to its receptor on the cell membrane, resulting in activation of a signal system that induces and activates nuclear regulatory factors to initiate DNA transcription, subsequently leading to cell division. Classes of oncoproteins known to affect the cell cycle controls include growth factors, growth factor receptors, intracellular signal transducers, nuclear transcription factors, and cell-cycle control proteins. Several types of cancer-specific genetic markers, such as tumor antigens and tumor suppressors, have also been identified.

Oncogenes

Oncoproteins are encoded by genes, called oncogenes, that are derived from genes that normally control cell growth and development. Many oncogenes have been identified and characterized. These include growth factors such as sis, receptors such as erbA, erbB, neu, and ros, intracellular receptors such as src, yes, fps, abl, and met, protein-serine/threonine kinases such as mos and raf nuclear transcription factors such as jun, fos, myc, N-myc, myb, ski, and rel, cell cycle control proteins such as RB and p53, mutated tumor-suppressor genes such as mdm2, Cip1, p16, and cyclin D, ras, set, can, sec, and gag R10.

Viral oncogenes are integrated into the human genome after infection of human cells by certain viruses. Examples of viral oncogenes include v-src, v-abl, and v-fps. Transformation of normal genes to oncogenes may also occur by chromosomal translocation. The Philadelphia chromosome, characteristic of chronic myeloid leukemia and a subset of acute lymphoblastic leukemias, results from a reciprocal translocation between chromosomes 9 and 22 that moves a truncated portion of the proto-oncogene c-abl to the breakpoint cluster region (bcr) on chromosome 22. The hybrid c-abl-bcr gene encodes a chimeric protein that has tyrosine kinase activity. In chronic myeloid leukemia, the chimeric protein has a molecular weight of 210 kd, whereas in acute leukemias a more active 180 kd tyrosine kinase is formed (Robbins, S. L. et al. (1994) Pathologic Basis of Disease, W.B. Saunders Co., Philadelphia Pa.).

The Ras superfamily of small GTPases is involved in the regulation of a wide range of cellular signaling pathways. Ras family proteins are membrane-associated proteins acting as molecular switches that bind GTP and GDP, hydrolyzing GTP to GDP. In the active GTP-bound state Ras family proteins interact with a variety of cellular targets to activate downstream signaling pathways. For example, members of the Ras subfamily are essential in transducing signals from receptor tyrosine kinases (RTKs) to a series of serine/threonine kinases which control cell growth and differentiation. Activated Ras genes were initially found in human cancers and subsequent studies confirmed that Ras function is critical in the determination of whether cells continue to grow or become terminally differentiated (Barbacid, M. (1987) Annu. Rev. Biochem. 56:779-827; Treisman, R. (1994) Curr. Opin. Genet. Dev. 4:96-98). Mutant Ras proteins, which bind but can not hydrolyze GTP, are permanently activated, and cause continuous cell proliferation or cancer.

Activation of Ras family proteins is catalyzed by guanine nucleotide exchange factors (GEFs) which catalyze the dissociation of bound GDP and subsequent binding of GTP. A recently discovered RalGEF-like protein, RGL3, interacts with both Ras and the related protein Rit. Constitutively active Rit, like Ras, can induce oncogenic transformation, although since Rit fails to interact with most known Ras effector proteins, novel cellular targets may be involved in Rit transforming activity. RGL3 interacts with both Ras and Rit, and thus may act as a downstream effector for these proteins (Shao, H. and D. A. Andres (2000) J. Biol. Chem. 275:26914-26924).

Tumor Antigens

Tumor antigens are cell surface molecules that are differentially expressed in tumor cells relative to non-tumor tissues. Tumor antigens make tumor cells immunologically distinct from normal cells and are potential diagnostics for human cancers. Several monoclonal antibodies have been identified which react specifically with cancerous cells such as T-cell acute lymphoblastic leukemia and neuroblastoma (Minegishi, M. et al. (1989) Leukemia Res. 13:43-51; Takagi, S. et al. (1995) Int. J. Cancer 61:706-715). In addition, the discovery of high level expression of the HER2 gene in breast tumors has led to the development of therapeutic treatments (Liu, E. et al. (1992) Oncogene 7: 1027-1032; Kern, J. A. (1993) Am. J. Respir. Cell Mol. Biol. 9:448-454). Tumor antigens are found on the cell surface and have been characterized either as membrane proteins or glycoproteins. For example, MAGE genes encode a family of tumor antigens recognized on melanoma cell surfaces by autologous cytolytic T lymphocytes. Among the 12 human MAGE genes isolated, half are differentially expressed in tumors of various histological types (De Plaen, E. et al. (1994) Immunogenetics 40:360-369). None of the 12 MAGE genes, however, is expressed in healthy tissues except testis and placenta.

Tumor Suppressors

Tumor suppressor genes are generally defined as genetic elements whose loss or inactivation contributes to the deregulation of cell proliferation and the pathogenesis and progression of cancer. Tumor suppressor genes normally function to control or inhibit cell growth in response to stress and to limit the proliferative life span of the cell. Several tumor suppressor genes have been identified including the genes encoding the retinoblastoma (Rb) protein, p53, and the breast cancer 1 and 2 proteins (BRCA1 and BRCA2). Mutations in these genes are associated with acquired and inherited genetic predisposition to the development of certain cancers.

The role of p53 in the pathogenesis of cancer has been extensively studied. (Reviewed in Aggarwal, M. L. et al. (1998) J. Biol. Chem 273:1-4; Levine, A. (1997) Cell 88:323-331.) About 50% of all human cancers contain mutations in the p53 gene. These mutations result in either the absence of functional p53 or, more commonly, a defective form of p53 which is overexpressed. p53 is a transcription factor that contains a central core domain required for DNA binding. Most cancer-associated mutations in p53 localize to this domain. In normal proliferating cells, p53 is expressed at low levels and is rapidly degraded. p53 expression and activity is induced in response to DNA damage, abortive mitosis, and other stressful stimuli. In these instances, p53 induces apoptosis or arrests cell growth until the stress is removed. Downstream effectors of p53 activity include apoptosis-specific proteins and cell cycle regulatory proteins, including Rb, oncogene products, cyclins, and cell cycle-dependent kinases.

The metastasis-suppressor gene KAI1 (CD82) has been reported to be related to the tumor suppressor gene p53. KAI1 is involved in the progression of human prostatic cancer and possibly lung and breast cancers when expression is decreased. KAI1 encodes a member of a structurally distinct family of leukocyte surface glycoproteins. The family is known as either the tetraspan transmembrane protein family or transmembrane 4 superfamily (TM4SF) as the members of this family span the plasma membrane four times. The family is composed of integral membrane proteins having a N-terminal membrane-anchoring domain which functions as both a membrane anchor and a translocation signal during protein biosynthesis. The N-terminal membrane-anchoring domain is not cleaved during biosynthesis. TM4SF proteins have three additional transmembrane regions, seven or more conserved cysteine residues, are similar in size (218 to 284 residues), and all have a large extracellular hydrophilic domain with three potential N-glycosylation sites. The promoter region contains many putative binding motifs for various transcription factors, including five AP2 sites and nine SpI sites. Gene structure comparisons of KAI1 and seven other members of the TM4SF indicate that the splicing sites relative to the different structural domains of the predicted proteins are conserved. This suggests that these genes are related evolutionarily and arose through gene duplication and divergent evolution (Levy, S. et al. (1991) J. Biol. Chem. 266:14597-14602; Dong, J. T. et al. (1995) Science 268:884-886; Dong, J. T. et al., (1997) Genomics 41:25-32).

The Leucine-rich gene-Glioma Inactivated (LGI1) protein shares homology with a number of transmembrane and extracellular proteins which function as receptors and adhesion proteins. LGI1 is encoded by an LLR (leucine-rich, repeat-containing) gene and maps to 10q24. LGI1 has four LLRs which are flanked by cysteine-rich regions and one transmembrane domain (Somerville, R. P. et al. (2000) Mamm. Genome 11:622-627). LGI1 expression is seen predominantly in neural tissues, especially brain. The loss of tumor suppressor activity is seen in the inactivation of the LGI1 protein which occurs during the transition from low to high-grade tumors in malignant gliomas. The reduction of LGI1 expression in low grade brain tumors and its significant reduction or absence of expression in malignant gliomas suggests that it could be used for diagnosis of glial tumor progression (Chemova, O. B. et al. (1998) Oncogene 17:2873-2881).

The ST13 tumor suppressor was identified in a screen for factors related to colorectal carcinomas by subtractive hybridization between cDNA of normal mucosal tissues and mRNA of colorectal carcinoma tissues (Cao, J. et al. (1997) J. Cancer Res. Clin. Oncol. 123:447-451). ST13 is down-regulated in human colorectal carcinomas.

Mutations in the von Hippel-Lindau (VHL) tumor suppressor gene are associated with retinal and central nervous system hemangioblastomas, clear cell renal carcinomas, and pheochromocytomas (Hoffman, M. et al. (2001) Hum. Mol. Genet. 10:1019-1027; Kamada, M. (2001) Cancer Res. 61:4184-4189). Tumor progression is linked to defects or inactivation of the VHL gene. VHL regulates the expression of transforming growth factor-α, the GLUT-1 glucose transporter and vascular endothelial growth factor. The VHL protein associates with elongin B, elongin C, Cul2 and Rbx1 to form a complex that regulates the transcriptional activator hypoxia-inducible factor (HIF). HIF induces genes involved in angiogenesis such as vascular endothelial growth factor and platelet-derived growth factor B. Loss of control of HIF caused by defects in VHL results in the excessive production of angiogenic peptides. VHL may play roles in inhibition of angiogenesis, cell cycle control, fibronectin matrix assembly, cell adhesion, and proteolysis.

Mutations in tumor suppressor genes are a common feature of many cancers and often appear to affect a critical step in the pathogenesis and progression of tumors. Accordingly, Chang, F. et al. (1995; J. Clin. Oncol. 13:1009-1022) suggest that it may be possible to use either the gene or an antibody to the expressed protein 1) to screen patients at increased risk for cancer, 2) to aid in diagnosis made by traditional methods, and 3) to assess the prognosis of individual cancer patients. In addition, Hamada, K. et al. (1996; Cancer Res. 56:3047-3054) are investigating the introduction of p53 into cervical cancer cells via an adenoviral vector as an experimental therapy for cervical cancer.

The PR-domain genes were recently recognized as playing a role in human tumorigenesis. PR-domain genes normally produce two protein products: the PR-plus product, which contains the PR domain, and the PR-minus product which lacks this domain. In cancer cells, PR-plus is disrupted or overexpressed, while PR-minus is present or overexpressed. The imbalance in the amount of these two proteins appears to be an important cause of malignancy (Jiang, G. L. and S. Huang (2000) Histol. Histopathol. 15:109-117).

Many neoplastic disorders in humans can be attributed to inappropriate gene transcription. Malignant cell growth may result from either excessive expression of tumor promoting genes or insufficient expression of tumor suppressor genes (Cleary, M. L. (1992) Cancer Surv. 15:89-104). Chromosomal translocations may also produce chimeric loci which fuse the coding sequence of one gene with the regulatory regions of a second unrelated gene. An important class of transcriptional regulators are the zinc finger proteins. The zinc finger motif, which binds zinc ions, generally contains tandem repeats of about 30 amino acids consisting of periodically spaced cysteine and histidine residues. Examples of this sequence pattern include the C2H2-type, C4-type, and C3HC4-type zinc fingers, and the PHD domain (Lewin, B. (1990) Genes IV, Oxford University Press, New York, N.Y., and Cell Press, Cambridge, Mass., pp. 554-570; Aasland, R., et al. (1995) Trends Biochem. Sci. 20:56-59). One clinically relevant zinc-finger protein is WT1, a tumor-suppressor protein that is inactivated in children with Wilm's tumor. The oncogene bcl-6, which plays an important role in large-cell lymphoma, is also a zinc-finger protein (Papavassiliou, A. G. (1995) N. Engl. J. Med. 332:45-47).

Tumor Responsive Proteins

Cancers, also called neoplasias, are characterized by continuous and uncontrolled cell proliferation. They can be divided into three categories: carcinomas, sarcomas, and leukemias. Carcinomas are malignant growths of soft epithelial cells that may infiltrate surrounding tissues and give rise to metastatic tumors. Sarcomas may be of epithelial origin or arise from connective tissue. Leukemias are progressive malignancies of blood-forming tissue characterized by proliferation of leukocytes and their precursors, and may be classified as myelogenous (granulocyte- or monocyte-derived) or lymphocytic (lymphocyte-derived). Tumorigenesis refers to the progression of a tumor's growth from its inception. Malignant cells may be quite similar to normal cells within the tissue of origin or may be undifferentiated (anaplastic). Tumor cells may possess few nuclei or one large polymorphic nucleus. Anaplastic cells may grow in a disorganized mass that is poorly vascularized and as a result contains large areas of ischemic necrosis. Differentiated neoplastic cells may secrete the same proteins as the tissue of origin. Cancers grow, infiltrate, invade, and destroy the surrounding tissue through direct seeding of body cavities or surfaces, through lymphatic spread, or through hematogenous spread. Cancer remains a major public health concern and current preventative measures and treatments do not match the needs of most patients. Understanding of the neoplastic process of tumorigenesis can be aided by the identification of molecular markers of prognostic and diagnostic importance.

Current forms of cancer treatment include the use of immunosuppressive drugs (Morisaki, T. et al. (2000) Anticancer Res. 20:3363-3373; Geoerger, B. et al. (2001) Cancer Res. 61:1527-1532). The identification of proteins involved in cell signaling, and specifically proteins that act as receptors for immunosuppressant drugs, may facilitate the development of anti-tumor agents. For example, immunophilins are a family of conserved proteins found in both prokaryotes and eukaryotes that bind to immunosuppressive drugs with varying degrees of specificity. One such group of immunophilic proteins is the peptidyl-prolyl cis-trans isomerase (EC 5.2.1.8) family (PPlase, rotamase). These enzymes, first isolated from porcine kidney cortex, accelerate protein folding by catalyzing the cis-trans isomerization of proline imidic peptide bonds in oligopeptides (Fischer, G. and F. X. Schmid (1990) Biochemistry 29:2205-2212). Included within the immunophilin family are the cyclophilins (e.g., peptidyl-prolyl isomerase A or PPIA) and FK-binding protein (e.g., FKBP) subfamilies. Cyclophilins are multifunctional receptor proteins which participate in signal transduction activities, including those mediated by cyclosporin (or cyclosporine). The PPIase domain of each family is highly conserved between species. Although structurally distinct, these multifunctional receptor proteins are involved in numerous signal transduction pathways, and have been implicated in folding and trafficking events.

The immunophilin protein cyclophilin binds to the immunosuppressant drug cyclosporin A. FKBP, another immunophilin, binds to FK506 (or rapamycin). Rapamycin is an immunosuppressant agent that arrests cells in the G₁ phase of growth, inducing apoptosis. Like cyclophilin, this macrolide antibiotic (produced by Streptomyces tsukubaensis) acts by binding to ubiquitous, predominantly cytosolic immunophilin receptors. These immunophilin/immunosuppressant complexes (e.g., cyclophilin A/cyclosporin A (CypA/CsA) and FKBP12/FK506) achieve their therapeutic results through inhibition of the phosphatase calcineurin, a calcium/calmodulin-dependent protein kinase that participates in T-cell activation (Hamilton, G. S. and J. P. Steiner (1998) J. Med. Chem. 41: 5119-5143). The murine fkbp51 gene is abundantly expressed in immunological tissues, including the thymus and T lymphocytes (Baughman, G. et al. (1995) Molec. Cell. Biol. 15: 4395-4402). FKBP12/rapamycin-directed immunosuppression occurs through binding to TOR (yeast) or FRAP (FKBP12-rapamycin-associated protein, in mammalian cells), the kinase target of rapamycin essential for maintaining normal cellular growth patterns. Dysfunctional TOR signaling has been linked to various human disorders including cancer (Metcalfe, S. M. et al. (1997) Oncogene 15:1635-1642; Emami, S. et al. (2001) FASEB J. 15:351-361), and autoimmunity (Damoiseaux, J. G. et al. (1996) Transplantation 62:994-1001).

Several cyclophilin isozymes have been identified, including cyclophilin B, cyclophilin C, mitochondrial matrix cyclophilin, bacterial cytosolic and periplasmic PPIases, and natural-killer cell cyclophilin-related protein possessing a cyclophilin-type PPIase domain, a putative tumor-recognition complex involved in the function of natural killer (NK) cells. These cells participate in the innate cellular immune response by lysing virally-infected cells or transformed cells. NK cells specifically target cells that have lost their expression of major histocompatibility complex (MHC) class I genes (common during tumorigenesis), endowing them with the potential for attenuating tumor growth. A 150-kDa molecule has been identified on the surface of human NK cells that possesses a domain which is highly homologous to cyclophilin/peptidyl-prolyl cis-trans isomerase. This cyclophilin-type protein may be a component of a putative tumor-recognition complex, a NK tumor recognition sequence (NK-TR) (Anderson, S. K. et al. (1993) Proc. Natl. Acad. Sci. USA 90:542-546). The NKTR tumor recognition sequence mediates recognition between tumor cells and large granular lymphocytes (LGLs), a subpopulation of white blood cells (comprised of activated cytotoxic T cells and natural killer cells) capable of destroying tumor targets. The protein product of the NKTR gene presents on the surface of LGLs and facilitates binding to tumor targets. More recently, a mouse Nktr gene and promoter region have been located on chromosome 9. The gene encodes a NK-cell-specific 150-kDa protein (NK-TR) that is homologous to cyclophilin and other tumor-responsive proteins (Simons-Evelyn, M. et al. (1997) Genomics 40:94-100).

Other proteins that interact with tumorigenic tissue include cytokines such as tumor necrosis factor (TNF). The TNF family of cytokines are produced by lymphocytes and macrophages, and can cause the lysis of transformed (tumor) endothelial cells. Endothelial protein 1 (Edp1) has been identified as a human gene activated transcriptionally by TNF-alpha in endothelial cells, and a TNF-alpha inducible Edp1 gene has been identified in the mouse (Swift, S. et al. (1998) Biochim. Biophys. Acta 1442:394-398).

Expression profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Breast Cancer

There are more than 180,000 new cases of breast cancer diagnosed each year, and the mortality rate for breast cancer approaches 10% of all deaths in females between the ages of 45-54 (Gish, K. (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou, C. M. et al. (2000) Nature 406:747-752).

BT-20 is a breast carcinoma cell line derived in vitro from the cells emigrating out of thin slices of the tumor mass isolated from a 74-year-old female. BT-474 is a breast ductal carcinoma cell line that was isolated from a solid, invasive ductal carcinoma of the breast obtained from a 60-year-old woman. BT-474 displays typical epithelial cellular structures such as desmosomes, microvilli, gap junctions, and tight junctions. This cell line has also discernable microtubules, tonofibrils, lysosomes, and osmiophilic secretory granules. BT-483 is a breast ductal carcinoma cell line that was isolated from a papillary invasive ductal tumor obtained from a 23-year-old normal, menstruating, parous female with a family history of breast cancer. BT-483 displays characteristic epithelial cellular structures such as desmosomes, microvilli, tight junctions, and gap junctions. Hs 578T is a breast ductal carcinoma cell line that was isolated from a 74-year-old female with breast carcinoma. These cells do not express any detectable estrogen receptors and do not form colonies in semi-solid culture medium. MCF7 is a nonmalignant breast adenocarcinoma cell line isolated from the pleural effusion of a 69-year-old female. MCF7 has retained characteristics of the mammary epithelium such as the ability to process estradiol via cytoplasmic estrogen receptors and the capacity to form domes in culture.

HMEC is a primary breast epithelial cell line isolated from a normal donor. MCF-10A is a breast mammary gland (luminal ductal characteristics) cell line that was isolated. from a 36-year-old woman with fibrocystic breast disease. MCF-10A expresses cytoplasmic keratins, epithelial sialomucins, and milkfat globule antigens. This cell lines exhibits three-dimensional growth in collagen and forms domes in confluent culture. MDA-MB-468 is breast adenocarcinoma cell line isolated from the pleural effusion of a 51-year-old female with metastatic adenocarcinoma of the breast. MCF7 is a nonmalignant breast adenocarcinoma cell line isolated from the pleural effusion of a 69-year-old female. MCF7 has retained characteristics of the mammary epithelium such as the ability to process estradiol via cytoplasmic estrogen receptors and the capacity to form domes in culture. T-47D is a breast carcinoma cell line isolated from a pleural effusion obtained from a 54-year-old female with an infiltrating ductal carcinoma of the breast. Sk-BR-3 is a breast adenocarcinoma cell line isolated from a malignant pleural effusion of a 43-year-old female. It forms poorly differentiated adenocarcinoma when injected into nude mice. BT-20 is a breast carcinoma cell line derived in vitro from cells emigrating out of thin slices of the tumor mass isolated from a 74-year-old female. MDA-mb-231 is a breast tumor cell line isolated from the pleural effusion of a 51-year old female. It forms poorly differentiated adenocarcinoma in nude mice and ALS treated BALB/c mice. It also expresses the Wnt3 oncogene, EGF, and TGF-α. MDA-mb-435S is a spindle shaped strain that evolved from the parent line (435) as isolated in 1976 by R. Cailleau from the pleural effusion of a 31-year-old female with metastatic, ductal adenocarcinoma of the breast.

Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to non-inherited mutations that occur in breast epithelial cells.

The relationship between expression of epidermal growth factor (EGF) and its receptor, EGFR, to human mammary carcinoma has been particularly well studied. (See Khazaie, K. et al. (1993) Cancer and Metastasis Rev. 12:255-274, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix G1a protein which is overexpressed is human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down regulated in mammary carcinoma cells relative to normal mammary epithelial cells (Zhou, Z. et al. (1998) Int. J. Cancer 78:95-99; Chen, L. et al. (1990) Oncogene 5:1391-1395; Ulrix, W. et al (1999) FEBS Lett 455:23-26; Sager, R. et al. (1996) Curr. Top. Microbiol. Immunol. 213:51-64; and Lee, S. W. et al. (1992) Proc. Natl. Acad. Sci. USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba, I. I. et al. (1998) Clin. Cancer Res. 4:2931-2938). Such a model is particularly useful for comparing phenotypic and molecular characteristics of human mammary epithelial cells at various stages of malignant transformation.

Colon Cancer

Colorectal cancer is the second leading cause of cancer deaths in the United States. Colon cancer is associated with aging, since 90% of the total cases occur in individuals over the age of 55. A widely accepted hypothesis is that several contributing genetic mutations must accumulate over time in an individual who develops the disease. To understand the nature of genetic alterations in colorectal cancer, a number of studies have focused on the inherited syndromes. The first known inherited syndrome, Familial Adenomatous Polyposis (FAP), is caused by mutations in the Adenomatous Polyposis Coli gene (APC), resulting in truncated or inactive forms of the protein. This tumor suppressor gene has been mapped to chromosome 5q. The second known inherited syndrome is hereditary nonpolyposis colorectal cancer (HNPCC), which is caused by mutations in mismatch repair genes.

Although hereditary colon cancer syndromes occur in a small percentage of the population and most colorectal cancers are considered sporadic, knowledge from studies of the hereditary syndromes can be generally applied. For instance, somatic mutations in APC occur in at least 80% of indiscriminate colon tumors. APC mutations are thought to be the initiating event in the disease. Other mutations occur subsequently. Approximately 50% of colorectal cancers contain activating mutations in ras, while 85% contain inactivating mutations in p53. Changes in these genes lead to gene expression changes in colon cancer. Less is understood about downstream targets of these mutations and the role they may play in cancer development and progression.

Lung Cancer

Lung cancer is the leading cause of cancer death for men and the second leading cause of cancer death for women in the U.S. The vast majority of lung cancer cases are attributed to smoking tobacco, and increased use of tobacco products in third world countries is projected to lead to an epidemic of lung cancer in these countries. Exposure of the bronchial epithelium to tobacco smoke appears to result in changes in tissue morphology, which are thought to be precursors of cancer. Lung cancers are divided into four histopathologically distinct groups. Three groups (squamous cell carcinoma, adenocarcinoma, and large cell carcinoma) are classified as non-small cell lung cancers (NSCLCs). The fourth group of cancers is referred to as small cell lung cancer (SCLC). Collectively, NSCLCs account for ˜70% of cases while SCLCs account for ˜18% of cases. The molecular and cellular biology underlying the development and progression of lung cancer are incompletely understood.

Deletions on chromosome 3 are common in this disease and are thought to indicate the presence of a tumor suppressor gene in this region. Activating mutations in K-ras are commonly found in lung cancer and are the basis of one of the mouse models for the disease.

Ovarian Cancer

Ovarian cancer is the leading cause of death from a gynecologic cancer. The majority of ovarian cancers are derived from epithelial cells, and 70% of patients with epithelial ovarian cancers present with late-stage disease. As a result, the long-term survival rates for this disease is very low. Identification of early-stage markers for ovarian cancer would significantly increase the survival rate. The molecular events that lead to ovarian cancer are poorly understood. Some of the known aberrations include mutation of p53 and microsatellite instability. Since gene expression patterns are likely to vary when normal ovary is compared to ovarian tumors, examination of gene expression in these tissues to identify possible markers for ovarian cancer is particularly relevant to improving diagnosis, prognosis, and treatment of this disease.

Sarcoma

The most common type of bone cancer is osteosarcoma, which develops in new tissue in growing bones. Chondrosarcoma, arises in cartilage. Ewing's sarcoma, another form of bone cancer, begins in immature nerve tissue in bone marrow. Osteosarcoma and Ewing's sarcoma tend to occur more frequently in children and adolescents, while chondrosarcoma occurs more often in adults. Currently, more than 50% of new patients diagnosed with a soft tissue sarcoma will die from it. Surgery is often the primary treatment (CancerNet http://imsdd.meb.uni-bonn.de/cancernet/600626.html). Osteosarcoma is the most common malignant bone tumor in children. With currently available treatment regimens, approximately 30-40% of patients with non-metastatic disease relapse after therapy. Currently, there is no prognostic factor that can be used at the time of initial diagnosis to predict which patients will have a high risk of relapse. The only significant prognostic factor predicting the outcome in a patient with non-metastatic osteosarcoma is the histopathologic response of the primary tumor resected at the time of definitive surgery.

C3A Cells

The C3A cell line is a clonal derivative of the Hep G2 hepatoma cell line isolated from a 15-year-old male with liver tumor, which was selected for its strong contact inhibition of growth. The use of a clonal population enhances the reproducibility of the cells. The C3A cells express insulin receptor and insulin-like growth factor II receptor.

Tangier Disease

Tangier disease (TD) is a genetic disorder characterized by near absence of circulating HDL and the accumulation of cholesterol esters in many tissues, including tonsils, lymph nodes, liver, spleen, thymus, and intestine. Low levels of HDL represent a clear predictor of premature coronary artery disease and homozygous TD correlates with a four- to six-fold increase in cardiovascular disease compared to controls. The major cardio-protective activity of HDL is ascribed to its role in reverse cholesterol transport, the flux of cholesterol from peripheral cells such as tissue macrophages, through plasma lipoproteins to the liver. The HDL protein, apolipoprotein A-I, plays a major role in this process, interacting with the cell surface to remove excess cholesterol and phospholipids. This pathway is severely impaired in TD; the defect lies in the ABC1 transporter, a member of the family of ATP-binding cassette transporters, which utilize ATP hydrolysis to transport a variety of substrates across membranes.

Jurkat Cells

Jurkat is an acute T cell leukemia cell line that grows actively in the absence of external stimuli. Jurkat has been extensively used to study signaling in human T cells. Phorbol myristate acetate (PMA) is a broad activator of the protein kinase C-dependent pathways. Ionomycin is a calcium ionophore that permits the entry of calcium in the cell, hence increasing the cytosolic calcium concentration. The combination of PMA and ionomycin activates two of the major signaling pathways used by mammalian cells to interact with their environment. In T cells, the combination of PMA and ionomycin mimics the type of secondary signaling events elicited during optimal B cell activation.

Steroids

Steroids are a class of lipid-soluble molecules, including cholesterol, bile acids, vitamin D, and hormones, that share a common four-ring structure based on cyclopentanoperhydrophenanthrene and that carrry out a wide variety of functions. Cholesterol, for example, is a component of cell membranes that controls membrane fluidity. It is also a precursor for bile acids which solubilize lipids and facilitate absorption in the small intestine during digestion. Vitamin D regulates the absorption of calcium in the small intestine and controls the concentration of calcium in plasma. Steroid hormones, produced by the adrenal cortex, ovaries, and testes, include glucocorticoids, mineralocorticoids, androgens, and estrogens. They control various biological processes by binding to intracellular receptors that regulate transcription of specific genes in the nucleus. Glucocorticoids, for example, increase blood glucose concentrations by regulation of gluconeogenesis in the liver, increase blood concentrations of fatty acids by promoting lipolysis in adipose tissues, modulate sensitivity to catcholamines in the central nervous system, and reduce inflammation. Glucocorticoids prevent or suppress inflammation and immune responses when administered at pharmacological doses. The anti-inflammatory actions of corticos-teroids are thought to involve phospholipase A 2 inhibitory proteins, collectively called lipocortins. Lipocortins, in turn, control the biosynthesis of potent mediators of inflammation such as prostaglan-dins and leukotrienes by inhibiting the release of the precursor molecule arachidonic acid. Steroid hormones are widely used for fertility control and in anti-inflammatory treatments for physical injuries and diseases such as arthritis, asthma, and auto-immune disorders.

Dexamethasone (DEX) is a synthetic glucocorticoid used as anti-inflammatory or immuno-suppressive agents. Due to its greater ability to reach the central nervous system, DEX is usually the treatment of choice to control cerebral edema. Medroxyprogesterone (MAH), also known as 6-methyl-17-hydroxyprogesterone, is a synthetic progestin with a pharmacological activity about 15 times greater than progesterone. MAH is usually used for the treatment of renal and endometrial carcinomas, amenorrhea, abnormal uterine bleeding, and endometriosis associated with hormonal imbalance. Beclomethasone is a synthetic glucocorticoid that is used for treating steroid-dependent asthma, relieving symptoms associated with allergic or nonallergic (vasomotor) rhinitis, or for pre-venting recurrent nasal polyps following surgical removal.

Adipocyte Maturation

The primary function of adipose tissue is the ability to store and release fat during periods of feeding and fasting. White adipose tissue is the major energy reserve in periods of fasting, and its reserve is mobilized during energy deprivation. Adipose tissue is one of the primary target tissues for insulin, and adipogenesis and insulin resistance are linked in type II diabetes, non-insulin dependent diabetes mellitus (NIDDM). Cytologically the conversion of a preadipocytes into mature adipocytes is characterized by deposition of fat droplets around the nuclei. The conversion process in vivo can be induced by thiazolidinediones and other PPARγ agonists (Adams et al. (1997) J Clin Invest 100:3149-3153) which also lead to increased sensitivity to insulin and reduced plasma glucose and blood pressure.

Pickup and Crook (1998; Diabetologia 41:1241-8) have suggested that NIDDM may result from the inability of an individual with hypersensitive acute-phase immune response to carry out normal cell signaling and repair. Steps in this process are highly correlated with long-term lifestyle and environment and include: 1) high glucose stimulation of insulin and cytokine production, 2) influence of various cytokines on tissue remodeling during adipocyte differentiation and their affect on signaling pathways, and 3) occurrence of tissue damage when cytokines continue to be produced, extracellular matrix components (ECM) are not recycled, and homeostasis is not timely restored. Many cytokines and the receptors with which they interact are implicated in this process. These cytokines include tumor necrosis factor, connective tissue growth factor, transforming growth factor-beta, interleukin (IL)-13 and their receptors. Tumor necrosis factor contributes to insulin resistance by inhibiting insulin-stimulated tyrosine phosphorylation of the insulin receptor. This, in turn, prevents the insulin receptor from participating in normal signaling processes (Skolnik and Marcusohn (1996) Cytokine Growth Factor Rev 7:161-173; Hotamisligil (1999) J Intern med 245:621-625). Connective tissue growth factor mediates the buildup of mesenglial matrix (Murphy et al. (2000) J Biol Chem 274:5830-5834). Transforming growth factor-beta mediates the buildup of mesenglial matrix of the kidney and affects vascular function through its interaction with the inositol trisphosphate receptor, a key intracellular calcium channel (Sharma and McGowan (2000) Cytokine Growth Factor Rev 11:115-123).

IL-13 and IL-4 are immuno-regulatory cytokines which share many overlapping biological properties. They both promote growth of B-cells (McKenzie et al. (1993) Proc Natl Acad Sci 90:3735-3739), induce expression of germ line Cε transcripts, and direct naive B cells to switch to the synthesis of IgE and IgG4 (Punnomen et al. (1993) Proc Natl Acad Sci 90:3730-3734). Similarly, different isoforms of the IL-13 and IL-4 receptors interact to form four types of IL-13 receptor complexes. In some instances, IL-13 utilizes a receptor complex composed of the IL-4 receptor-α chain (Rα) and the IL-13Rα. Although the specific role of each chain in IL-13 signaling is unclear, Ba/F3 cells transfected with IL-13Rα1 display a mitogenic response to IL-13, but cells transfected with mouse IL-13Rα2 do not. In addition, a soluble IL-13Rα2/Fc fusion protein blocks the mitogenic response to IL-13 (Donaldson et al. (1998) J Immunol 161:2317-2324). This suggests that IL-13Rα2 could serve as a dominant negative inhibitor or decoy receptor for IL-13. However, in colonic carcinoma cell lines, the receptor complex displayed growth inhibition which was associated with tyrosine phosphorylation of insulin receptor substrate-1. It is evident that more research is needed to establish 1) which isoforms of the receptor complex promote cell growth and which inhibit cell growth and 2) whether this varies by cell or tissue type.

Most adipocyte research has been carried out using mouse cell lines. Recent evidence, however, indicates that culture conditions which stimulate mouse preadipocyte differentiation are different from those which induce human preadipocytes. In addition to the known genetic differences between these species, diploid human primary cells respond differently than aneuploid mouse cells.

Prostate Cancer

Prostate cancer develops through a multistage progression ultimately resulting in an aggressive tumor phenotype. The initial step in tumor progression involves the hyper-proliferation of normal luminal and/or basal epithelial cells. Androgen responsive cells become hyperplastic and evolve into early-stage tumors. Although early-stage tumors are often androgen sensitive and respond to androgen ablation, a population of androgen independent cells evolve from the hyperplastic population. These cells represent a more advanced form of prostate tumor that may become invasive and potentially become metastatic to the bone, brain, or lung.

Endometrial Cancer

Endometrial cancer is the most common gynecologic cancer. Approximately 90% of endometrial cancers are epithelial in origin, and 90% of these cancers are classified as endometrial adenocarcinomas. Estrogen appears to act as a tumor promoter in endometrial tissue. Evidence indicates that p53 and Ki-ras are mutated in endometrial cancer. However, these mutations occur in a small percentage of cases and do not appear to be the initiating events in the disease. In addition, most chromosomes contain regions of allelic loss in endometrial cancer, indicating that many genes may be affected in this disease.

Biopharmacological Tools for Microarray Analysis

Human umbilical vein endothelial cells (HUVECs) are a primary cell line derived from the endothelium of the human umbilical vein. HUVECs are used to study the functional biology of human endothelial cells in vitro. Activation of vascular endothelium is observed in physiological and pathophysiological processes including vascular tone regulation, coagulation and thrombosis, atherosclerosis, and inflammation.

Tumor necrosis factor-alpha (TNF-α) is a pleiotropic cytokine that plays a central role in mediation of the inflammatory response through activation of multiple signal transduction pathways. TNF-α is produced by activated lymphocytes, macrophages, and other white blood cells, and activates endothelial cells.

PMA is an agonist of protein kinase C (PKC). PKC is a calcium-activated, phospholipid-dependent serine- and threonine-specific kinase that, upon activation, phosphorylates a broad range of secondary targets. TNF-α causes translocation of PKC from the cytosol to the membrane where it phosphorylates a variety of targets.

Interleukin 1 beta (IL-1β)is a cytokine associated with acute inflammatory responses and is involved in processes such as fever induction, metabolic regulation, and bone remodeling. Both cells of the immune system (monocytes, dendritic cells, NK cells, platelets, and neutrophils) and somatic cells (osteoblasts, neurons, Schwann cells, oligodendrocytes, and adrenal cortical cells) can produce IL-1β. IL-1β can induce its own production in monocytes, the production of adhesion molecules and chemokines in endothelial cells, and interferon gamma (IFN-γ) production by NK cells in conjunction with IL-12. IL-1 is produced as a single chain pro-molecule that must be cleaved by a specialized protease—IL-1 Converting Enzyme (ICE)—to acquire its function. Interleukin 10 (IL-10) is produced by CD4+ T cell clones and some CD8+ T cell clones. Human B cells, EBV-transformed lymphoblastoid cell lines, and monocytes can also produce IL-10 upon activation. IL-10 is a pleiotrophic cytokine that can exert either imnunostimulatory or immunosupressive effects on a variety of cell types. It is a potent immunosuppressant of macrophage functions. In vitro, IL-10 can inhibit the accessory function and antigen-presenting capacity of monocytes by, among other effects, downregulating class II MHC expression. Thus, IL-10 can inhibit monocyte/macrophage-dependent, antigen specific proliferation of mouse Th1 clones as well as human Th0-, Th1-, and Th2- like T cells. IL-10 can also inhibit the monocyte/macrophage-dependent, antigen stimulated cytokine synthesis (especially IFN-g) by human PBMNC and NK. Additionally, IL-10 is a potent inhibitor of monocyte/macrophage activation and its resultant cytotoxic effects. It can suppress the production of numerous cytokines including TNF-a, IL-1, IL-6, and IL-10, as well as the synthesis of superoxide anion, reactive oxygen intermediates, and reactive nitrogen intermediates by activated monocytes/macrophages. As an immunostimulatory cytokine, IL-10 can act on B cells to enhance their viability, cell proliferation, Ig secretion, and class II MHC expression. Aside from B-lymphocytes, IL-10 is also a growth co-stimulator for thymocytes and mast cells, as well as an enhancer of cytotoxic T cell development.

Thiazolidinediones or peroxisome proliferator-activated receptor γ (PPAR-γ) agonists are a new class of antidiabetic agents that improve insulin sensitivity and reduce plasma glucose and blood pressure in subjects with type II diabetes. These agents can bind and activate an orphan nuclear receptor and some of them induce human adipocyte differentiation.

There is a need in the art for new compositions, including nucleic acids and proteins, for the diagnosis, prevention, and treatment of cell proliferative disorders including cancer, developmental disorders, neurological disorders, autoimmune/inflammatory disorders, reproductive disorders, and disorders of the placenta.

### SUMMARY OF THE INVENTION

Various embodiments of the invention provide purified polypeptides, proteins associated with cell growth, differentiation, and death, referred to collectively as ‘CGDD’ and individually as ‘CGDD-l,’ ‘CGDD-2,’ ‘CGDD-3,’ ‘CGDD4,’ ‘CGDD-5,’ ‘CGDD-6,’ ‘CGDD-7,’ ‘CGDD-8,’ ‘CGDD-9,’ ‘CGDD-10,’ ‘CGDD-11,’ ‘CGDD-12,’ ‘CGDD-13,’ ‘CGDD-14,’ ‘CGDD-15,’‘CGDD-16,’ ‘CGDD-17,’ ‘CGDD-18,’ ‘CGDD-19,’ ‘CGDD-20,’ ‘CGDD-21,’ ‘CGDD-22,’ ‘CGDD-23,’ ‘CGDD-24,’ ‘CGDD-25,’ ‘CGDD-26,’ ‘CGDD-27,’ ‘CGDD-28,’ ‘CGDD-29,’ ‘CGDD-30,’ ‘CGDD-31,’ ‘CGDD-32,’ ‘CGDD-33,’ ‘CGDD-34,’ ‘CGDD-35,’ ‘CGDD-36,’ ‘CGDD-37,’ ‘CGDD-38,’ ‘CGDD-39,’ ‘CGDD-40,’ ‘CGDD-41,’ ‘CGDD-42,’ ‘CGDD-43,’ ‘CGDD-44,’ ‘CGDD-45,’ ‘CGDD-46,’ ‘CGDD-47,’ ‘CGDD-48,’ ‘CGDD-49,’ ‘CGDD-50,’ ‘CGDD-51,’ ‘CGDD-52,’ ‘CGDD-53,’ ‘CGDD-54,’ ‘CGDD-55,’ ‘CGDD-56,’ and ‘CGDD-57’ and methods for using these proteins and their encoding polynucleotides for the detection, diagnosis, and treatment of diseases and medical conditions. Embodiments also provide methods for utilizing the purified proteins associated with cell growth, differentiation, and death and/or their encoding polynucleotides for facilitating the drug discovery process, including determination of efficacy, dosage, toxicity, and pharmacology. Related embodiments provide methods for utilizing the purified proteins associated with cell growth, differentiation, and death and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO:1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57. Another embodiment provides an isolated polypeptide comprising an amino acid sequence of SEQ ID NO:1-57.

Still another embodiment provides an isolated polynucleotide encoding a polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO:1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57. In another embodiment, the polynucleotide encodes a polypeptide selected from the group consisting of SEQ ID NO:1-57. In an alternative embodiment, the polynucleotide is selected from the group consisting of SEQ ID NO:58-114.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO:1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO:1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO:1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57.

Still yet another embodiment provides an isolated polynucleotide selected from the group consisting of a) a polynucleotide comprising a polynucleotide sequence selected from the group consisting of SEQ ID NO:58-114, b) a polynucleotide comprising a naturally occurring polynucleotide sequence at least 90% identical or at least about 90% identical to a polynucleotide sequence selected from the group consisting of SEQ ID NO:58-114, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:58-114, b) a polynucleotide comprising a naturally occurring polynucleotide sequence at least 90% identical or at least about 90% identical to a polynucleotide sequence selected from the group consisting of SEQ ID NO:58-114, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:58-114, b) a polynucleotide comprising a naturally occurring polynucleotide sequence at least 90% identical or at least about 90% identical to a polynucleotide sequence selected from the group consisting of SEQ ID NO:58-1 14, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO:1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57, and a pharmaceutically acceptable excipient. In one embodiment, the composition can comprise an amino acid sequence selected from the group consisting of SEQ ID NO:1-57. Other embodiments provide a method of treating a disease or condition associated with decreased or abnormal expression of functional CGDD, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO:1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional CGDD, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO:1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional CGDD, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO: 1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-57, b) a polypeptide comprising a naturally occurring amino acid sequence at least 90% identical or at least about 90% identical to an amino acid sequence selected from the group consisting of SEQ ID NO: 1-57, c) a biologically active fragment of a polypeptide having an amino acid sequence selected from the group consisting of SEQ ID NO:1-57, and d) an immunogenic fragment of a polypeptide having an amino acid sequence selected from the group consisting of SEQ ID NO:1-57. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:58-114, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:58-114, ii) a polynucleotide comprising a naturally occurring polynucleotide sequence at least 90% identical or at least about 90% identical to a polynucleotide sequence selected from the group consisting of SEQ ID NO:58-114, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:58-114, ii) a polynucleotide comprising a naturally occurring polynucleotide sequence at least 90% identical or at least about 90% identical to a polynucleotide sequence selected from the group consisting of SEQ ID NO:58-114, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“CGDD” refers to the amino acid sequences of substantially purified CGDD obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of CGDD. Agonists may include proteins, nucleic acids, carbohydrates, small molecules, or any other compound or composition which modulates the activity of CGDD either by directly interacting with CGDD or by acting on components of the biological pathway in which CGDD participates.

An “allelic variant” is an alternative form of the gene encoding CGDD. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding CGDD include those sequences with deletions, insertions, or substitutions of different nucleotides, resulting in a polypeptide the same as CGDD or a polypeptide with at least one functional characteristic of CGDD. Included within this definition are polymorphisms which may or may not be readily detectable using a particular oligonucleotide probe of the polynucleotide encoding CGDD, and improper or unexpected hybridization to allelic variants, with a locus other than the normal chromosomal locus for the polynucleotide encoding CGDD. The encoded protein may also be “altered,” and may contain deletions, insertions, or substitutions of amino acid residues which produce a silent change and result in a functionally equivalent CGDD. Deliberate amino acid substitutions may be made on the basis of one or more similarities in polarity, charge, solubility, hydrophobicity, hydrophilicity, and/or the amphipathic nature of the residues, as long as the biological or immunological activity of CGDD is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of CGDD. Antagonists may include proteins such as antibodies, anticalins, nucleic acids, carbohydrates, small molecules, or any other compound or composition which modulates the activity of CGDD either by directly interacting with CGDD or by acting on components of the biological pathway in which CGDD participates.

The term “antibody” refers to intact immunoglobulin molecules as well as to fragments thereof, such as Fab, F(ab′)², and Fv fragments, which are capable of binding an epitopic determinant. Antibodies that bind CGDD polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic CGDD, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding CGDD or fragments of CGDD may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g;, sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| | |
|---|---|
| Original Residue | Conservative Substitution |

| | |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample. A “Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of CGDD or a polynucleotide encoding CGDD which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:58-114 can comprise a region of unique polynucleotide sequence that specifically identifies SEQ ID NO:58-114, for example, as distinct from any other sequence in the genome from which the fragment was obtained. A fragment of SEQ ID NO:58-114 can be employed in one or more embodiments of methods of the invention, for example, in hybridization and amplification technologies and in analogous methods that distinguish SEQ ID NO:58-114 from related polynucleotides. The precise length of a fragment of SEQ ID NO:58-114 and the region of SEQ ID NO:58-114 to which the fragment corresponds are routinely determinable by one of ordinary skill in the art based on the intended purpose for the fragment.

A fragment of SEQ ID NO:1-57 is encoded by a fragment of SEQ ID NO:58-114. A fragment of SEQ ID NO:1-57 can comprise a region of unique amino acid sequence that specifically identifies SEQ ID NO:1-57. For example, a fragment of SEQ ID NO:1-57 can be used as an immunogenic peptide for the development of antibodies that specifically recognize SEQ ID NO:1-57. The precise length of a fragment of SEQ ID NO:1-57 and the region of SEQ ID NO:1-57 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wiss.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, MD, and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” too] Version 2.0.12 (April-21-2000) with blastp set at default parameters. Such default parameters may be, for example:

Matrix: BLOSUM62

Open Gap: 11 and Extension Gap: 1 penalties

Gap x drop-off 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tm) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of CGDD which is capable of eliciting an immune response when introduced into a living organism, for example, a mammal. The term “immunogenic fragment” also includes any polypeptide or oligopeptide fragment of CGDD which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of CGDD. For example, modulation may cause an increase or a decrease in protein activity, binding characteristics, or any other biological, functional, or immunological properties of CGDD.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an CGDD may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of CGDD.

“Probe” refers to nucleic acids encoding CGDD, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4th ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing CGDD, nucleic acids encoding CGDD, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 07, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May 07, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

The Invention

Various embodiments of the invention include new human proteins associated with cell growth, differentiation, and death (CGDD), the polynucleotides encoding CGDD, and the use of these compositions for the diagnosis, treatment, or prevention of cell proliferative disorders including cancer, developmental disorders, neurological disorders, autoimmune/inflammatory disorders, reproductive disorders, and disorders of the placenta.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Accelrys, Burlington Mass.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are proteins associated with cell growth, differentiation, and death. For example, SEQ ID NO:3 is 36% identical, from residue I428 to residue I184, to human B aggressive lymphoma short isoform (Genbank ID g12751141) as determined by the Basic Local Alignment Search Tool (BLAST). (See Table 2). The BLAST probability score is 8.0e-39, which indicates the probability of obtaining the observed polypeptide sequence alignment by chance. SEQ ID NO:3 also contains a Appr-“1”-p processing enzyme family domain and a WWE domain as determined by searching for statistically significant matches in the hidden Markov model (HMM)-based PFAM database of conserved protein family domains. (See Table 3.) Data from MOTIFS and further BLAST analyses provide corroborative evidence that SEQ ID NO:3 is a B aggressive lymphoma short isoform.

In another example, SEQ ID NO:6 is 95% identical, from residue M1 to residue K199, to human BCL7A (GenBank ID g929615) as determined by the Basic Local Alignment Search Tool (BLAST). (See Table 2.) The BLAST probability score is 1.2e-96, which indicates the probability of obtaining the observed polypeptide sequence alignment by chance. Data from MOTIFS and other BLAST analyses provide further corroborative evidence that SEQ ID NO:6 is a BCL7A, a gene with homology to caldesmon. (See Table 3.)

In another example, SEQ ID NO:9 is 95% identical, from residue L8 to residue W158, to human prostate carcinoma tumor antigen (GenBank ID g1932712) as determined by the Basic Local Alignment Search Tool (BLAST). (See Table 2.) The BLAST probability score is 1.5e-73, which indicates the probability of obtaining the observed polypeptide sequence alignment by chance. SEQ ID NO:9 is localized to the organelle or subcellular region, has a galactoside-binding function, and is a member of the galectin gene family, as determined by BLAST analysis using the PROTEOME database. SEQ ID NO:9 also contains a galactoside-binding lectin domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:9 is a galactoside-binding lectin.

In another example, SEQ ID NO:17 is 100% identical, from residue E19 to residue K139, to human natural killer cell transcript 4 (GenBank ID g14424787) as determined by the Basic Local Alignment Search Tool (BLAST). (See Table 2.) The BLAST probability score is 1.2e-72, which indicates the probability of obtaining the observed polypeptide sequence alignment by chance. SEQ ID NO:17 is localized to the extracellular region, has cell adhesion function, and is an RGD motif, as determined by BLAST analysis using the PROTEOME database. Data from BLAST-PRODOM and MOTIFS analyses provide further corroborative evidence that SEQ ID NO:17 is a natural killer cell protein.

In another example, SEQ ID NO:23 is 97% identical, from residue M1 to residue T281 and is 100% identical from residue V272 to residue E472, to human RTN2-A (GenBank ID g3435086) as determined by the Basic Local Alignment Search Tool (BLAST). (See Table 2.) The BLAST probability score is 3.0e-248, which indicates the probability of obtaining the observed polypeptide sequence alignment by chance. As determined by BLAST analysis using the PROTEOME database, SEQ ID NO:23 is homologous to Reticulon 2, a member of the reticulon family of proteins which are associated with the endoplasmic reticulum (PROTEOME ID 342756|RTN2); SEQ ID NO:23 is also homologous to a rat neuroendocrine-specific protein which may play a role in neuroendocrine secretory events (PROTEOME ID 331592|Rn.11075). SEQ ID NO:23 also contains a Reticulon domain as determined by searching for statistically significant matches in the hidden Markov model (HMM)-based PFAM database of conserved protein family domains. (See Table 3.) Data from MOTIFS and additional BLAST analyses provide further corroborative evidence that SEQ ID NO:23 is a member of the reticulon family of proteins.

In another example, SEQ ID NO:30 is 51% identical, from residue R55 to residue A273, to human breast cancer antigen NY-BR-1 (GenBank ID g13469729) as determined by the Basic Local Alignment Search Tool (BLAST). (See Table 2.) The BLAST probability score is 1.4e-62, which indicates the probability of obtaining the observed polypeptide sequence alignment by chance. SEQ ID NO: 30 is a protien containing ankyrin (Ank) repeats and may mediate protein-protein interactions, as determined by BLAST analysis using the PROTEOME database. SEQ ID NO:30 also contains a Ank repeat domain as determined by searching for statistically significant matches in the hidden Markov model (HMM)-based PFAM database of conserved protein family domains. (See Table 3.) Data from BLIMPS analysis provides further corroborative evidence that SEQ ID NO:30 is a human breast cancer antigen NY-BR-1, which contains Ank repeats.

In another example, SEQ ID NO:32 is 76% identical from residue M2 to residue F206, and 76% identical from residue S194 to residue R413, to Mus musculus clusterin (GenBank ID g192597) as determined by the Basic Local Alignment Search Tool (BLAST). (See Table 2.) The BLAST probability score is 2.3e-173, which indicates the probability of obtaining the observed polypeptide sequence alignment by chance. SEQ ID NO:32 also has homology to proteins that are clusterins, as determined by BLAST analysis using the PROTEOME database. SEQ ID NO:32 also contains a clusterin domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:32 shares structural characteristics of the glycoprotein clusterin.

In another example, SEQ ID NO:44 is 100% identical from residue M1 to residue V226 of SEQ ID NO:44 (residues M1-V226 of g292843), and 100% identical from residue H227 to residue E413 of SEQ ID NO:44 (residues H263-E449 of g292843), to human TRPM-2 gene product (GenBank ID g292843) as determined by the Basic Local Alignment Search Tool (BLAST). (See Table 2.) The BLAST probability score is 1.5e-222, which indicates the probability of obtaining the observed polypeptide sequence alignment by chance. SEQ ID NO:44 also has homology to clusterin, as determined by BLAST analysis using the PROTEOME database. SEQ ID NO:44 also contains a clusterin domain as determined by searching for statistically significant matches in the hidden Markov model (HMM)-based PFAM database of conserved protein family domains. (See Table 3.) Data from BLIMPS, MOTIFS, BLAST, and PROFILESCAN analyses provide further corroborative evidence that SEQ ID NO:44 is a clusterin.

In another example, SEQ ID NO:55 is 100% identical, from residue M1 to residue M20 and from residue V19 to residue K168, to human natural killer transcript 4 (GenBank ID g14424787, residues M1-M20 and residues V85 to K234 respectively) as determined by the Basic Local Alignment Search Tool (BLAST). (See Table 2.) The BLAST probability score is 5.6e-89, which indicates the probability of obtaining the observed polypeptide sequence alignment by chance. SEQ ID NO:55 is extracellular and is a natural killer cell transcript, as determined by BLAST analysis using the PROTEOME database. Data from further BLAST and MOTIFS analyses provide corroborative evidence that SEQ ID NO:55 is a natural killer cell transcript 4.

SEQ ID NO:1-2, SEQ ID DNO:4-5, SEQ ID NO:7-8, SEQ ID MB-10-16, SEQ ID MB-18-22, SEQ ID NO:24-29, SEQ ID NO:31, SEQ ID NO:33-43, SEQ ID NO:45-54, and SEQ ID NO:56-57 were analyzed and annotated in a similar manner. The algorithms and parameters for the analysis of SEQ ID NO:1-57 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:58-114 or that distinguish between SEQ ID NO:58-114 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (ie., those sequences including the designation “NP”). Alternatively, the polynucleotide fragments described in column 2 may refer to assemblages of both cDNA and Genscan-predicted exons brought together by an “exon stitching” algorithm For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3} . . . , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, GBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (ie., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for |
| GFG, | example, GENSCAN (Stanford University, CA, USA) or |
| ENST | FGENES (Computer Genomics Group, The Sanger Centre, |
| | Cambridge, UK). |
| GBI | Hand-edited analysis of genomic sequences. |
| FL | Stitched or stretched genomic sequences (see Example V). |
| INCY | Full length transcript and exon prediction from mapping |
| | of EST sequences to the genome. Genomic location and EST |
| | composition data are combined to predict the exons and |
| | resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

The invention also encompasses CGDD variants. Various embodiments of CGDD variants can have at least about 80%, at least about 90%, or at least about 95% amino acid sequence identity to the CGDD amino acid sequence, and can contain at least one functional or structural characteristic of CGDD.

Various embodiments also encompass polynucleotides which encode CGDD. In a particular embodiment, the invention encompasses a polynucleotide sequence comprising a sequence selected from the group consisting of SEQ ID NO:58-114, which encodes CGDD. The polynucleotide sequences of SEQ ID NO:58-114, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding CGDD. In particular, such a variant polynucleotide will have at least about 70%, or alternatively at least about 85%, or even at least about 95% polynucleotide sequence identity to a polynucleotide encoding CGDD. A particular aspect of the invention encompasses a variant of a polynucleotide comprising a sequence selected from the group consisting of SEQ ID NO:58-114 which has at least about 70%, or alternatively at least about 85%, or even at least about 95% polynucleotide sequence identity to a nucleic acid sequence selected from the group consisting of SEQ ID NO:58-114. Any one of the polynucleotide variants described above can encode a polypeptide which contains at least one functional or structural characteristic of CGDD.

In addition, or in the alternative, a polynucleotide variant of the invention is a splice variant of a polynucleotide encoding CGDD. A splice variant may have portions which have significant sequence identity to a polynucleotide encoding CGDD, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding CGDD over its entire length; however, portions of the splice variant will have at least about 70%, or alternatively at least about 85%, or alternatively at least about 95%, or alternatively 100% polynucleotide sequence identity to portions of the polynucleotide encoding CGDD. For example, a polynucleotide comprising a sequence of SEQ ID NO:73, a polynucleotide comprising a sequence of SEQ ID NO:110, and a polynucleotide comprising a sequence of SEQ ID NO:111 are splice variants of each other; a polynucleotide comprising a sequence of SEQ ID NO:74, a polynucleotide comprising a sequence of SEQ ID NO:112, and a polynucleotide comprising a sequence of SEQ ID NO:113 are splice variants of each other; a polynucleotide comprising a sequence of SEQ ID NO:87, a polynucleotide comprising a sequence of SEQ ID NO:99, and a polynucleotide comprising a sequence of SEQ ID NO:114 are splice variants of each other; a polynucleotide comprising a sequence of SEQ ID NO:69, and a polynucleotide comprising a sequence of SEQ ID NO:70 are splice variants of each other; a polynucleotide comprising a sequence of SEQ ID NO:71, and a polynucleotide comprising a sequence of SEQ ID NO:72 are splice variants of each other; a polynucleotide comprising a sequence of SEQ ID NO:90, a polynucleotide comprising a sequence of SEQ ID NO:91, a polynucleotide comprising a sequence of SEQ ID NO:101, and a polynucleotide comprising a sequence of SEQ ID NO:102 are splice variants of each other; a polynucleotide comprising a sequence of SEQ ID NO:94, and a polynucleotide comprising a sequence of SEQ ID NO:104 are splice variants of each other; and a polynucleotide comprising a sequence of SEQ ID NO:95, and a polynucleotide comprising a sequence of SEQ ID NO:96 are splice variants of each other. Any one of the splice variants described above can encode a polypeptide which contains at least one functional or structural characteristic of CGDD.

It will be appreciated by those skilled in the art that as a result of the degeneracy of the genetic code, a multitude of polynucleotide sequences encoding CGDD, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring CGDD, and all such variations are to be considered as being specifically disclosed.

Although polynucleotides which encode CGDD and its variants are generally capable of hybridizing to polynucleotides encoding naturally occurring CGDD under appropriately selected conditions of stringency, it may be advantageous to produce polynucleotides encoding CGDD or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding CGDD and its derivatives without altering the encoded amino acid sequences include the production of RNA transcripts having more desirable properties, such as a greater half-life, than transcripts produced from the naturally occurring sequence.

The invention also encompasses production of polynucleotides which encode CGDD and CGDD derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding CGDD or any fragment thereof.

Embodiments of the invention can also include polynucleotides that are capable of hybridizing to the claimed polynucleotides, and, in particular, to those having the sequences shown in SEQ ID NO:58-114 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding CGDD may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosysterns), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode CGDD may be cloned in recombinant DNA molecules that direct expression of CGDD, or fragments or functional equivalents thereof, in appropriate host cells. Due to the inherent degeneracy of the genetic code, other polynucleotides which encode substantially the same or a functionally equivalent polypeptides may be produced and used to express CGDD.

The polynucleotides of the invention can be engineered using methods generally known in the art in order to alter CGDD-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of CGDD, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding CGDD may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, CGDD itself or a fragment thereof may be synthesized using chemical methods known in the art. For example, peptide synthesis can be performed using various solution-phase or solid-phase techniques (Creighton, T. (1984) Proteins, Structures and Molecular Properties, WH Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of CGDD, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active CGDD, the polynucleotides encoding CGDD or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding CGDD. Such elements may vary in their strength and specificity. Specific initiation signals may also be used to achieve more efficient translation of polynucleotides encoding CGDD. Such signals include the ATG initiation codon and adjacent sequences, e.g. the Kozak sequence. In cases where a polynucleotide sequence encoding CGDD and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding CGDD and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding CGDD. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding CGDD. For example, routine cloning, subcloning, and propagation of polynucleotides encoding CGDD can be achieved using a multifunctional E. coli vector such as PBLUESCRIPT (Stratagene, La Jolla Calif.) or PSPORT1 plasmid (Invitrogen). Ligation of polynucleotides encoding CGDD into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of CGDD are needed, e.g. for the production of antibodies, vectors which direct high level expression of CGDD may be used. For example, vectors containing the strong, inducible SP6 or T7 bacteriophage promoter may be used.

Yeast expression systems may be used for production of CGDD. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of CGDD. Transcription of polynucleotides encoding CGDD may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding CGDD may be ligated into an adenovirus transcription/translation complex consisting of the late promoter and tripartite leader sequence. Insertion in a non-essential E1 or E3 region of the viral genome may be used to obtain infective virus which expresses CGDD in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of CGDD in cell lines is preferred. For example, polynucleotides encoding CGDD can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk and apr cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding CGDD is inserted within a marker gene sequence, transformed cells containing polynucleotides encoding CGDD can be identified by the absence of marker gene function. Alternatively, a marker gene can be placed in tandem with a sequence encoding CGDD under the control of a single promoter. Expression of the marker gene in response to induction or selection usually indicates expression of the tandem gene as well.

In general, host cells that contain the polynucleotide encoding CGDD and that express CGDD may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of CGDD using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on CGDD is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding CGDD include oligolabeling, nick translation, end-labeling, or PCR amplification using a labeled nucleotide. Alternatively, polynucleotides encoding CGDD, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding CGDD may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode CGDD may be designed to contain signal sequences which direct secretion of CGDD through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding CGDD may be ligated to a heterologous sequence resulting in translation of a fusion protein in any of the aforementioned host systems. For example, a chimeric CGDD protein containing a heterologous moiety that can be recognized by a commercially available antibody may facilitate the screening of peptide libraries for inhibitors of CGDD activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the CGDD encoding sequence and the heterologous protein sequence, so that CGDD may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled CGDD may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

CGDD, fragments of CGDD, or variants of CGDD may be used to screen for compounds that specifically bind to CGDD. One or more test compounds may be screened for specific binding to CGDD. In various embodiments, 1, 2, 3, 4, 5, 10, 20, 50, 100, or 200 test compounds can be screened for specific binding to CGDD. Examples of test compounds can include antibodies, anticalins, oligonucleotides, proteins (e.g., ligands or receptors), or small molecules.

In related embodiments, variants of CGDD can be used to screen for binding of test compounds, such as antibodies, to CGDD, a variant of CGDD, or a combination of CGDD and/or one or more variants CGDD. In an embodiment, a variant of CGDD can be used to screen for compounds that bind to a variant of CGDD, but not to CGDD having the exact sequence of a sequence of SEQ ID NO:1-57. CGDD variants used to perform such screening can have a range of about 50% to about 99% sequence identity to CGDD, with various embodiments having 60%, 70%, 75%, 80%, 85%, 90%, and 95% sequence identity.

In an embodiment, a compound identified in a screen for specific binding to CGDD can be closely related to the natural ligand of CGDD, e.g., a ligand or fragment thereof, a natural substrate, a structural or functional mimetic, or a natural binding partner (Coligan, J. E. et al. (1991) Current Protocols in Immunology 1(2): Chapter 5). In another embodiment, the compound thus identified can be a natural ligand of a receptor CGDD (Howard, A. D. et al. (2001) Trends Pharmacol. Sci. 22:132-140; Wise, A. et al. (2002) Drug Discovery Today 7:235-246).

In other embodiments, a compound identified in a screen for specific binding to CGDD can be closely related to the natural receptor to which CGDD binds, at least a fragment of the receptor, or a fragment of the receptor including all or a portion of the ligand binding site or binding pocket. For example, the compound may be a receptor for CGDD which is capable of propagating a signal, or a decoy receptor for CGDD which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to CGDD, fragments of CGDD, or variants of CGDD. The binding specificity of the antibodies thus screened can thereby be selected to identify particular fragments or variants of CGDD. In one embodiment, an antibody can be selected such that its binding specificity allows for preferential identification of specific fragments or variants of CGDD. In another embodiment, an antibody can be selected such that its binding specificity allows for preferential diagnosis of a specific disease or condition having increased, decreased, or otherwise abnormal production of CGDD.

In an embodiment, anticalins can be screened for specific binding to CGDD, fragments of CGDD, or variants of CGDD. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit CGDD involves producing appropriate cells which express CGDD, either as a secreted protein or on the cell membrane. Preferred cells can include cells from mammals, yeast, Drosophila, or E. coli. Cells expressing CGDD or cell membrane fractions which contain CGDD are then contacted with a test compound and binding, stimulation, or inhibition of activity of either CGDD or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with CGDD, either in solution or affixed to a solid support, and detecting the binding of CGDD to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

CGDD, fragments of CGDD, or variants of CGDD may be used to screen for compounds that modulate the activity of CGDD. Such compounds may include agonists, antagonists, or partial or inverse agonists. In one embodiment, an assay is performed under conditions permissive for CGDD activity, wherein CGDD is combined with at least one test compound, and the activity of CGDD in the presence of a test compound is compared with the activity of CGDD in the absence of the test compound. A change in the activity of CGDD in the presence of the test compound is indicative of a compound that modulates the activity of CGDD. Alternatively, a test compound is combined with an in vitro or cell-free system comprising CGDD under conditions suitable for CGDD activity, and the assay is performed. In either of these assays, a test compound which modulates the activity of CGDD may do so indirectly and need not come in direct contact with the test compound. At least one and up to a plurality of test compounds may be screened.

In another embodiment, polynucleotides encoding CGDD or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding CGDD may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding CGDD can also be used to create “knockin” humanized animals (pigs) or transgenic animals (mice or rats) to model human disease. With knockin technology, a region of a polynucleotide encoding CGDD is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress CGDD, e.g., by secreting CGDD in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of CGDD and proteins associated with cell growth, differentiation, and death. In addition, examples of tissues expressing CGDD can be found in Table 6 and can also be found in Example XI. Therefore, CGDD appears to play a role in cell proliferative disorders including cancer, developmental disorders, neurological disorders, autoimmune/inflammatory disorders, reproductive disorders, and disorders of the placenta. In the treatment of disorders associated with increased CGDD expression or activity, it is desirable to decrease the expression or activity of CGDD. In the treatment of disorders associated with decreased CGDD expression or activity, it is desirable to increase the expression or activity of CGDD.

Therefore, in one embodiment, CGDD or a fragment or derivative thereof may be administered to a subject to treat or prevent a disorder associated with decreased expression or activity of CGDD. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a reproductive disorder such as a disorder of prolactin production, infertility, including tubal disease, ovulatory defects, endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, ectopic pregnancy, teratogenesis; cancer of the breast, fibrocystic breast disease, galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, gynecomastia, hypergonadotropic and hypogonadotropic hypogonadism, pseudohermaphroditism, azoospermia, premature ovarian failure, acrosin deficiency, delayed puperty, retrograde ejaculation and anejaculation, haemangioblastomas, cystsphaeochromocytomas, paraganglioma, cystadenomas of the epididymis, and endolymphatic sac tumors; and a disorder of the placenta such as preeclampsia, choriocarcinoma, abruptio placentae, placenta previa, placental or maternal floor infarction, placenta accreta, increate, and percreta, extrachorial placentas, chorangioma, chorangiosis, chronic villitis, placental villous endema, widespread fibrosis of the terminal villi, intervillous thrombi, hemorraghic endovasculitis, erythroblastosis fetalis, and nonimmune fetal hydrops.

In another embodiment, a vector capable of expressing CGDD or a fragment or derivative thereof may be administered to a subject to treat or prevent a disorder associated with decreased expression or activity of CGDD including, but not limited to, those described above.

In a further embodiment, a composition comprising a substantially purified CGDD in conjunction with a suitable pharmaceutical carrier may be administered to a subject to treat or prevent a disorder associated with decreased expression or activity of CGDD including, but not limited to, those provided above.

In still another embodiment, an agonist which modulates the activity of CGDD may be administered to a subject to treat or prevent a disorder associated with decreased expression or activity of CGDD including, but not limited to, those listed above.

In a further embodiment, an antagonist of CGDD may be administered to a subject to treat or prevent a disorder associated with increased expression or activity of CGDD. Examples of such disorders include, but are not limited to, those cell proliferative disorders including cancer, developmental disorders, neurological disorders, autoimmune/inflammatory disorders, reproductive disorders, and disorders of the placenta described above. In one aspect, an antibody which specifically binds CGDD may be used directly as an antagonist or indirectly as a targeting or delivery mechanism for bringing a pharmaceutical agent to cells or tissues which express CGDD.

In an additional embodiment, a vector expressing the complement of the polynucleotide encoding CGDD may be administered to a subject to treat or prevent a disorder associated with increased expression or activity of CGDD including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of CGDD may be produced using methods which are generally known in the art. In particular, purified CGDD may be used to produce antibodies or to screen libraries of pharmaceutical agents to identify those which specifically bind CGDD. Antibodies to CGDD may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with CGDD or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to CGDD have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of CGDD amino acids may be fused with those of another protein, such as KLH, and antibodies to the chimeric molecule may be produced.

Monoclonal antibodies to CGDD may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:3142; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce CGDD-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for CGDD may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between CGDD and its specific antibody. A two-site, monoclonal-based immunoassay utilizing monoclonal antibodies reactive to two non-interfering CGDD epitopes is generally used, but a competitive binding assay may also be employed (Pound, supra).

Various methods such as Scatchard analysis in conjunction with radioimmunoassay techniques may be used to assess the affinity of antibodies for CGDD. Affinity is expressed as an association constant, Kₐ, which is defined as the molar concentration of CGDD-antibody complex divided by the molar concentrations of free antigen and free antibody under equilibrium conditions. The Kₐ determined for a preparation of polyclonal antibodies, which are heterogeneous in their affinities for multiple CGDD epitopes, represents the average affinity, or avidity, of the antibodies for CGDD. The Kₐ determined for a preparation of monoclonal antibodies, which are monospecific for a particular CGDD epitope, represents a true measure of affinity. High-affinity antibody preparations with Kₐ ranging from about 10⁹ to 10¹² L/mole are preferred for use in immunoassays in which the CGDD-antibody complex must withstand rigorous manipulations. Low-affinity antibody preparations with Kₐ ranging from about 10⁶ to 10⁷ L/mole are preferred for use in immunopurification and similar procedures which ultimately require dissociation of CGDD, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of CGDD-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding CGDD, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding CGDD. Such technology is well known in the art, and antisense oligonucleotides or larger fragments can be designed from various locations along the coding or control regions of sequences encoding CGDD (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding CGDD may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immnunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in CGDD expression or regulation causes disease, the expression of CGDD from an appropriate population of transduced cells may alleviate the clinical manifestations caused by the genetic deficiency.

In a further embodiment of the invention, diseases or disorders caused by deficiencies in CGDD are treated by constructing mammalian expression vectors encoding CGDD and introducing these vectors by mechanical means into CGDD-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of CGDD include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). CGDD may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding CGDD from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to CGDD expression are treated by constructing a retrovirus vector consisting of (i) the polynucleotide encoding CGDD under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding CGDD to cells which have one or more genetic abnormalities with respect to the expression of CGDD. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding CGDD to target cells which have one or more genetic abnormalities with respect to the expression of CGDD. The use of herpes simplex virus (HSV)-based vectors may be especially valuable for introducing CGDD to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding CGDD to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for CGDD into the alphavirus genome in place of the capsid-coding region results in the production of a large number of CGDD-coding RNAs and the synthesis of high levels of CGDD in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of CGDD into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding CGDD.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding CGDD. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. SiRNA are shorter segments of dsRNA (typically about 21 to 23 nucleotides in length) that result in vivo from cleavage of introduced dsRNA by the action of an endogenous ribonuclease. SiRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

SiRNA can either be generated indirectly by introduction of dsRNA into the targeted cell, or directly by mammalian transfection methods and agents described herein or known in the art (such as liposome-mediated transfection, viral vector methods, or other polynucleotide delivery/introductory methods). Suitable SiRNAs can be selected by examining a transcript of the target polynucleotide (e.g., nRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected SiRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene, can be determined by northern analysis methods using, for example, the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined by Western analysis using standard techniques known in the art.

An additional embodiment of the invention encompasses a method for screening for a compound which is effective in altering expression of a polynucleotide encoding CGDD. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased CGDD expression or activity, a compound which specifically inhibits expression of the polynucleotide encoding CGDD may be therapeutically useful, and in the treatment of disorders associated with decreased CGDD expression or activity, a compound which specifically promotes expression of the polynucleotide encoding CGDD may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding CGDD is exposed to at least one test compound thus obtained. The sample may comprise, for example, an intact or permeabilized cell, or an in vitro cell-free or reconstituted biochemical system. Alterations in the expression of a polynucleotide encoding CGDD are assayed by any method commonly known in the art. Typically, the expression of a specific nucleotide is detected by hybridization with a probe having a nucleotide sequence complementary to the sequence of the polynucleotide encoding CGDD. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of CGDD, antibodies to CGDD, and mimetics, agonists, antagonists, or inhibitors of CGDD.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising CGDD or fragments thereof. For example, liposome preparations containing a cell-impermeable macromolecule may promote cell fusion and intracellular delivery of the macromolecule. Alternatively, CGDD or a fragment thereof may be joined to a short cationic N-terminal portion from the HIV Tat-I protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example CGDD or fragments thereof, antibodies of CGDD, and agonists, antagonists or inhibitors of CGDD, which ameliorates the symptoms or condition. Therapeutic efficacy and toxicity may be determined by standard pharmaceutical procedures in cell cultures or with experimental animals, such as by calculating the ED50 (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind CGDD may be used for the diagnosis of disorders characterized by expression of CGDD, or in assays to monitor patients being treated with CGDD or agonists, antagonists, or inhibitors of CGDD. Antibodies useful for diagnostic purposes may be prepared in the same manner as described above for therapeutics. Diagnostic assays for CGDD include methods which utilize the antibody and a label to detect CGDD in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring CGDD, including ELISAs, RIAs, and FACS, are known in the art and provide a basis for diagnosing altered or abnormal levels of CGDD expression. Normal or standard values for CGDD expression are established by combining body fluids or cell extracts taken from normal mammalian subjects, for example, human subjects, with antibodies to CGDD under conditions suitable for complex formation. The amount of standard complex formation may be quantitated by various methods, such as photometric means. Quantities of CGDD expressed in subject, control, and disease samples from biopsied tissues are compared with the standard values. Deviation between standard and subject values establishes the parameters for diagnosing disease.

In another embodiment of the invention, polynucleotides encoding CGDD may be used for diagnostic purposes. The polynucleotides which may be used include oligonucleotides, complementary RNA and DNA molecules, and PNAs. The polynucleotides may be used to detect and quantify gene expression in biopsied tissues in which expression of CGDD may be correlated with disease. The diagnostic assay may be used to determine absence, presence, and excess expression of CGDD, and to monitor regulation of CGDD levels during therapeutic intervention.

In one aspect, hybridization with PCR probes which are capable of detecting polynucleotides, including genomic sequences, encoding CGDD or closely related molecules may be used to identify nucleic acid sequences which encode CGDD. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding CGDD, allelic variants, or related sequences.

Probes may also be used for the detection of related sequences, and may have at least 50% sequence identity to any of the CGDD encoding sequences. The hybridization probes of the subject invention may be DNA or RNA and may be derived from the sequence of SEQ ID NO:58-114 or from genomic sequences including promoters, enhancers, and introns of the CGDD gene.

Means for producing specific hybridization probes for polynucleotides encoding CGDD include the cloning of polynucleotides encoding CGDD or CGDD derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²p or ³⁵S, or by enzymatic labels, such as alkaline phosphatase coupled to the probe via avidin/biotin coupling systems, and the like.

Polynucleotides encoding CGDD may be used for the diagnosis of disorders associated with expression of CGDD. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a reproductive disorder such as a disorder of prolactin production, infertility, including tubal disease, ovulatory defects, endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, ectopic pregnancy, teratogenesis; cancer of the breast, fibrocystic breast disease, galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, gynecomastia, hypergonadotropic and hypogonadotropic hypogonadism, pseudohermaphroditism, azoospermia, premature ovarian failure, acrosin deficiency, delayed puperty, retrograde ejaculation and anejaculation, haemangioblastomas, cystsphaeochromocytomas, paraganglioma, cystadenomas of the epididymis, and endolymphatic sac tumors; and a disorder of the placenta such as preeclampsia, choriocarcinoma, abruptio placentae, placenta previa, placental or maternal floor infarction, placenta accreta, increate, and percreta, extrachorial placentas, chorangioma, chorangiosis, chronic villitis, placental villous endema, widespread fibrosis of the terminal villi, intervillous thrombi, hemorraghic endovasculitis, erythroblastosis fetalis, and nonimmune fetal hydrops. Polynucleotides encoding CGDD may be used in Southern or northern analysis, dot blot, or other membrane-based technologies; in PCR technologies; in dipstick, pin, and multiformat ELISA-like assays; and in microarrays utilizing fluids or tissues from patients to detect altered CGDD expression. Such qualitative or quantitative methods are well known in the art.

In a particular embodiment, polynucleotides encoding CGDD may be used in assays that detect the presence of associated disorders, particularly those mentioned above. Polynucleotides complementary to sequences encoding CGDD may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding CGDD in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of CGDD, a normal or standard profile for expression is established. This may be accomplished by combining body fluids or cell extracts taken from normal subjects, either animal or human, with a sequence, or a fragment thereof, encoding CGDD, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding CGDD may involve the use of PCR. These oligomers may be chemically synthesized, generated enzymatically, or produced in vitro. Oligomers will preferably contain a fragment of a polynucleotide encoding CGDD, or a fragment of a polynucleotide complementary to the polynucleotide encoding CGDD, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding CGDD may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding CGDD are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of CGDD include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, CGDD, fragments of CGDD, or antibodies specific for CGDD may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for CGDD to quantify the levels of CGDD expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding CGDD may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding CGDD on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, CGDD, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between CGDD and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with CGDD, or fragments thereof, and washed. Bound CGDD is then detected by methods well known in the art. Purified CGDD can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding CGDD specifically compete with a test compound for binding CGDD. In this manner, antibodies can be used to detect the presence of any peptide which shares one or more antigenic determinants with CGDD.

In additional embodiments, the nucleotide sequences which encode CGDD may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications, and publications mentioned above and below, including U.S. Ser. No. 60/326,389, U.S. Ser. No. 60/328,186, U.S. Ser. No. 60/329,690, U.S. Ser. No. 60/348,165, U.S. Ser. No. 60/350,219, U.S. Ser. No. 60/344,518, U.S. Ser. No. 60/332,375, U.S. Ser. No. 60/336,908, U.S. Ser. No. 60/327,380, U.S. Ser. No. 60/345,384, U.S. Ser. No. 60/340,747 and U.S. Ser. No.60/345,143, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S 1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:58-114. Fragments from about 20 to about 4000 nucleotides which are useful in hybridization and amplification technologies are described in Table 4, column 2.

IV. Identification and Editing of Coding Sequences from Genomic DNA

Putative proteins associated with cell growth, differentiation, and death were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode proteins associated with cell growth, differentiation, and death, the encoded polypeptides were analyzed by querying against PFAM models for proteins associated with cell growth, differentiation, and death. Potential proteins associated with cell growth, differentiation, and death were also identified by homology to Incyte cDNA sequences that had been annotated as proteins associated with cell growth, differentiation, and death. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of CGDD Encoding Polynucleotides

The sequences which were used to assemble SEQ ID NO:58-114 were compared with sequences from the Incyte LIFESEQ database and public domain databases using BLAST and other implementations of the Smith-Waterman algorithm. Sequences from these databases that matched SEQ ID NO:58-114 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST were used to search for identical or related molecules in databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and 4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding CGDD are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding CGDD. cDNA sequences and cDNA library/tissue information are found in the LIFESEQ GOLD database (Incyte Genomics, Palo Alto Calif.).

VIII. Extension of CGDD Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10,μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2x carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in CGDD Encoding Polynucleotides

Common DNA sequence variants known as single nucleotide polymorphisms (SNPs) were identified in SEQ ID NO:58-114 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:58-114 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte Genomics). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Array elements that exhibit at least about a two-fold change in expression, a signal-to-background ratio of at least about 2.5, and an element spot size of at least about 40%, are considered to be differentially expressed.

Expression

For example, SEQ ID NO:64 showed differential expression in tumorous tissue versus non-tumorous tissues and cell lines, as determined by microarray analysis. The expression of SEQ ID NO:64 was decreased by at least two fold in breast tumor cell lines that were harvested from donors at different stages of tumor progression and malignant transformation when grown under optimal growth conditions in the presence of growth factors and nutrients or grown in basal media in the absence of growth factors and hormones for 24 hours prior to comparison with a human mammary epithelial cell line. Therefore, SEQ ID NO:64 is useful in diagnostic assays for breast cancer.

Normal breast cell lines are obtained as follows. Primary mammary gland cells are isolated from a donor with fibrocystic breast disease. Alternatively, primary breast epithelial cells are isolated from a normal donor. Tumorous breast cell lines are obtained as follows. Breast carcinoma cells are derived in vitro from cells emigrating from a tumor. Alternately, breast tumor cells are isolated from invasive tumor of donors. Further, nonmalignant or malignant primary breast adenocarcinoma cells are obtained from the pleural effusion of donors.

The expression of SEQ ID NO:64 also was decreased at least two-fold in human colon adenocarcinoma tissue matched with normal tissue from the same donor. The colon adenocarcinoma tissue was obtained from an 83-year-old male with moderately well differentiated adenocarcinoma from the ascending colon which had metasticized to a peritumoral lymph node. Normal colon tissue was obtained from grossly uninvolved colon tissue (pooled normal tissue) from the same donor. Therefore, SEQ ID NO:64 is useful in diagnostic assays for colon cancer.

The expression of SEQ ID NO:64 also was decreased at least 2.8-fold in a lung adenocarcinoma when matched with normal tissue from the same donor. The tumorous lung tissue was obtained from the right lung of a 60-year-old donor (gender unknown) with moderately differentiated adenocarcinoma. Normal lung tissue was obtained from grossly uninvolved tissue from the right lung of the same donor. Therefore, SEQ ID NO:64 is useful in diagnostic assays for lung adenocarcinoma.

Further, the expression of SEQ ID NO:64 was decreased at least 2.9-fold in an ovarian adenocarcinoma when matched with normal tissue from the same donor. The tumorous ovary tissue was obtained from ovarian adenocarcinoma from a 79-year-old female. Normal ovary tissue was obtained from ovary from the same donor. Therefore, SEQ ID NO:64 is useful in diagnostic assays for ovarian adenocarcinoma.

Matched normal and tumorigenic colon, lung, and ovary tissue samples are provided by the Huntsman Cancer Institute, (Salt Lake City, Utah).

The expression of SEQ ID NO:66 was differentially expressed in breast tumor tissue using a variety of experimental protocols. Histological and molecular evaluation of breast tumors reveals that breast cancer develops through a multi-step process whereby pre-malignant mammary epithelial cells undergo a relatively defined sequence of events leading to tumor formation. An early event in tumor development is ductal hyperplasia. Cells undergoing rapid neoplastic growth gradually progress to invasive carcinoma and become metastatic to the lung, bone, and potentially other organs. Several factors participate in the process of tumor progression and malignant transformation, including genetic factors, environmental factors, growth factors, and hormones. The complexity of this process makes it critical to study a population of human mammary epithelial cells undergoing the process of malignant transformation, and to associate specific stages of progression with phenotypic and molecular characteristics.

In one set of experiments, human breast tumor cell lines were evaluated using a cross-comparison study design against a nonmalignant breast epithelial cell line (MCF-10A). The gene expression profiles were examined by comparing five breast carcinoma cell lines to the nonmalignant MCF-10A, all cells being grown in defined serum-free H14 medium to 70-80% confluence prior to RNA harvest. Four out of the five breast tumor cell lines tested exhibited at least a two-fold decrease in expression as compared to the nonmalignant cell line: MCF7 (breast adenocarcinoma), T47D (breast ductal carcinoma), SKBr3 (breast adenocarcinoma, also tumorigenic in nude mice), and BT-20 (breast adenocarcinoma, also tumorigenic in nude mice) all being underexpressed.

In another experiment involving the BT-20 breast adenocarcinoma cell line, the cells were grown by embedding single cell suspensions in Matrigel matrix. RNA was harvested when modestly sized colonies formed. Normal tissue homeostasis is maintained by dynamic interactions between epithelial cells and their microenvironment. As tissue becomes cancerous, reciprocal interactions occur between neoplastic cells, adjacent normal cells such as stroma and endothelium, and their microenvironment. The microenvironments were defined as insoluble extracellular matrix (EMC); stroma consisting of fibroblasts, adipose, vasculature, and resident immune cells; and the conventional milieu of cytokines and growth factors. Epithelial parenchyma are physically separated from stroma by a basement membrane: a highly organized, special ECM, whose composition is different from stromal ECM and to which epithelial cells attach. The complex interactions between epithelial cells and their microenvironment are critical for maintaining normal, balanced homeostasis. Disruption of this balance can induce aberrant cell proliferation, adhesion, function, and migration that might promote malignant and metastatic behavior. The expression of SEQ ID NO:66 was significantly overexpressed, by at least two-fold, when exposed to the Matrigel matrix.

Lung cancers are divided into four histopathologically distinct groups. Three groups (squamous cell carcinoma, adenocarcinoma, and large cell carcinoma) are classified as non-small cell lung cancers (NSCLCs). The fourth group of cancers is referred to as small cell lung cancer (SCLC). Collectively, NSCLCs account for approximately 70% of cases while SCLCs account for approximately 18% of cases. The molecular and cellular biology underlying the development and progression of lung cancer are incompletely understood. Analysis of gene expression patterns associated with the development and progression of the disease will yield insight into the biology underlying this disease. In three pair comparison experiments, normal lung tissue was compared to lung tumor tissue from the same donor. In all three experiments, the expression of SEQ ID NO:67 was significantly overexpressed by at least two-fold in the lung tumor tissue as compared to the normal lung tissue.

These experiments indicate that SEQ ID NO:66 and SEQ ID NO:67 exhibited significant differential expression patterns using microarray techniques, and further establish the utility of SEQ ID NO:66 and SEQ ID NO:67 as diagnostic markers or therapeutic targets useful in a variety of conditions and diseases involving proteins associated with cell growth, differentiation, and death.

SEQ ID NO:74 was differentially expressed in human tumor colon tissue compared to normal colon tissue. Approximately 90% of colorectal cancer occurs in individuals over the age of 55. A widely accepted hypothesis is that several mutations must accumulate over time before an individual develops the disease. Inherited syndromes, such as familial adenomatous polyposis (FAP) is caused by mutations in the adenomatous polyposis coli gene (APC), resulting in truncated or inactive forms of the protein. This tumor suppressor gene has been mapped to chromosome 5q. The second known inherited syndrome is hereditary nonpolyposis colorectal cancer (HNPCC), which is caused by mutations in mismatch repair genes. Although hereditary colon cancer syndromes occur in a small percentage of the population, most colorectal cancers are considered sporadic. Somatic mutations in APC occur in at least 80% of sporadic colon tumors. APC mutations are thought to be the initiating event in the disease. Other mutations occur subsequently. Approximately 50% of colorectal cancers contain activating mutations in ras, while 85% contain inactivating mutations in p53. Changes in all of these genes lead to gene expression changes in colon cancer. Less is understood about downstream targets of these mutations and the role they may play in cancer development and progression.

SEQ ID NO:74 was significantly underexpressed by at least two-fold in colon tissue containing polyps from a 24-year old donor as compared to normal colon tissue. In another set of experiments, the expression of SEQ ID NO:74 was significantly underexpressed by at least two-fold in colon tumor tissue as compared to normal colon tissue.

SEQ ID NO:74 was differentially expressed in several pair comparison experiments in which various types of lung tumor tissue (squamous cell carcinoma) was compared to uninvolved lung tissue from the same donor. In all cases, the expression of SEQ ID NO:74 was underexpressed by at least two-fold in the lung tumor tissue.

These experiments indicate that SEQ ID NO:74 exhibits significant differential expression patterns using microarray techniques, and further establish the utility of SEQ ID NO:74 as a diagnostic marker or therapeutic agent which may be useful in a variety of conditions and diseases involving proteins associated with cell growth, differentiation, and death, including cancers.

For example, SEQ ID NO:75 showed differential expression in lung squamous cell carcinoma versus normal lung tissues as determined by microarray analysis. The expression of SEQ ID NO:75 was decreased by at least two fold in lung squamous cell carcinoma relative to grossly uninvolved normal lung tissue from the same donors. Therefore, SEQ ID NO:75 is useful as a diagnostic marker for disease staging or as a potential therapeutic target for lung cancer.

In an alternative example, SEQ ID NO:75 and SEQ ID NO:79 showed differential expression in certain breast carcinoma cell lines versus primary mammary epithelial cells as determined by microarray analysis. The breast carcinoma cell lines include MCF7, a breast adenocarcinoma cell line derived from the pleural effusion of a 69-year-old female; T-47D, a breast carcinoma cell line derived from a pleural effusion from a 54-year-old female with an infiltrating ductal carcinoma of the breast; Sk-BR-3, a breast adenocarcinoma cell line isolated from a malignant pleural effusion of a 43-year-old female; MDA-mb-231, a metastatic breast tumor cell line derived from the pleural effusion of a 51-year-old female with metastatic breast carcinoma; and MDA-mb-435S, a spindle shaped strain that evolved from a cell line isolated from the pleural effusion of a 31 year old female with metastatic, ductal adenocarcinoma of the breast. The primary mammary epithelial cell line HMEC was derived from normal human mammary tissue (Clonetics, San Diego, Calif.). All cell cultures were propagated in a chemically-defined medium, according to the supplier's recommendations and grown to 70-80% confluence prior to RNA isolation. The microarray experiments showed that the expression of SEQ ID NO:75 was increased by at least two fold in T-47D and Sk-BR-3 breast carcinoma lines relative to primary mammary epithelial cells; and the expression of SEQ ID NO:79 was increased by at least two fold in Sk-BR-3 breast carcinoma lines relative to primary mammary epithelial cells. Therefore, SEQ ID NO:75 and SEQ ID NO:79 are useful as diagnostic markers or as potential therapeutic targets for certain breast cancers.

For example, gene expression patterns of matched normal and lung tumor tissues were compared. SEQ ID NO: 83 was found to be downregulated by at least two-fold in six out of six donors studied. Analysis of gene expression patterns associated with the development and progression of lung cancer will yield tremendous insight into the biology underlying this disease, and will lead to the development of improved diagnostics and therapeutics. SEQ ID NO:83, encoding SEQ ID NO:26, can be used in these diagnostics and therapeutics.

In an alternative example, gene expression patterns of matched normal and breast tumor tissues were compared. SEQ ID NO:83 was found to be downregulated by at least two-fold in one donor studied. Analysis of gene expression patterns associated with the development and progression of breast cancer will yield tremendous insight into the biology underlying this disease, and will lead to the development of improved diagnostics and therapeutics. SEQ ID NO:83, encoding SEQ ID NO:26, can be used in these diagnostics and therapeutics.

SEQ ID NO:88 showed increased expression in sigmoid colon tumor tissue versus normal sigmoid colon tissue as determined by microarray analysis. Gene expression profiles were obtained by comparing normal sigmoid colon tissue to a sigmoid colon tumor originating from a metastatic gastric sarcoma (stromal tumor) from a 48-year-old female (Huntsman Cancer Institute, Salt Lake City, Utah). Therefore, SEQ ID NO:88 is useful in monitoring treatment of, and diagnostic assays for, sigmoid colon cancer and other cell proliferative disorders.

For example, SEQ ID NO:89, SEQ ID NO:91, and SEQ ID NO:92 showed increased expression in breast carcinoma cells versus nonmalignant mammary epithelial cells as determined by microarray analysis. The gene expression profile of a nonmalignant mammary epithelial cell line was compared to the gene expression profiles of breast carcinoma lines at different stages of tumor progression. Cell lines compared for SEQ ID NO:89 and SEQ ID NO:92 included: a) MCF-10A, a breast mammary gland cell line isolated from a 36-year-old woman with fibrocystic breast disease; b)MCF7, a nonmalignant breast adenocarcinoma cell line isolated from the pleural effusion of a 69-year-old female; c)T-47D, a breast carcinoma cell line isolated from a pleural effusion obtained from a 54-year-old female with an infiltrating ductal carcinoma of the breast; d)Sk-BR-3, a breast adenocarcinoma cell line isolated from a malignant pleural effusion of a 43-year-old female; e)BT-20, a breast carcinoma cell line derived in vitro from tumor mass isolated from a 74-year-old female; f)MDA-mb-231, a breast tumor cell line isolated from the pleural effusion of a 51-year old female; and g) MDA-mb435S, a spindle shaped strain that evolved from the parent line (435) isolated from the pleural effusion of a 31-year-old female with metastatic, ductal adenocarcinoma of the breast. Cell lines compared for SEQ ID NO:91 included: a) BT-20, a breast carcinoma cell line derived in vitro from the cells emigrating out of thin slices of tumor mass isolated from a 74-year-old female, b) BT-474, a breast ductal carcinoma cell line that was isolated from a solid, invasive ductal carcinoma of the breast obtained from a 60-year-old woman, c) BT-483, a breast ductal carcinoma cell line that was isolated from a papillary invasive ductal tumor obtained from a 23-year-old normal, menstruating, parous female with a family history of breast cancer, d) Hs 578T, a breast ductal carcinoma cell line isolated from a 74-year-old female with breast carcinoma, e) MCF7, a nonmalignant breast adenocarcinoma cell line isolated from the pleural effusion of a 69-year-old female, f) MCF-10A, a breast mammary gland (luminal ductal characteristics) cell line isolated. from a 36-year-old woman with fibrocystic breast disease, g) MDA-MB-468, a breast adenocarcinoma cell line isolated from the pleural effusion of a 51-year-old female with metastatic adenocarcinoma of the breast, and h) HMEC, a primary breast epithelial cell line isolated from a normal donor. Therefore, SEQ ID NO:89, SEQ ID NO:91, and SEQ ID NO:92 are useful in monitoring treatment of, and diagnostic assays for, breast cancer and other cell proliferative disorders.

In another example, SEQ ID NO:91 showed increased expression in fibroblasts affected by Tangier disease versus normal fibroblasts as determined by microarray analysis. Normal and Tangier disease derived fibroblasts were compared. In addition, both types of cells were cultured in the presence of cholesterol and compared with the same cell type cultured in the absence of cholesterol. Human fibroblasts were obtained from skin explants from both normal subjects and two patients with homozygous Tangier disease. Cell lines were immortalized by transfection with human papillomavirus 16 genes E6 and E7 and a neomycin resistance selectable marker. TD derived cells are deficient in an assay of apoA-I mediated tritiated cholesterol efflux. Therefore, SEQ ID NO:91 is useful in monitoring treatment of, and diagnostic assays for, Tangier disease and other autoimmune/inflammation disorders.

SEQ ID NO:89, SEQ ID NO:91, and SEQ ID NO:97 showed increased expression in osteoblasts affected by osteosarcoma versus normal osteoblasts as determined by microarray analysis. Messenger RNA from normal human osteoblast (primary culture, NHOst 5488) was compared with mRNA from biopsy specimens, osteosarcoma tissues, or primary cultures or metastasized tissues. Approximately 2.0×10 6 cells in single cell suspension were seeded into T75 flasks in duplicates or triplicates. Cell lines were subcultured on average every 6-8 days at a ratio of 1:6-8. Therefore, SEQ ID NO:89, SEQ ID NO:91, and SEQ ID NO:97 are useful in monitoring treatment of, and diagnostic assays for, osteosarcoma and other cell proliferative disorders.

For example, SEQ ID NO:94 showed decreased expression in activated Jurkat cells versus untreated Jurkat cells as determined by microarray analysis. Jurkat cells are used as a model for signaling in human T cells. Jurkat cells were treated with combinations of graded doses of PMA (5 nM-1 μM) and ionomycin (50 ng/ml-10μ/ml). Treatment with PMA and ionomycin mimics secondary signaling during optimal B cell activation. Cells were collected at a 1 hour time point. The treated cells were compared to untreated Jurkat cells kept in culture in the absence of stimuli. Therefore, SEQ ID NO:94 is useful in monitoring treatment of, and diagnostic assays for, autoimmune/inflammation disorders.

SEQ ID NO:96 showed decreased expression in C3A cells treated with steroids versus untreated C3A cells, as determined by microarray analysis. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor III receptor; ii) secretion of a high ratio of serum albumin compared with α-fetoprotein iii) conversion of ammonia to urea and glutamine; iv) metabolism of aromatic amino acids; and v) proliferation in glucose-free and insulin-free medium. The C3A cell line is now well established as an in vitro model of the mature human liver (Mickelson et al. (1995) Hepatology 22:866-875; Nagendra et al. (1997) Am. J. Physiol. 272:G408-G416). In one experiment, early confluent C3A cells were treated with dexamethasone at 1, 10, and 100 μM for 1, 3, and 6 hours. In a second experiment, early confluent C3A cells were treated with medroxyprogesterone (MAH) at 1, 10, and 100 μM for 1, 3, and 6 hours. In a third experiment, early confluent C3A cells were treated with beclomethasone at 1, 10, and 100 μM for 1, 3, and 6 hours. The treated cells were compared to untreated early confluent C3A cells. Therefore, SEQ ID NO:96 is useful in monitoring treatment of, and diagnostic assays for, autoimmune/inflammation disorders.

For example, SEQ ID NO:101 and SEQ ID NO:102 showed differential expression associated with breast cancer as determined by microarray analysis. Gene expression profiles of nonmalignant mammary epithelial cell lines, HEMEC and MCF10A, were compared to the gene expression profiles of breast carcinoma cell lines representing different stages of tumor progression. The cell lines compared included: a) T-47D, a breast carcinoma cell line derived from a pleural effusion from a 54-year old female with an infiltrating ductal carcinoma of the breast, b) Sk-BR3, a breast adenocarcinoma cell line isolated from a malignant pleural effusion of a 43-year old female, c) MDA-mb435S and MDA-mb-231, metastatic breast tumor cell lines derived from the pleural effusion of a 51-year old female with metastatic breast carcinoma, d) BT-20, a breast carcinoma cell line derived in vitro from the cells emigrating out of thin slices of tumor mass isolated from a 74-year-old female, e) MCF7, a nonmalignant breast adenocarcinoma cell line isolated from the pleural effusion of a 69-year-old female, f) MCF-10A, a breast mammary gland (luminal ductal characteristics) cell line isolated from a 36-year-old woman with fibrocystic breast disease, g) BT-483, a breast ductal carcinoma cell line that was isolated from a papillary invasive ductal tumor obtained from a 23-year-old normal, menstruating, parous female with a family history of breast cancer, h) Hs 578T, a breast ductal carcinoma cell line isolated from a 74-year-old female with breast carcinoma, i) HMEC, a primary breast epithelial cell line isolated from a normal donor. The expression of SEQ ID NO:101 and SEQ ID NO:102 was at least two-fold higher in the breast carcinoma cell lines, T-47D, SK-BR3, MCF7, BT-483, and Hs578T, compared to the noncancerous HMEC or MCF10A cell lines.

In addition SEQ ID NO:101 and SEQ ID NO:102 showed differential expression associated with lung cancer. The expression of SEQ ID NO:101 and SEQ ID NO:102 was compared in normal and cancerous tissue samples from a patient with lung adenocarcinoma. SEQ ID NO:101 and SEQ ID NO:102 showed at least a two-fold decrease in expression in cancerous lung tissue compared to matched microscopically normal tissue from the same donor as determined by microarray analysis.

SEQ ID NO:101 and SEQ ID NO:102 also showed differential expression associated with colon polyps. The expression of SEQ ID NO:101 and SEQ ID NO:102 was compared in normal and polyp tissue. SEQ ID NO:101 and SEQ ID NO:102 showed at least a two-fold decrease in expression in colon polyps compared to matched microscopically normal tissue from the same donor as determined by microarray analysis. Therefore SEQ ID NO:101 and SEQ ID NO:102 are useful in diagnostic assays and disease staging assays for cell proliferative disorders, including breast cancer, lung cancer, and colon polyposis.

SEQ ID NO:101 and SEQ ID NO:102 also showed decreased expression in human umbilical vein endothelial cells (HUVECs) exposed to tumor necrosis factor alpha (TNF-α), versus untreated HUVECs as determined by microarray analysis. HUVECs were grown to 85% confluence and then treated with TNF-alpha for 0.33, 0.66, 1, 4, 8, 24, 48, and 72 hours. These TNF-α treated cells were compared to untreated HUVECs collected at 85% confluence (0 hour). Therefore, SEQ ID NO:101 and SEQ ID NO:102 are useful in monitoring treatment of, and diagnostic assays for, autoimmune/inflammation disorders.

In another example, SEQ ID NO:104 showed decreased expression associated with cell proliferative disorders, including breast carcinoma, prostate carcinoma, familial polyposis syndrome (FAP), and colon adenocarcinoma. Expression of SEQ ID NO:104 in breast carcinoma cell lines MCF7, T-47D, Sk-BR-3, BT-20, and MDA-mb-435S was compared to that in HMEC cells. SEQ ID NO:104 showed at least two-fold lower expression in all of the tested carcinoma cell lines compared to the HMEC cell line. SEQ ID NO:104 also showed at least two-fold lower expression in MDA-mb-231, MCF7, BT-20, Sk-BR-3, and T-47D breast carcinoma cell lines compared to the nonmalignant MCF-10A cell line. Furthermore, expression of SEQ ID NO:104 was compared in normal and cancerous tissue samples from a patient with breast lobular carcinoma. SEQ ID NO:104 showed at least a two-fold decrease in expression in cancerous breast tissue compared to matched microscopically normal tissue from the same donor as determined by microarray analysis. Therefore SEQ ID NO:104 is useful in diagnostic assays and disease staging assays for breast cancer.

In addition, gene expression profiles of SEQ ID NO:104 in the prostate carcinoma lines CA-HPV-10, LNCaP, PC-3, and DU 145, MDAPCa2b were compared to that in nontumorigenic prostate epithelial PrEC or PZ-HPV-7 cell lines. PZ-HPV-7 was derived from epithelial cells cultured from normal tissue from the peripheral zone of the prostate. CA-HPV-10 was derived from cells from a 63-year-old male with prostatic adenocarcinoma of Gleason Grade 4/4. DU 145 is a prostate carcinoma cell line isolated from a metastatic site in the brain of 69-year old male with widespread metastatic prostate carcinoma. LNCaP is a prostate carcinoma cell line isolated from a lymph node biopsy of a 50-year-old male with metastatic prostate carcinoma. PC-3 is a prostate adenocarcinoma cell line that was isolated from a metastatic site in the bone of a 62-year-old male with grade IV prostate adenocarcinoma. MDAPCa2b is a prostate adenocarcinoma cell line isolated from a metastatic site in the bone of a 63-year-old male. SEQ ID NO:104 showed at least a two-fold decrease in expression in MDAPCa2b and LNCaP cell lines compared to PrEC or PZ-HPV-7 cell lines. Therefore, SEQ ID NO:104 is useful in diagnostic assays and disease staging assays for prostate cancer.

In addition, gene expression of SEQ ID NO:104 in normal colon tissue was compared to that in two colon polyps from a 23-year-old donor diagnosed with FAP and colon adenocarcinoma (Huntsman Cancer Institute, Salt Lake City, Utah). SEQ ID NO:104 showed at least a two-fold decrease in expression in colon polyp tissue compared to matched microscopically normal tissue from the same donor as determined by microarray analysis. Therefore, SEQ ID NO:104 is useful in monitoring treatment of, and diagnostic assays for prostate cancer and colon polyposis.

In another example, SEQ ID NO:104 showed decreased expression in preadipocytes exposed to differentiation factors versus untreated preadipocytes as determined by microarray analysis. Human preadipocytes were treated with human insulin and PPAR-γ agonist for 3 days followed by further treatment with insulin. Differentiated adipocytes were compared to untreated preadipocytes maintained in culture in the absence of inducing agents. Therefore, SEQ ID NO:104 is useful in monitoring treatment of and diagnostic assays for disorders associated with adipocyte differentiation, including obesity, type II diabetes, lipodystrophy, and hyperinsulinemia.

In another example, SEQ ID NO:107 showed differential expression associated with breast cancer. SEQ ID NO:107 showed at least two-fold higher expression in MDA-mb-231, Sk-BR-3, and T-47D breast carcinoma cell lines compared to the HMEC cell line. Therefore SEQ ID NO:107 is useful in diagnostic assays and disease staging assays for breast cancer.

In another example, SEQ ID NO:108 showed at least two-fold decreased expression in tissue affected by cancer versus normal tissue, as determined by microarray analysis. Cancers studied include lung adenocarcinoma, endometrial adenocarcinoma, and ovarian cancer. In one experiment moderately differentiated adenocarcinoma of the right lung was compared to grossly uninvolved lung tissue from a 60 year-old donor. (Huntsman Cancer Institute). In a second experiment, endometrial cancer (adenocarcinoma) was compared to grossly uninvolved endometrial tissue from the same donor, a 72 year-old female (Huntsman Cancer Institute). Cervical tissue from the same donor was compared to endometrium. In a third experiment, normal ovarian tissue from a 79 year-old female donor was compared to an ovarian tumor from the same donor. Therefore, SEQ ID NO:108 is useful in monitoring treatment of, and diagnostic assays for, lung adenocarcinoma, endometrial adenocarcinoma, ovarian tumor, and other cell proliferative disorders.

The expression of SEQ ID NO:113 was found to be differentially expressed in human lung tumor tissue as compared to normal lung tissue from the same donor in several pair-comparison studies. SEQ ID NO:113 was underexpressed by at least two-fold in lung tumor tissue as compared to normal lung tissues in all pairs tested. SEQ ID NO:113 was also differentially expressed in vascular tissue These experiments indicate that SEQ ID NO:113 exhibited significant differential expression patterns using microarray techniques, and further establishes its utility as a diagnostic marker or therapeutic agent which may be useful in a variety of conditions and diseases involving lung cancer and vascular tissue.

XII. Complementary Polynucleotides

Sequences complementary to the CGDD-encoding sequences, or any parts thereof, are used to detect, decrease, or inhibit expression of naturally occurring CGDD. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of CGDD. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the CGDD-encoding transcript.

XIII. Expression of CGDD

Expression and purification of CGDD is achieved using bacterial or virus-based expression systems. For expression of CGDD in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express CGDD upon induction with isopropyl beta-D-thiogalactopyranoside (IPTG). Expression of CGDD in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding CGDD by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. NatI. Acad. Sci. USA 91:3224-3227; Sandig, V. et al. (1996) Hum. Gene Ther. 7:1937-1945).

In most expression systems, CGDD is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from CGDD at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified CGDD obtained by these methods can be used directly in the assays shown in Examples XVII, and XIII, where applicable.

XIV. Functional Assays

CGDD function is assessed by expressing the sequences encoding CGDD at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of CGDD on gene expression can be assessed using highly purified populations of cells transfected with sequences encoding CGDD and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding CGDD and other genes of interest can be analyzed by northern analysis or microarray techniques.

XV. Production of CGDD Specific Antibodies

CGDD substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the CGDD amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-CGDD activity by, for example, binding the peptide or CGDD to a substrate, blocking with 1% BSA, reacting with rabbit antisera, washing, and reacting with radio-iodinated goat anti-rabbit IgG.

XVI. Purification of Naturally Occurring CGDD Using Specific Antibodies

Naturally occurring or recombinant CGDD is substantially purified by immunoaffinity chromatography using antibodies specific for CGDD. An immunoaffinity column is constructed by covalently coupling anti-CGDD antibody to an activated chromatographic resin, such as CNBr-activated SEPHAROSE (Amersham Biosciences). After the coupling, the resin is blocked and washed according to the manufacturer's instructions.

Media containing CGDD are passed over the immunoaffinity column, and the column is washed under conditions that allow the preferential absorbance of CGDD (e.g., high ionic strength buffers in the presence of detergent). The column is eluted under conditions that disrupt antibody/CGDD binding (e.g., a buffer of pH 2 to pH 3, or a high concentration of a chaotrope, such as urea or thiocyanate ion), and CGDD is collected.

XVII. Identification of Molecules which Interact with CGDD

CGDD, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled CGDD, washed, and any wells with labeled CGDD complex are assayed. Data obtained using different concentrations of CGDD are used to calculate values for the number, affinity, and association of CGDD with the candidate molecules.

Alternatively, molecules interacting with CGDD are analyzed using the yeast two-hybrid system as described in Fields, S. and O. Song (1989; Nature 340:245-246), or using commercially available kits based on the two-hybrid system, such as the MATCHMAKER system (Clontech).

CGDD may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of CGDD Activity

CGDD activity is demonstrated by measuring the induction of terminal differentiation or cell cycle progression when CGDD is expressed at physiologically elevated levels in mammalian cell culture systems. cDNA is subcloned into a mammalian expression vector containing a strong promoter that drives high levels of cDNA expression. Vectors of choice include PCMV SPORT (Life Technologies, Gaithersburg, Md.) and PCR 3.1 (Invitrogen, Carlsbad, Calif.), both of which contain the cytomegalovirus promoter. 5-10 μg of recombinant vector are transiently transfected into a human cell line, preferably of endothelial or hematopoietic origin, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP) (Clontech, Palo Alto, Calif.), CD64, or a CD64-GFP fusion protein. Flow cytometry detects and quantifies the uptake of fluorescent molecules that diagnose events preceding or coincident with cell cycle progression or terminal differentiation. These events include changes in nuclear DNA content as measured by staining of DNA with propidium iodide; changes in cell size and granularity as measured by forward light scatter and 90 degree side light scatter; up or down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York, N.Y.

Alternatively, an in vitro assay for CGDD activity measures the transformation of normal human fibroblast cells overexpressing antisense CGDD RNA (Garkavtsev, I. and K. Riabowol (1997) Mol. Cell Biol. 17:2014-2019). cDNA encoding CGDD is subcloned into the pLNCX retroviral vector to enable expression of antisense CGDD RNA. The resulting construct is transfected into the ecotropic BOSC23 virus-packaging cell line. Virus contained in the BOSC23 culture supernatant is used to infect the amphotropic CAK8 virus-packaging cell line. Virus contained in the CAK8 culture supernatant is used to infect normal human fibroblast (Hs68) cells. Infected cells are assessed for the following quantifiable properties characteristic of transformed cells: growth in culture to high density associated with loss of contact inhibition, growth in suspension or in soft agar, formation of colonies or foci, lowered serum requirements, and ability to induce tumors when injected into immunodeficient mice. The activity of CGDD is proportional to the extent of transformation of Hs68 cells.

Alternatively, CGDD can be expressed in a mammalian cell line by transforming the cells with a eukaryotic expression vector encoding CGDD. Eukaryotic expression vectors are commercially available, and the techniques to introduce them into cells are well known to those skilled in the art. To assay the cellular localization of CGDD, cells are fractionated as described by Jiang, H. P. et al. (1992; Proc. Natl. Acad. Sci. 89:7856-7860). Briefly, cells pelleted by low-speed centrifugation are resuspended in buffer (10 mM TRIS-HCl, pH 7.4/10 mM NaCl/3 mM MgCl₂/5 mM EDTA with 10 ug/ml aprotinin, 10 ug/ml leupeptin, 10 ug/ml pepstatin A, 0.2 mM phenylmethylsulfonyl fluoride) and homogenized. The homogenate is centrifuged at 600×g for 5 minutes. The particulate and cytosol fractions are separated by ultracentrifugation of the supernatant at 100,000×g for 60 minutes. The nuclear fraction is obtained by resuspending the 600×g pellet in sucrose solution (0.25 M sucrose/10 mM TRIS-HCl, pH 7.4/2 mM MgCl₂) and recentrifuged at 600×g. Equal amounts of protein from each fraction are applied to an SDS/10% polyacrylamide gel and blotted onto membranes. Western blot analysis is performed using CGDD anti-serum. The localization of CGDD is assessed by the intensity of the corresponding band in the nuclear fraction relative to the intensity in the other fractions. Alternatively, the presence of CGDD in cellular fractions is examined by fluorescence microscopy using a fluorescent antibody specific for CGDD.

Alternatively, CGDD activity may be demonstrated as the ability to interact with its associated Ras superfamily protein, in an in vitro binding assay. The candidate Ras superfamily proteins are expressed as fusion proteins with glutathione S-transferase (GST), and purified by affinity chromatography on glutathione-Sepharose. The Ras superfamily proteins are loaded with GDP by incubating 20 mM Tris buffer, pH 8.0, containing 100 mM NaCl, 2 mM EDTA, 5 mM MgCl2, 0.2 mM DTT, 100 μM AMP-PNP and 10 μM GDP at 30° C. for 20 minutes. CGDD is expressed as a FLAG fusion protein in a baculovirus system. Extracts of these baculovirus cells containing CGDD-FLAG fusion proteins are precleared with GST beads, then incubated with GST-Ras superfamily fusion proteins. The complexes formed are precipitated by glutathione-Sepharose and separated by SDS-polyacrylamide gel electrophoresis. The separated proteins are blotted onto nitrocellulose membranes and probed with commercially available anti-FLAG antibodies. CGDD activity is proportional to the amount of CGDD-FLAG fusion protein detected in the complex.

Alternatively, as demonstrated by Li and Cohen (Li, L. and S. N. Cohen (1995) Cell 85:319-329), the ability of CGDD to suppress tumorigenesis can be measured by designing an antisense sequence to the 5′ end of the gene and transfecting NIH 3T3 cells with a vector transcribing this sequence. The suppression of the endogenous gene will allow transformed fibroblasts to produce clumps of cells capable of forming metastatic tumors when introduced into nude mice.

Alternatively, an assay for CGDD activity measures the effect of injected CGDD on the degradation of maternal transcripts. Procedures for oocyte collection from Swiss albino mice, injection, and culture are as described in Stutz et al., (supra). A decrease in the degradation of maternal RNAs as compared to control oocytes is indicative of CGDD activity. In the alternative, CGDD activity is measured as the ability of purified CGDD to bind to RNAse as measured by the assays described in Example XVII.

Alternatively, an assay for CGDD activity measures syncytium formation in COS cells transfected with an CGDD expression plasmid, using the two-component fusion assay described in Mi (supra). This assay takes advantage of the fact that human interleukin 12 (IL-12) is a heterodimer comprising subunits with molecular weights of 35 kD (p35) and 40 kD (p40). COS cells transfected with expression plasmids carrying the gene for p35 are mixed with COS cells cotransfected with expression plasmids carrying the genes for p40 and CGDD. The level of IL-12 activity in the resulting conditioned medium corresponds to the activity of CGDD in this assay. Syncytium formation may also be measured by light microscopy (Mi et al., supra).

An alternative assay for CGDD activity measures cell proliferation as the amount of newly initiated DNA synthesis in Swiss mouse 3T3 cells. A plasmid containing polynucleotides encoding CGDD is transfected into quiescent 3T3 cultured cells using methods well known in the art. The transiently transfected cells are then incubated in the presence of [³H]thymidine or a radioactive DNA precursor such as [α³²P]ATP. Where applicable, varying amounts of CGDD ligand are added to the transfected cells. Incorporation of [³H]thymidine into acid-precipitable DNA is measured over an appropriate time interval, and the amount incorporated is directly proportional to the amount of newly synthesized DNA and CGDD activity.

Alternatively, CGDD activity is measured by the cyclin-ubiquitin ligation assay (Townsley, F. M. et al. (1997) Proc. Natl. Acad. Sci. USA 94:2362-2367). The reaction contains in a volume of 10 μl, 40 mM Tris.HCl (pH 7.6), 5 MM MgCl₂, 0.5 mM ATP, 10 mM phosphocreatine, 50 μg of creatine phosphokinase/ml, 1 mg reduced carboxymethylated bovine serum alburin/ml, 50 μM ubiquitin, 1 μM ubiquitin aldehyde, 1-2 pmol ¹²⁵I-labeled cyclin B, 1 pmol E1, 1 μM okadaic acid, 10 μg of protein of M-phase fraction 1A (containing active E3-C and essentially free of E2-C), and varying amounts of CGDD. The reaction is incubated at 18° C. for 60 minutes. Samples are then separated by electrophoresis on an SDS polyacrylamide gel. The amount of ¹²⁵I-cyclin-ubiquitin formed is quantified by PHOSPHORIMAGER analysis. The amount of cyclin-ubiquitin formation is proportional to the activity of CGDD in the reaction.

Alternatively, an assay for CGDD activity uses radiolabeled nucleotides, such as [α³²P]ATP, to measure either the incorporation of radiolabel into DNA during DNA synthesis, or fragmentation of DNA that accompanies apoptosis. Manmalian cells are transfected with plasmid containing cDNA encoding CGDD by methods well known in the art. Cells are then incubated with radiolabeled nucleotide for various lengths of time. Chromosomal DNA is collected, and radioactivity is detected using a scintillation counter. Incorporation of radiolabel into chromosomal DNA is proportional to the degree of stimulation of the cell cycle. To determine if CGDD promotes apoptosis, chromosomal DNA is collected as above, and analyzed using polyacrylamide gel electrophoresis, by methods well known in the art. Fragmentation of DNA is quantified by comparison to untransfected control cells, and is proportional to the apoptotic activity of CGDD.

Alternatively, cyclophilin activity of CGDD is measured using a chymotrypsin-coupled assay to measure the rate of cis to trans interconversion (Fischer, G. et al. (1984) Biomed. Biochim. Acta 43:1101-1111). The chymotrypsin is used to estimate the trans-substrate cleavage activity at Xaa-Pro peptide bonds, wherein the rate constant for the cis to trans isomerization can be obtained by measuring the rate constant of the substrate hydrolysis at the slow phase. Samples are incubated in the presence or absence of the immunosuppressant drugs CsA or FK506, reactions initiated by addition of chymotrypsin, and the fluorescent reaction measured. The enzymatic rate constant is calculated from the equation kₐₚₚ=k_{H20}+k_{enz}, wherein first order kinetics are displayed, and where one unit of PPIase activity is defined as k_{enz}(s⁻¹).

Alternatively, cyclophilin activity of CGDD is monitored by a quantitative immunoassay that measures its affinity for stereospecific binding to the immunosuppressant drug cyclosporin (Quesniaux, V. F. et al. (1987) Eur. J. Immunol. 17:1359-1365). In this assay, the cyclophilin-cyclosporin complex is coated on a solid phase, with binding detected using anti-cyclophilin rabbit antiserum enhanced by an antiglobulin-enzyme conjugate.

Alternatively, activity of CGDD is monitored by a binding assay developed to measure the non-covalent binding between FKBPs and immunosuppressant drugs in the gas phase using electrospray ionization mass spectrometry (Trepanier, D. J. et al. (1999) Ther. Drug Monit. 21:274-280). In electrospray ionization, ions are generated by creating a fine spray of highly charged droplets in the presence of a strong electric field; as the droplet decreases in size, the charge density on the surface increases. Ions are electrostatically directed into a mass analyzer, where ions of opposite charge are generated in spatially separate sources and then swept into capillary inlets where the flows are merged and where reactions occur. By comparing the charge states of bound versus unbound CGDD/immunosuppressive drug complexes, relative binding affinities can be established and correlated with in vitro binding and immunosuppressive activity.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | Incyte | | Incyte | Incyte |
|---|---|---|---|---|---|
| Incyte | Polypeptide | Polypeptide | Polynucleotide | Polynucleotide | Full Length |
| Project ID | SEQ ID NO: | ID | SEQ ID NO: | ID | Clones |
| 1318936 | 1 | 1318936CD1 | 58 | 1318936CB1 | |
| 033784 | 2 | 033784CD1 | 59 | 033784CB1 | 5800088CA2, |
| | | | | | 8168280CA2, |
| | | | | | 90133301CA2, |
| | | | | | 90133317CA2, |
| | | | | | 90133325CA2, |
| | | | | | 90133341CA2, |
| | | | | | 90133401CA2, |
| | | | | | 90133417CA2, |
| | | | | | 90133441CA2 |
| 4160218 | 3 | 4160218CD1 | 60 | 4160218CB1 | |
| 7501989 | 4 | 7501989CD1 | 61 | 7501989CB1 | 2722492CA2 |
| 7500695 | 5 | 7500695CD1 | 62 | 7500695CB1 | |
| 7502088 | 6 | 7502088CD1 | 63 | 7502088CB1 | |
| 7198825 | 7 | 7198825CD1 | 64 | 7198825CB1 | |
| 71863981 | 8 | 71863981CD1 | 65 | 71863981CB1 | |
| 7500513 | 9 | 7500513CD1 | 66 | 7500513CB1 | 90021353CA2, |
| | | | | | 90021385CA2, |
| | | | | | 90021453CA2, |
| | | | | | 90021571CA2 |
| 7501080 | 10 | 7501080CD1 | 67 | 7501080CB1 | |
| 3144878 | 11 | 3144878CD1 | 68 | 3144878CB1 | |
| 7502112 | 12 | 7502112CD1 | 69 | 7502112CB1 | |
| 7502788 | 13 | 7502788CD1 | 70 | 7502788CB1 | |
| 7500171 | 14 | 7500171CD1 | 71 | 7500171CB1 | |
| 7500172 | 15 | 7500172CD1 | 72 | 7500172CB1 | 90066751CA2, |
| | | | | | 90151039CA2, |
| | | | | | 90151047CA2 |
| 7500899 | 16 | 7500899CD1 | 73 | 7500899CB1 | 90156633CA2, |
| | | | | | 90156733CA2 |
| 7500916 | 17 | 7500916CD1 | 74 | 7500916CB1 | 2418490CA2 |
| 7502479 | 18 | 7502479CD1 | 75 | 7502479CB1 | 1901027CA2, |
| | | | | | 5721658CA2, |
| | | | | | 8725531CA2, |
| | | | | | 8725532CA2, |
| | | | | | 90184011CA2, |
| | | | | | 90184019CA2, |
| | | | | | 90184043CA2, |
| | | | | | 90184103CA2, |
| | | | | | 90184111CA2, |
| | | | | | 90184119CA2, |
| | | | | | 90184127CA2, |
| | | | | | 90184135CA2, |
| | | | | | 90184143CA2 |
| 3015057 | 19 | 3015057CD1 | 76 | 3015057CB1 | |
| 6303570 | 20 | 6303570CD1 | 77 | 6303570CB1 | |
| 7501579 | 21 | 7501579CD1 | 78 | 7501579CB1 | 6179508CA2 |
| 7503812 | 22 | 7503812CD1 | 79 | 7503812CB1 | |
| 7503970 | 23 | 7503970CD1 | 80 | 7503970CB1 | 1399685CA2 |
| 2578986 | 24 | 2578986CD1 | 81 | 2578986CB1 | 6825193CA2 |
| 7503786 | 25 | 7503786CD1 | 82 | 7503786CB1 | 90174724CA2 |
| 7505684 | 26 | 7505684CD1 | 83 | 7505684CB1 | 4656026CA2, |
| | | | | | 90177755CA2, |
| | | | | | 90178450CA2, |
| | | | | | 90178574CA2, |
| | | | | | 90178590CA2, |
| | | | | | 90178651CA2, |
| | | | | | 90178659CA2, |
| | | | | | 90178667CA2, |
| | | | | | 90178691CA2, |
| | | | | | 90178751CA2, |
| | | | | | 90178759CA2, |
| | | | | | 90178775CA2, |
| | | | | | 90178783CA2, |
| | | | | | 90178791CA2 |
| 302655 | 27 | 302655CD1 | 84 | 302655CB1 | 5281285CA2 |
| 4757551 | 28 | 4757551CD1 | 85 | 4757551CB1 | |
| 7503157 | 29 | 7503157CD1 | 86 | 7503157CB1 | |
| 7503702 | 30 | 7503702CD1 | 87 | 7503702CB1 | 90185662CA2 |
| 72096217 | 31 | 72096217CD1 | 88 | 72096217CB1 | |
| 7503620 | 32 | 7503620CD1 | 89 | 7503620CB1 | |
| 7503621 | 33 | 7503621CD1 | 90 | 7503621CB1 | |
| 7503624 | 34 | 7503624CD1 | 91 | 7503624CB1 | |
| 7503625 | 35 | 7503625CD1 | 92 | 7503625CB1 | |
| 7503627 | 36 | 7503627CD1 | 93 | 7503627CB1 | |
| 7505744 | 37 | 7505744CD1 | 94 | 7505744CB1 | 90032204CA2, |
| | | | | | 90032212CA2, |
| | | | | | 90032220CA2, |
| | | | | | 90032304CA2, |
| | | | | | 90032312CA2, |
| | | | | | 90032344CA2 |
| 7503712 | 38 | 7503712CD1 | 95 | 7503712CB1 | |
| 7503713 | 39 | 7503713CD1 | 96 | 7503713CB1 | |
| 7502225 | 40 | 7502225CD1 | 97 | 7502225CB1 | |
| 7502778 | 41 | 7502778CD1 | 98 | 7502778CB1 | 90170542CA2, |
| | | | | | 90170642CA2 |
| 7503699 | 42 | 7503699CD1 | 99 | 7503699CB1 | 90176937CA2 |
| 6026715 | 43 | 6026715CD1 | 100 | 6026715CB1 | 6026715CA2 |
| 7503631 | 44 | 7503631CD1 | 101 | 7503631CB1 | |
| 7503664 | 45 | 7503664CD1 | 102 | 7503664CB1 | |
| 7504812 | 46 | 7504812CD1 | 103 | 7504812CB1 | |
| 7504996 | 47 | 7504996CD1 | 104 | 7504996CB1 | 90032451CA2 |
| 7377279 | 48 | 7377279CD1 | 105 | 7377279CB1 | |
| 2060984 | 49 | 2060984CD1 | 106 | 2060984CB1 | 6637745CA2 |
| 4085993 | 50 | 4085993CD1 | 107 | 4085993CB1 | |
| 6756305 | 51 | 6756305CD1 | 108 | 6756305CB1 | |
| 7504842 | 52 | 7504842CD1 | 109 | 7504842CB1 | 90069638CA2 |
| 7500901 | 53 | 7500901CD1 | 110 | 7500901CB1 | 90133480CA2 |
| 7500898 | 54 | 7500898CD1 | 111 | 7500898CB1 | 6312095CA2, |
| | | | | | 90146901CA2, |
| | | | | | 90156709CA2, |
| | | | | | 90156741CA2 |
| 7500912 | 55 | 7500912CD1 | 112 | 7500912CB1 | 4005381CA2, |
| | | | | | 5699251CA2, |
| | | | | | 5771654CA2, |
| | | | | | 90147007CA2, |
| | | | | | 90147031CA2, |
| | | | | | 90147203CA2, |
| | | | | | 90179571CA2 |
| 7505851 | 56 | 7505851CD1 | 113 | 7505851CB1 | 2418490CA2 |
| 7503696 | 57 | 7503696CD1 | 114 | 7503696CB1 | |

TABLE 2

| Poly- | | | | |
|---|---|---|---|---|
| pep- | | GenBank | | |
| tide | Incyte | ID NO: or | Proba- | |
| SEQ | Polypeptide | PROTEOME | bility | |
| ID NO: | ID | ID NO: | Score | Annotation |
| 1 | 1318936CD1 | g14595658 | 2.3E−155 | [Xenopus laevis] (AF387815) LIM protein prickle |
| 2 | 33784CD1 | g3719323 | 1.3E−26 | [Perca flavescens] D7-like protein |
| 3 | 4160218CD1 | g12751141 | 8.0E−39 | [Homo sapiens] B aggressive lymphoma short isoform (Aguiar, R. C. T. et al. |
| | | | | (2000) Blood 96 (13), 4328-4334) |
| 4 | 7501989CD1 | g7670836 | 7.2E−280 | [Homo sapiens] hepatocellular carcinoma-associated antigen 66 |
| 5 | 7500695CD1 | g2213934 | 2.7E−89 | [Homo sapiens] cancer associated surface antigen Nakashima, M. et al. (1999) |
| | | | | Inhibition of cell growth and induction of apoptotic cell death by the human tumor |
| | | | | associated antigen RCAS1. Nat. Med. 5: 938-942 |
| 6 | 7502088CD1 | g929615 | 1.2E−96 | [Homo sapiens] BCL7A Zani, V. J. et al. (1996) Molecular cloning of complex |
| | | | | chromosomal translocation t(8; 14; 12) (q24.1; q32.3; q24.1) in a Burkitt |
| | | | | lymphoma cell line defines a new gene (BCL7A) with homology to caldesmon. |
| | | | | Blood 87: 3124-3134 |
| 7 | 7198825CD1 | g10178967 | 3.8E−100 | [Mus musculus] stretch response protein 553 |
| 8 | 71863981CD1 | g4587965 | 0.0 | [Homo sapiens] JAW1-related protein MRVI1A long isoform (Shaughnessy, J. D. |
| | | | | Jr. (1999)Oncogene 18: 2069-2084) |
| | | 430036\|Mrvi1 | 0.0 | [Mus musculus][Endoplasmic reticulum; Cytoplasmic] Putative tumor suppressor |
| | | | | that possibly functions in myeloid cell differentiation and/or growth control; |
| | | | | corresponding gene is disrupted by non-ecotropic MRV viruses in BXH2 |
| | | | | leukemias. |
| | | 343110\|MRVI1 | 0.0 | [Homo sapiens][Endoplasmic reticulum; Cytoplasmic] Protein with similarity to |
| | | | | human MLRP, which is a membrane protein expressed in the endoplasmic |
| | | | | reticulum of lymphoid cells; gene is a target for murine retrovirus integration in |
| | | | | leukemia, suggesting it may act as a tumor suppressor |
| | | 343510\|LRMP | 2.5E−30 | [Homo sapiens][Endoplasmic reticulum; Cytoplasmic; Plasma membrane] |
| | | | | Transmembrane protein that has similarity to proteins involved in vesicle targeting |
| | | | | and fusion, localized to the cytoplasmic side of the endoplasmic reticulum, |
| | | | | expression is developmentally regulated in lymphocytes |
| | | 319044\|Lrmp | 4.0E−26 | [Mus musculus][Endoplasmic reticulum; Cytoplasmic] Transmembrane protein |
| | | | | that has similarity to proteins involved in vesicle targeting and fusion, localized to |
| | | | | the endoplasmic reticulum, expression is developmentally regulated in |
| | | | | lymphocytes |
| 9 | 7500513CD1 | g1932712 | 1.5E−73 | [Homo sapiens] prostate carcinoma tumor antigen (Hadari, Y. R (1995) J. Biol. |
| | | | | Chem. 270: 3447-3453) |
| | | 346874\|LGALS8 | 5.9E−74 | [Homo sapiens][Small molecule-binding protein][Cytoplasmic; Extracellular |
| | | | | (excluding cell wall)] Galectin 8, member of a family of lectins that bind to beta |
| | | | | galactoside and are involved in biological processes such as cell adhesion, cell |
| | | | | growth regulation, inflammation, immunomodulation, apoptosis and metastasis. |
| | | | | (Su, Z. Z. et al. Surface-epitope masking and expression cloning identifies the |
| | | | | human prostate carcinoma tumor antigen gene PCTA-1 a member of the galectin |
| | | | | gene family. Proc Natl. Acad. Sci. U.S.A. 93, 7252-7 (1996). |
| | | | | Gopalkrishnan, R. V. et al. Molecular characterization of prostate carcinoma |
| | | | | tumor antigen-1, PCTA-1, a human galectin-8 related gene. Oncogene 19, 4405-16 |
| | | 608278\|Lgals8 | 7.8E−63 | [Mus musculus][Small molecule-binding protein] Member of the galectin |
| | | | | galactoside-binding lectin family, has strong similarity to rat Rn. 10069 and human |
| | | | | LGALS8, galectin 8 |
| | | 329910\|Rn.10069 | 1.0E−62 | [Rattus norvegicus][Small molecule-binding protein][Cytoplasmic] Galectin 8, |
| | | | | member of a family of lectins that bind to beta galactoside and are involved in |
| | | | | biological processes such as cell adhesion, cell growth regulation, inflammation, |
| | | | | immunomodulation, apoptosis and metastasis |
| | | 418664\|Lgals4 | 9.3E−28 | [Mus musculus][Small molecule-binding protein] Galectin-4, a member of the |
| | | | | galectin family of lectins that bind to beta galactoside and are involved in |
| | | | | biological processes such as cell adhesion, cell growth regulation, inflammation, |
| | | | | immunomodulation, apoptosis and metastasis, expressed in intestine |
| | | 585199\|Lgals6 | 9.3E−28 | [Mus musculus][Small molecule-binding protein][Cytoplasmic]Galectin 6, |
| | | | | member of a family of lectins that bind to beta galactoside and are involved in cell |
| | | | | adhesion, cell growth regulation, inflammation, immunomodulation, apoptosis and |
| | | | | metastasis; expressed throughout the gastrointestinal tract |
| 10 | 7501080CD1 | g7637838 | 4.7E−206 | [Homo sapiens] programmed cell death 9 (Carim, L. et al. (1999) Cytogenet. Cell |
| | | | | Genet. 87: 85-88 |
| | | 570834\|PDCD9 | 4.2E−207 | [Homo sapiens] programmed cell death 9, homolog of chick pro-apoptotic protein |
| | | | | p52. (Carim, L. et al. supra) |
| | | 688536\|Pdcd9 | 1.0E−144 | [Mus musculus] Unknown protein has strong similarity to a region of human |
| | | | | PCD9 (programmed cell death 9), which is a homolog of the chick pro-apoptotic |
| | | | | protein p52 |
| 11 | 3144878CD1 | g12711386 | 2.2E−72 | [Rattus norvegicus] Ajuba protein |
| | | 433068\|D9Ertd192e | 1.6E−80 | [Mus musculus][Small molecule-binding protein] Protein containing three LIM |
| | | | | domains, which mediate protein-protein interactions, ubiquitously expressed |
| | | 434942\|LIMD1 | 1.3E−78 | [Homo sapiens][Small molecule-binding protein] Protein with LIM domains, |
| | | | | ubiquitously expressed |
| | | 429958\|Jub | 2.5E−73 | [Mus musculus][Activator][Cytoplasmic] Ajuba, a cytosolic LIM protein that |
| | | | | promotes MAP kinase activity, stimulates meiotic maturation when artificially |
| | | | | expressed in Xenopus oocytes |
| | | 342606\|LPP | 1.5E−50 | [Homo sapiens][DNA-binding protein] Member of the LIM family of proteins |
| | | | | which are involved in regulating development, protein-protein interactions and |
| | | | | perhaps binding to nucleic acids, contains a leucine-zipper motif and three LIM |
| | | | | domains |
| | | 430364\|Trip6 | 2.0E−48 | [Mus musculus][Ligand] Protein with very strong similarity to human TRIP6, |
| | | | | thyroid receptor interacting protein-6, which is a ligand-dependent LIM protein |
| | | | | that acts as a binding partner for the thyroid hormone receptor |
| 12 | 7502112CD1 | g10641052 | 6.2E−290 | [Mus musculus] neuronal differentiaion related protein (Kato, H. et al. (2000) J. |
| | | | | Biochem. 128: 923-932) |
| | 7502112CD1 | 710093\|Ndrp | 5.6E−291 | [Mus musculus] Neuronal differentiation related protein, has 6 WD repeats, |
| | | | | predominantly expressed in developing and regenerating neurons. (Millward, T. |
| | | | | et al. Molecular cloning and characterization of a conserved nuclear serine |
| | | | | (threonine) protein kinase. Proc. Natl. Acad. Sci. U.S.A. 92, 5022-6 (1995). |
| | 7502112CD1 | 342554\|KATNB1 | 1.0E−17 | [Homo sapiens] [Regulatory subunit; Hydrolase; ATPase] [Nuclear; Cytoplasmic; |
| | | | | Cytoskeletal; Centrosome/spindle pole body] Regulatory subunit of katanin, a |
| | | | | microtubule-stimulated ATPase that severs and disassembles microtubules into |
| | | | | tubulin dimers, subunit targets the enzyme to the centrosome |
| | 7502112CD1 | 377624\|pof11 | 1.4E−17 | [Schizosaccharomyces pombe][Protein conjugation factor] Protein containing |
| | | | | seven WD domains (WD-40 repeat) and an F-box domain, has low similarity to |
| | | | | human BTRC, which mediates endoplasmic reticulum-associated degradation of |
| | | | | human CD4 and I kappa B alpha by the ubiquitin pathway |
| | 7502112CD1 | 372635\|tup12 | 2.2E−17 | [Schizosaccharomyces pombe][Inhibitor or represser] Transcriptional represser |
| | | | | functioning redundantly with Tup11p; homolog of S. cerevisiae Tup1p |
| 13 | 7502788CD1 | g10641052 | 0.0 | [Mus musculus] neuronal differentiaion related protein (Kato, H. et al. (2000) J. |
| | | | | Biochem. 128: 923-932) |
| | 7502788CD1 | 710093\|Ndrp | 0.0 | [Mus musculus] Neuronal differentiation related protein, has 6 WD repeats, |
| | | | | predominantly expressed in developing and regenerating neurons |
| | 7502788CD1 | 377624\|pof11 | 2.6E−17 | [Schizosaccharomyces pombe][Protein conjugation factor] Protein containing |
| | | | | seven WD domains (WD-40 repeat) and an F-box domain, has low similarity to |
| | | | | human BTRC, which mediates endoplasmic reticulum-associated degradation of |
| | | | | human CD4 and Ikappa B alpha by the ubiquitin pathway |
| | 7502788CD1 | 372635\|tup12 | 3.0E−17 | [Schizosaccharomyces pombe][Inhibitor or represser] Transcriptional represser |
| | | | | functioning redundantly with Tup11p; homolog of S. cerevisiae Tup1p |
| | 7502788CD1 | 342554\|KATNB1 | 3.3E−17 | [Homo sapiens][Regulatory subunit; Hydrolase; ATPase][Nuclear; |
| | | | | Cytoplasmic; Cytoskeletal; Centrosome/spindle pole body] Regulatory subunit of |
| | | | | katanin, a microtubule-stimulated ATPase that severs and disassembles |
| | | | | microtubules into tubulin dimers, subunit targets the enzyme to the centrosome |
| 14 | 7500171CD1 | g21724162 | 1.0E−173 | [5′ incom][Homo sapiens] (AY039239) gastric cancer antigen Ga55 |
| | 7500171CD1 | 343832\|TACC1 | 1.9E−288 | [Homo sapiens] Protein localized to mitotic centrosomes, over expression causes |
| | | | | cell transformation. (Still, I. H. et al., supra) |
| | | | | Gergely, F. et al. The TACC domain identifies a family of centrosomal proteins |
| | | | | that can interact with microtubules Proc. Natl. Acad. Sci. U.S.A. 97, 14352-7 |
| | | | | (2000). |
| | 7500171CD1 | 623588\|TACC2 | 1.1E−104 | [Homo sapiens] Protein localized to centrosomes that acts as a tumor suppressor. |
| | | | | (Still, I. H. et al. Genomics 58, 165-70 (1999). |
| | 7500171CD1 | 343834\|TACC3 | 5.9E−57 | [Homo sapiens] Protein localized to mitoic centrosomes that may act to increase |
| | | | | microtubule number or stability; increased expression is associated with cancer. |
| | | | | (Gergely, F. et al. Proc. Natl. Acad. Sci. U.S.A. 97, 14352-7 (2000). |
| | 7500171CD1 | 430322\|Tacc3 | 2.8E−46 | [Mus musculus] Acidic coiled-coil protein, possibly involved in growth and |
| | | | | differentiation control |
| 15 | 7500172CD1 | g21724162 | 0.0 | [5′ incom][Homo sapiens] (AY039239) gastric cancer antigen Ga55 |
| | 7500172CD1 | g3435157 | 6.5E−176 | [Homo sapiens] TACC1 (Still, I. H. et al. (1999) Oncogene 18: 4032-4038) |
| | 7500172CD1 | 343832\|TACC1 | 5.8E−177 | [Homo sapiens] Protein localized to mitotic centrosomes, over expression causes |
| | | | | cell transformation |
| | 7500172CD1 | 623588\|TACC2 | 7.4E−83 | [Homo sapiens] Protein localized to centrosomes that acts as a tumor suppressor. |
| | | | | (Still, I. H. et al. supra) |
| | 7500172CD1 | 343834\|TACC3 | 4.8E−51 | [Homo sapiens] Protein localized to mitoic centrosomes that may act to increase |
| | | | | microtubule number or stability; increased expression is associated with cancer. |
| | | | | (Gergely, F. et al. supra) |
| | 7500172CD1 | 430322\|Tacc3 | 6.3E−45 | [Mus musculus] Acidic coiled-coil protein, possibly involved in growth and |
| | | | | differentiation control |
| 16 | 7500899CD1 | g10130019 | 1.8E−11 | [Homo sapiens] PIDD Lin, Y., et al (2000) Nat. Genet. 26: 122-127 Pidd, a new |
| | | | | death-domain-containing protein, is induced by p53 and promotes apoptosis |
| | 7500899CD1 | 424294\|KIAA0231 | 7.1E−14 | [Homo sapiens] Protein containing leucine rich repeats, which mediate protein- |
| | | | | protein interactions, has low similarity to regulators of the Ras pathway |
| 17 | 7500916CD1 | g14424787 | 1.2E−72 | [Homo sapiens] natural killer cell transcript 4 |
| | 7500916CD1 | 623570\|NK4 | 1.8E−73 | [Homo sapiens][Extracellular (excluding cell wall)] Protein with an RGD motif |
| | | | | that may play a role in cell adhesion, expressed by lymphocytes and upregulated |
| | | | | in mitogen-activated T cells and IL-2 treated NK cells |
| 18 | 7502479CD1 | g14250164 | 1.6E−64 | [Homo sapiens] Similar to RIKEN cDNA 2310030G06 gene |
| 19 | 3015057CD1 | g10120319 | 9.0E−133 | [Rattus norvegicus] kelch related protein 1 (Spence, H. J. et al. (2000) Krp1, a |
| | | | | novel kelch related protein that is involved in pseudopod elongation in |
| | | | | transformed cells. Oncogene 19: 1266-1276.) |
| | 3015057CD1 | 343178\|SARCOSIN | 5.7E−131 | [Homo sapiens] Protein expressed in sarcomeric muscle |
| | | | | (Taylor, A. et al. (1988) DNA sequence and muscle-specific expression of human |
| | | | | sarcosin transcripts. Mol. Cell. Biochem. 183: 105-112.) |
| | 3015057CD1 | 568936\|AB026190 | 1.2E−47 | [Homo sapiens][Cytoplasmic; Cytoskeletal] Kelch-like protein, a member of a |
| | | | | family of actin-organizing protein, contains a POZ (BTB) domain, which may |
| | | | | mediate protein-protein interactions, and Kelch motifs, highly expressed in neuron |
| | | | | cell bodies and neurite processes |
| | | | | (Nemes, J. P. et al. (2000) The SCA8 transcript is an antisense RNA to a brain- |
| | | | | specific transcript encoding a novel actin-binding protein (KLHL1). Hum. Mol. |
| | | | | Genet. 9: 1543-1551.) |
| 20 | 6303570CD1 | g179331 | 8.8E−35 | [Homo sapiens] B94 protein (Sarma, V. et al. (1992) Cloning of a novel tumor |
| | | | | necrosis factor-alpha-inducible primary response gene that is differentally |
| | | | | expressed in development and capillary tube-like formation in vitro. J. Immunol. |
| | | | | 148: 3302-3312.) |
| | 6303570CD1 | 343862\|TNFAIP2 | 7.7E−36 | [Homo sapiens][Extracellular (excluding cell wall)] Protein secreted by vascular |
| | | | | endothelium whose expression is induced by tumor necrosis factor alpha (TNF), |
| | | | | interleukin-1 beta, and lipopolysaccharide (Sarma, V. et al. (1992) supra.) |
| | 6303570CD1 | 321766\|Tnfaip2 | 4.1E−29 | [Mus musculus] Tumor necrosis factor alpha-induced protein, expressed in |
| | | | | vascular endothelium, embryonic cardiac tissues, acrosomes of mature sperm, and |
| | | | | cells of monocytic lineage |
| | | | | (Wolf, F. W. et al. (1994) B94, a primary response gene inducible by tumor |
| | | | | necrosis factor-alpha, is expressed in developing hematopoietic tissues and the |
| | | | | sperm acrosome. J. Biol. Chem. 269: 3633-3640; Sarma, V. et al. (1992) supra.) |
| 21 | 7501579CD1 | g15811123 | 1.1E−141 | [Homo sapiens] (AF308609) NDR1-related development protein NDR3 |
| | 7501579CD1 | 742714\|NDRG3 | 9.9E−143 | [Homo sapiens] Protein with high similarity to human NDRG1, which is induced |
| | | | | by nickel, homocysteine, 2-mercaptoethanol, and tunicamycin and is induced |
| | | | | during colon carcinoma cell line differentiation |
| | 7501579CD1 | 587185\|Ndr3 | 5.1E−139 | [Mus musculus] Member of the Ndr family, strongly expressed in the spinal cord |
| | | | | and thymus rudiment |
| | | | | (Okuda, T., and Kondoh, H. (1999) Identification of new genes ndr2 and ndr3 |
| | | | | which are related to Ndr1/RTP/Drg1 but show distinct tissue specificity and |
| | | | | response to N-myc. Biochem. Biophys. Res. Commun. 266: 208-215.) |
| 22 | 7503812CD1 | g951279 | 2.7E−216 | [Homo sapiens] MLN 64 (Tomasetto, C. et al. (1995) Identification of four novel |
| | | | | human genes amplified and overexpressed in breast carcinoma and localized to |
| | | | | the q11-q21.3 region of chromosome 17. Genomics 28: 367-376; Moog-Lutz, C. et |
| | | | | al. (1997) MLN64 exhibits homology with the steroidogenic acute regulatory |
| | | | | protein (STAR) and is over-expressed in human breast carcinomas. Int. J. Cancer |
| | | | | 71: 183-191.) |
| | 7503812CD1 | 428194\|MLN64 | 2.3E−217 | [Homo sapiens][Cytoplasmic] Protein that is over-expressed in malignant tissues, |
| | | | | contains a putative trans-membrane region and a StAR Homology Domain (SHD), |
| | | | | may function in steroidogenesis and contribute to tumor progression through |
| | | | | increased intra-tumoral steroidogenesis |
| | | | | (Watari, H. et al. (1997) MLN64 contains a domain with homology to the |
| | | | | steroidogenic acute regulatory protein (StAR) that stimulates steroidogenesis. |
| | | | | Proc. Natl. Acad. Sci. USA 94: 8462-8467; Tomasetto, C. et al. (1995) supra; |
| | | | | Moog-Lutz, C. et al. (1997) supra.) |
| | 7503812CD1 | 618940\|es64 | 4.3E−205 | [Mus musculus][Cytoplasmic] Protein with strong similarity to human MLN64, |
| | | | | which has similarity to the steroidogenic acute regulatory protein (STAR) and is |
| | | | | over-expressed in breast carcinomas |
| | | | | (Moog-Lutz, C. et al. (1997) supra.) |
| 23 | 7503970CD1 | g3435086 | 3.0E−248 | [Homo sapiens] RTN2-A (Roebroek, A. J. M. et al. (1998) cDNA cloning, |
| | | | | genomic organization, and expression of the human RTN2 gene, a member of a |
| | | | | gene family encoding reticulons. Genomics 51: 98-106; Senden, N. et al. (1997) |
| | | | | Neuroendocrine-specific protein (NSP)-reticulons as independent markers for non- |
| | | | | small cell lung cancer with neuroendocrine differentiation. An in vitro |
| | | | | histochemical study. Histochem. Cell. Biol. 108: 155-165.) |
| | 7503970CD1 | 342756\|RTN2 | 2.7E−249 | [Homo sapiens] Reticulon 2, member of the reticulon family of proteins, which |
| | | | | are associated with the endoplasmic reticulum |
| | | | | (Roebroek, A. J. M. et al. (1998) supra.) |
| | 7503970CD1 | 331592\|Rn.11075 | 2.5E−52 | [Rattus norvegicus] Neuroendocrine-specific protein, a member of the |
| | | | | neuroendocrine-specific reticulon family of proteins, may play a role in |
| | | | | neuroendocrine secretory events |
| | | | | (Baka, I. D. et al. (1996) Intracellular compartmentalization of two differentially |
| | | | | spliced s- rex/NSP mRNAs in neurons. Mol. Cell. Neurosci. 7: 289-303.) |
| 24 | 2578986CD1 | g14582295 | 8.5E−34 | [Mus musculus] response gene to complement 32 |
| | 2578986CD1 | 328424\|Rn.3504 | 2.0E−34 | [Rattus norvegicus][Cytoplasmic] Protein expressed in oligodendrocytes, |
| | | | | upregulated by complement activation associated with cell cycle activation |
| | 2578986CD1 | 426983\|RGC32 | 1.2E−25 | [Homo sapiens][Cytoplasmic] Protein with strong similarity to rat Rn.3504, which |
| | | | | is expressed in oligodendrocytes and upregulated by complement activation |
| | | | | associated with cell cycle activation |
| 25 | 7503786CD1 | g4099597 | 5.6E−205 | [Homo sapiens] cell division control-related 2a protein |
| | 7503786CD1 | 341082\|PNUTL2 | 4.8E−206 | [Homo sapiens][Structural protein; Activator; Hydrolase; GTP-binding |
| | | | | protein/GTPase; Small molecule-binding protein][Nuclear; Cytoplasmic; |
| | | | | Mitochondrial] Peanut (Drosophila)-Like 2, a member of the septin family, which |
| | | | | are filamentous proteins with GTP-binding domains and that may interact during |
| | | | | cytokinesis; alternative form ARTS mediates TGF-beta-dependent apoptosis |
| | 7503786CD1 | 320094\|Sept4 | 1.3E−201 | [Mus musculus] Septin 4, a member of the septin family, which are filamentous |
| | | | | proteins with GTP-binding domains and that may interact during cytokinesis |
| | 7503786CD1 | 337064\|PNUTL1 | 4.7E−152 | [Homo sapiens][Structural protein; Hydrolase; GTP-bindingprotein/GTPase; |
| | | | | Small molecule-binding protein] Peanut-like, a member of the septin family, |
| | | | | which are filamentous proteins with GTP-binding domains, and that may interact |
| | | | | during cytokinesis; geneis a translocation breakpoint in some cases of acute |
| | | | | myeloid leukemia |
| | 7503786CD1 | 418690\|Sept5 | 6.1E−150 | [Mus musculus][Structural protein] Putative ortholog of human PNUTL1, a |
| | | | | member of the septin family, which are filamentous proteins with GTP-binding |
| | | | | domains, and that may interact during cytokinesis; human gene is a translocation |
| | | | | breakpoint in some cases of acute myeloid leukemia |
| | 7503786CD1 | 318522\|Sept1 | 5.8E−122 | [Mus musculus][Nuclear; Cytoplasmic] Protein that has elevated expression in |
| | | | | lymphocytes that have high expression of the cell surface protein gp90MEL-14, |
| | | | | which is important for lymphocyte adhesion to high endothelial venule cells in |
| | | | | lymphoid organs |
| 26 | 7505684CD1 | g6137108 | 2.6E−25 | [Homo sapiens] RGC32 |
| | 7505684CD1 | 426983\|RGC32 | 2.2E−26 | [Homo sapiens][Cytoplasmic] Protein with strong similarity to rat Rn.3504, which |
| | | | | is expressed in oligo dendrocytes and upregulated by complement activation |
| | | | | associated with cell cycle activation |
| | 7505684CD1 | 328424\|Rn.3504 | 2.6E−23 | [Rattus norvegicus][Cytoplasmic] Protein expressed in oligodendrocytes, |
| | | | | upregulated by complement activation associated with cell cycle activation |
| 27 | 302655CD1 | g13603412 | 5.2E−34 | [Homo sapiens] B29 (Yanaihara, N. et al. (2001) Genomics 72 (2), 169-179) |
| 28 | 4757551CD1 | g3335399 | 1.5E−240 | [Homo sapiens] AIBC1 |
| | 4757551CD1 | 339764\|BCAS1 | 1.3E−241 | [Homo sapiens] Breast carcinoma amplified sequence 1, amplification and over |
| | | | | expression of the corresponding gene are associated with breast cancer, suggesting |
| | | | | a possible oncogenic role, decreased expression is associated with colorectal |
| | | | | cancer |
| | 4757551CD1 | 617824\|NEFH | 3.0E−13 | [Homo sapiens][Structural protein][Cytoplasmic; Cytoskeletal]Heavy subunit of |
| | | | | neurofilament, an intermediate filament protein that may regulate the caliber of |
| | | | | axons; mutation of the corresponding gene may be associated with some cases of |
| | | | | amyotrophic lateral sclerosis |
| 29 | 7503157CD1 | g12060822 | 1.8E−250 | [Homo sapiens] serologically defined breast cancer antigen NY-BR-16 |
| | 7503157CD1 | 692116\|FLJ20288 | 1.2E−231 | [Homo sapiens] Protein containing fifteen ankyrin (Ank) repeats, which may |
| | | | | mediate protein-protein interactions |
| | 7503157CD1 | 610225\|KIAA1223 | 5.4E−53 | [Homo sapiens] Protein containing thirteen ankyrin (Ank) repeats, which may |
| | | | | mediate protein-protein interactions |
| | 7503157CD1 | 321132\|Ank3 | 3.8E−45 | [Mus musculus][AnchorProtein][Lysosome/vacuole; Cytoplasmic; |
| | | | | Plasmamembrane; Cytoskeletal; Axon] Ankyrin 3 (ankyrinG), member of a family |
| | | | | of proteins that link integral membrane proteins to the cytoskeleton, has a spectrin- |
| | | | | binding domain and associates with lysosomes, expressed in epithelial cells |
| | 7503157CD1 | 618146\|ANK1 | 1.1E−43 | [Homo sapiens][Anchor Protein; Structuralprotein][Cytoplasmic; Plasma |
| | | | | membrane; Cytoskeletal] Ankyrin 1 (ankyrinR), membrane protein that attaches |
| | | | | cytoskeletal elements to the plasma membrane; mutation of the corresponding |
| | | | | gene causes hereditary spherocytosis |
| 30 | 7503702CD1 | g13469729 | 1.4E−62 | [Homo sapiens] breast cancer antigen NY-BR-1 (Jager, D. et al. (2001) Cancer |
| | | | | Res. 61 (5), 2055-2061) |
| | 7503702CD1 | 425084\|KIAA1074 | 4.4E−58 | [Homo sapiens] Protein containing ankyrin (Ank) repeats, which may mediate |
| | | | | protein-protein interactions |
| | 7503702CD1 | 606250\|LOC56311 | 1.2E−22 | [Homo sapiens] Testis-specific ankyrin motif containing protein 806, a putative |
| | | | | protein-binding protein that may play a role in male gonad development |
| | 7503702CD1 | 737411\|UACA | 2.0E−16 | [Homo sapiens] Uveal autoantigen with coiled-coil domains and ankyrin repeats, a |
| | | | | common autoantigen in people with panuveitis |
| | 7503702CD1 | 439135\|unc-44 | 3.7E−16 | [Caenorhabditis elegans] Set of ankyrin-related proteins generated by alternate |
| | | | | splicing, required for axonal guidance and proper axon fasciculation |
| | 7503702CD1 | 697436\|RAI14 | 8.7E−16 | [Homo sapiens][Cytoplasmic; Cytoskeletal] has weak similarity to a region of |
| | | | | murine DXSmh7dystrophin, which is a member of the spectrin family of |
| | | | | membrane-associated cytoskeletal proteins that is associated |
| | | | | withDuchenne/Becker Muscular Dystrophy |
| 31 | 72096217CD1 | g3242004 | 1.3E−238 | [Homo sapiens] bridging-integrator protein-1 isoform BIN1 + 12A Wechsler- |
| | | | | Reya, R. J. et al. (1997) J. Biol. Chem. 272: 31453-31458 |
| | | | | Structural analysis of the human BIN1 gene. Evidence for tissue-specific |
| | | | | transcriptional regulation and alternate RNA splicing. |
| | 72096217CD1 | 584235\|Bin1 | 6.4E−218 | [Mus musculus] [Small molecule-binding protein] [Cytoskeletal] Amphiphysin II, |
| | | | | associates with dynamin and alpha-adaptin, may connect the endocytic and |
| | | | | signaling pathways, has an SH3 domain; putative human ortholog AMPHL is a |
| | | | | tumor suppressor |
| | 72096217CD1 | 332316\|Rn.17098 | 1.2E−216 | [Rattus norvegicus] [Regulatory subunit; Small molecule-binding protein] |
| | | | | [Endosome/Endosomal vesicles; plasma membrane] Amphiphysin II, a predicted |
| | | | | cytoskeletal regulatory protein which plays a role in endocytosis of synaptic |
| | | | | vesicles |
| | 72096217CD1 | 339980\|BIN1 | 7.7E−175 | [Homo sapiens] [Activator] [Cytoplasmic; Cytoskeletal] AmphiphysinII, a tumor |
| | | | | suppressor that interacts with MYC and colocalizes with ankyrin3 (ANK3), may |
| | | | | have a role in endocytosis |
| | 72096217CD1 | 334158\|AMPH | 1.6E−74 | [Homo sapiens] [Cytoplasmic; plasma membrane; Cytoskeletal; Other vesicles of |
| | | | | the secretory/endocytic pathways] Amphiphysin, binds dynamin I (DNM1) and |
| | | | | synaptojanin 1 (SYNJ1) via its SH3 domain, also binds the AP-2 complex, likely |
| | | | | involved in synaptic vesicle endocytosis; autoantibodies are associated with |
| | | | | neurological disorders such as Stiff-man syndrome |
| | 72096217CD1 | 476363\|BIN2 | 6.8E−72 | [Homo sapiens] Bridging integrator 2, a member of the BAR family of adaptor |
| | | | | proteins, expressed predominantly in hematopoietic cells and is upregulated |
| | | | | during differentiation of granulocytes |
| 32 | 7503620CD1 | g192597 | 2.3E−173 | [Mus musculus] clusterin French, L. L. et al. (1993) J. Cell Biol. 122: 1119-1130 |
| | | | | Marine clusterin: molecular cloning and mRNA localization of a gene associated |
| | | | | with epithelial differentiation processes during embryogenesis |
| | 7503620CD1 | 327964\|Trpm2 | 2.9E−173 | [Rattus norvegicus] Clusterin, glycoprotein that may be involved in immune |
| | | | | regulation and inhibition of apoptosis |
| | 7503620CD1 | 586923\|Clu | 6E−173 | [Mus musculus] Clusterin, glycoprotein that may be involved in immune |
| | | | | regulation, cell adhesion, stress responses, and inhibition of apoptosis |
| | 7503620CD1 | 334716\|CLU | 1.4E−118 | [Homo sapiens] [Structural protein] Clusterin, glycoprotein that may be involved |
| | | | | in immune regulation, lipid transport, stress responses, and inhibition of apoptosis |
| | 7503620CD1 | 424860\|CLUL1 | 6.2E−35 | [Homo sapiens] Protein with weak similarity to several clusterin glycoproteins, |
| | | | | contains a clusterin domain |
| 33 | 7503621CD1 | g292843 | 9.9E−159 | [Homo sapiens] TRPM-2 gene product Wong, P. et al. (1993) J. Biol. Chem. |
| | | | | 268: 5021-5031 |
| | | | | Genomic organization and expression of the rat TRPM-2 (clusterin) gene, a gene |
| | | | | implicated in apoptosis. |
| | 7503621CD1 | 334716\|CLU | 8.6E−160 | [Homo sapiens] [Structural protein] Clusterin, glycoprotein that may be involved |
| | | | | in immune regulation, lipid transport, stress responses, and inhibition of apoptosis |
| | 7503621CD1 | 327964\|Trpm2 | 1.6E−117 | [Rattus norvegicus] Clusterin, glycoprotein that may be involved in immune |
| | | | | regulation and inhibition of apoptosis |
| | 7503621CD1 | 586923\|Clu | 3.1E−116 | [Mus musculus] Clusterin, glycoprotein that may be involved in immune |
| | | | | regulation, cell adhesion, stress responses, and inhibition of apoptosis |
| | 7503621CD1 | 424860\|CLUL1 | 8.3E−32 | [Homo sapiens] Protein with weak similarity to several clusterin glycoproteins, |
| | | | | contains a clusterin domain |
| 34 | 7503624CD1 | g292843 | 5.4E−135 | [Homo sapiens] TRPM-2 gene product Wong, P. et al., supra |
| | 7503624CD1 | 334716\|CLU | 4.7E−136 | [Homo sapiens] [Structural protein] Clusterin, glycoprotein that may be involved |
| | | | | in immune regulation, lipid transport, stress responses, and inhibition of apoptosis |
| | 7503624CD1 | 327964\|Trpm2 | 5.4E−131 | [Rattus norvegicus] Clusterin, glycoprotein that may be involved in immune |
| | | | | regulation and inhibition of apoptosis |
| | 7503624CD1 | 586923\|Clu | 2.2E−99 | [Mus musculus] Clusterin, glycoprotein that may be involved in immune |
| | | | | regulation, cell adhesion, stress responses, and inhibition of apoptosis |
| | 7503624CD1 | 424860\|CLUL1 | 4.2E−22 | [Homo sapiens] Protein with weak similarity to several clusterin glycoproteins, |
| | | | | contains a clusterin domain |
| 35 | 7503625CD1 | g292843 | 1.2E−235 | [Homo sapiens] TRPM-2 gene product Wong, P. et al., supra |
| | 7503625CD1 | 334716\|CLU | 1E−236 | [Homo sapiens] [Structural protein] Clusterin, glycoprotein that may be involved |
| | | | | in immune regulation, lipid transport, stress responses, and inhibition of apoptosis |
| | 7503625CD1 | 327964\|Trpm2 | 1.4E−182 | [Rattus norvegicus] Clusterin, glycoprotein that may be involved in immune |
| | | | | regulation and inhibition of apoptosis |
| | 7503625CD1 | 586923\|Clu | 7E−181 | [Mus musculus] Clusterin, glycoprotein that may be involved in immune |
| | | | | regulation, cell adhesion, stress responses, and inhibition of apoptosis |
| | 7503625CD1 | 424860\|CLUL1 | 6.5E−18 | [Homo sapiens] Protein with weak similarity to several clusterin glycoproteins, |
| | | | | contains a clusterin domain |
| 36 | 7503627CD1 | g292843 | 4.7E−134 | [Homo sapiens] TRPM-2 gene product Wong, P. et al., supra |
| | 7503627CD1 | 334716\|CLU | 4.1E−135 | [Homo sapiens] [Structural protein] Clusterin, glycoprotein that may be involved |
| | | | | in immune regulation, lipid transport, stress responses, and inhibition of apoptosis |
| | 7503627CD1 | 327964\|Trpm2 | 8.4E−98 | [Rattus norvegicus] Clusterin, glycoprotein that may be involved in immune |
| | | | | regulation and inhibition of apoptosis |
| | 7503627CD1 | 586923\|Clu | 1.4E−97 | [Mus musculus] Clusterin, glycoprotein that may be involved in immune |
| | | | | regulation, cell adhesion, stress responses, and inhibition of apoptosis |
| | 7503627CD1 | 424860\|CLUL1 | 3.4E−14 | [Homo sapiens] Protein with weak similarity to several clusterin glycoproteins, |
| | | | | contains a clusterin domain |
| 37 | 7505744CD1 | g3764055 | 2.3E−69 | [Homo sapiens] follistatin-related protein FLRG Hayette, S. et al. (1998) |
| | | | | Oncogene 16: 2949-2954 |
| | | | | FLRG (follistatin-related gene), a new target of chromosomal rearrangement in |
| | | | | malignant blood disorders. |
| | 7505744CD1 | 342464\|FSTL3 | 2E−70 | [Homo sapiens] [Extracellular (excluding cell wall)] Follistatin-like 3, a secreted |
| | | | | glycoprotein of the follistatin module protein family; corresponding gene is |
| | | | | involved in a chromosomal translocation associated with malignant blood |
| | | | | disorders |
| | 7505744CD1 | 709935\|Fstl3 | 2E−48 | [Mus musculus] Follistatin-like 3, a secreted glycoprotein of the follistatin module |
| | | | | protein family, binds to and inhibits the activity of members of the TGF-beta |
| | | | | family; human FSTL3 is involved in a chromosomal translocation associated with |
| | | | | malignant blood disorders |
| | 7505744CD1 | 430560\|Fst | 3.5E−21 | [Rattus norvegicus] [Inhibitor or represser] Follistatin, binds and neutralizes |
| | | | | activin, regulates pituitary FSH secretion, embryogenesis, cell proliferation, and |
| | | | | ovarian and testicular function; human FST may contribute to polycystic ovarian |
| | | | | syndrome |
| | | | | Michel, U. et al. (2000) Eur. J. Endocrinol. 143: 809-816 |
| | | | | Follistatin (FS) in human cerebrospinal fluid and regulation of FS expression in a |
| | | | | mouse model of meningitis. |
| | 7505744CD1 | 322496\|Fst | 5.7E−21 | [Mus musculus] [Inhibitor or represser] Follistatin, binds and neutralizes activin, |
| | | | | regulates pituitary FSH secretion, functions in embryogenesis, cell proliferation, |
| | | | | ovarian and testicular function, deficiency is lethal soon after birth; human FST |
| | | | | may contribute to polycystic ovarian syndrome |
| | 7505744CD1 | 344208\|FST | 1.2E−20 | [Homo sapiens] [Inhibitor or represser] Follistatin, binds and neutralizes activin, |
| | | | | regulates pituitary FSH secretion, embryogenesis, cell proliferation, and possibly |
| | | | | ovarian follicular development, may contribute to polycystic ovarian syndrome |
| 38 | 7503712CD1 | g11494125 | 4.0E−194 | [Homo sapiens] chordin Millet, C. et al. (2001) Mech. Dev. 106: 85-96 The |
| | | | | human chordin gene encodes several differentially expressed splice variants with |
| | | | | distinct BMP opposing activities. |
| | 7503712CD1 | 584507\|Chrd | 1.1E−169 | [Mus musculus] [Inhibitor or repressor] Chordin, a member of a family of bone |
| | | | | morphogenetic protein inhibitors that are involved in dorsalization of the early |
| | | | | embryo and is highly expressed in the embryo on day 7 of development |
| | 7503712CD1 | 347908\|CHRD | 2.1E−124 | [Homo sapiens] [Inhibitor or repressor] Chordin, a member of a family of bone |
| | | | | morphogenetic protein inhibitors that are involved in dorsalization of the early |
| | | | | embryo and is widely expressed in embryos and adults |
| 39 | 7503713CD1 | g11494125 | 0.0 | [Homo sapiens] chordin Millet, C. et al., supra |
| | 7503713CD1 | 347908\|CHRD | 0.0 | [Homo sapiens] [Inhibitor or repressor] Chordin, a member of a family of bone |
| | | | | morphogenetic protein inhibitors that are involved in dorsalization of the early |
| | | | | embryo and is widely expressed in embryos and adults |
| | 7503713CD1 | 584507\|Chrd | 0.0 | [Mus musculus] [Inhibitor or repressor] Chordin, a member of a family of bone |
| | | | | morphogenetic protein inhibitors that are involved in dorsalization of the early |
| | | | | embryo and is highly expressed in the embryo on day 7 of development |
| | 7503713CD1 | 704331\|Nrln1 | 0.00000041 | [Mus musculus] Neuralin 1, a member of the chordin family of secreted proteins |
| | | | | involved in signaling during development, contains three cysteine-rich repeats |
| | 7503713CD1 | 476867\|Crim1 | 0.0000025 | [Mus musculus] [Unspecified membrane; plasma membrane] Cysteine-rich motor |
| | | | | neuron 1, a putative transmembrane protein with an insulin-like growth factor |
| | | | | binding protein motif and multiplechordin like cysteine-rich repeats, may have a |
| | | | | role in both metanephric development and central nervous system development |
| 40 | 7502225CD1 | g5360111 | 1.0E−94 | [Homo sapiens] NY-REN-41 antigen Scanlan, et al. (1999) Int. J. Cancer 83: 456-464 |
| | | | | Antigens recognized by autologous antibody in patients with renal-cell carcinoma. |
| | 7502225CD1 | 8283\|YNL091W | 1.3E−17 | [Saccharomyces cerevisiae] Protein with similarity to Usolp and human NF2 |
| | | | | neurofibromatosis type 2 gene product |
| | 7502225CD1 | 377633\|SPAC30.14c | 1E−15 | [Schizosaccharomyces pombe] Coiled-coil protein containing troponindomain |
| | 7502225CD1 | 132\|MNN4 | 5.5E−15 | [Saccharomyces cerevisiae] [Unspecified membrane] Protein required for transfer |
| | | | | of mannosylphosphate to core and outer chain portions of N-linked |
| | | | | oligosaccharides |
| | 7502225CD1 | 239154\|C11G6.3 | 3.4E−14 | [Caenorhabditis elegans] Protein containing aputative PHD-type zinc finger |
| | | | | domain, has weak similarity to human protein kinase STK10 |
| 41 | 7502778CD1 | g5360119 | 6.2E−96 | [Homo sapiens] NY-REN-50 antigen Scanlan, M. J. et al., supra |
| 42 | 7503699CD1 | g13469729 | 4.3E−29 | [Homo sapiens] breast cancer antigen NY-BR-1 Jager, D. et al. (2001) Cancer |
| | | | | Res. 61: 2055-2061 |
| | | | | Identification of a tissue-specific putative transcription factor in breast tissue by |
| | | | | serological screening of a breast cancer library. |
| | 7503699CD1 | 425084\|KIAA1074 | 7.7E−44 | [Homo sapiens] Protein containing ankyrin (Ank) repeats, which may mediate |
| | | | | protein-protein interactions |
| | 7503699CD1 | 434390\|KIAA0379 | 1.2E−13 | [Homo sapiens] Protein containing twenty-three ankyrin (Ank) repeats, which may |
| | | | | mediate protein-protein interactions |
| | 7503699CD1 | 685397\|MGC5540 | 3.3E−13 | [Homo sapiens] Protein of unknown function, has a region of moderate similarity |
| | | | | to a region of human PSMD10, which is a non-ATPase subunit of the 26S |
| | | | | proteasome (prosome, macropain) that may play a role in hepatocellular |
| | | | | carcinoma development by destabilizing human RB1 |
| | | | | Higashitsuji, H. et al. (2000) Nat. Med. 6: 96-99 Reduced stability of |
| | | | | retinoblastoma protein by gankyrin, an oncogenic ankyrin-repeat protein |
| | | | | overexpressed in hepatomas. |
| | 7503699CD1 | 609058\|Ankrd2 | 1.2E−12 | [Mus musculus] [Inhibitor or repressor; Transcription factor] Ankyrin repeat |
| | | | | domain 2, a protein with an ATP/GTP binding domain, a nuclear localization |
| | | | | signal, two PEST protein-destabilization motifs and ankyrin repeats, may play a |
| | | | | role in skeletal muscle hypertrophy |
| | 7503699CD1 | 475935\|LOC51239 | 2.2E−12 | [Homo sapiens] Protein containing three ankyrin (Ank)repeats, which may |
| | | | | mediate protein-protein interactions |
| 43 | 6026715CD1 | g6165419 | 8.5E−105 | [Mus musculus] septin-like protein Sint1 Sorensen, A. B. et al. (2000) J. Virol. |
| | | | | 74: 2161-2168 |
| | | | | Sint1, a common integration site in SL3-3-induced T-cell lymphomas, harbors a |
| | | | | putative proto-oncogene with homology to the septin gene family. |
| | 6026715CD1 | 477111\|Sept9 | 7.40E−106 | [Mus musculus] Member of the septin family, which are involved in cytokinesis |
| | | | | and establishment of cell polarity |
| | 6026715CD1 | 711864\|LOC83788 | 3.20E−105 | [Rattus norvegicus] Myeloid/lymphoid leukemia gene septin-like fusion protein, a |
| | | | | GTP-binding protein; human MSF is fused to MLL in acute myeloid leukemia |
| | | | | (AML) and therapy-related AML, and is commonly found to be deleted in |
| | | | | sporadic epithelial ovarian tumors |
| | 6026715CD1 | 432874\|MSF | 6.70E−105 | [Homo sapiens] [Hydrolase; GTP-binding protein/GTPase; Small molecule- |
| | | | | binding protein] [Cytoplasmic] Myeloid/lymphoid leukemia gene septin-like |
| | | | | fusion protein, member of the septin family; corresponding gene is fused to MLL |
| | | | | in acute myeloid leukemia (AML)and therapy-related AML, and is commonly |
| | | | | found to be deleted in sporadic epithelial ovarian tumors |
| | 6026715CD1 | 597863\|Sep3 | 4.70E−104 | [Rattus norvegicus] [Hydrolase; GTP-binding protein/GTPase; Small molecule- |
| | | | | binding protein] G-septin alpha, a neuronal member of the septin family of a GTP- |
| | | | | binding/GTPase proteins, lacks a C-terminal coiled-coil domain characteristic of |
| | | | | mammalian septins, may be regulated by PKG in neurons |
| | 6026715CD1 | 594860\|SEPT3 | 3.40E−101 | [Homo sapiens] Septin 3, a member of the septin protein family |
| 44 | 7503631CD1 | g292843 | 1.5E−222 | [Homo sapiens] TRPM-2 gene product Wong, P. et al. (1994) Eur. J. Biochem. |
| | | | | 221: 917-925 |
| | | | | Molecular characterization of human TRPM-2/clusterin, a gene associated with |
| | | | | sperm maturation, apoptosis and neurodegeneration. |
| | 7503631CD1 | 334716\|CLU | 1.30E−223 | [Homo sapiens] [Structural protein] Clusterin, glycoprotein that may be involved |
| | | | | in immune regulation, lipid transport, stress responses, and inhibition of apoptosis |
| | 7503631CD1 | 586923\|Clu | 8.60E−174 | [Mus musculus] Clusterin, glycoprotein that may be involved in immune |
| | | | | regulation, cell adhesion, stress responses, and inhibition of apoptosis |
| | 7503631CD1 | 327964\|Trpm2 | 1.40E−173 | [Rattus norvegicus] Clusterin, glycoprotein that may be involved in immune |
| | | | | regulation and inhibition of apoptosis |
| 45 | 7503664CD1 | g292843 | 1.0E−181 | [Homo sapiens] TRPM-2 gene product |
| | 7503664CD1 | 334716\|CLU | 8.70E−183 | [Homo sapiens] [Structural protein] Clusterin, glycoprotein that may be involved |
| | | | | in immune regulation, lipid transport, stress responses, and inhibition of apoptosis |
| | 7503664CD1 | 327964\|Trpm2 | 3.80E−141 | [Rattus norvegicus] Clusterin, glycoprotein that may be involved in immune |
| | | | | regulation and inhibition of apoptosis |
| | 7503664CD1 | 586923\|Clu | 8.20E−139 | [Mus musculus] Clusterin, glycoprotein that may be involved in immune |
| | | | | regulation, cell adhesion, stress responses, and inhibition of apoptosis |
| 46 | 7504812CD1 | g307151 | 1.3E−125 | [Homo sapiens] MAC25 Murphy, M. et al. (1993) Cell Growth Differ. 4: 715-722 |
| | | | | Identification and characterization of genes differentially expressed in |
| | | | | meningiomas. |
| | 7504812CD1 | 335964\|IGFBP7 | 3.70E−132 | [Homo sapiens] [Inhibitor or repressor] Member of the insulin-like growth factor |
| | | | | binding family of proteins that binds to and modulates insulin-like growth factor |
| | | | | activity and may act as a cell growth inhibitor; decreased expression of the |
| | | | | corresponding gene is associated with meningiomas |
| | 7504812CD1 | 429392\|Igfbp7 | 7.60E−119 | [Mus musculus] [Inhibitor or repressor] Protein with strong homology to |
| | | | | follistatin that is an activin-binding protein, may function as a tumor suppressor |
| | | | | and modulate signaling of the TGF-beta family; has strong similarity to human |
| | | | | IGFBP7 which is associated with meningiomas |
| | 7504812CD1 | 608144\|IGFBPL | 3.30E−32 | [Mus musculus] [Inhibitor or repressor; Protease (other than proteasomal)] Protein |
| | | | | with moderate similarity to Fstl2 follistatin-like 2, which is an activin-binding |
| | | | | protein that may act as a tumor suppressor, contains an immunoglobulin (Ig) |
| | | | | domain, and a Kazal-type serine protease inhibitor domain |
| | 7504812CD1 | 608602\|Prss11 | 9.00E−25 | [Mus musculus] [Hydrolase; Protease (other than proteasomal)] [Extra cellular |
| | | | | (excluding cell wall)] Protease (serine, 11 (IGF-binding)), has strong similarity to |
| | | | | human PRSS11, which is a serine protease that interacts with alpha1-antitrypsin |
| | | | | and is overexpressed in the cartilage of patients with osteoarthritis; contains |
| | | | | trypsin and PDZ domains |
| | 7504812CD1 | 337258\|PRSS11 | 1.50E−24 | [Homo sapiens] [Hydrolase; Protease (other than proteasomal)] [Extra cellular |
| | | | | (excluding cell wall)] Protease(serine, 11 (IGF-binding)), member of a family of |
| | | | | serine proteases which interacts with alpha1-antitrypsin; increased expression is |
| | | | | detected in the cartilage of patients with osteoarthritis |
| 47 | 7504996CD1 | g13543353 | 1.8E−95 | [Homo sapiens] follistatin-like 3 (secreted glycoprotein) |
| | 7504996CD1 | 342464\|FSTL3 | 1.60E−96 | [Homo sapiens] [Extra cellular (excluding cell wall)] Follistatin-like 3, a secreted |
| | | | | glycoprotein of the follistatin module protein family; corresponding gene is |
| | | | | involved in a chromosomal translocation associated with malignant blood |
| | | | | disorders |
| | | | | Hayette, S., Gadoux, M., Martel, S., Bertrand, S., Tigaud, I., Magaud, J. P., and |
| | | | | Rimokh, R. FLRG (follistatin-related |
| | | | | gene), a new target of chromosomal rearrangement in malignant blood disorders. |
| | | | | Oncogene 16, 2949-54 (1998). |
| | 7504996CD1 | 709935\|Fstl3 | 2.20E−81 | [Mus musculus] Follistatin-like 3, a secreted glycoprotein of the follistatin module |
| | | | | protein family, binds to and inhibits the activity of members of the TGF-beta |
| | | | | family; human FSTL3 is involved in a chromosomal translocation associated with |
| | | | | malignant blood disorders |
| | 7504996CD1 | 430560\|Fst | 3.80E−47 | [Rattus norvegicus] [Inhibitor or repressor] Follistatin, binds and neutralizes |
| | | | | activin, regulates pituitary FSH secretion, embryogenesis, cell proliferation, and |
| | | | | ovarian and testicular function; human FST may contribute to polycystic ovarian |
| | | | | syndrome |
| | 7504996CD1 | 322496\|Fst | 6.20E−47 | [Mus musculus] [Inhibitor or repressor] Follistatin, binds and neutralizes activin, |
| | | | | regulates pituitary FSH secretion, functions in embryogenesis, cell proliferation, |
| | | | | ovarian and testicular function, deficiency is lethal soon after birth; human FST |
| | | | | may contribute to polycystic ovarian syndrome |
| | 7504996CD1 | 344208\|FST | 6.20E−47 | [Homo sapiens] [Inhibitor or repressor] Follistatin, binds and neutralizes activin, |
| | | | | regulates pituitary FSH secretion, embryogenesis, cell proliferation, and possibly |
| | | | | ovarian follicular development, may contribute to polycystic ovarian syndrome |
| 48 | 7377279CD1 | g16117380 | 0.0E+00 | [fl][Homo sapiens] gastric cancer-related protein FKSG9 |
| | 7377279CD1 | g10241843 | 1.70E−200 | [Mus musculus] gasdermin |
| | | | | Saeki, N. et al. (2000) Mamm. Genome 11: 718-724 |
| | | | | Gasdermin (Gsdm) localizing to mouse chromosome 11 is predominantly |
| | | | | expressed in upper gastrointestinal tract but significantly suppressed in human |
| | | | | gastric cancer cells. |
| | 7377279CD1 | 618646\|Gsdm | 1.50E−201 | [Mus musculus] Gasdermin, expressed in upper the gastrointestinal tract and skin; |
| | | | | human homolog is down-regulated ingastric cancer cells, may have a role in |
| | | | | blocking carcinogenesis in gastric tissue |
| | 7377279CD1 | 709931\|Mlze | 2.40E−38 | [Mus musculus] Protein that is preferentially expressed in metastatic melanomas |
| | 7377279CD1 | 600122\|PRO2521 | 1.10E−31 | [Homo sapiens] Protein of unknown function, has a region of moderate similarity |
| | | | | to a region of DFNA5, which is expressed in the cochlea and associated with non- |
| | | | | syndromic hearing impairment |
| 49 | 2060984CD1 | g12659142 | 9.3E−115 | [Mus musculus] mage-g1 Saeki, N. et al. (2000) Mamm. Genome 11: 718-724 |
| | | | | Gasdermin (Gsdm) localizing to mouse chromosome 11 is predominantly |
| | | | | expressed in upper gastrointestinal tract but significantly suppressed in human |
| | | | | gastric cancer cells. |
| | 2060984CD1 | 617934\|MAGED2 | 1.60E−55 | [Homo sapiens] Member of the MAGE (melanoma antigen-encoding gene) |
| | | | | family, which are expressed in a variety of tumors, has region of moderate |
| | | | | similarity to necdin (NDN), which is a neuronal growth suppressor associated with |
| | | | | Prader-Willi syndrome |
| | | | | Muscatelli, F. et al. (2000) Mol. Genet. 9: 3101-3110 |
| | | | | Disruption of the mouse necdin gene results in hypothalamic and behavioral |
| | | | | alterations reminiscent of the human prader-willi syndrome Hum. |
| | 2060984CD1 | 559775\|MAGEL2 | 1.80E−55 | [Homo sapiens] MAGE-like 2, corresponding gene is subject to imprinting and |
| | | | | maps to the critical chromosomal region associated with Prader-Willi syndrome |
| | 2060984CD1 | 344560\|MAGEB4 | 8.00E−54 | [Homo sapiens] Melanoma antigen family B 4, a member of a family of tumor |
| | | | | antigens |
| | 2060984CD1 | 742738\|MAGED1 | 4.00E−53 | [Homo sapiens] Neurotrophin-receptor interacting MAGE homolog, a novel |
| | | | | member of the melanoma antigen family D, which, unlike other members of the |
| | | | | MAGE family, is expressed in many normal tissues |
| 50 | 4085993CD1 | g12060849 | 1.0E−155 | [5′ incom][Homo sapiens] serologically defined breast cancer antigen NY-BR-85 |
| 51 | 6756305CD1 | g1297319 | 0.0 | [Homo sapiens] DOC1 Mok, S. C. (1994)Gynecol. Oncol. 52: 247-252 Molecular |
| | | | | cloning of differentially expressed genes in human epithelial ovarian cancer. |
| | 6756305CD1 | 743416\|DOC1 | 0.0 | [Homo sapiens] Downregulated in ovarian cancer 1, a putative protein expressed |
| | | | | by normal ovarian surface epithelial cells but not by ovarian cancer cell lines |
| | 6756305CD1 | 632626\|orf6.1434 | 2.00E−23 | [Candida albicans] [Unknown] Protein with low similarity to S. cerevisiae Usolp, |
| | | | | which is a coiled-coil protein with similarity to mammalian integrin that is |
| | | | | required for protein transport from ER to Golgi |
| | 6756305CD1 | 335126\|EEA1 | 7.60E−23 | [Homo sapiens] [Small molecule-binding protein] [Endosome/Endosomal |
| | | | | vesicles; Nuclear; Cytoplasmic; Plasma membrane] Protein present in early |
| | | | | endosomes and required for endosome fusion, acts as an effector of the endosomal |
| | | | | small GTPaseRAB5 |
| | 6756305CD1 | 623900\|MYH3 | 4.00E−22 | [Homo sapiens] [Motorprotein; Hydrolase; ATPase] [Cytoplasmic; Cytoskeletal] |
| | | | | Skeletal musclemyosin heavy chain, member of a family of motor proteins that |
| | | | | provide the force for muscle contraction, expressed only during embryogenesis |
| 52 | 7504842CD1 | g4091819 | 3.4E−229 | [Homo sapiens] leucine-rich glioma-inactivated protein precursor Chernova, O. B. |
| | | | | (1998) Oncogene 17: 2873-2881 |
| | | | | A novel gene, LGI1, from 10q24 is rearranged and downregulated in malignant |
| | | | | brain tumors. |
| | 7504842CD1 | 341690\|LGI1 | 2.90E−230 | [Homo sapiens] Leucine-rich glioma inactivated 1, a member of the LRR-protein |
| | | | | superfamily that contains four leucine-rich repeats, may function in neurogenesis, |
| | | | | may be a tumor suppressor; Gene rearrangement and low expression are |
| | | | | associated with malignant glioma |
| | 7504842CD1 | 607582\|Lgi1 | 3.60E−225 | [Mus musculus] Leucine-rich glioma inactivated 1, contains four leucine-rich |
| | | | | repeats, putative glial tumor suppressor; gene rearrangement and low expression |
| | | | | of the human LGI1 gene are associated with malignant gliomas |
| 53 | 7500901CD1 | g7899272 | 5.0E−22 | [Caenorhabditis elegans] LET-413 protein Legouis R, et al. (2000) Nature Cell |
| | | | | Biol. 2: 415-22 |
| | | | | LET-413 is a basolateral protein required for the assembly of adherens junctions |
| | | | | in Caenorhabditis elegans. |
| 55 | 7500912CD1 | g14424787 | 5.6E−89 | [Homo sapiens] natural killer cell transcript 4 |
| | 7500912CD1 | 623570\|NK4 | 8E−90 | [Homo sapiens][Extracellular (excluding cell wall)] Protein with an RGD motif |
| | | | | that may play a role in cell adhesion, expressed by lymphocytes and upregulated |
| | | | | in mitogen-activated T cells and IL-2 treated NK cells |
| 56 | 7505851CD1 | g14424787 | 1.3E−72 | [Homo sapiens] natural killer cell transcript 4 |
| | 7505851CD1 | 623570\|NK4 | 1.80E−73 | [Homo sapiens][Extracellular (excluding cell wall)] Natural killer cell transcript 4, |
| | | | | protein with an RGD motif that may play a role in cell adhesion, expressed by |
| | | | | lymphocytes and is upregulated in mitogen-activated T cells and IL2 treated |
| | | | | natural killer cells. |
| | | | | Dahl, C. A. et al. (1992) Identification of a novel gene expressed in activated |
| | | | | natural killer cells |
| | | | | and T cells. J. Immunol. 148: 597-603. |

TABLE 3

| | | Amino | | Potential | | |
|---|---|---|---|---|---|---|
| SEQ | Incyte | Acid | Potential | Glyco- | | Analytical |
| ID | Polypeptide | Resi- | Phosphorylation | sylation | | Methods |
| NO: | ID | dues | Sites | Sites | Signature Sequences, Domains and Motifs | and Databases |
| 1 | 1318936CD1 | 565 | S24 S26 S66 S92 | N23 N317 | LIM domain: C195-A252, C130-K192, C255-D315 | HMMER_PFAM |
| | | | S106 S311 S334 | N410 | | |
| | | | S337 S362 S396 | | | |
| | | | S453 S482 S490 | | | |
| | | | S526 T25 T208 | | | |
| | | | T536 Y401 Y473 | | | |
| | | | | | LIM domain signature and profile: R88-E189 | PROFILESCAN |
| | | | | | LIM PROTEIN MOTIF METAL-BINDING ZINC | BLAST_PRODOM |
| | | | | | TESTIN TRIPLE DOMAIN REPEAT SIMILARITY | |
| | | | | | PD010685: K15-G127 | |
| | | | | | LIM TESTIN PROTEIN TRIPLE DOMAIN MOTIF | BLAST_PRODOM |
| | | | | | METAL-BINDING ZINC REPEAT SIMILARITY | |
| | | | | | PD014918: Y251-G313 | |
| | | | | | LIM METAL-BINDING REPEAT | BLAST_DOMO |
| | | | | | DM00055\|P47226\|295-353: K192-C246 | |
| | | | | | DM00055\|I48841\|176-239: C130-H187 | |
| | | | | | DM00055\|P47226\|231-293: C130-H186 | |
| | | | | | Leucine zipper pattern: L358-L379 | MOTIFS |
| | | | | | LIM domain signature: C130-L169, C195-L229 | MOTIFS |
| 2 | 033784CD1 | 167 | S69 S70 S75 S87 | N83 | PROTEIN D7 DEVELOPMENTAL D7-LIKE | BLAST_PRODOM |
| | | | T4 T92 | | PD125586: E12-D80 | |
| 3 | 4160218CD1 | 1397 | S78 S110 S183 | N372 N538 | Appr-1″-p processing enzyme family: | HMMER_PFAM |
| | | | S202 S437 S540 | N580 N816 | V537-V655, V749-I866 | |
| | | | S603 S680 S766 | N963 N1039 | | |
| | | | S821 S840 S873 | N1152 N1202 | | |
| | | | S891 S913 S930 | N1259 N1260 | | |
| | | | S1079 S1176 S1243 | N1354 | | |
| | | | S1318 S1342 T3 | | | |
| | | | T82 T125 T129 | | | |
| | | | T145 T174 T358 | | | |
| | | | T374 T405 T462 | | | |
| | | | T698 T718 T880 | | | |
| | | | T954 T1145 T1154 | | | |
| | | | T1175 T1283 | | | |
| | | | T1301 Y1189 | | | |
| | | | | | WWE domain: E1240-S1318 | HMMER_PFAM |
| | | | | | POLYPROTEIN PROTEIN NONSTRUCTURAL | BLAST_PRODOM |
| | | | | | CONTAINS: PROTEINS NSP1 NSP4 RNA | |
| | | | | | BINDING HELICASE HISTONE PD002016: | |
| | | | | | V519-1658 | |
| | | | | | do NONSTRUCTURAL; POLYPROTEIN; ORF2; | BLAST_DOMO |
| | | | | | RUBELLA; | |
| | | | | | DM04159\|I39569\|1-173: G518-E660 | |
| | | | | | do NONSTRUCTURAL; POLYPROTEIN; ORF2; | |
| | | | | | RUBELLA; | |
| | | | | | DM04159\|Q02874\|190-362: G730-R905 | |
| | | | | | ATP/GTP-binding site motif A (P-loop): | MOTIFS |
| | | | | | A711-T718 | |
| 4 | 7501989CD1 | 553 | S26 S38 S78 S100 | | | |
| | | | S138 S160 S286 | | | |
| | | | S443 S453 S499 | | | |
| | | | T73 T293 T338 | | | |
| | | | T475 Y42 Y372 | | | |
| | | | Y485 | | | |
| 5 | 7500695CD1 | 175 | S29 S90 T63 T67 | | signal_cleavage: M1-L22 | SPSCAN |
| | | | T113 | | | |
| | | | | | Signal Peptide: M1-S20 | HMMER |
| | | | | | CANCER ASSOCIATED SURFACE ANTIGEN | BLAST_PRODOM |
| | | | | | PD111520: K34-S175, M1-T44 | |
| 6 | 7502088CD1 | 210 | S2 S5 S42 S98 | N80 N95 | PROTEIN BCL7C BCL7B MRNA SHORT | BLAST_PRODOM |
| | | | S156 S165 S195 | N109 N163 | ISOFORM BCL7A ALTERNATIVE SPLICING | |
| | | | T37 T76 | | PD009381: E32-E151 | |
| | | | | | BCL7A PROTEIN ALTERNATIVE SPLICING | BLAST_PRODOM |
| | | | | | PD082200: A137-K199 | |
| | | | | | do BCL7A; BCL7B; DM03990 | BLAST_DOMO |
| | | | | | \|S58237\|33-136: K33-A137 | |
| | | | | | \|S58284\|33-135: K33-Q136 | |
| | | | | | do BCL7A; IGH; BCL7B; DM03044 | BLAST_DOMO |
| | | | | | \|S58237\|1-31: M1-E32 | |
| | | | | | \|S58284\|1-31: M1-E32 | |
| 7 | 7198825CD1 | 915 | S58 S74 S140 S224 | N426 N633 | SPRY domain (SPla and the RYanodine Receptor): | HMMER_PFAM |
| | | | S241 S291 S308 | | C788-D910 | |
| | | | S314 S329 S353 | | | |
| | | | S367 S382 S406 | | | |
| | | | S414 S428 S455 | | | |
| | | | S503 S513 S529 | | | |
| | | | S538 S641 S740 | | | |
| | | | S767 S911 T20 T56 | | | |
| | | | T100 T179 T240 | | | |
| | | | T292 T320 T343 | | | |
| | | | T361 T369 T375 | | | |
| | | | T422 T480 T502 | | | |
| | | | T608 T668 T674 | | | |
| | | | T775 T796 T849 | | | |
| | | | Y119 Y360 | | | |
| | | | | | Fibronectin type III domain: V550-S641, | HMMER_PFAM |
| | | | | | P650-S734 | |
| 8 | 71863981CD1 | 907 | S59 S132 S164 | N306 N501 | Signal Peptide: M24-A49 | HMMER |
| | | | S166 S184 S198 | N541 N558 | | |
| | | | S218 S263 S308 | N659 | | |
| | | | S371 S392 S494 | | | |
| | | | S609 S610 S625 | | | |
| | | | S660 S692 S761 | | | |
| | | | S807 S826 S827 | | | |
| | | | S872 T110 T116 | | | |
| | | | T144 T189 T228 | | | |
| | | | T297 T332 T524 | | | |
| | | | T564 T674 T745 | | | |
| | | | T817 Y293 Y779 | | | |
| | | | | | Cytosolic domain: N868-Q907 | TMHMMER |
| | | | | | Transmembrane domain: Q845-Y867 | |
| | | | | | Non-cytosolic domain: M1-C844 | |
| | | | | | LYMPHOID RESTRICTED MEMBRANE | BLAST_PRODOM |
| | | | | | PROTEIN PD043313: E525-K698 | |
| | | | | | ATP/GTP-binding site motif A (P-loop): | MOTIFS |
| | | | | | A738-T745 | |
| 9 | 7500513CD1 | 158 | S89 S139 | N96 | Galactoside-binding lectin: P27-S157 | HMMER_PFAM |
| | | | | | Vertebrate galactoside-binding lectin proteins | BLIMPS_BLOCKS |
| | | | | | BL00309: P35-L59, G62-N76, F80-P104 | |
| | | | | | Vertebrate galactoside-binding lectin signature: | PROFILESCAN |
| | | | | | R31-A122 | |
| | | | | | LECTIN GALAPTIN REPEAT PROTEIN BETA- | BLAST_PRODOM |
| | | | | | GALACTOSIDE BINDING BINDING | |
| | | | | | ACETYLATION LACTOSE BINDING GALECTIN | |
| | | | | | MULTIGENE PD000676: G46-D149 | |
| | | | | | GALECTIN 8 GALAPTIN LECTIN REPEAT | BLAST_PRODOM |
| | | | | | PROSTATE CARCINOMA TUMOR ANTIGEN | |
| | | | | | PCTA1 S-TYPE PD020379: L8-E47 | |
| | | | | | VERTEBRATE GALACTOSIDE-BINDING | BLAST_DOMO |
| | | | | | LECTIN DM00426 | |
| | | | | | \|A55975\|181-315: L24-W158 | |
| | | | | | \|A55664\|188-323: L26-R156 | |
| | | | | | \|P47967\|11-143: L24-S157 | |
| | | | | | \|P38552\|190-323: L26-R156 | |
| | | | | | Lectin_Galactoside: W90-M110 | MOTIFS |
| 10 | 7501080CD1 | 380 | S75 S288 T27 T32 | | Signal Peptide: M1-A28, M1-S33 | HMMER |
| | | | T263 T322 T345 | | | |
| | | | Y231 | | | |
| | | | | | P52 PROAPOTOTIC PROTEIN | BLAST_PRODOM |
| | | | | | PD129433: E128-G285, I282-E372 | |
| | | | | | PD069653: M1-L115 | |
| 11 | 3144878CD1 | 189 | S150 T125 | | LIM domain: C49-A106, C109-Q175, M1-F42 | HMMER_PFAM |
| | | | | | LIM domain signature and profile: Y25-R173 | PROFILESCAN |
| | | | | | LIM METAL-BINDING REPEAT DM00055 | BLAST_DOMO |
| | | | | | \|Q04584\|464-533: Y101-H170 | |
| | | | | | \|Q04584\|405-462: Y39-D100 | |
| | | | | | LIM domain signature: C49-L82 | MOTIFS |
| 12 | 7502112CD1 | 827 | S6 S62 S69 S285 | N60 N437 | WD domain, G-beta repeat: L89-K124, | HMMER_PFAM |
| | | | S307 S311 S366 | N809 N819 | M178-S214, K360-R396, P262-Q302, | |
| | | | S418 S419 S614 | | L220-C256, L460-D497, N509-G544, | |
| | | | S629 S653 S682 | | E420-N454, F317-F352 | |
| | | | S696 S781 S811 | | | |
| | | | S817 S824 T114 | | | |
| | | | T203 T223 T292 | | | |
| | | | T318 T368 T391 | | | |
| | | | T687 T730 T777 | | | |
| | | | T797 Y769 | | | |
| | | | | | Trp-Asp (WD) repeat protein BLOO678: | BLIMPS_BLOCKS |
| | | | | | T203-W213 | |
| | | | | | Trp-Asp (WD) repeats signature: V441-S455, | MOTIFS |
| | | | | | M484-I498 | |
| 13 | 7502788CD1 | 1010 | S6 S62 S69 S285 | N60 N437 | WD domain, G-beta repeat: L89-K124, | HMMER_PFAM |
| | | | S307 S311 S366 | N809 N819 | M178-S214, K360-R396, P262-Q302, | |
| | | | S418 S419 S614 | | L220-C256, L460-D497, N509-G544, | |
| | | | S629 S653 S682 | | E420-N454, F317-F352 | |
| | | | S696 S781 S811 | | | |
| | | | S817 S824 S839 | | | |
| | | | S840 S847 S853 | | | |
| | | | S857 S862 S883 | | | |
| | | | S893 S894 S895 | | | |
| | | | T114 T203 T223 | | | |
| | | | T292 T318 T368 | | | |
| | | | T391 T687 T730 | | | |
| | | | T777 T797 T829 | | | |
| | | | T889 T928 T951 | | | |
| | | | Y769 Y1001 | | | |
| | | | | | Trp-Asp (WD) repeat protein BLOO678: | BLIMPS_BLOCKS |
| | | | | | T203-W213 | |
| | | | | | Eukaryotic putative RNA-binding region RNP-1 | MOTIFS |
| | | | | | signature: K124-L131 | |
| | | | | | Trp-Asp (WD) repeats signature: V441-S455, | MOTIFS |
| | | | | | M484-I498 | |
| 14 | 7500171CD1 | 776 | S44 S55 S110 S118 | N303 | Signal Peptide: M1-G24 | HMMER |
| | | | S131 S179 S186 | | | |
| | | | S218 S225 S267 | | | |
| | | | S287 S305 S381 | | | |
| | | | S382 S387 S454 | | | |
| | | | S483 S497 S547 | | | |
| | | | S562 S570 S572 | | | |
| | | | S634 S658 T350 | | | |
| | | | T436 T504 T602 | | | |
| | | | Y701 | | | |
| | | | | | TACC1 PD185469: M1-Q647 | BLAST_PRODOM |
| 15 | 7500172CD1 | 395 | S44 S52 S73 S87 | | Signal Peptide: M1-G24 | HMMER |
| | | | S139 S166 S181 | | | |
| | | | S189 S191 S253 | | | |
| | | | S277 T94 T221 | | | |
| | | | Y320 | | | |
| | | | | | TACC1 PD185469: P35-Q259 | BLAST_PRODOM |
| 16 | 7500899CD1 | 298 | S52 S55 S234 T196 | | Leucine Rich Repeat: | HMMER_PFAM |
| | | | Y138 | | N66-V88, A112-R134, Y42-P65, | |
| | | | | | K89-R111, E135-V158 | |
| | | | | | Leucine zipper pattern: L46-L67 | MOTIFS |
| 17 | 7500916CD1 | 139 | S54 S105 S137 | | NATURAL KILLER CELLS PROTEIN 4 | BLAST_PRODOM |
| | | | | | PRECURSOR SIGNAL | |
| | | | | | PD116715: E19-K139, M1-L31 | |
| | | | | | Cell attachment sequence: R121-D123 | MOTIFS |
| 18 | 7502479CD1 | 123 | T23 T27 T30 T105 | N99 N119 | | |
| | | | T112 | | | |
| 19 | 3015057CD1 | 621 | S100 S173 S223 | N307 | BTB/POZ domain: T17-L128 | HMMER_PFAM |
| | | | S236 S400 S439 | | Kelch motif: V451-Q497, A499-P544, | HMMER_PFAM |
| | | | S459 S472 S525 | | P401-P449, P348-P399, E546-I600 | |
| | | | S531 T219 T267 | | BTB (also known as BR-C/Ttk or ZiN) | BLIMPS_PFAM |
| | | | T287 T309 T498 | | PF00651: C46-F58 | |
| | | | T506 | | SARCOSIN PD094973: E249-H460 | BLAST_PRODOM |
| | | | | | SARCOSIN PD094945: R511-M621 | BLAST_PRODOM |
| | | | | | PROTEIN REPEAT MATRIX RING CANAL | BLAST_PRODOM |
| | | | | | KELCH R12E2.1 C47D12.7 KIAA0132 KIAA0469 | |
| | | | | | PD001473: S131-H261 | |
| | | | | | POZ DOMAIN | BLAST_DOMO |
| | | | | | DM00509\|Q04652\|131-335: D21-W201 | |
| | | | | | DM00509\|A45773\|130-334: D21-W201 | |
| | | | | | DM00509\|P34371\|194-386: E9-D184 | |
| | | | | | DM00509\|P21073\|1-198: G30-S204 | |
| | | | | | Methylated-DNA--protein-cysteine | MOTIFS |
| | | | | | methyltransferase active site: F44-V50 | |
| 20 | 6303570CD1 | 722 | S15 S29 S33 S34 | | | |
| | | | S52 S61 S69 S80 | | | |
| | | | S86 S135 S219 | | | |
| | | | S227 S252 S384 | | | |
| | | | S392 S601 S654 | | | |
| | | | T141 T237 T448 | | | |
| | | | T499 T569 T623 | | | |
| | | | T639 Y338 | | | |
| 21 | 7501579CD1 | 283 | S36 T8 T13 T20 | N155 N189 | Ndr family: I2-S254 | HMMER_PFAM |
| | | | T116 T159 Y216 | N258 | PROTEIN ZK1073.1 DRG1 RTP COMPLETE CDS | BLAST_PRODOM |
| | | | | | CYTOPLASMIC NDR1 NMYC DOWNSTREAM | |
| | | | | | PD011661: Y16-S242 | |
| 22 | 7503812CD1 | 412 | S94 S184 S188 | N186 N278 | signal_cleavage: M1-S29 | SPSCAN |
| | | | S199 S310 S337 | | START domain: I206-R411 | HMMER_PFAM |
| | | | T342 T393 | | Cytosolic domains: M1-R51, Q117-W122 | TMHMMER |
| | | | | | Transmembrane domains: T52-T74, S94-L116, | |
| | | | | | V123-L145 | |
| | | | | | Non-cytosolic domains: N75-T93, S146-A412 | |
| | | | | | Steroidogenic acute regulatory signature | BLIMPS_PRINTS |
| | | | | | PR00978: D299-R319, W371-L390, A391-A412 | |
| | | | | | MLN64 MRNA ES64 PROTEIN COSMID F26F4 | BLAST_PRODOM |
| | | | | | PD023778: M1-W183, S184-V216 | |
| | | | | | PROTEIN STEROIDOGENIC ACUTE | BLAST_PRODOM |
| | | | | | REGULATORY PRECURSOR MITOCHONDRION | |
| | | | | | STEROIDOGENESIS TRANSIT PEPTIDE | |
| | | | | | F52F12.7 PD006339: D217-R411 | |
| 23 | 7503970CD1 | 472 | S20 S44 S81 S92 | | Reticulon: P268-E472 | HMMER_PFAM |
| | | | S104 S133 S156 | | Cytosolic domain: Y324-L390 | TMHMMER |
| | | | S198 S229 S320 | | Transmembrane domains: F301-V323, L391-I413 | |
| | | | S385 T39 T52 T58 | | Non-cytosolic domains: M1-H300, G414-E472 | |
| | | | T82 T166 T355 | | RETICULON NSPLIKE RTN2A RTN2C | BLAST_PRODOM |
| | | | T363 | | PD124617: M1-L271 | |
| | | | | | PROTEIN NEUROENDOCRINE-SPECIFIC | BLAST_PRODOM |
| | | | | | RETICULON NSP-LIKE F27L4.17 T3F17.18 | |
| | | | | | RTN2A RTN2C A B PD006298: D274-E472 | |
| | | | | | do NEUROENDOCRINE; TROPOMYOSIN; | BLAST_DOMO |
| | | | | | NEURONAL; | |
| | | | | | DM03029\|A60021\|1-199: D274-S459 | |
| | | | | | DM03029\|A46583\|577-776: D274-G453 | |
| | | | | | DM03029\|I60903\|157-356: D274-G453 | |
| | | | | | DM03029\|I60904\|10-208: D274-G453 | |
| | | | | | Cell attachment sequence: R124-D126, R333-D335 | MOTIFS |
| 24 | 2578986CD1 | 84 | S64 S65 S67 S75 | | signal_cleavage: M1-A25 | SPSCAN |
| | | | | | Signal Peptide: M1-A25 | HMMER |
| | | | | | Signal Peptide: M1-A24 | HMMER |
| 25 | 7503786CD1 | 379 | S11 S39 S226 S333 | N118 | signal_cleavage: M1-R37 | SPSCAN |
| | | | T106 T196 T252 | | | |
| | | | T303 T346 T350 | | | |
| | | | | | GTP_CDC Cell division protein: K42-K322 | HMMER_PFAM |
| | | | | | GTPBINDING PROTEIN CELL DIVISION SEPTIN | BLAST_PRODOM |
| | | | | | HOMOLOG CONTROL CYCLE BRAIN H5 | |
| | | | | | PD002565: K42-M316 | |
| | | | | | CELL DIVISION PROTEIN GTPBINDING BRAIN | BLAST_PRODOM |
| | | | | | H5 PEANUTLIKE PUTATIVE SEPTIN CONTROL | |
| | | | | | PD014145: D336-E377 | |
| | | | | | do HCDC10; PEANUT; DM00875\|P28661\|125-322: | BLAST_DOMO |
| | | | | | V26-C224 | |
| | | | | | do HCDC10; PEANUT; DM00875\|P42208\|18-215: | BLAST_DOMO |
| | | | | | V26-D223 | |
| | | | | | do SPAC8A4.07; HCDC10; PEANUT; SPR28; | BLAST_DOMO |
| | | | | | DM00647\|P28661\|324-461: D225-R363 | |
| | | | | | do HCDC10; PEANUT; DM00875\|P42207\|16-212: | BLAST_DOMO |
| | | | | | V26-D223 | |
| | | | | | ATP/GTP-binding site motif A (P-loop): G52-S59 | MOTIFS |
| 26 | 7505684CD1 | 67 | S19 S27 Y14 | | signal_cleavage: M1-A45 | SPSCAN |
| 27 | 302655CD1 | 529 | S58 S128 S217 | N260 N478 | | |
| | | | S255 S309 S459 | | | |
| | | | T71 T152 T262 | | | |
| | | | T321 T338 T362 | | | |
| | | | T493 | | | |
| 28 | 4757551CD1 | 562 | S53 S62 S118 S125 | N26 N284 | signal_cleavage: M1-T39 | SPSCAN |
| | | | S192 S211 S323 | N327 | | |
| | | | S381 S399 S477 | | | |
| | | | S483 S492 T22 | | | |
| | | | T150 T270 T302 | | | |
| | | | T309 T317 T329 | | | |
| | | | T353 T389 T455 | | | |
| | | | T460 | | | |
| | | | | | AIBC1 PD185226: M1-P524, S125-K562 | BLAST_PRODOM |
| | | | | | do NEUROFILAMENT; TRIPLET; | BLAST_DOMO |
| | | | | | DM04498\|P12036\|434-1019: T61-S530 | |
| 29 | 7503157CD1 | 627 | S11 S93 S95 S101 | | Ank repeat: E237-N269, T504-S536, V204-E236, | HMMER_PFAM |
| | | | S128 S159 S208 | | E470-E502, N337-N369, T567-D599, T304-E336, | |
| | | | S214 S230 T10 T43 | | F371-D403, G271-A303, E404-D436, S437-D469, | |
| | | | T120 T176 T602 | | T537-A566 | |
| 30 | 7503702CD1 | 445 | S8 S147 S191 S268 | N388 N391 | Ank repeat: Q66-K98, Y132-K164, E99-I131, | HMMER_PFAM |
| | | | S311 S333 S367 | | D165-R197, L198-M230 | |
| | | | S377 S393 T338 | | | |
| | | | T356 T390 | | | |
| | | | | | Ank repeat proteins. PF00023: L71-L86, | BLIMPS_PFAM |
| | | | | | G133-Y142 | |
| 31 | 72096217CD1 | 453 | S71 S79 S152 S238 | N363 N447 | BAR domain (found in amphiphysin and clathrin | HMMER_PFAM |
| | | | S300 T23 T39 | | binding protein): Q19-L203 | |
| | | | T162 T220 T392 | | | |
| | | | T394 T449 Y68 | | | |
| | | | | | SH3 domain: F383-V452 | HMMER_PFAM |
| | | | | | Src homology 3 (SH3) domain proteins profile | BLIMPS_BLOCKS |
| | | | | | BL50002: A387-D405, E438-R451 | |
| | | | | | AMPHIPHYSIN PROTEIN SH3 BOX- | BLAST_PRODOM |
| | | | | | DEPENDENT MYC-INTERACTING DOMAIN | |
| | | | | | ISOFORM II REDUCED VIABILITY PD005419: | |
| | | | | | N17-L210; PD003208: K194-T328, S271-K384 | |
| | | | | | ISOFORM I BIN1 + 12A PD149887: L275-Q317 | |
| | | | | | SRC HOMOLOGY 3 (SH3) DOMAIN | BLAST_DOMO |
| | | | | | DM00025\|P49418\|623-691: F381-T449 | |
| | | | | | DM00025\|S62400\|623-691: F381-T449 | |
| | | | | | DM00025\|P50478\|610-678: F381-T449 | |
| 32 | 7503620CD1 | 510 | S27 S72 S161 S185 | N86 N103 | Signal_cleavage: M1-G18 | SPSCAN |
| | | | S199 S323 S360 | N145 N257 | | |
| | | | S390 S421 S506 | N320 N340 | | |
| | | | T25 T58 T63 T304 | | | |
| | | | T342 T399 T425 | | | |
| | | | | | Signal Peptide: M2-S17, M2-V20, M2-G22, | HMMER |
| | | | | | M1-G22 | |
| | | | | | Clusterin: M2-D193, S194-S415 | HMMER_PFAM |
| | | | | | Clusterin proteins BL00492: M2-G18, V26-N48, | BLIMPS_BLOCKS |
| | | | | | G52-L85, N86-M122, V128-M176, S184-D225, | |
| | | | | | C251-A300, D379-S415 | |
| | | | | | Clusterin signatures: T93-E141, I241-R291 | PROFILESCAN |
| | | | | | PRECURSOR GLYCOPROTEIN CLUSTERIN | BLAST_PRODOM |
| | | | | | SIGNAL PROTEIN PLASMA COMPLEMENT | |
| | | | | | CYTOLYSIS INHIBITOR CLI PD006991: S194-D414, | |
| | | | | | M2-F213 | |
| | | | | | CLUSTERIN DM07724\|P17697\|1-438: M2-F213, | BLAST_DOMO |
| | | | | | F191-D414 | |
| | | | | | CLUSTERIN DM07724\|P14018\|1-450: L5-R242, | BLAST_DOMO |
| | | | | | L195-H412 | |
| | | | | | Clusterin signature 1: C113-C121 | MOTIFS |
| | | | | | Clusterin signature 2: C261-C271 | MOTIFS |
| 33 | 7503621CD1 | 425 | S27 S72 S161 S209 | N86 N103 | Signal_cleavage: M1-G18 | SPSCAN |
| | | | S333 S370 S400 | N267 N330 | | |
| | | | T25 T58 T63 T314 | N350 | | |
| | | | T352 T409 Y182 | | | |
| | | | | | Signal Peptide: M2-S17, M2-V20, M2-G22, | HMMER |
| | | | | | M1-G22 | |
| | | | | | Clusterin: M2-G137, L138-E425 | HMMER_PFAM |
| | | | | | Clusterin proteins BL00492: M2-G18, V26-N48, | BLIMPS_BLOCKS |
| | | | | | G52-L85, N86-M122, V128-P176, F194-D235, | |
| | | | | | C261-A310, D389-E425 | |
| | | | | | Clusterin signatures: T93-G137, I251-R301 | PROFILESCAN |
| | | | | | PRECURSOR GLYCOPROTEIN CLUSTERIN | BLAST_PRODOM |
| | | | | | SIGNAL PROTEIN PLASMA COMPLEMENT | |
| | | | | | CYTOLYSIS INHIBITOR CLI PD006991: Q144-E425, | |
| | | | | | M2-G137 | |
| | | | | | CLUSTERIN | BLAST_DOMO |
| | | | | | DM07724\|P17697\|1-438: K40-E425, | |
| | | | | | M2-P390 DM07724\|P14018\|1-450: | |
| | | | | | N29-E425, L5-R252 | |
| | | | | | Clusterin signature 1: C113-C121 | MOTIFS |
| | | | | | Clusterin signature 2: C271-C281 | MOTIFS |
| 34 | 7503624CD1 | 330 | S27 S72 S114 S238 | N172 N235 | Signal_cleavage: M1-G18 | SPSCAN |
| | | | S275 S305 T25 T58 | N255 | | |
| | | | T63 T219 T257 | | | |
| | | | T314 Y85 Y87 | | | |
| | | | | | Signal Peptide: M2-S17, M2-V20, M2-G22, | HMMER |
| | | | | | M1-G22 | |
| | | | | | Clusterin: M2-D83, T84-E330 | HMMER_PFAM |
| | | | | | Clusterin proteins BL00492: F99-D140, C166-A215, | BLMPS_BLOCKS |
| | | | | | D294-E330 | |
| | | | | | Clusterin signatures: I156-R206 | PROFILESCAN |
| | | | | | PRECURSOR GLYCOPROTEIN CLUSTERIN | BLAST_PRODOM |
| | | | | | SIGNAL PROTEIN PLASMA COMPLEMENT | |
| | | | | | CYTOLYSIS INHIBITOR CLI PD006991: E82- | |
| | | | | | E330, M2-D83 | |
| | | | | | CLUSTERIN DM07724\|P17697\|1-438: N29-E330, | BLAST_DOMO |
| | | | | | M2-D83 | |
| | | | | | CLUSTERIN DM07724\|P14018\|1-450: F90-E330, | BLAST_DOMO |
| | | | | | L5-D83 | |
| | | | | | Clusterin signature 2: C176-C186 | MOTIFS |
| 35 | 7503625CD1 | 535 | S27 S72 S161 S185 | N86 N103 | Signal_cleavage: M1-G18 | SPSCAN |
| | | | S224 S348 S385 | N145 N282 | | |
| | | | S415 S446 S531 | N345 N365 | | |
| | | | T25 T58 T63 T329 | | | |
| | | | T367 T424 T450 | | | |
| | | | Y206 | | | |
| | | | | | Signal Peptide: M2-S17, M2-V20, M2-G22, | HMMER |
| | | | | | M1-G22 | |
| | | | | | Clusterin: M2-S440 | HMMER_PFAM |
| | | | | | Clusterin proteins BL00492: M2-G18, V26-N48, | BLIMPS_BLOCKS |
| | | | | | G52-L85, N86-M122, V128-M176, F209-D250, | |
| | | | | | C276-A325, D404-S440 | |
| | | | | | Clusterin signatures: T93-E141, I266-R316 | PROFILESCAN |
| | | | | | PRECURSOR GLYCOPROTEIN CLUSTERIN | BLAST_PRODOM |
| | | | | | SIGNAL PROTEIN PLASMA COMPLEMENT | |
| | | | | | CYTOLYSIS INHIBITOR CLI PD006991: Q168-D439, | |
| | | | | | M2-F231 | |
| | | | | | CLUSTERIN DM07724\|P17697\|1-438: M2-D439 | BLAST_DOMO |
| | | | | | CLUSTERIN DM07724\|P14018\|1-450: L5-H437 | BLAST_DOMO |
| | | | | | Clusterin signature 1: C113-C121 | MOTIFS |
| | | | | | Clusterin signature 2: C286-C296 | MOTIFS |
| 36 | 7503627CD1 | 350 | S39 S163 S200 | N97 N160 | Signal_cleavage: M1-P18 | SPSCAN |
| | | | S230 S261 S346 | N180 | | |
| | | | T144 T182 T239 | | | |
| | | | T265 | | | |
| | | | | | Signal Peptide: M2-P18, M1-P18, M2-P22 | HMMER |
| | | | | | Clusterin: P14-S255, M2-L13 | HMMER_PFAM |
| | | | | | Clusterin proteins BL00492: F24-D65, C91-A140, | BLIMPS_BLOCKS |
| | | | | | D219-S255 | |
| | | | | | Clusterin signatures: I81-R131 | PROFILESCAN |
| | | | | | PRECURSOR GLYCOPROTEIN CLUSTERIN | BLAST_PRODOM |
| | | | | | SIGNAL PROTEIN PLASMA COMPLEMENT | |
| | | | | | CYTOLYSIS INHIBITOR CLI PD006991: P14-D254 | |
| | | | | | CLUSTERIN DM07724\|P17697\|1-438: P14-D254, | BLAST_DOMO |
| | | | | | M2-L13 | |
| | | | | | CLUSTERIN DM07724\|P14018\|1-450: F8-H252 | BLAST_DOMO |
| | | | | | Clusterin signature 2: C101-C111 | MOTIFS |
| 37 | 7505744CD1 | 129 | S121 T112 | N81 | Signal_cleavage: M1-S26 | SPSCAN |
| | | | | | Signal Peptide: M1-S26, M1-A20 | HMMER |
| | | | | | Kazal-type serine protease inhibitor domain: | HMMER_PFAM |
| | | | | | C61-C109 | |
| | | | | | Osteonectin domain signatures: Q46-C88 | PROFILESCAN |
| | | | | | KAZAL PROTEINASE INHIBITOR | BLAST_DOMO |
| | | | | | DM00123\|P50291\|186-238: S55-C109 | |
| | | | | | OSTEONECTIN DOMAIN DM02833\|S60062\|383-663: | BLAST_DOMO |
| | | | | | A33-P113 | |
| 38 | 7503712CD1 | 391 | S38 S98 S130 S132 | N217 N351 | Signal_cleavage: M1-P23 | SPSCAN |
| | | | S151 S153 S195 | | | |
| | | | S201 S245 S306 | | | |
| | | | T304 Y152 | | | |
| | | | | | Signal Peptide: M1-G20, P5-G26, M1-G26 | HMMER |
| | | | | | von Willebrand factor type C (homeostasis related) | HMMER_PFAM |
| | | | | | domain: C51-C125 | |
| | | | | | CHORDIN FACTOR DEVELOPMENTAL | BLAST_PRODOM |
| | | | | | PROTEIN REPEAT PRECURSOR ORGANIZER- | |
| | | | | | SPECIFIC SECRETED DORSALIZING | |
| | | | | | GLYCOPROTEIN PD018424: F141-P292, G232-V352 | |
| | | | | | VON WILLEBRAND FACTOR TYPE C REPEAT | BLAST_DOMO |
| | | | | | DM00551\|A55195\|22-117: P31-P126 | |
| | | | | | Leucine zipper pattern: L315-L336 | MOTIFS |
| 39 | 7503713CD1 | 750 | S38 S98 S130 S132 | N217 N351 | Signal_cleavage: M1-P23 | SPSCAN |
| | | | S151 S153 S195 | N365 N434 | | |
| | | | S201 S245 S306 | | | |
| | | | S353 S488 S596 | | | |
| | | | T304 T367 T592 | | | |
| | | | Y152 | | | |
| | | | | | Signal Peptide: M1-G20, P5-G26, M1-G26 | HMMER |
| | | | | | von Willebrand factor type C domain: | HMMER_PFAM |
| | | | | | C51-C125, C665-C722 | |
| | | | | | CHORDIN FACTOR DEVELOPMENTAL | BLAST_PRODOM |
| | | | | | PROTEIN REPEAT PRECURSOR ORGANIZER- | |
| | | | | | SPECIFIC SECRETED DORSALIZING | |
| | | | | | GLYCOPROTEIN PD018424: Q440-L618, | |
| | | | | | G232-Y439, F141-P292, A413-V605 | |
| | | | | | PROTEIN PRECURSOR REPEAT | BLAST_PRODOM |
| | | | | | GLYCOPROTEIN SIGNAL NEL EGF-LIKE | |
| | | | | | DOMAIN CHORDIN B0024.14 PD015143: | |
| | | | | | P662-D727 | |
| | | | | | VON WILLEBRAND FACTOR TYPE C REPEAT | BLAST_DOMO |
| | | | | | DM00551\|A55195\|22-117: P31-P126 | |
| | | | | | VON WILLEBRAND FACTOR TYPE C REPEAT | BLAST_DOMO |
| | | | | | DM00551\|A55195\|637-751: R660-C722, | |
| | | | | | T592-L618 | |
| | | | | | Leucine zipper pattern: L315-L336 | MOTIFS |
| | | | | | Cell attachment sequence: R165-D167 | MOTIFS |
| | | | | | VWFC domain signature: C686-C722 | MOTIFS |
| 40 | 7502225CD1 | 373 | S62 S109 S309 | N61 | PROTEIN COILED COIL CHAIN MYOSIN | BLAST_PRODOM |
| | | | S336 S340 T121 | | REPEAT HEAVY ATP-BINDING FILAMENT | |
| | | | T143 Y227 | | HEPTAD PD000002: E79-E278 | |
| | | | | | PROTEIN REPEAT TROPOMYOSIN COILED | BLAST_PRODOM |
| | | | | | COIL ALTERNATIVE SPLICING SIGNAL | |
| | | | | | PRECURSOR CHAIN PD000023: E98-S293, | |
| | | | | | E85-K274 | |
| | | | | | TROPOMYOSIN DM00077\|P53935\|580-755: | BLAST_DOMO |
| | | | | | E124-E278, N169-R288, D92-K269 | |
| | | | | | DM00077\|P22793\|389-528: E131-K275 | |
| | | | | | CALDESMON DM06224\|P12957\|1-755: Q73-E278 | BLAST_DOMO |
| | | | | | TRICHOHYALIN DM03839\|Q07283\|91-443: | BLAST_DOMO |
| | | | | | G84-E278, E85-R288, F75-E278 | |
| 41 | 7502778CD1 | 551 | S94 S127 S134 | N502 | Zinc finger, C2H2 type, domain proteins BL00028: | BLIMPS_BLOCKS |
| | | | S162 S191 S196 | | C199-H215 | |
| | | | S232 S275 S294 | | | |
| | | | S304 S477 S491 | | | |
| | | | S531 T11 T121 | | | |
| | | | T239 T318 T379 | | | |
| | | | T538 T540 S8 | | | |
| 42 | 7503699CD1 | 173 | S147 | | Ank repeat: Q66-K98, Y132-K164, E99-I131 | HMMER_PFAM |
| 43 | 6026715CD1 | 358 | S354 T109 T200 | N316 | Cell division protein: M46-G324 | HMMER_PFAM |
| | | | T340 T343 | | | |
| | | | | | GTP-BINDING PROTEIN CELL DIVISION | BLAST_PRODOM |
| | | | | | SEPTIN HOMOLOG CONTROL CYCLE BRAIN | |
| | | | | | H5 PD002565: G47-N316 | |
| | | | | | HCDC10; PEANUT; DM00875\|P39827\|18-215: | BLAST_DOMO |
| | | | | | V30-P224 | |
| | | | | | HCDC10; PEANUT; DM00875\|JC2352\|12-207: | BLAST_DOMO |
| | | | | | V30-Y223 | |
| | | | | | HCDC10; PEANUT; DM00875\|P42208\|18-215: | BLAST_DOMO |
| | | | | | V30-Y223 | |
| | | | | | HCDC10; PEANUT; DM00875\|P42207\|16-212: | BLAST_DOMO |
| | | | | | V30-P224 | |
| | | | | | ATP/GTP-binding site motif A (P-loop): | MOTIFS |
| | | | | | G56-S63 | |
| 44 | 7503631CD1 | 413 | S27 S72 S161 S185 | N86 N103 | Signal_cleavage: M1-G18 | SPSCAN |
| | | | S321 S358 S388 | N145 N255 | | |
| | | | T25 T58 T63 T302 | N318 N338 | | |
| | | | T340 T397 Y206 | | | |
| | | | | | Signal Peptide: M2-S17, M2-V20, M2-G22, | HMMER |
| | | | | | M1-G22 | |
| | | | | | Clusterin: H227-E413, M2-V226 | HMMER_PFAM |
| | | | | | Clusterin proteins BL00492: M2-G18, V26-N48, | BLIMPS_BLOCKS |
| | | | | | G52-L85, N86-M122, V128-M176, C249-A298, | |
| | | | | | D377-E413 | |
| | | | | | Clusterin signatures: T93-E141, I239-R289 | PROFILESCAN |
| | | | | | PRECURSOR GLYCOPROTEIN CLUSTERIN | BLAST_PRODOM |
| | | | | | SIGNAL PROTEIN PLASMA COMPLEMENT | |
| | | | | | CYTOLYSIS INHIBITOR CLI PD006991: M2-F238, | |
| | | | | | V226-E413 | |
| | | | | | CLUSTERIN DM07724\|P17697\|1-438: M2-S228, | BLAST_DOMO |
| | | | | | H233-E413 | |
| | | | | | CLUSTERIN DM07724\|P14018\|1-450: L5-R240, | BLAST_DOMO |
| | | | | | A230-E413 | |
| | | | | | Clusterin signature 1: C113-C121 | MOTIFS |
| | | | | | Clusterin signature 2: C259-C269 | MOTIFS |
| 45 | 7503664CD1 | 349 | S27 S72 S161 S185 | N86 N103 | Signal_cleavage: M1-G18 | SPSCAN |
| | | | S233 T25 T58 T63 | N145 N291 | | |
| | | | Y206 | | | |
| | | | | | Signal Peptide: M2-S17, M2-V20, M2-G22, | HMMER |
| | | | | | M1-G22 | |
| | | | | | Clusterin: M2-V333 | HMMER_PFAM |
| | | | | | Clusterin proteins BL00492: M2-G18, V26-N48, | BLIMPS_BLOCKS |
| | | | | | G52-L85, N86-M122, V128-M176, F218-D259, | |
| | | | | | C285-E334 | |
| | | | | | Clusterin signatures: T93-E141, I275-R325 | PROFILESCAN |
| | | | | | PRECURSOR GLYCOPROTEIN CLUSTERIN | BLAST_PRODOM |
| | | | | | SIGNAL PROTEIN PLASMA COMPLEMENT | |
| | | | | | CYTOLYSIS INHIBITOR CLI PD006991: M2-D279, | |
| | | | | | Q168-D335 | |
| | | | | | CLUSTERIN DM07724\|P17697\|1-438: M2-D335 | BLAST_DOMO |
| | | | | | CLUSTERIN DM07724\|P14018\|1-450: L5-L331 | BLAST_DOMO |
| | | | | | Clusterin signature 1: C113-C121 | MOTIFS |
| | | | | | Clusterin signature 2: C295-C305 | MOTIFS |
| 46 | 7504812CD1 | 243 | S5 S27 S90 S137 | N171 | Signal_cleavage: M1-S28 | SPSCAN |
| | | | S142 S200 S221 | | | |
| | | | T225 | | | |
| | | | | | Signal Peptide: M1-P22, M1-S26, M1-S28, | HMMER |
| | | | | | M1-S25, M1-S29 | |
| | | | | | Insulin-like growth factor binding proteins | BLIMPS_BLOCKS |
| | | | | | BL00222: P34-P44, D55-P70, Y117-G144 | |
| | | | | | Kazal serine protease inhibitors family | BLIMPS_BLOCKS |
| | | | | | proteins BL00282: V112-R134 | |
| | | | | | Insulin-like growth factor binding proteins | PROFILESCAN |
| | | | | | signature: P34-R94 | |
| | | | | | Kazal serine protease inhibitors family | PROFILESCAN |
| | | | | | signature: C87-G159 | |
| | | | | | MAC25 FOLLISTATIN-LIKE PROTEIN | BLAST_PRODOM |
| | | | | | PROSTACYCLIN-STIMULATING FACTOR | |
| | | | | | PD151365: W193-K237, D172-V196 | |
| | | | | | PROTEASE SERINE MAC25 FOLLISTATIN-LIKE | BLAST_PRODOM |
| | | | | | PROTEIN PROSTACYCLIN-STIMULATING | |
| | | | | | FACTOR NOVEL PD015439: S108-N171 | |
| | | | | | GROWTH FACTOR PROTEIN PRECURSOR | BLAST_PRODOM |
| | | | | | BINDING SIGNAL INSULIN-LIKE IGF-BINDING | |
| | | | | | GLYCOPROTEIN CONNECTIVE PD001622: C32-C87 | |
| | | | | | PROTEIN FOLLISTATIN-LIKE MAC25 | BLAST_PRODOM |
| | | | | | PD029693: R78-P105 | |
| | | | | | Aldehyde dehydrogenases cysteine active site: | MOTIFS |
| | | | | | A64-G75 | |
| 47 | 7504996CD1 | 201 | S193 T79 T184 | N153 | Signal_cleavage: M1-S26 | SPSCAN |
| | | | | | Signal Peptide: M1-S26, M1-S20 | HMMER |
| | | | | | Kazal-type serine protease inhibitor domain: | HMMER_PFAM |
| | | | | | C59-C105, C133-C181 | |
| | | | | | Kazal serine protease inhibitors family proteins | BLIMPS_BLOCKS |
| | | | | | BL00282: G65-R87 | |
| | | | | | Osteonectin domain signatures: Q118-C160 | PROFILESCAN |
| | | | | | Kazal serine protease inhibitors family | PROFILESCAN |
| | | | | | signature: M27-S108 | |
| | | | | | KAZAL PROTEINASE INHIBITOR | BLAST_DOMO |
| | | | | | DM00123\|P50291\|186-238: S127-C181 | |
| | | | | | KAZAL PROTEINASE INHIBITOR | BLAST_DOMO |
| | | | | | DM00123\|P50291\|112-163: R54-C105 | |
| 48 | 7377279CD1 | 445 | S26 S45 S69 S111 | N6 N110 | Cell attachment sequence: R18-D20 | MOTIFS |
| | | | S368 S391 S407 T2 | | | |
| | | | T22 T46 T59 T87 | | | |
| | | | T98 T127 T159 | | | |
| | | | T259 T398 | | | |
| | | | | | Leucine zipper pattern: L282-L303 | MOTIFS |
| 49 | 2060984CD1 | 304 | S81 T50 T192 | | MAGE family: M1-Y208 | HMMER_PFAM |
| | | | T210 T254 T258 | | | |
| | | | Y136 | | | |
| | | | | | ANTIGEN MELANOMA-ASSOCIATED | BLAST_PRODOM |
| | | | | | MULTIGENE FAMILY PROTEIN TUMOR | |
| | | | | | RELATED POLYMORPHISM MAGE4 MAGEB1 | |
| | | | | | PD003141: Q101-G206; PD003293: V207-P275 | |
| | | | | | ANTIGEN; MELANOMA; NECDIN; MAGE-X2; | BLAST_DOMO |
| | | | | | DM01441\|P43366\|123-314: P105-A293 | |
| | | | | | ANTIGEN; MELANOMA; NECDIN; MAGE-X2; | BLAST_DOMO |
| | | | | | DM01441\|P43363\|149-340: Q101-A293 | |
| | | | | | ANTIGEN; MELANOMA; NECDIN; MAGE-X2; | BLAST_DOMO |
| | | | | | DM01441\|P43364\|127-318: 1106-E289 | |
| | | | | | ANTIGEN; MELANOMA; NECDIN; MAGE-X2; | BLAST_DOMO |
| | | | | | DM01441\|P25233\|117-308: D100-A293 | |
| | | | | | Cell attachment sequence: R168-D170 | MOTIFS |
| 50 | 4085993CD1 | 561 | S10 S14 S38 S120 | N49 N312 | Cell attachment sequence: R495-D497 | MOTIFS |
| | | | S129 S190 S194 | N354 N362 | | |
| | | | S207 S256 S275 | N446 | | |
| | | | S303 S307 S330 | | | |
| | | | S356 S367 S374 | | | |
| | | | S394 S396 S440 | | | |
| | | | S459 S461 S519 | | | |
| | | | T268 T385 T429 | | | |
| | | | T479 | | | |
| | | | | | Legume lectins beta-chain signature: | MOTIFS |
| | | | | | L169-S175 | |
| 51 | 6756305CD1 | 893 | S103 S119 S181 | N101 N195 | MYOSIN HEAVY CHAIN HOMOLOG DOC1 | BLAST_PRODOM |
| | | | S197 S203 S224 | N526 N711 | PD184558: L378-T453 | |
| | | | S237 S268 S309 | N810 N850 | | |
| | | | S371 S540 S551 | N861 | | |
| | | | S606 S722 S772 | | | |
| | | | S786 T49 T59 T74 | | | |
| | | | T100 T214 T245 | | | |
| | | | T266 T294 T296 | | | |
| | | | T303 T311 T412 | | | |
| | | | T455 T701 T721 | | | |
| | | | T816 T864 Y318 | | | |
| | | | | | PROTEIN COILED COIL CHAIN MYOSIN | BLAST_PRODOM |
| | | | | | REPEAT HEAVY ATPBINDING FILAMENT | |
| | | | | | HEPTAD PD000002: Q82-K316 | |
| | | | | | MYOSIN HEAVY CHAIN HOMOLOG DOC1 | BLAST_PRODOM |
| | | | | | PD075340: S119-F173 | |
| | | | | | SYNAPTONEMAL COMPLEX PROTEIN ZIP1 | BLAST_PRODOM |
| | | | | | NUCLEAR MEIOSIS COILED COIL PD074848: | |
| | | | | | Q7-L395 | |
| | | | | | TRICHOHYALIN DM03839\|P22793\|921-1475: | BLAST_DOMO |
| | | | | | E5-E532 | |
| | | | | | TRICHOHYALIN DM03839\|P37709\|632-1103: | BLAST_DOMO |
| | | | | | Q42-D480 | |
| | | | | | Leucine zipper pattern: L83-L104, L90-L111, | MOTIFS |
| | | | | | L218-L239, L225-L246, L232-L253 | |
| 52 | 7504842CD1 | 509 | S3 S6 S167 S265 | N144 N229 | Signal_cleavage: M1-G34 | SPSCAN |
| | | | S303 S357 S391 | N374 | | |
| | | | S416 S492 T118 | | | |
| | | | T146 T209 T223 | | | |
| | | | T289 T332 T345 | | | |
| | | | Y186 Y336 | | | |
| | | | | | Signal Peptide: M9-G34, M1-G34 | HMMER |
| | | | | | Leucine rich repeat C-terminal domain: | HMMER_PFAM |
| | | | | | N125-I174 | |
| 53 | 7500901CD1 | 312 | S52 S55 S181 T143 | | Leucine Rich Repeat: | HMMER_PFAM |
| | | | T148 T265 Y207 | | A112-K134, N66-V88, Y42-P65, S158-P180, | |
| | | | | | K89-R111, E135-L157, S181-R203, E204-V227 | |
| | | | | | Leucine zipper pattern: | MOTIFS |
| | | | | | L46-L67 | |
| 54 | 7500898CD1 | 61 | S52 | | | |
| 55 | 7500912CD1 | 168 | S22 S83 S134 S166 | | NATURAL KILLER CELLS PROTEIN 4 | BLAST_PRODOM |
| | | | | | PRECURSOR SIGNAL | |
| | | | | | PD116715: V19-K168, M1-M20 | |
| | | | | | Cell attachment sequence: R150-D152 | MOTIFS |
| 56 | 7505851CD1 | 139 | S54 S105 S137 | | NATURAL KILLER CELLS PROTEIN 4 | MOTIFS |
| | | | | | PRECURSOR SIGNAL PD116715: E19-K139 | |
| | | | | | Cell attachment sequence: R121-K139 | |
| 57 | 7503696CD1 | 77 | S8 | | | MOTIFS |

TABLE 4

| Polynucleotide | |
|---|---|
| SEQ ID NO:/ | |
| Incyte ID/Sequence | |
| Length | Sequence Fragments |
| 58/1318936CB1/ | 1-258, 18-574, 19-382, 21-468, 26-375, 77-475, 101-1894, 156-382, 283-1380, 286-1803, 335-1028, 364-1028, 420- |
| 2567 | 995, 607-1017, 607-1052, 607-1539, 784-1381, 785-1371, |
| | 806-1535, 887-1381, 896-1535, 982-1647, 982-1687, 982-1725, 1027-1725, 1047-1725, 1200-1527, 1206-1950, |
| | 1774-2243, 1774-2271, 2129-2567 |
| 59/033784CB1/ | 1-659, 9-239, 9-288, 9-375, 10-565, 10-669, 14-92, 14-270, 14-567, 15-248, 15-478, 16-222, 17-512, 36-268, |
| 941 | |
| | 36-307, 42-299, 48-250, 48-332, 91-350, 191-578, 191-585, 204-470, 230-488, 337-795, 341-793, 346-793, 347- |
| | 790, 360-795, 367-795, 369-800, 379-630, 380-791, 384-800, 390-795, 393-793, 396-792, 396- |
| | 793, 423-799, 431-793, 462-792, 463-617, 467-941, 488-794, 494-792, 496-792, 512-792, 553-927, 562-792 |
| 60/4160218CB1/ | 1-611, 74-890, 151-654, 209-828, 268-858, 370-953, 444-2672, 546-1020, 1330-1643, 1330-2675, 1503-1769, 1551- |
| 6795 | 2145, 1566-6795, 1577-2176, 1623-2265, 1670-2297, 1985-2249, 1985-2257, 1985-2429, 2039-2478, 2147-2668, |
| | 2381-2961, 2437-2675, 2438-3084, 2457-2780, 2501-2921, 2588-2787, 2619-2787, 2625-3214, 2658-2913, 2672- |
| | 2871, 2674-3134, 2712-3231, 2728-3117, 2750-3062, 2750-3290, 2750-3308, 2985-3612, 2995-3167, 2995-3243, |
| | 2997-3213, 3074-3687, 3093-3360, 3121-3542, 3212-3544, 3251-3562, 3280-3821, 3293-3438, 3293-3562, 3354- |
| | 3595, 3354-3653, 3396-3815, 3403-3530, 3403-4234, 3484-3680, 3528-3829, 3541-3842, 3550-3831, 3554-4222, |
| | 3593-4137, 3659-3809, 3667-4265, 3680-4270, 3712-4218, 3831-4102, 3835-4288, 3840-4289, 3851-4529, 3855- |
| | 4274, 3873-4256, 3879-4274, 3905-4405, 3924-4365, 3945-4194, 3992-4290, 4043-4274, 4122-4387, 4166-4478, |
| | 4191-4271, 4380-4607, 4511-4714, 4511-4785, 4724-5239, 4800-5232, 4991-5023, 4993-5026, 5062-5324, 5075- |
| | 5558, 5108-5397, 5126-5429, 5169-5429, 5176-5618, 5178-5618, 5185-5320, 5197-5618, 5454-5509, 5528-5630 |
| 61/7501989CB1/ | 1-179, 1-216, 1-236, 1-246, 1-248, 1-272, 1-273, 1-274, 1-287, 1-474, 1-489, 1-490, 1-500, 1-507, 1-512, 1-753, 9- |
| 2144 | 864, 10-567, 30-503, 36-163, 41-593, 49-300, 55-1634, 69-322, 148-425, 148-503, 185-897, 188-882, 223-774, 224- |
| | 761, 282-615, 282-699, 346-740, 428-1127, 451-718, 464-1050, 482-1128, 492-745, 499-1059, 519-948, 519-1064, |
| | 572-965, 581-1326, 601-829, 623-1231, 634-879, 679-1231, 688-1339, 751-1251, 751-1273, 752-1381, 767-1060, |
| | 770-1046, 770-1173, 770-1194, 770-1215, 770-1258, 770-1261, 770-1372, 770-1373, 771-1343, 784-1399, 788- |
| | 1352, 797-1496, 808-1343, 826-1311, 829-1227, 830-1069, 847-1343, 855-1150, 870-1615, 888-1423, 906-1729, |
| | 909-1569, 945-1467, 950-1570, 966-1620, 988-1634, 999-1600, 999-1616, 1001-1536, 1016-1576, 1024-1291, 1036- |
| | 1301, 1046-1342, 1090-1588, 1094-1736, 1104-1618, 1105-1384, 1107-1634, 1126-1634, 1138-1394, 1138-1395, |
| | 1147-1366, 1147-1633, 1176-1633, 1207-1634, 1222-1633, 1232-1634, 1234-1634, 1235-1634, 1243-1634, 1245- |
| | 1544, 1253-1634, 1270-1634, 1320-1634, 1324-2050, 1328-1634, 1349-1634, 1376-1634, 1377-2107, |
| | 1384-1634, 1391-1632, 1395-1634, 1416-1634, 1423-1632, 1423-1634, 1470-2064, 1486-1634, 1496-1634, 1501- |
| | 1634, 1507-2054, 1548-1612, 1552-1634, 1700-2112, 1702-2116, 1705-2113, 1708-2107, |
| | 1708-2117, 1709-2109, 1710-2130, 1716-2086, 1720-1967, 1727-2008, 1730-2113, 1735-1978, 1738-1989, 1745- |
| | 2032, 1768-2144, 1769-2111, 1777-2113, 1778-2111, 1779-2042 |
| 62/7500695CB1/ | 1-225, 1-1272, 18-227, 20-298, 26-292, 27-261, 27-284, 35-199, 35-359, 42-321, 44-259, 44-359, 46-286, 46-302, |
| 1345 | 46-624, 47-236, 49-306, 49-307, 52-248, 54-307, 54-360, 62-225, 62-319, 87-236, 476-739, |
| | 476-756, 476-918, 504-706, 505-659, 507-1072, 508-1074, 511-789, 523-1069, 533-735, 533-739, 533-871, 533- |
| | 980, 550-765, 574-794, 574-1144, |
| | 579-781, 585-1055, 615-1144, 622-1069, 641-1272, 648-1266, 673-938, 681-1266, 693-939, 695-931, 695-961, 714- |
| | 1263, 720-1282, 726-1274, 748-871, 754-1012, 759-1023, 760-1206, 771-1210, 774-1235, |
| | 775-1232, 782-1285, 783-1007, 785-1017, 788-1013, 791-1282, 803-1206, 818-1280, 847-1102, 849-1212, 864-992, |
| | 864-1265, 873-1281, 883-1030, 887-1039, 887-1178, |
| | 908-1282, 909-1181, 913-1194, 913-1198, 918-1252, 966-1282, 974-1282, 989-1225, 989-1228, 1001-1281, 1005- |
| | 1256, 1005-1285, 1010-1245, 1019-1282, 1053-1223, 1053-1281, 1053-1345, 1083-1295, 1088-1283, 1106-1282, |
| | 1142-1294, 1201-1296 |
| 63/7502088CB1/ | 1-515, 72-831, 80-337, 80-398, 88-358, 88-387, 90-775, 100-376, 100-377, 110-832, 112-679, 125-747, 131-468, |
| 1929 | 139-378, 241-711, 481-994, 487-1050, 610-1209, 837-1203, 978-1929, 1035-1623 |
| 64/7198825CB1/ | 1-657, 241-506, 295-890, 324-603, 324-954, 325-722, 533-749, 533-825, 603-1209, 746-963, 757-1034, 763-1382, |
| 3581 | 765-1351, 862-1106, 862-1304, 863-1131, 863-1326, 863-1343, 863-1433, 863-1445, 863-1527, 863-1652, 863- |
| | 1672, 924-1652, 951-1200, 986-1614, 1006-1626, 1013-1508, 1016-1290, 1023-1655, 1029-1339, 1049-1325, 1122- |
| | 1406, 1122-1648, 1124-1750, 1201-1859, 1227-1419, 1233-1666, 1236-1491, 1263-1864, 1273-1524, 1273-1719, |
| | 1274-1864, 1289-1861, 1325-1864, 1328-1546, 1336-1772, 1346-1864, 1353-1864, 1358-1864, 1361-1864, 1367- |
| | 1874, 1385-1864, 1390-1864, 1397-1435, 1426-1509, 1446-1607, 1450-1845, 1454-2112, 1459-1636, 1460-1864, |
| | 1470-2277, 1474-2277, 1484-1767, 1505-1864, 1509-2125, 1509-2142, 1523-1721, 1587-1870, 1587-2140, 1591- |
| | 1841, 1593-1855, 1609-1868, 1639-1745, 1643-1894, 1651-1895, 1661-2313, 1666-1971, 1670-1948, 1683-2226, |
| | 1696-2009, 1701-2327, 1714-2309, 1724-2166, 1757-1874, 1764-2010, 1794-1865, 1797-2405, 1803-2296, 1827- |
| | 2224, 1837-2100, 1851-2058, 1872-2082, 1898-2157, 1904-2223, 1935-2612, 1955-2537, 1955-2554, |
| | 1971-2136, 1984-2215, 2028-2166, 2050-2306, 2052-2243, 2052-2246, 2081-2416, 2084-2301, 2132-2408, 2138- |
| | 2601, 2147-2361, 2151-2802, 2175-2367, 2179-2351, 2187-2448, 2187-2506, 2193-2484, |
| | 2200-2645, 2214-2419, 2214-2424, 2225-2477, 2253-2477, 2254-2543, 2263-2374, 2264-2926, 2306-2939, 2311- |
| | 2449, 2311-2501, 2311-2769, 2321-2827, 2326-2460, 2326-2533, 2326-2554, 2326-2770, |
| | 2331-2643, 2337-2766, 2340-2635, 2355-2659, 2356-2642, 2360-2735, 2380-2657, 2383-2635, 2384-2618, 2390- |
| | 2671, 2397-2912, 2400-3120, 2400-3156, 2406-2668, 2406-2980, 2411-2662, 2419-2701, |
| | 2448-3104, 2451-3064, 2459-2654, 2459-2696, 2460-2506, 2460-2510, 2460-2596, 2489-2740, 2512-2730, 2521- |
| | 2777, 2522-3121, 2542-2831, |
| | 2546-2816, 2547-2710, 2549-2824, 2560-2755, 2566-3041, 2570-2884, 2573-2879, 2575-2867, 2578-2781, 2589- |
| | 2800, 2601-2850, 2602-2881, 2602-2889, |
| | 2603-3177, 2607-2801, 2613-3410, 2626-3410, 2646-2691, 2648-2952, 2666-2860, 2678-2862, 2681-2918, 2690- |
| | 3195, 2690-3259, 2697-2998, 2710-2997, 2721-3196, 2724-3324, 2724-3442, 2733-2994, |
| | 2734-3017, 2737-3043, 2745-3038, 2745-3279, 2746-3129, 2755-3480, 2781-2990, 2787-2957, 2794-3313, 2801- |
| | 3496, 2820-3471, 2821-3436, |
| | 2823-3395, 2852-3138, 2852-3146, 2852-3153, 2852-3272, 2852-3298, 2852-3500, 2852-3567, 2852-3581, 2855- |
| | 3274, 2859-3500, 2875-3164, 2887-3142 |
| 65/71863981CB1/ | 1-155, 1-2818, 190-679, 211-754, 341-754, 347-733, 347-768, 351-938, 351-1069, 354-495, 1116-1840, 1126-1732, |
| 3338 | 1142-1674, 1182-2000, 1209-1440, 1210-1840, 1257-1476, 1280-1809, 1309-1659, 1344-1968, 1344-1992, 1370- |
| | 1859, 1422-1856, 1426-1879, 1436-1651, 1459-1709, 1478-1735, 1478-1803, 1514-2110, 1525-1977, 1552-2000, |
| | 1589-2062, 1647-2002, 1647-2063, 1674-2095, 1723-2027, 1730-1985, 1796-1998, 1815-2464, 1815-2601, 1819- |
| | 2534, 1848-2502, 1855-2473, 1888-2483, 1896-2594, 1906-2474, 1908-2611, 1909-2646, 1937-2528, 1953-2616, |
| | 1957-2467, 1960-2533, 1975-2381, 1975-2422, 1981-2434, 1981-2523, 1985-2536, 1995-2495, 2014-2776, 2021- |
| | 2646, 2058-2690, 2060-2886, 2067-2598, 2086-2505, 2086-2519, 2093-2666, 2100-2442, 2100-2652, 2101-2544, |
| | 2103-2731, 2113-2704, 2116-2643, 2124-2709, 2132-2551, 2143-2845, 2144-2731, 2144-2792, 2145-2405, 2145- |
| | 2716, 2145-2745, 2145-2899, 2146-2701, 2154-2834, 2156-2821, 2170-2718, 2187-2857, 2189-2925, 2211-3026, |
| | 2216-2937, 2228-2418, 2228-2832, 2248-2891, 2251-2780, 2259-2789, 2304-2926, 2312-2522, 2320-2915, 2324- |
| | 2624, 2324-2632, 2324-3025, 2330-2801, 2333-2635, 2333-3017, 2344-2974, 2345-2715, |
| | 2347-3067, 2405-2900, 2414-3150, 2417-2715, 2417-2951, 2434-2953, 2441-3055, 2455-3082, 2457-3009, 2460- |
| | 3067, 2473-2895, 2483-2973, 2518-2804, 2522-3049, 2529-3214, 2530-3212, 2549-3337, 2581-3338, 2591-3050, |
| | 2609-2907, 2610-2898 |
| 66/7500513CB1/ | 1-161, 1-1004, 87-929, 89-929, 233-894, 236-704, 236-837, 253-707, 255-511, 258-749, 259-533, 261-759, 262- |
| 1004 | 502, 278-763, 285-911, 298-704, 301-763, 302-553, 309-762, 310-763, 313-552, 316-707, |
| | 316-762, 317-710, 319-897, 331-590, 331-623, 333-899, 347-850, 348-586, 348-614, 348-855, 349-735, 365-797, |
| | 366-583, 368-763, 375-763, 377-762, 393-704, 411-622, 411-689, 411-929, 432-974, 434-720, |
| | 452-773, 484-763, 484-898, 484-924, 489-912, 517-723, 524-853, 542-820, 588-930, 594-927, 597-893, 713-1004 |
| 67/7501080CB1 | 1-511, 1-608, 1-619, 1-630, 1-678, 2-232, 7-683, 9-129, 9-288, 9-399, 9-412, 9-424, 9-437, 9-443, 9-502, 9-527, 9- |
| 1280 | 536, 9-537, 9-538, 9-548, 9-580, 9-614, 9-616, 9-620, 9-654, 9-657, 9-658, 9-661, 9-662, 9-672, 9-688, 9-694, 9-717, |
| | 9-718, 9-750, 9-782, 10-558, 10-749, 11-627, 11-723, 1-3-298, 13-441, 13-516, 13-596, 13-651, 13-730, 15-687, 16- |
| | 310, 20-300, 21-257, 21-505, 21-550, 21-564, 21-630, 22-186, 23-670, 24-217, 25-641, 27-591, 32-750, 34-278, 34- |
| | 618, 34-786, 34-802, 41-679, 52-684, 62-772, 68-320, 89-609, 96-685, 119-679, 119-797, 126-730, 127-775, 132- |
| | 772, 181-707, 219-849, 221-800, 229-846, 244-766, 249-836, 271-757, 272-822, 288-632, 290-821, 290-871, 299- |
| | 779, 318-514, 355-838, 396-631, 409-849, 410-647, 425-760, 430-700, 431-622, 471-718, 491-736, 492-753, 531- |
| | 785, 539-1134, 539-1237, 541-1107, 550-762, 550-795, 560-809, 565-847, 570-822, 579-1033, 875-1143, 881-1266, |
| | 887-1266, 888-1266, 896-1262, 897-1262, 902-1269, 915-1269, 928-1266, 931-1159, 933-1262, 936-1154, 940- |
| | 1158, 947-1280, 955-1266, 958-1278, 961-1266, 983-1241, 994-1266, 1022-1268, 1023-1266, |
| | 1024-1189, 1058-1253, 1072-1269, 1099-1264, 1151-1280 |
| 68/3144878CB1/ | 1-456, 12-643, 286-792, 287-456, 287-462, 287-475, 295-823, 308-456, 309-564, 312-480, 314-379, 393-519, 411- |
| 1968 | 682, 513-851, 526-768, 540-1019, 586-788, 586-885, 772-909, 774-1040, 844-1089, 848-1008, |
| | 848-1095, 851-1093, 894-1127, 911-1445, 950-1203, 979-1634, 995-1259, 996-1031, 996-1217, 1074-1347, 1082- |
| | 1353, 1133-1811, 1139-1440, 1149-1438, 1151-1420, 1221-1821, 1293-1801, 1323-1527, |
| | 1342-1587, 1342-1591, 1360-1858, 1361-1813, 1364-1813, 1377-1627, 1382-1814, 1396-1816, 1399-1813, 1404- |
| | 1812, 1408-1800, 1412-1810, 1428-1779, 1440-1812, 1441-1695, 1441-1815, 1442-1812, |
| | 1446-1687, 1448-1821, 1450-1807, 1451-1812, 1469-1812, 1471-1670, 1471-1814, 1477-1739, 1479-1821, 1486- |
| | 1812, 1509-1812, 1510-1821, 1512-1747, 1515-1820, 1519-1968, 1521-1811, 1527-1803, |
| | 1540-1807, 1547-1812, 1560-1778, 1567-1813, 1588-1813, 1595-1815, 1597-1862, 1598-1812, 1599-1774, 1599- |
| | 1812, 1599-1814, 1604-1812, 1622-1815, 1683-1704, 1691-1816, 1732-1812 |
| 69/7502112CB1/ | 1-6962, 740-1139, 740-1143, 2020-2630, 2118-2755, 2497-2925, 2582-2920, 3129-3581, 4815-4864, 5712-5761, |
| 6962 | 5751-6289, 6617-6666 |
| 70/7502788CB1/ | 1-7127, 1998-2497, 2507-3121, 4728-5068, 4792-5068, 4977-5026, 5874-5923, 5897-6109, 5913-6156, 5913-6671, |
| 7127 | 5913-6702, 5913-6711, 5913-6740, 5918-6957, 5937-6874, 5980-6770, 6036-6829, 6152-6447, 6312-6620, 6375- |
| | 6558, 6456-7089, 6783-6832 |
| 71/7500171CB1/ | 1-221, 1-222, 1-286, 1-303, 1-338, 1-432, 1-2663, 4-86, 4-153, 9-217, 10-385, 27-313, 27-412, 41-510, 59-316, 189- |
| 2663 | 841, 197-808, 231-495, 353-830, 425-934, 463-952, 530-1034, 536-1134, 563-1106, 567-1148, |
| | 592-1041, 654-1293, 671-1208, 711-1224, 774-1422, 776-1385, 867-1149, 924-1396, 1117-1521, 1156-1392, 1266- |
| | 1542, 1466-1908, 1584-2128, 1974-2663, 1978-2315, 1980-2542, 2020-2287, 2024-2577, |
| | 2042-2595, 2055-2593, 2067-2595, 2104-2580, 2105-2582, 2138-2584, 2139-2659, 2179-2597, 2194-2573, 2207- |
| | 2400, 2211-2584, 2216-2584, 2225-2429, 2234-2594, 2285-2544, 2312-2600, 2332-2584, 2345-2529, 2345-2595, |
| | 2374-2662, 2457-2656, 2488-2660, 2491-2663 |
| 72/7500172CB1/ | 1-221, 1-222, 1-286, 1-303, 1-313, 1-1520, 4-86, 4-153, 9-217, 27-313, 59-317, 83-615, 83-767, 83-803, 83-951, 191- |
| 1520 | 799, 349-671, 370-678, 375-774, 376-1079, 384-949, 385-776, 423-797, 473-739, 573-826, |
| | 574-1072, 648-1520, 707-1520, 708-1520, 709-1520, 757-1520, 775-1227, 789-1031, 796-1519, 805-1062, 819- |
| | 1106, 831-1520, 835-1172, 837-1399, 841-1414, 877-1144, 881-1434, 899-1452, 912-1450, 924-1452, |
| | 961-1437, 962-1439, 995-1441, 996-1516, 1036-1454, 1037-1452, 1051-1430, 1064-1257, 1068-1441, 1073-1441, |
| | 1082-1286, 1091-1451, 1142-1401, 1169-1457, 1189-1441, 1202-1386, 1202-1452, 1231-1519, 1258-1452, 1314- |
| | 1513, 1345-1517, 1348-1520 |
| 73/7500899CB1/ | 1-227, 1-267, 1-1212, 7-227, 7-272, 7-284, 7-307, 8-228, 8-276, 11-270, 11-494, 15-268, 42-284, 46-336, 59-346, |
| 1212 | 286-494, 290-459, 383-939, 493-750, 504-875, 511-737, 514-1068, 540-772, 540-1128, 567-767, |
| | 582-740, 596-790, 598-1138, 604-1063, 623-907, 647-1205, 658-924, 684-1206, 684-1208, 719-772, 721-1210, 743- |
| | 1198, 746-1079, 746-1180, 746-1212, 759-1194, 759-1199, 760-1201, 768-1180, 773-1199, |
| | 779-1202, 781-1212, 797-1189, 801-1198, 817-1199, 821-1179, 822-1018, 822-1159, 824-1205, 829-1198, 831- |
| | 1208, 841-1196, 844-1206, 852-1207, 867-1198, 876-1194, 880-1202, 884-1198, 889-1208, 890-1180, |
| | 932-1199, 932-1212, 943-1199, 954-1198, 957-1105, 957-1189, 963-1211, 963-1212, 993-1212, 1016-1192, 1017- |
| | 1111, 1026-1156, 1073-1193, 1113-1207 |
| 74/7500916CB1/ | 1-234, 1-323, 1-358, 1-493, 3-641, 10-676, 13-76, 52-366, 97-539, 98-396, 98-596, 98-598, 98-657, 100-491, 100- |
| 702 | 644, 101-367, 101-486, 104-670, 105-638, 107-389, 109-477, 114-212, 114-217, 117-341, 120-247, 128-310, 128- |
| | 338, 128-378, 130-642, 131-393, 132-362, 132-438, 138-593, 141-383, 141-546, 143-473, 144-231, 145-638, 150- |
| | 395, 150-642, 151-638, 153-528, 162-528, 165-386, 165-487, 165-561, 165-677, 171-302, 175-461, 175-587, 175- |
| | 644, 180-521, 181-391, 183-636, 183-638, 187-644, 189-663, 192-455, 192-638, 192-659, 192-660, 192-686, 195- |
| | 627, 195-655, 195-658, 196-671, 197-692, 198-644, 202-503, 203-458, 203-485, 204-537, 205-644, 207-689, 210- |
| | 540, 210-555, 211-410, 211-637, 212-460, 213-663, 217-644, 218-342, 223-476, 223-624, 223-676, 224-641, 225- |
| | 641, 230-636, 230-641, 232-472, 232-674, 234-442, 234-638, 235-658, 237-672, 238-702, 239-653, 244-459, 247- |
| | 451, 247-488, 247-638, 249-455, 251-661, 256-635, 262-519, 262-641, 263-492, 264-637, 264-661, 265-650, 266- |
| | 517, 266-638, 267-468, 267-512, 270-482, 270-483, 270-499, 270-638, 272-400, 272-655, 273-699, 277-474, 277- |
| | 657, 281-517, 282-641, 282-642, 283-660, 285-454, 285-534, 289-556, 290-549, 293-547, 297-641, |
| | 297-643, 301-518, 302-637, 302-639, 303-499, 307-641, 308-574, 308-655, 316-613, 324-589, 330-530, 331-641, |
| | 335-569, 341-595, 341-603, 365-638, 366-644, 376-596, 376-637, 376-644, 380-655, 381-602, |
| | 391-636, 394-672, 397-658, 399-642, 403-640, 408-664, 409-619, 413-616, 416-634, 416-638, 416-666, 417-525, |
| | 429-638, 437-598, 446-668, 448-679, 449-658, 452-644, 455-637, 457-559, 457-658, 457-663, |
| | 463-644, 477-630, 479-698, 494-613, 496-612, 497-659, 498-654, 516-699, 522-658, 534-639, 539-632, 539-645, |
| | 546-641, 563-655, 566-637 |
| 75/7502479CB1/ | 1-245, 1-254, 1-261, 1-282, 1-472, 1-1050, 2-636, 4-206, 5-262, 5-554, 8-659, 17-813, 17-933, 21-644, 24-646, 28- |
| 1565 | 632, 31-666, 33-322, 35-270, 35-272, 37-655, 39-369, 40-108, 40-282, 40-285, 40-741, 42-312, |
| | 44-330, 45-300, 45-308, 45-611, 69-285, 69-514, 11 1-375, 133-400, 138-543, 209-413, 235-1009, 256-704, 279- |
| | 1050, 281-487, 305-522, 311-611, 311-629, 311-651, 322-1024, 326-1026, 367-625, 453-587, |
| | 455-1050, 481-1049, 491-1049, 594-1050, 605-832, 605-937, 621-646, 623-1050, 628-886, 628-906, 628-922, 628- |
| | 928, 628-930, 628-940, 628-951, 628-958, 630-940, 632-1050, 650-1050, 689-1049, 711-993, |
| | 758-1050, 764-1050, 768-1050, 790-1050, 821-1010, 865-1565, 878-1050, 1008-1050 |
| 76/3015057CB1/ | 1-611, 2-292, 2-524, 2-646, 3-454, 3-549, 3-586, 3-599, 5-339, 5-429, 28-678, 69-1934, 524-1205, 604-1205, 845- |
| 2536 | 997, 905-1205, 929-1148, 1167-1455, 1182-1281, 1232-1803, 1280-1383, 1364-2051, |
| | 1403-1703, 1403-1764, 1403-1829, 1403-1890, 1403-1928, 1403-1949, 1403-2003, 1451-1900, 1471-1750, 1472- |
| | 2314, 1525-2072, 1562-1744, 1564-2380, 1595-2390, 1597-1949, 1597-2361, 1614-1703, |
| | 1666-2338, 1670-2012, 1672-2307, 1686-1976, 1693-2185, 1709-2326, 1733-1950, 1733-2038, 1756-2318, 1795- |
| | 1897, 1831-2370, 1896-2274, 1905-2292, 1907-2371, 1908-2393, 1910-2206, 1930-2119, |
| | 1930-2120, 1939-2366, 1943-2536, 1949-2131, 1957-2381, 1977-2155, 1978-2353, 1990-2327, 2004-2381, 2019- |
| | 2341, 2023-2233, 2035-2389, 2045-2381, 2091-2381, 2168-2381, 2231-2368 |
| 77/6303570CB1/ | 1-519, 1-665, 82-2568, 794-1006, 1615-2062, 1615-2098, 1619-2099, 1623-2099, 1624-2226, 1734-1818, 1839- |
| 2568 | 2048 |
| 78/7501579CB1/ | 1-291, 1-499, 1-513, 1-540, 1-604, 1-609, 1-637, 1-661, 1-686, 1-736, 1-762, 1-769, 2-567, 2-688, 2-692, 2-754, 2- |
| 2150 | 812, 5-773, 23-826, 175-811, 200-765, 200-797, 209-801, 210-793, 231-817, 252-879, 259-493, 259-497, 259-499, |
| | 272-547, 279-431, 279-446, 279-722, 279-727, 279-750, 279-763, 279-886, 282-545, 282-796, 288-977, 306-596, |
| | 309-1041, 310-820, 326-567, 355-894, 367-634, 378-888, 416-1035, 426-715, 437-1063, 442-958, 484-603, 519- |
| | 1064, 523-784, 526-776, 531-792, 543-1026, 545-1102, 568-1068, 578-791, 587-813, 595-721, 595-1002, 595-1097, |
| | 609-826, 640-899, 659-1299, 667-993, 669-1064, 672-1284, 677-1239, 681-1115, 699-1265, 720-1259, 720-1264, |
| | 720-1297, 722-978, 723-1108, 728-1110, 730-946, 763-1112, 776-933, 780-1208, 797-1047, 797-1579, 800-1071, |
| | 800-1292, 804-1239, 806-1299, 807-1028, 818-1699, 820-1421, 844-1297, 858-1299, 860-1348, 870-1173, 874- |
| | 1313, 890-1331, 894-1064, 904-1170, 927-1414, 1035-1091, 1063-1298, 1070-1689, 1079-1562, 1082-1876, 1097- |
| | 1669, 1137-1568, 1147-1789, 1157-1531, 1167-1449, 1167-1669, 1167-1677, 1199-1477, |
| | 1204-1443, 1204-1584, 1206-1605, 1214-1669, 1214-1687, 1273-1692, 1273-1804, 1275-1539, 1288-1920, 1314- |
| | 1613, 1358-1972, 1361-1635, 1399-1667, 1422-1665, 1422-1954, 1422-2068, 1449-2071, |
| | 1452-2064, 1465-1741, 1470-1761, 1473-1717, 1487-2041, 1500-1747, 1513-1781, 1514-1853, 1529-1794, 1529- |
| | 1827, 1532-2081, 1533-1783, 1542-2085, 1546-2112, 1564-1916, 1566-2150, 1588-2015, |
| | 1594-1786, 1599-1805, 1616-1980, 1620-1916, 1631-2042, 1636-1914, 1652-2023, 1654-2075, 1670-2072, 1673- |
| | 2134, 1689-1801, 1689-1964, 1703-2144, 1719-1938, 1750-2083 |
| 79/7503812CB1/ | 1-223, 1-2074, 109-363, 132-649, 149-729, 151-378, 153-685, 153-999, 154-434, 158-430, 160-580, 160-650, 165- |
| 2088 | 718, 166-696, 166-831, 173-743, 185-744, 186-455, 186-644, 186-806, 186-822, 187-658, 195-660, 199-462, 200- |
| | 485, 200-538, 206-649, 222-499, 253-804, 291-575, 323-910, 329-596, 370-671, 457-824, 477-647, 484-746, 484- |
| | 785, 495-771, 525-785, 614-893, 666-923, 702-974, 727-952, 823-1429, 830-1444, 830-1474, 830-1632, 835-1531, |
| | 840-1417, 841-1347, 844-1528, 853-1437, 871-1314, 875-1417, 886-1192, 905-1178, 909-1476, 909-1553, 926- |
| | 1206, 931-1179, 933-1565, 943-1352, 944-1408, 951-1191, 956-1507, 970-1490, 973-1507, 973-1640, 975-1208, |
| | 978-1231, 983-1269, 998-1234, 1012-1267, 1020-1295, 1028-1132, 1028-1312, 1032-1312, 1032-1722, 1052-1298, |
| | 1076-1331, 1082-1677, 1095-1672, 1130-1394, 1133-1257, 1133-1387, 1134-1512, 1138-1330, 1138-1384, 1142- |
| | 1377, 1147-1342, 1149-1407, 1151-1507, 1151-1545, 1174-1423, 1191-1444, 1192-1810, 1194-1637, 1199-1436, |
| | 1203-1593, 1203-1619, 1224-1482, 1235-1491, 1235-1496, 1241-1515, 1241-1519, 1244-1524, |
| | 1245-1858, 1272-1528, 1280-1483, 1293-1899, 1299-1570, 1301-1567, 1313-2004, 1315-2074, 1321-1569, 1325- |
| | 1679, 1332-1834, 1347-1632, 1351-1591, 1352-1615, 1352-1674, 1365-1982, 1367-2018, 1384-1644, 1384-1691, |
| | 1387-1628, 1391-1657, 1408-1633, 1409-2065, 1410-1685, 1417-2018, 1420-1716, 1422-1710, 1431-1646, 1433- |
| | 1685, 1440-1656, 1444-1640, 1445-1695, 1453-1681, 1456-1752, 1462-1722, 1470-1671, 1470-1738, 1471-2087, |
| | 1484-2014, 1487-2056, 1492-2003, 1493-1753, 1498-2063, 1504-1786, 1509-2008, 1515-2088, 1516-2021, 1529- |
| | 2018, 1543-2066, 1556-2088, 1569-1830, 1582-1808, 1582-1863, 1583-1765, 1584-2088, 1597-1963, 1599-2072, |
| | 1603-1833, 1604-1843, 1608-2088, 1609-1876, 1610-2088, 1618-1904, 1618-2028, 1618-2088, 1621-2074, 1621- |
| | 2088, 1628-1888, 1631-2034, 1631-2088, 1632-2088, 1640-2088, 1642-1892, 1646-2075, 1648-2070, 1651-2077, |
| | 1654-2074, 1656-2072, 1661-2074, 1663-1942, 1670-2052, 1681-2080, 1686-2080, 1687-1942, 1687-2085, 1688- |
| | 2075, 1691-2068, 1691-2088, 1692-1968, 1694-2046, 1694-2074, 1697-2074, 1697-2076, 1700-2074, 1701-2074, |
| | 1704-2074, 1712-2004, 1712-2074, 1715-2070, 1718-2074, 1731-2078, 1737-2080, |
| | 1245-1858, 1272-1528, 1280-1483, 1293-1899, 1299-1570, 1301-1567, 1313-2004, 1315-2074, 1321-1569, 1325- |
| | 1679, 1332-1834, 1347-1632, 1351-1591, 1352-1615, 1352-1674, 1365-1982, 1367-2018, |
| | 1384-1644, 1384-1691, 1387-1628, 1391-1657, 1408-1633, 1409-2065, 1410-1685, 1417-2018, 1420-1716, 1422- |
| | 1710, 1431-1646, 1433-1685, 1440-1656, 1444-1640, 1445-1695, 1453-1681, 1456-1752, |
| | 1462-1722, 1470-1671, 1470-1738, 1471-2087, 1484-2014, 1487-2056, 1492-2003, 1493-1753, 1498-2063, 1504- |
| | 1786, 1509-2008, 1515-2088, 1516-2021, 1529-2018, 1543-2066, 1556-2088, 1569-1830, |
| | 1582-1808, 1582-1863, 1583-1765, 1584-2088, 1597-1963, 1599-2072, 1603-1833, 1604-1843, 1608-2088, 1609- |
| | 1876, 1610-2088, 1618-1904, 1618-2028, 1618-2088, 1621-2074, 1621-2088, 1628-1888, |
| | 1631-2034, 1631-2088, 1632-2088, 1640-2088, 1642-1892, 1646-2075, 1648-2070, 1651-2077, 1654-2074, 1656- |
| | 2072, 1661-2074, 1663-1942, 1670-2052, 1681-2080, 1686-2080, 1687-1942, 1687-2085, |
| | 1688-2075, 1691-2068, 1691-2088, 1692-1968, 1694-2046, 1694-2074, 1697-2074, 1697-2076, |
| | 1700-2074, 1701-2074, 1704-2074, 1712-2004, 1712-2074, 1715-2070, 1718-2074, 1731-2078, 1737-2080, 1744- |
| | 1998, 1747-1912, 1749-2074, 1756-2061, 1760-2030, 1777-2075, 1782-2078, 1783-2077, |
| | 1806-2074, 1807-2081, 1809-2043, 1811-2017, 1829-2074, 1840-2077, 1842-2017, 1845-2072, 1867-2075, 1912- |
| | 2088, 1913-2074, 1915-2068, 1915-2074, 1928-2064 |
| 80/7503970CB1/ | 1-234, 1-354, 1-581, 1-611, 1-616, 1-618, 1-1973, 27-220, 29-557, 55-343, 84-609, 84-658, 86-613, 135-833, 317- |
| 2017 | 568, 374-643, 374-649, 397-11 16, 404-678, 421-715, 421-779, 434-1003, 462-750, 466-736, 477-651, 493-1128, |
| | 517-1165, 543-987, 551-810, 572-997, 594-834, 594-1011, 594-1059, 594-1141, 594-1217, 607-1245, 632-882, 645- |
| | 747, 658-1000, 661-1056, 690-1434, 694-1180, 697-1057, 717-1318, 756-1238, 833-1441, 885-1421, 892-1366, 896- |
| | 1412, 903-1444, 935-1149, 935-1580, 953-1160, 953-1181, 953-1185, 953-1197, 953-1200, 953-1234, 953-1267, |
| | 953-1450, 953-1486, 956-1195, 963-1591, 965-1233, 975-1550, 978-1483, 983-1212, 1003-1590, 1030-1446, 1030- |
| | 1483, 1035-1398, 1056-1588, 1057-1340, 1067-1326, 1070-1172, 1070-1369, 1070-1372, 1079-1593, 1139-1595, |
| | 1144-1773, 1145-1270, 1149-1420, 1149-1432, 1149-1457, 1158-1419, 1170-1415, 1176-1661, 1181-1423, 1191- |
| | 1561, 1203-1558, 1229-1826, 1236-1595, 1252-1592, 1263-1919, 1284-1543, 1298-1542, 1298-1587, 1310-1756, |
| | 1310-1960, 1312-1595, 1315-1970, 1328-1592, 1330-1592, 1330-1865, 1340-1604, 1349-1595, |
| | 1360-1899, 1375-1968, 1383-1621, 1384-1991, 1430-1992, 1448-1678, 1452-1587, 1464-1649, 1487-1998, 1489- |
| | 1972, 1500-1997, 1514-1992, 1520-1969, 1535-1973, 1535-1987, 1538-1973, 1541-1990, |
| | 1552-2017, 1553-1975, 1563-1842, 1589-1712, 1597-1970, 1597-1974, 1603-1969, 1606-1973, 1610-1969, 1617- |
| | 1975, 1618-1894, 1629-1972, 1634-1973, 1635-1969, 1637-1973, 1639-1992, 1650-1831, |
| | 1651-1974, 1664-1881, 1665-1969, 1681-1969, 1709-1876, 1720-1969, 1723-1970, 1725-1982, 1744-1969, 1767- |
| | 1852, 1785-1967, 1786-1970, 1789-2009, 1795-1973 |
| 81/2578986CB1 | 1-304, 1-1270, 88-264, 118-752, 342-789, 342-911, 359-761, 359-831, 542-1202, 572-1228, 667-1210, 763-1341, |
| 1341 | 856-1016, 956-1210 |
| 82/7503786CB1/ | 1-329, 51-1571, 72-1589, 140-787, 141-386, 141-408, 142-302, 142-415, 142-712, 143-329, 146-285, 161-309, 171- |
| 1600 | 329, 203-458, 319-956, 328-685, 329-556, 330-540, 330-617, 331-643, 334-623, 335-983, 338-612, 347-849, 348- |
| | 685, 354-683, 357-848, 362-590, 378-645, 387-639, 392-637, 393-1051, 396-650, 398-668, 400-1051, 419-608, 422- |
| | 661, 430-783, 431-773, 432-647, 434-718, 460-704, 472-1066, 475-994, 482-893, 482-1076, 483-697, 483-1092, |
| | 490-748, 514-633, 514-748, 540-816, 546-1067, 551-809, 551-1176, 571-736, 571-1034, 575-746, 575-749, 575- |
| | 840, 575-1193, 583-1035, 614-871, 614-874, 614-918, 620-883, 620-939, 635-902, 641-925, 641-927, 642-697, 647- |
| | 918, 657-920, 662-1176, 663-960, 670-1224, 675-1124, 679-1278, 683-946, 685-1161, 689-1287, 693-958, 697-933, |
| | 710-1002, 716-1281, 775-1088, 811-1072, 817-1035, 824-1074, 861-1375, 873-1378, 903-1371, 903-1511, 904- |
| | 1221, 906-1504, 922-1547, 926-1398, 929-1504, 932-1200, 932-1501, 959-1213, 964-1501, 987-1249, 987-1272, |
| | 998-1272, 1001-1247, 1008-1242, 1008-1561, 1016-1210, 1016-1299, 1023-1285, 1055-1570, |
| | 1063-1287, 1063-1315, 1063-1322, 1063-1325, 1063-1327, 1063-1328, 1063-1331, 1063-1332, 1063-1335, 1063- |
| | 1336, 1063-1337, 1063-1339, 1063-1341, 1063-1342, 1063-1343, 1063-1344, 1063-1346, |
| | 1063-1347, 1063-1348, 1063-1349, 1063-1352, 1063-1353, 1063-1359, 1063-1360, 1063-1362, 1063-1363, 1063- |
| | 1365, 1063-1366, 1063-1367, 1063-1369, 1063-1371, 1063-1373, 1063-1375, 1063-1589, |
| | 1063-1591, 1063-1592, 1067-1350, 1080-1349, 1098-1572, 1117-1571, 1135-1573, 1140-1572, 1141-1345, 1141- |
| | 1600, 1188-1571, 1205-1543, 1328-1571, 1344-1572 |
| 83/7505684CB1/ | 1-306, 50-281, 50-535, 73-337, 79-483, 135-413, 490-740, 491-708, 496-708, 498-693, 500-709, 515-709, 525-725 |
| 740 | |
| 84/302655CB1/ | 1-229, 1-541, 1-564, 1-593, 1-597, 10-573, 164-362, 223-862, 253-895, 318-851, 319-814, 326-959, 363-508, 383- |
| 2446 | 995, 384-996, 400-911, 421-960, 429-947, 430-914, 492-1069, 572-2402, 646-1321, 653-1225, |
| | 693-1283, 715-1198, 743-1319, 852-1406, 902-1504, 911-1521, 912-1128, 1018-1502, 1061-1697, 1065-1381, 1081- |
| | 1606, 1086-1547, 1098-1691, 1107-1391, 1133-1220, 1136-1760, 1146-1670, 1177-1746, |
| | 1241-1760, 1241-1821, 1254-1818, 1303-1853, 1336-1734, 1390-1889, 1394-1927, 1394-2022, 1400-2024, 1415- |
| | 1624, 1476-1536, 1496-2031, 1516-2393, 1563-2130, 1595-1913, 1619-2263, 1647-2229, |
| | 1648-2275, 1717-2262, 1754-2286, 1835-2435, 1882-2446, 1898-2424, 1967-2443, 2057-2401, 2101-2304, 2102- |
| | 2419, 2267-2417 |
| 85/4757551CB1/ | 1-275, 1-581, 1-858, 15-2000, 50-492, 124-462, 238-875, 295-796, 295-805, 318-1165, 380-1027, 382-824, 391- |
| 2020 | 788, 398-953, 401-897, 423-939, 425-997, 426-945, 481-1077, 483-1062, 525-785, 554-1048, 564-818, 564-822, |
| | 566-1059, 578-828, 578-852, 611-746, 612-1256, 616-708, 620-1212, 621-883, 623-870, 624-883, 625-873, 627- |
| | 885, 627-940, 630-791, 630-887, 630-1187, 631-900, 633-827, 634-904, 635-924, 637-873, 639-890, 640-919, 641- |
| | 771, 641-827, 641-884, 641-889, 641-890, 641-894, 641-903, 641-919, 641-937, 641-960, 651-1127, 686-920, 717- |
| | 786, 720-995, 786-1032, 796-1120, 815-1059, 824-1032, 825-945, 840-1086, 845-1350, 853-1124, 856-1477, 869- |
| | 1448, 885-1443, 889-1510, 909-1609, 978-1608, 998-1637, 1004-1603, 1011-1479, 1028-1615, 1058-1574, 1061- |
| | 1682, 1064-1427, 1183-1599, 1186-1760, 1241-1765, 1241-1958, 1318-1837, 1429-1653, 1441-1701, 1454-1732, |
| | 1492-1958, 1495-1774, 1497-1792, 1505-2020, 1506-2010, 1510-1807, 1576-1953, 1603-1951, 1623-2009, 1632- |
| | 1926, 1658-1958, 1659-1958, 1703-1966, 1749-2003, 1754-2006, 1840-2020 |
| 86/7503157CB1/ | 1-629, 17-725, 20-784, 21-242, 29-240, 31-689, 34-418, 35-780, 36-423, 36-864, 37-903, 41-625, 42-844, 50-724, |
| 2160 | 67-814, 74-585, 74-710, 74-714, 80-688, 96-766, 96-851, 123-714, 129-655, 150-780, 215-906, 351-751, 381-821, |
| | 383-1075, 406-595, 406-907, 406-972, 406-1084, 416-682, 432-810, 447-1061, 449-708, 449-862, 449-939, 449- |
| | 968, 449-1021, 470-937, 473-840, 487-1120, 509-1052, 524-875, 524-1218, 540-1077, 559-1365, 636-1265, 639- |
| | 1113, 640-1305, 644-959, 650-961, 651-961, 655-961, 656-961, 661-961, 663-961, 665-959, 665-961, 665-964, 665- |
| | 965, 665-969, 672-888, 672-916, 733-1101, 733-1188, 733-1190, 733-1212, 733-1215, 733-1217, 733-1221, 733- |
| | 1227, 733-1229, 735-1404, 738-1221, 738-1573, 741-1356, 774-1221, 791-1316, 799-1075, 808-1221, 833-1515, |
| | 846-1064, 856-1510, 859-1248, 862-1549, 866-1165, 888-1518, 892-1609, 932-1461, 977-1542, 991-1489, 998- |
| | 1261, 1023-1626, 1027-1347, 1027-1493, 1027-1504, 1039-1435, 1042-1626, 1057-1294, 1084-1638, 1093-1691, |
| | 1101-1685, 1120-1523, 1134-1730, 1141-1762, 1155-1784, 1205-1435, 1211-1580, 1222-1988, |
| | 1224-1513, 1224-1604, 1226-1487, 1247-1468, 1259-1675, 1272-1817, 1291-1585, 1297-1764, 1297-1807, 1298- |
| | 1597, 1307-1784, 1332-1583, 1343-1707, 1370-1754, 1381-1973, 1394-1973, 1394-2011, |
| | 1414-1611, 1422-1917, 1424-1691, 1434-2090, 1462-1718, 1473-1708, 1489-1973, 1498-2160, 1499-2154, 1516- |
| | 2101, 1523-1927, 1532-1771, 1558-1973, 1592-2103, 1610-2148, 1615-2154, 1639-1837, |
| | 1685-2160, 1690-1973, 1695-1973, 1695-2154, 1718-1968, 1744-1973, 1744-2143, 1744-2151, 1784-1974, 1787- |
| | 1973, 1823-1973, 1928-2151 |
| 87/7503702CB1/ | 1-275, 1-509, 1-3600, 242-525, 242-937, 500-1022, 787-910, 799-1607, 1071-1334, 1071-1590, 1071-1725, 1071- |
| 3685 | 1773, 1119-1458, 1122-1608, 1153-1609, 1320-1456, 1397-2251, 1644-2181, 1644-2354, |
| | 1645-2267, 1645-2275, 1645-2293, 1645-2365, 1646-2315, 1719-2540, 1818-2531, 1825-2540, 1858-2531, 1887- |
| | 2473, 2239-2532, 2872-3130, 2872-3387, 2882-3542, 2885-3296, 2888-3506, 2892-3422, |
| | 2909-3530, 2911-3502, 2931-3505, 2939-3576, 2948-3569, 2952-3427, 2969-3459, 2972-3486, 2974-3579, 2989- |
| | 3566, 2994-3490, 2994-3582, 3008-3551, 3010-3606, 3014-3405, 3044-3631, 3044-3660, |
| | 3052-3551, 3054-3629, 3056-3664, 3124-3685, 3128-3586, 3137-3659, 3192-3644, 3199-3590, 3252-3603, 3261- |
| | 3624, 3310-3569 |
| 88/72096217CB1/ | 1-490, 1-511, 1-522, 1-525, 1-614, 1-615, 1-623, 1-641, 1-717, 1-718, 1-776, 2-540, 2-613, 5-715, 39-902, 63-745, |
| 1860 | 130-844, 202-965, 224-897, 471-999, 476-965, 521-1041, 554-1120, 632-1187, 693-1102, 717- |
| | 838, 791-1463, 797-1099, 809-1254, 828-1442, 874-1403, 878-1436, 885-1387, 914-1457, 919-1108, 922-1342, 923- |
| | 1342, 956-1099, 967-1159, 967-1365, 967-1374, 967-1511, 967-1516, 967-1757, 971-1510, |
| | 988-1380, 1031-1499, 1052-1670, 1055-1281, 1064-1684, 1086-1674, 1091-1298, 1106-1757, 1118-1324, 1118- |
| | 1356, 1150-1382, 1164-1293, 1172-1851, 1174-1856, 1176-1856, 1188-1851, 1236-1488, 1239- |
| | 1442, 1241-1487, 1246-1524, 1266-1493, 1287-1852, 1303-1559, 1305-1559, 1305-1574, 1320-1831, 1347-1817, |
| | 1368-1612, 1374-1624, 1381-1597, 1400-1446, 1404-1644, 1404-1810, 1419-1697, 1433- |
| | 1678, 1469-1694, 1474-1700, 1476-1856, 1479-1713, 1491-1719, 1550-1812, 1560-1769, 1620-1829, 1663-1856, |
| | 1708-1860 |
| 89/7503620CB1/ | 1-1610, 48-284, 49-147, 49-166, 49-185, 49-196, 49-201, 49-215, 49-218, 49-227, 49-228, 49-231, 49-232, 49-234, |
| 1610 | 49-235, 49-237, 49-239, 49-240, 49-244, 49-247, 49-248, 49-252, 49-253, 49-255, 49-256, 49-258, 49-261, 49-263, |
| | 49-265, 49-270, 49-273, 49-277, 49-278, 49-279, 49-280, 49-283, 49-284, 49-286, 49-288, 49-289, 49-291, 49-292, |
| | 49-295, 49-296, 49-298, 49-303, 49-305, 49-311, 49-314, 49-333, 49-335, 49-345, 49-379, 49-423, 49-446, 49-449, |
| | 49-551, 49-612, 49-616, 49-641, 49-649, 50-246, 56-304, 59-336, 62-344, 64-423, 69-254, 70-412, 71-490, 72-332, |
| | 73-349, 80-658, 83-358, 103-361, 103-528, 105-574, 106-380, 107-505, 112-253, 117-566, 119-517, 119-584, 122- |
| | 603, 124-448, 130-423, 130-549, 133-338, 135-230, 138-511, 148-362, 154-374, 164-623, 171-417, 172-371, 174- |
| | 435, 177-285, 179-404, 179-441, 179-450, 182-333, 183-385, 183-389, 190-420, 197-490, 200-389, 200-495, 209- |
| | 440, 209-475, 209-613, 213-493, 214-379, 224-509, 225-469, 230-467, 234-458, 236-438, 242-437, 244-412, 245- |
| | 476, 251-550, 253-474, 253-479, 255-545, 258-481, 258-484, 258-657, 259-453, 259-569, 262-374, 263-587, 265- |
| | 391-634, 393-615, 397-658, 407-603, 419-652, 421-511, 421-617, 426-625, 429-655, 433-608, 433-658, 547-658, |
| | 604-1224, 656-816, 656-820, 656-835, 656-877, 656-879, 656-883, 656-896, 656-903, 656-922, |
| | 656-930, 656-995, 660-896, 673-943, 679-910, 682-913, 682-1242, 687-1225, 689-953, 692-932, 692-962, 694-939, |
| | 697-955, 699-917, 699-926, 700-924, 700-936, 702-950, 717-925, 717-926, 717-935, 717-967, |
| | 717-981, 729-958, 731-1083, 735-1074, 739-1149, 740-974, 748-952, 748-996, 748-1011, 748-1038, 749-992, 749- |
| | 1011, 749-1023, 750-1188, 754-970, 754-1013, 765-999, 770-1047, 781-1010, 781-1015, |
| | 784-1069, 791-1020, 791-1023, 793-992, 801-1074, 802-1056, 808-960, 809-1042, 815-1219, 817-941, 817-971, |
| | 817-1000, 818-1086, 819-1090, 827-1042, 829-1031, 830-1031, 832-1072, 832-1090, 837-1250, |
| | 840-1096, 842-1105, 844-1212, 852-1099, 869-1182, 880-1148, 881-1078, 882-1117, 882-1139, 883-1152, 887- |
| | 1108, 892-1172, 894-1186, 899-1137, 902-1227, 902-1264, 902-1300, |
| | 906-1191, 906-1199, 911-1311, 912-1161, 916-1143, 920-1158, 921-1267, 941-1123, 942-1262, 948-1206, 971- |
| | 1221, 995-1182, 1000-1256, 1002-1159, 1005-1195, 1024-1279, 1031-1222, 1032-1155, 1032- |
| | 1264, 1032-1318, 1033-1302, 1034-1255, 1035-1318, 1036-1235, 1039-1256, 1040-1280, 1052-1301, 1058-1315, |
| | 1065-1294, 1070-1198, 1082-1209, 1082-1318, 1084-1303, 1084-1317, 1085-1314, 1087-1318, 1091-1291, 1091- |
| | 1298, 1103-1318 |
| 90/7503621CB1/ | 1-1369, 48-284, 49-147, 49-166, 49-185, 49-196, 49-201, 49-215, 49-218, 49-227, 49-228, 49-231, 49-232, 49-234, |
| 1369 | 49-235, 49-237, 49-239, 49-240, 49-244, 49-247, 49-248, 49-252, 49-253, 49-255, 49-256, 49-258, 49-261, 49-263, |
| | 49-265, 49-270, 49-273, 49-277, 49-278, 49-279, 49-280, 49-283, 49-284, 49-286, 49-288, 49-289, 49-291, 49-292, |
| | 49-295, 49-296, 49-298, 49-303, 49-305, 49-311, 49-314, 49-333, 49-335, 49-345, 49-379, 49-423, 49-446, 49-449, |
| | 50-246, 56-304, 59-336, 62-344, 64-423, 69-254, 70-412, 71-489, 72-332, 73-349, 83-358, 93-255, 103-361, 106- |
| | 380, 112-253, 124-448, 130-423, 133-338, 135-230, 148-362, 154-374, 171-417, 172-371, 174-435, 177-285, 179- |
| | 404, 179-441, 179-450, 182-333, 183-385, 183-389, 190-420, 197-489, 200-389, 200-489, 209-440, 209-475, 213- |
| | 489, 214-379, 218-515, 225-469, 230-467, 234-458, 236-438, 242-437, 244-412, 245-476, 253-474, 253-479, 258- |
| | 481, 258-484, 259-453, 262-374, 265-489, 270-485, 275-489, 290-443, 308-489, 318-452, 344-484, 354-449, 488- |
| | 511, 488-518, 488-605, 488-610, 488-672, 488-682, 488-683, 488-694, 488-700, 488-805, 488-940, 494-744, |
| | 497-759, 499-653, 504-700, 511-772, 514-801, 514-811, 514-1046, 521-787, 537-823, 538-740, 547-856, 547-878, |
| | 557-994, 566-784, 566-787, 567-821, 567-954, 575-830, 579-815, 580-854, 586-767, 586-834, 606-864, 628-1038, |
| | 629-830, 632-893, 638-874, 644-877, 650-895, 652-849, 659-850, 659-865, 659-1025, 662-907, 664-960, 667-846, |
| | 671-909, 674-913, 675-855, 676-952, 680-933, 681-926, 684-926, 703-973, 709-940, 712-943, 712-1272, 717-1255, |
| | 719-983, 722-962, 722-992, 724-967, 727-985, 729-947, 729-956, 730-954, 730-966, 732-980, 747-955, 747-956, |
| | 747-965, 747-997, 747-1011, 759-988, 761-1113, 765-1104, 769-1179, 770-1004, 778-982, 778-1026, 778-1041, |
| | 778-1068, 779-1022, 779-1041, 779-1053, 780-1218, 784-1000, 784-1043, 795-1029, 800-1073, 811-1040, 811- |
| | 1045, 814-1099, 821-1050, 821-1053, 823-1022, 831-1104, 832-1086, 838-990, 839-1072, 845-1249, 847-971, 847- |
| | 1001, 847-1030, 848-1116, 849-1120, 857-1072, 859-1061, 860-1061, 862-1102, 862-1120, 867-1280, 870-1126, |
| | 872-1135, 874-1242, 882-1129, 899-1212, 910-1178, 911-1108, 912-1147, 912-1169, 913-1173, 917-1138, 922- |
| | 1202, 924-1216, 929-1167, 932-1257, 932-1294, 932-1330, 936-1221, 936-1229, 941-1341, 942-1191, |
| | 946-1173, 950-1188, 951-1297, 971-1153, 972-1292, 978-1236, 1001-1251, 1025-1212, 1030-1286, 1032-1189, |
| | 1035-1225, 1054-1309, 1061-1252, 1062-1185, 1062-1294, 1062-1360, 1063-1332, 1064-1285, |
| | 1065-1362, 1066-1265, 1069-1286, 1070-1310, 1082-1331, 1088-1345, 1095-1324, 1100-1228, 1112-1239, 1112- |
| | 1367, 1114-1333, 1114-1347, 1115-1344, 1117-1365, 1121-1321, 1121-1328, 1133-1367 |
| 91/7503624CB1 | 1-309, 2-198, 2-211, 2-227, 2-244, 2-267, 2-274, 2-1065, 3-231, 3-236, 3-260, 3-279, 3-294, 4-218, 4-223, 4-266, 5- |
| 1067 | 201, 5-210, 5-214, 5-222, 5-261, 5-267, 5-272, 5-281, 6-149, 6-220, 6-248, 6-256, 6-263, 7-215, 7-217, 7-218, 7-309, |
| | 8-241, 8-260, 8-269, 9-227, 9-235, 9-238, 9-246, 9-261, 9-262, 10-238, 11-168, 11-286, 12-236, 12-288, 13-253, 18- |
| | 220, 18-238, 19-179, 21-220, 21-230, 21-263, 23-184, 23-241, 23-271, 24-241, 24-275, 24-278, 24-281, 24-297, 25- |
| | 239, 25-261, 25-262, 31-130, 31-279, 31-286, 33-229, 39-287, 42-309, 45-309, 52-237, 55-309, 95-236, 116-309, |
| | 118-213, 157-421, 160-268, 165-309, 305-465, 305-513, 305-532, 305-552, 305-562, 326-736, 327-528, 330-591, |
| | 336-572, 342-575, 348-593, 350-547, 357-548, 357-563, 357-723, 360-605, 362-658, 365-544, 369-607, 372-611, |
| | 373-553, 374-650, 378-631, 379-624, 382-624, 401-671, 407-638, 410-641, 410-970, 415-953, 417-681, 420-660, |
| | 420-690, 422-667, 425-683, 427-645, 427-654, 428-652, 428-664, 430-678, 445-653, 445-654, 445-663, 445-695, |
| | 445-709, 457-686, 459-811 , 463-802, 467-877, 468-702, 476-680, 476-724, 476-739, 476-766, |
| | 477-720, 477-739, 477-751, 478-916, 482-698, 482-741, 493-727, 498-775, 509-738, 509-743, 512-797, 519-748, |
| | 519-751, 521-720, 529-802, 530-784, 536-688, 537-770, 543-947, 545-669, 545-699, 545-728, |
| | 546-814, 547-818, 555-770, 557-759, 558-759, 560-800, 560-818, 565-978, 568-824, 570-833, 572-940, 580-827, |
| | 597-910, 608-876, 609-806, 610-845, 610-867, 611-880, 615-836, 620-900, 622-914, 627-865, |
| | 630-955, 630-992, 630-1028, 634-919, 634-927, 639-1039, 640-889, 644-871, 648-886, 649-995, 669-851, 670-990, |
| | 676-934, 699-949, 723-910, 728-984, 730-887, 733-923, 752-1007, 759-950, 760-883, 760-992, |
| | 760-1058, 761-1030, 762-983, 763-1060, 764-963, 767-984, 768-1008, 780-1029, 786-1043, 793-1022, 798-926, |
| | 810-937, 810-1065, 812-1031, 812-1045, 813-1042, 815-1063, 819-1019, 819-1026, 831-1067 |
| 92/7503625CB1/ | 1-1685, 48-284, 49-147, 49-166, 49-185, 49-196, 49-201, 49-215, 49-218, 49-227, 49-228, 49-231, 49-232, 49-234, |
| 1685 | 49-235, 49-237, 49-239, 49-240, 49-244, 49-247, 49-248, 49-252, 49-253, 49-255, 49-256, 49-258, 49-261, 49-263, |
| | 49-265, 49-270, 49-273, 49-277, 49-278, 49-279, 49-280, 49-283, 49-284, 49-286, 49-288, 49-289, 49-291, 49-292, |
| | 49-295, 49-296, 49-298, 49-303, 49-305, 49-311, 49-314, 49-333, 49-335, 49-345, 49-379, 49-423, 49-446, 49-449, |
| | 49-551, 49-612, 49-616, 49-641, 49-649, 50-246, 56-304, 59-336, 62-344, 64-423, 69-254, 70-412, 71-490, 72-332, |
| | 73-349, 80-659, 83-358, 103-361, 103-528, 105-574, 106-380, 107-505, 112-253, 117-566, 119-517, 119-584, 122- |
| | 603, 124-448, 130-423, 130-549, 133-338, 135-230, 138-511, 148-362, 154-374, 164-623, 171-417, 172-371, 174- |
| | 435, 177-285, 179-404, 179-441, 179-450, 182-333, 183-385, 183-389, 190-420, 197-490, 200-389, 200-495, 209- |
| | 440, 209-475, 209-613, 213-493, 214-379, 224-509, 225-469, 230-467, 234-458, 236-438, 242-437, 244-412, 245- |
| | 476, 251-550, 253-474, 253-479, 255-545, 258-481, 258-484, 258-657, 259-453, 259-569, 262-374, 263-587, |
| | 265-499, 270-485, 275-505, 285-515, 288-551, 290-443, 290-514, 290-540, 295-542, 297-514, 299-520, 300-539, |
| | 300-576, 308-503, 316-609, 317-549, 317-580, 323-545, 329-543, 330-585, 337-613, 342-597, |
| | 342-612, 343-542, 344-484, 346-649, 349-615, 354-449, 365-657, 383-641, 390-602, 391-634, 393-615, 397-661, |
| | 407-603, 419-652, 421-617, 426-625, 429-655, 433-608, 433-660, 456-697, 464-687, 473-690, 473-691, 544-797 |
| 93/7503627CB1/ | 1-478, 36-1117, 108-741, 161-656, 187-666, 201-475, 211-493, 224-488, 229-499, 230-505, 235-492, 236-494, 236- |
| 1117 | 609, 238-506, 239-422, 239-499, 241-500, 241-501, 247-518, 250-497, 251-531, 253-487, |
| | 255-496, 255-503, 255-505, 255-506, 255-518, 256-499, 256-518, 256-558, 257-517, 260-494, 260-538, 261-520, |
| | 261-560, 262-529, 272-576, 280-615, 290-494, 291-546, 291-554, 307-772, 308-591, 308-592, |
| | 310-586, 314-505, 314-544, 314-555, 314-587, 314-764, 315-595, 319-566, 321-728, 323-563, 325-593, 325-602, |
| | 326-548, 326-569, 327-559, 327-583, 334-544, 334-549, 334-606, 336-538, 339-541, 348-624, |
| | 370-515, 375-657, 376-689, 379-510, 379-630, 381-582, 387-650, 387-653, 388-585, 389-646, 391-640, 396-477, |
| | 397-633, 406-825, 417-575, 420-749, 423-650, 427-622, 429-684, 443-730, 448-630, 462-597, 494-756, 505-749, |
| | 512-702, 567-815 |
| 94/7505744CB1/ | 1-586, 50-458, 50-461, 72-461, 153-418, 153-586, 156-423, 178-453, 197-332, 199-453, 253-514, 261-409, 272- |
| 586 | 571, 274-495, 274-530, 301-579, 302-586, 307-540, 348-586, 444-586 |
| 95/7503712CB1/ | 1-3261, 1-3280, 393-523, 756-862, 789-1077, 1037-1427, 1438-1711, 1450-1707, 1450-1757, 1450-1759, 1450- |
| 3394 | 1762, 1450-1763, 1450-1955, 1450-2042, 1450-2083, 1450-2112, 1451-2250, 1573-2240, |
| | 1604-2223, 1637-2012, 1755-2015, 1940-2228, 1952-2471, 2032-2307, 2041-2334, 2128-2418, 2141-2771, 2223- |
| | 2695, 2281-2583, 2281-2588, 2284-2531, 2294-2973, 2324-2900, 2336-2592, 2428-2904, |
| | 2433-3013, 2465-3102, 2468-2733, 2471-3180, 2503-3184, 2507-3190, 2517-3119, 2517-3145, 2517-3193, 2524- |
| | 3046, 2524-3061, 2524-3177, 2545-2959, 2545-3055, 2548-3221, 2582-2957, 2644-3249, |
| | 2698-3017, 2713-3242, 2726-2997, 2747-3026, 2758-3273, 2781-3063, 2786-3266, 2792-3238, 2798-2872, 2799- |
| | 2865, 2799-3198, 2799-3242, 2799-3291, 2802-3243, 2817-3302, 2827-3053, 2830-3211, |
| | 2841-3121, 2863-3261, 2874-3288, 2888-3258, 2890-3180, 2903-3275, 2906-3177, 3007-3259, 3007-3274, 3026- |
| | 3287, 3026-3301, 3037-3277, 3038-3275, 3042-3261, 3042-3296, 3070-3267, 3077-3261, 3078-3275, 3079-3273, |
| | 3103-3394 |
| 96/7503713CB1/ | 1-3080, 1-3099, 393-523, 737-1338, 756-862, 789-1077, 793-1350, 916-1350, 1361-1634, 1373-1630, 1373-1680, |
| 3213 | 1373-1682, 1373-1685, 1373-1686, 1373-1878, 1373-1965, 1373-2006, 1373-2035, |
| | 1374-2173, 1496-2163, 1527-2146, 1560-1935, 1678-1938, 1863-2151, 1955-2230, 1964-2260, 2034-2620, 2194- |
| | 2618, 2233-2820, 2256-2723, 2256-2832, 2256-2857, 2284-2921, 2287-2552, 2290-2999, |
| | 2322-3003, 2326-3009, 2336-2938, 2336-2964, 2336-3012, 2343-2865, 2343-2880, 2343-2996, 2364-2778, 2364- |
| | 2874, 2367-3040, 2401-2776, 2460-2712, 2463-3068, 2517-2836, 2532-3061, 2545-2816, |
| | 2566-2845, 2577-3092, 2600-2882, 2605-3085, 2611-3057, 2617-2691, 2618-2684, 2618-3017, 2618-3061, 2618- |
| | 3110, 2621-3062, 2636-3121, 2646-2872, 2649-3030, 2660-2940, 2682-3080, 2693-3107, |
| | 2707-3077, 2709-2999, 2722-3094, 2725-2996, 2826-3078, 2826-3093, 2845-3106, 2845-3120, 2856-3096, 2857- |
| | 3094, 2861-3080, 2861-3115, 2889-3086, 2896-3080, 2897-3094, 2898-3092, 2922-3213 |
| 97/7502225CB1/ | 1-331, 36-487, 50-573, 67-329, 67-389, 67-549, 72-488, 78-398, 82-342, 100-183, 100-206, 100-368, 101-357, 116- |
| 1435 | 406, 119-417, 119-420, 119-426, 119-427, 119-429, 119-530, 119-543, 119-594, 119-647, |
| | 119-694, 126-422, 128-458, 128-614, 137-514, 138-616, 142-524, 155-524, 168-424, 174-725, 185-591, 210-429, |
| | 210-435, 214-500, 229-619, 229-816, 229-847, 263-591, 283-907, 285-589, 321-586, 321-647, |
| | 321-675, 324-635, 324-652, 324-658, 324-667, 324-671, 333-605, 360-623, 371-518, 381-647, 388-647, 431-787, |
| | 445-1080, 484-712, 530-757, 533-805, 665-931, 720-965, 743-997, 765-1131, 774-1168, 774-1178, 817-1235, 817- |
| | 1435, 837-1041, 843-1163, 851-1033 |
| 98/7502778CB1/ | 1-846, 326-915, 439-1029, 607-1043, 617-1253, 655-1209, 675-1253, 703-999, 711-1253, 743-1312, 743-1405, 743- |
| 2371 | 1449, 747-1489, 792-1396, 852-1682, 878-1176, 890-1254, 913-1199, 916-1711, 926-1451, |
| | 935-1662, 940-1662, 948-1622, 965-1620, 967-1458, 969-1472, 974-1568, 978-1520, 987-1866, 1029-1255, 1029- |
| | 1581, 1033-1566, 1108-1866, 1131-1394, 1166-1866, 1221-1845, 1240-1866, 1244-1866, |
| | 1263-2038, 1270-1900, 1288-1855, 1288-1858, 1485-1861, 1489-1866, 1510-2159, 1515-2173, 1523-1866, 1528- |
| | 1866, 1607-1861, 1641-2123, 1660-2371, 1716-2174, 1721-2174, 1802-2172 |
| 99/7503699CB1/ | 1-275, 1-509, 1-3739, 242-525, 284-667, 597-1132, 599-1183, 624-1323, 892-1506, 964-1227, 964-1483, 964-1502, |
| 3760 | 1012-1351, 1015-1501, 1041-1309, 1046-1535, 1115-1514, 1213-1349, 1231-1579, 1265-1720, 1265-1721, 1265- |
| | 1722, 1265-1725, 1265-1734, 1265-1740, 1265-1743, 1265-1745, 1265-1747, 1265-1749, 1265-1753, 1265-1755, |
| | 1266-1742, 1268-1740, 1270-1751, 1273-1742, 1273-1755, 1274-1726, 1274-1747, 1279-1744, 1280-1725, 1281- |
| | 1742, 1282-1742, 1283-1725, 1300-1516, 1389-1791, 1543-1791, 1612-1766, 1626-1800, 1626-1936, 1626-2242, |
| | 1626-2246, 1626-2248, 1626-2276, 1626-2305, 1626-2368, 1626-2394, 1626-2412, 1626-2438, 1626-2452, 1626- |
| | 2465, 1627-2242, 1650-2106, 1650-2139, 1683-1799, 1683-1843, 1718-2256, 1718-2429, 1719-2342, 1719-2350, |
| | 1719-2368, 1719-2440, 1720-2390, 1850-2615, 1889-2428, 1893-2606, 1905-2615, 1914-2615, 1933-2606, 1933- |
| | 2615, 1962-2548, 2132-2539, 2215-2302, 2251-2606, 2314-2607, 2947-3205, 2947-3462, 2957-3617, 2960-3371, |
| | 2963-3581, 2967-3497, 2984-3605, 2986-3577, 3006-3580, 3014-3651, 3023-3644, 3027-3502, 3044-3534, |
| | 3047-3561, 3049-3654, 3064-3641, 3069-3565, 3069-3657, 3083-3626, 3085-3681, 3089-3480, 3119-3706, 3119- |
| | 3735, 3127-3626, 3129-3704, 3131-3739, 3199-3760, 3203-3661, 3212-3734, 3267-3719, 3274-3665, 3327-3678, |
| | 3336-3699, 3385-3644 |
| 100/6026715CB1/ | 1-1522, 201-624, 201-725, 201-852, 202-869, 206-490, 238-948, 405-628, 428-918, 477-1107, 722-1182, 995-1461, |
| 1522 | 999-1466, 1012-1467, 1025-1467, 1048-1260, 1051-1466, 1059-1462, 1139-1463, 1168-1463, 1215-1467, 1222- |
| | 1466, 1236-1486, 1236-1501, 1236-1522 |
| 101/7503631CB1/ | 1-197, 1-210, 1-226, 1-243, 1-266, 1-273, 1-315, 1-533, 1-1313, 2-230, 2-235, 2-259, 2-278, 2-293, 2-405, 2-594, 3- |
| 1315 | 217, 3-222, 3-265, 4-200, 4-209, 4-213, 4-221, 4-260, 4-266, 4-271, 4-280, 4-361, 4-598, 4-623, 4-631, 5-148, 5-219, |
| | 5-247, 5-255, 5-262, 6-214, 6-216, 6-217, 6-317, 6-327, 7-240, 7-259, 7-268, 8-226, 8-234, 8-237, 8-245, 8-260, 8- |
| | 261, 8-361, 9-237, 10-167, 10-285, 11-287, 12-252, 22-237, 30-129, 30-178, 30-183, 30-219, 30-229, 30-235, 30- |
| | 238, 30-240, 30-260, 30-261, 30-262, 30-270, 30-274, 30-277, 30-278, 30-280, 30-285, 30-296, 30-428, 30-431, 32- |
| | 228, 38-286, 41-318, 44-326, 46-405, 51-236, 52-394, 53-472, 54-314, 55-331, 62-641, 65-340, 85-343, 85-510, 87- |
| | 556, 88-362, 89-487, 94-235, 99-548, 101-499, 101-566, 104-585, 106-430, 112-405, 112-531, 115-320, 117-212, |
| | 120-493, 130-344, 136-356, 146-605, 153-399, 154-353, 156-417, 159-267, 161-386, 161-423, 161-432, 164-315, |
| | 165-367, 165-371, 172-402, 179-472, 182-371, 182-475, 191-422, 191-457, 191-595, 195-474, 196-361, 206-491, |
| | 207-451, 212-449, 216-440, 218-420, 224-419, 226-394, 227-458, 233-532, 235-456, 235-461, |
| | 237-527, 240-463, 240-466, 240-639, 241-435, 241-551, 244-356, 245-569, 247-481, 252-467, 257-487, 267-497, |
| | 270-533, 272-425, 272-496, 272-522, 277-524, 279-496, 281-502, 282-521, 282-558, 290-485, 298-591, 299-531, |
| | 299-562, 305-527, 311-525, 312-567, 319-595, 324-579, 324-594, 325-524, 326-466, 326-585, 328-631, 329-731, |
| | 331-597, 336-431, 347-639, 365-623, 367-720, 372-584, 373-616, 375-597, 379-643, 389-585, 396-704, 401-634, |
| | 403-493, 403-599, 408-607, 411-637, 414-671, 415-590, 415-642, 438-679, 445-719, 446-669, 455-672, 455-673, |
| | 485-699, 491-736, 502-730, 502-743, 523-726, 526-1069, 529-659, 529-664, 532-743, 534-736, 534-737, 535-743, |
| | 553-707, 558-743, 736-928, 736-946, 736-950, 736-968, 736-972, 736-987, 736-989, 736-999, 736-1014, 736-1050, |
| | 736-1125, 736-1164, 741-975, 746-1023, 757-986, 757-991, 760-1045, 767-996, 767-999, 769-968, 777-1050, 778- |
| | 1032, 784-936, 785-1018, 791-1195, 793-917, 793-947, 793-976, 794-1062, 795-1066, 803-1018, 805-1007, 806- |
| | 1007, 808-1048, 808-1066, 813-1226, 816-1072, 818-1081, 820-1188, 828-1075, 845-1158, 856-1124, 857-1054, |
| | 858-1093, 858-1115, 859-1128, 863-1084, 868-1148, 870-1162, 875-1113, 878-1203, 878-1240, 878-1276, |
| | 882-1167, 882-1175, 887-1287, 888-1137, 892-1119, 896-1134, 897-1243, 917-1099, 918-1238, 924-1182, 947- |
| | 1197, 971-1158, 976-1232, 978-1135, 981-1171, 1000-1255, 1007-1198, 1008-1131, 1008-1240, |
| | 1008-1306, 1009-1278, 1010-1231, 1011-1308, 1012-1211, 1015-1232, 1016-1256, 1028-1277, 1034-1291, 1041- |
| | 1270, 1046-1174, 1058-1185, 1058-1313, 1060-1279, 1060-1293, 1061-1290, 1063-1311, 1067-1267, 1067-1274, |
| | 1079-1315 |
| 102/7503664CB1/ | 1-197, 1-210, 1-226, 1-243, 1-266, 1-273, 1-315, 1-533, 1-1373, 2-230, 2-235, 2-259, 2-278, 2-293, 2-405, 2-594, 3- |
| 1373 | 217, 3-222, 3-265, 4-200, 4-209, 4-213, 4-221, 4-260, 4-266, 4-271, 4-280, 4-361, 4-598, 4-623, 4-631, 5-148, 5-219, |
| | 5-247, 5-255, 5-262, 6-214, 6-216, 6-217, 6-317, 6-327, 7-240, 7-259, 7-268, 8-226, 8-234, 8-237, 8-245, 8-260, 8- |
| | 261, 8-361, 9-237, 10-167, 10-285, 11-287, 12-252, 21-178, 21-219, 21-229, 21-262, 21-428, 21-431, 30-129, 30- |
| | 183, 30-235, 30-237, 30-238, 30-240, 30-260, 30-261, 30-270, 30-274, 30-277, 30-278, 30-280, 30-285, 30-296, 32- |
| | 228, 38-286, 41-318, 44-326, 46-405, 5 1-236, 52-394, 53-472, 54-314, 55-331, 62-641, 65-340, 85-343, 85-510, 87- |
| | 556, 88-362, 89-487, 94-235, 99-548, 101-499, 101-566, 104-585, 106-430, 112-405, 112-531, 115-320, 117-212, |
| | 120-493, 130-344, 136-356, 146-605, 153-399, 154-353, 156-417, 159-267, 161-386, 161-423, 161-432, 164-315, |
| | 165-367, 165-371, 172-402, 179-472, 182-371, 182-475, 191-422, 191-457, 191-595, 195-474, 196-361, 206-491, |
| | 207-451, 212-449, 216-440, 218-420, 224-419, 226-394, 227-458, 233-532, 235-456, 235-461, 237-527, 240-463, |
| | 326-585, 328-631, 329-731, 329-771, 331-597, 336-431, 347-639, 357-844, 365-623, 367-720, 372-584, 373-616, |
| | 375-597, 379-643, 389-585, 396-704, 401-634, 403-493, 403-599, 408-607, 411-637, 414-671, 415-590, 415-642, |
| | 438-679, 445-719, 446-669, 455-672, 455-673, 468-760, 473-814, 485-699, 491-736, 502-730, 502-751, 523-726, |
| | 529-659, 529-664, 531-994, 532-748, 534-736, 534-737, 535-754, 536-859, 548-798, 551-813, 553-707, 558-754, |
| | 565-826, 568-855, 568-865, 575-841, 591-877, 592-794, 601-910, 601-932, 611-1048, 620-838, 620-850, 621-875, |
| | 621-1008, 629-884, 633-869, 634-908, 640-821, 640-888, 660-918, 683-884, 686-947, 692-928, 698-931, 704-949, |
| | 706-903, 713-904, 713-919, 716-961, 718-1014, 721-900, 725-963, 728-967, 729-909, 730-1006, 734-987, 735-980, |
| | 738-980, 757-1027, 763-994, 766-997, 773-1037, 776-1016, 776-1046, 778-1023, 781-1039, 783-1001, 783-1010, |
| | 784-1008, 784-1020, 786-1034, 801-1009, 801-1010, 801-1019, 801-1051, 813-1042, 824-1051, 832-1036, 838- |
| | 1051, 892-1044, 901-1025, 901-1051, 997-1241, 1049-1193, 1049-1216, 1049-1229, 1049-1290, 1058-1313, 1065- |
| | 1256, 1066-1189, 1066-1298, 1066-1364, 1067-1336, 1068-1289, 1069-1366, 1070-1269, 1073-1290, |
| | 1074-1314, 1086-1335, 1092-1349, 1099-1328, 1104-1232, 1116-1243, 1116-1371, 1118-1337, 1118-1351, 1119- |
| | 1348, 1121-1369, 1125-1325, 1125-1332, 1137-1371 |
| 103/7504812CB1/ | 1-110, 1-138, 1-201, 1-260, 1-283, 1-297, 1-314, 1-478, 1-540, 1-547, 1-573, 1-575, 1-583, 1-585, 1-600, 1-601, 1- |
| 1002 | 602, 1-605, 1-1002, 2-267, 2-297, 2-610, 3-241, 3-530, 3-596, 3-598, 4-243, 4-584, 5-228, 6-259, 6-288, 6-491, 6- |
| | 600, 6-605, 7-309, 7-607, 10-269, 11-491, 14-295, 19-488, 20-600, 28-605, 157-408, 173-437, 175-402, 176-433, |
| | 180-471, 182-413, 187-471, 194-440, 215-464, 220-393, 228-460, 237-524, 242-485, 243-586, 245-511, 246-446, |
| | 246-479, 253-481, 255-508, 261-428, 266-540, 267-473, 268-498, 269-514, 270-393, 270-475, 270-483, 281-599, |
| | 282-609, 283-517, 285-528, 287-501, 289-532, 289-546, 289-561, 289-564, 289-578, 291-546, 292-515, 292-564, |
| | 293-581, 295-579, 297-527, 298-553, 300-558, 300-587, 301-533, 302-488, 302-499, 302-521, 303-551, 308-591, |
| | 309-491, 309-549, 310-531, 314-570, 317-550, 318-488, 319-566, 321-545, 322-605, 323-583, 326-559, 326-606, |
| | 327-583, 327-585, 328-610, 340-461, 345-584, 345-585, 345-605, 345-607, 345-610, 346-558, 354-480, 354-595, |
| | 358-559, 362-608, 383-602, 384-610, 410-589, 428-565, 462-610, 471-610, 489-720, 541-917, 546-917, |
| | 615-873, 630-1002, 635-869, 715-917, 716-917, 748-917, 757-974, 769-1002, 851-1002, 853-989, 855-989, 855- |
| | 991, 855-1002, 856-1002, 900-994, 903-1002, 910-1002, 913-969, 913-979, 922-1002 |
| 104/7504996CB1/ | 1-530, 1-627, 1-753, 8-628, 103-384, 153-695, 219-592, 242-476, 242-1062, 293-886, 307-771, 320-585, 323-590, |
| 1062 | 345-620, 364-499, 366-620, 420-681, 428-576, 439-738, 441-662, 441-697, 468-746, 469-763, 474-707, 515-765, |
| | 611-775 |
| 105/7377279CB1/ | 1-204, 14-123, 18-133, 30-488, 30-1364, 252-481, 780-1327, 780-1460, 823-1466, 910-1179, 910-1283, 910-1362, |
| 1794 | 910-1371, 910-1389, 910-1413, 910-1443, 910-1463, 910-1464, 910-1469, 910-1470, 910-1500, |
| | 910-1523, 910-1768, 916-1466, 924-1518, 933-1582, 1003-1513, 1043-1269, 1045-1379, 1053-1565, 1149-1646, |
| | 1162-1299, 1169-1628, 1172-1435, 1201-1423, 1225-1317, 1240-1664, 1241-1768, |
| | 1248-1794, 1261-1672, 1263-1548, 1413-1614, 1413-1621, 1413-1681, 1417-1616, 1485-1768 |
| 106/2060984CB1/ | 1-302, 7-200, 17-611, 18-130, 18-682, 25-204, 25-417, 26-200, 27-272, 29-118, 39-286, 42-574, 100-1019, 212-463, |
| 1667 | 359-802, 376-641, 378-622, 378-894, 417-669, 430-679, 450-684, 450-716, 450-1015, |
| | 473-720, 485-728, 493-754, 535-786, 540-797, 559-718, 580-789, 580-1364, 585-779, 602-903, 642-916, 666-1113, |
| | 666-1204, 695-1128, 703-929, 713-979, 723-991, 726-957, 726-958, 734-999, 750-1217, |
| | 755-1020, 765-998, 791-1038, 791-1322, 799-1066, 804-1105, 806-1418, 808-1259, 844-1095, 845-1646, 847-1259, |
| | 887-1138, 887-1250, 923-1208, 953-1404, 974-1181, 987-1650, 988-1266, 988-1398, |
| | 1022-1432, 1028-1223, 1037-1655, 1041-1436, 1044-1168, 1044-1300, 1076-1428, 1081-1325, 1088-1336, 1090- |
| | 1286, 1116-1434, 1146-1428, 1150-1381, 1195-1654, 1198-1484, 1201-1506, 1214-1487, |
| | 1214-1512, 1217-1435, 1229-1502, 1234-1461, 1251-1658, 1266-1659, 1278-1596, 1281-1658, 1281-1662, 1287- |
| | 1667, 1317-1567, 1476-1656 |
| 107/4085993CB1/ | 1-273, 1-281, 18-74, 23-656, 37-708, 279-1147, 348-985, 484-729, 513-1121, 605-801, 605-881, 605-1018, 605- |
| 2290 | 1081, 605-1139, 605-1184, 673-1133, 708-1252, 790-1147, 949-1666, 963-1637, 1000-1299, |
| | 1000-1497, 1026-1536, 1162-1761, 1212-1754, 1213-1761, 1218-1823, 1238-1831, 1243-1766, 1263-1652, 1276- |
| | 1706, 1278-1497, 1309-1686, 1316-1735, 1322-1809, 1334-1969, 1356-1991, 1356-2007, |
| | 1389-1881, 1404-2002, 1410-1862, 1410-1910, 1410-2002, 1421-1883, 1424-1955, 1434-2062, 1436-1734, 1484- |
| | 1838, 1487-2148, 1506-2018, 1517-2083, 1531-1851, 1539-2095, 1553-2173, 1585-2136, |
| | 1608-2162, 1617-1861, 1629-1836, 1638-2166, 1653-2264, 1657-2045, 1657-2256, 1677-1869, 1725-2290, 1732- |
| | 2288, 1793-2285, 1809-2285, 1871-2285, 1899-2290, 2003-2290 |
| 108/6756305CB1/ | 1-40, 1-237, 49-3053, 50-293, 53-357, 76-359, 76-368, 77-329, 78-318, 83-292, 83-319, 83-329, 83-347, 83-631, 83- |
| 3053 | 745, 83-800, 85-428, 90-367, 90-374, 94-122, 113-364, 134-393, 145-346, 163-393, 189-401, 253-479, 253-634, 286- |
| | 555, 335-676, 354-916, 354-1014, 356-629, 392-1061, 440-687, 441-657, 445-1018, 492-764, 499-1185, 561-907, |
| | 574-845, 644-949, 652-833, 668-1241, 671-1201, 689-1092, 776-1073, 798-993, 826-1053, 828-11 17, 855-1093, |
| | 879-1174, 973-1250, 1020-1545, 1027-1224, 1077-1344, 1080-1371, 1085-1368, 1087-1287, 1098-1379, 1190- |
| | 1456, 1294-1606, 1354-1752, 1358-1639, 1438-1946, 1440-1705, 1475-1670, 1475-2071, 1525-1851, 1537-1794, |
| | 1559-1733, 1583-1821, 1640-1883, 1670-1925, 1717-1955, 1733-1981, 1790-2109, 1847-2063, 1847-2124, 1859- |
| | 2542, 1860-2080, 1860-2084, 1860-2438, 1878-2183, 1953-2185, 1971-2265, 1984-2195, 1984-2450, 2026-2221, |
| | 2067-2352, 2097-2353, 2134-2363, 2153-2676, 2156-2433, 2160-2400, 2165-2416, 2167-2405, 2174-2440, 2190- |
| | 2446, 2199-2464, 2213-2851, 2274-2547, 2274-2550, 2277-2585, 2285-2559, 2296-2545, 2296-2549, |
| | 2304-2908, 2314-2566, 2317-2557, 2319-2572, 2324-2632, 2331-2606, 2336-2526, 2338-2526, 2339-2789, 2370- |
| | 2578, 2374-2696, 2376-2544, 2383-2641, 2384-2625, 2386-2673, 2400-2635, 2403-2982, |
| | 2415-2674, 2421-3051, 2436-2855, 2439-2597, 2450-3041, 2453-2688, 2460-2653, 2463-2716, 2496-2727, 2502- |
| | 2630, 2502-2781, 2502-2785, 2506-2747, 2506-3053, 2510-2807, 2522-2781 |
| 109/7504842CB1/ | 1-276, 1-420, 1-515, 1-2132, 72-366, 81-541, 83-346, 83-357, 89-362, 89-609, 89-761, 91-450, 92-488, 95-334, 135- |
| 2850 | 391, 155-400, 156-598, 180-356, 185-356, 197-541, 198-664, 203-633, 206-734, 235-467, |
| | 287-517, 316-768, 360-812, 360-933, 509-856, 640-902, 642-902, 647-1063, 669-1241, 703-960, 703-1203, 714- |
| | 1299, 727-975, 727-1222, 862-1160, 879-1153, 914-1462, 1088-1294, 1088-1301, 1088-1517, |
| | 1088-1520, 1088-1522, 1088-1557, 1088-1562, 1088-1578, 1088-1580, 1088-1592, 1088-1663, 1088-1671, 1098- |
| | 1353, 1132-1683, 1193-1850, 1248-1880, 1313-1908, 1316-1908, 1365-2127, 1402-1575, |
| | 1408-1575, 1418-2053, 1434-2068, 1447-2068, 1449-1737, 1454-1847, 1455-2095, 1467-2067, 1503-2095, 1534- |
| | 2116, 1535-2123, 1568-2122, 1681-2132, 1695-2129, 1696-2137, 1697-2137, 1703-2159, |
| | 1708-2141, 1725-2171, 1736-1990, 1736-1996, 1738-2149, 1745-2129, 1809-2161, 1978-2141, 2005-2131, 2014- |
| | 2131, 2034-2132, 2052-2850 |
| 110/7500901CB1/ | 1-227, 1-267, 1-1243, 7-227, 7-272, 7-284, 7-307, 8-228, 8-276, 11-270, 11-527, 11-548, 11-611, 11-615, 15-268, 42- |
| 1247 | 284, 42-638, 45-670, 46-336, 49-726, 59-346, 173-741, 199-687, 203-624, 222-518, 286-548, |
| | 286-551, 290-459, 298-875, 326-905, 343-645, 400-833, 407-950, 420-968, 429-939, 430-652, 438-872, 440-721, |
| | 445-667, 472-763, 493-704, 493-975, 509-713, 509-972, 517-696, 520-765, 524-714, 525-738, |
| | 558-863, 590-677, 617-937, 617-1173, 617-1205, 637-924, 672-957, 718-944, 747-985, 773-999, 774-974, 789-947, |
| | 803-976, 824-1243, 894-1243, 978-1243, 979-1245, 990-1243, 1001-1243, 1004-1152, |
| | 1004-1236, 1010-1243, 1040-1243, 1063-1247, 1064-1158, 1073-1203, 1120-1240, 1160-1247 |
| 111/7500898CB1/ | 1-227, 1-267, 1-1378, 6-593, 7-227, 7-272, 7-276, 8-228, 8-288, 11-270, 15-268, 42-276, 110-360, 277-418, 277- |
| 1379 | 834, 285-864, 302-604, 359-792, 366-909, 379-927, 388-898, 389-611, 397-831, 399-680, |
| | 404-626, 431-722, 441-1164, 452-663, 452-934, 468-672, 468-997, 476-655, 477-1075, 479-724, 483-673, 484-697, |
| | 517-822, 517-1036, 549-636, 576-896, 596-883, 605-1210, 631-916, 670-1041, 677-903, |
| | 680-1234, 706-938, 706-1294, 733-933, 748-906, 762-956, 764-1304, 770-1229, 789-1073, 813-1371, 824-1090, |
| | 850-1372, 850-1374, 885-938, 887-1376, 909-1364, 912-1245, 912-1346, 912-1379, 925-1360, |
| | 925-1365, 926-1367, 934-1346, 939-1365, 945-1368, 947-1378, 963-1355, 967-1364, 983-1365, 987-1345, 988- |
| | 1184, 988-1325, 990-1371, 995-1364, 997-1374, 1007-1362, 1010-1372, 1018-1373, 1033-1364, |
| | 1042-1360, 1046-1368, 1050-1364, 1055-1374, 1056-1346, 1098-1365, 1098-1379, 1109-1365, 1120-1364, 1123- |
| | 1271, 1123-1355, 1129-1377, 1129-1379, 1159-1379, 1182-1358, 1183-1277, 1192-1322, 1239-1359, 1279-1373 |
| 112/7500912CB1/ | 1-627, 82-215, 98-881, 103-192, 132-810, 203-762, 215-318, 215-325, 215-345, 215-385, 215-414, 215-420, 215- |
| 907 | 431, 215-449, 215-458, 215-459, 215-465, 215-467, 215-470, 215-476, 215-490, 215-664, 215-695, 215-720, 215- |
| | 803, 215-810, 215-855, 216-434, 217-810, 218-606, 219-771, 220-801, 221-864, 222-876, 232-803, 234-782, 244- |
| | 793, 245-557, 249-712, 253-657, 255-789, 267-498, 277-529, 284-581, 293-803, 295-480, 295-570, 300-556, 300- |
| | 604, 300-862, 303-744, 304-593, 305-849, 306-691, 309-875, 310-843, 312-594, 312-608, 318-585, 319-417, 321- |
| | 602, 323-586, 325-600, 333-543, 333-583, 335-847, 336-598, 337-567, 338-598, 343-798, 346-588, 346-751, 349- |
| | 436, 350-843, 355-847, 356-619, 356-629, 356-843, 358-733, 360-603, 361-592, 367-733, 370-591, 370-766, 370- |
| | 882, 380-570, 380-792, 380-849, 386-582, 386-596, 388-841, 388-843, 392-849, 394-868, 397-660, 397-843, 397- |
| | 864, 397-865, 397-891, 400-832, 400-860, 400-863, 401-876, 402-849, 402-897, 403-849, 405-614, 407-708, 409- |
| | 742, 412-894, 415-664, 415-745, 415-760, 416-842, 417-665, 418-868, 419-864, 423-547, 426-878, |
| | 428-681, 428-829, 428-881, 429-846, 432-635, 437-677, 437-879, 439-647, 439-843, 440-863, 442-877, 443-907, |
| | 444-858, 448-683, 449-664, 452-656, 452-796, 452-843, 452-864, 454-660, 456-866, 461-840, |
| | 467-724, 467-846, 469-842, 469-866, 470-855, 471-722, 471-843, 472-673, 472-717, 475-687, 475-688, 475-704, |
| | 475-843, 477-605, 477-860, 478-904, 482-679, 482-862, 486-722, 487-846, 488-865, 490-739, |
| | 494-761, 495-754, 498-752, 502-846, 502-848, 506-723, 507-842, 507-844, 508-704, 512-846, 513-779, 513-860, |
| | 519-720, 519-758, 521-818, 529-794, 535-735, 540-774, 546-800, 546-808, 566-712, 570-843, |
| | 581-801, 585-860, 586-807, 596-841, 599-877, 602-863, 604-847, 608-866, 613-869, 618-821, 621-839, 621-843, |
| | 621-871, 622-730, 634-843, 642-803, 651-873, 653-884, 654-863, 662-863, 662-868, 668-853, |
| | 684-903, 699-818, 701-817, 702-864, 721-904, 727-863, 739-801, 739-844, 744-837, 744-850, 751-846, 768-860 |
| 113/7505851CB1/ | 1-271, 2-1198, 63-242, 99-243, 106-243, 109-365, 117-243, 126-243, 126-275, 430-516, 433-657, 433-746, 433- |
| 1391 | 781, 433-916, 433-1064, 438-520, 475-789, 520-962, 521-634, 521-698, 521-720, 521-722, 521-724, 521-731, 521- |
| | 734, 521-743, 521-747, 521-748, 521-758, 521-771, 521-774, 521-787, 521-788, 521-790, 521-799, 521-801, 521- |
| | 809, 521-812, 521-819, 521-822, 521-846, 521-1000, 521-1007, 521-1019, 521-1021, 521-1080, 521-1082, 522-753, |
| | 522-811, 523-914, 523-1067, 524-790, 524-909, 527-1080, 528-1061, 530-812, 530-826, 532-900, 536-803, 537- |
| | 635, 537-640, 539-820, 540-764, 541-804, 543-670, 543-818, 543-865, 549-748, 551-733, 551-761, 551-801, 553- |
| | 776, 553-1065, 554-660, 554-810, 554-816, 555-785, 555-861, 556-816, 560-765, 560-817, 561-1016, 564-806, 564- |
| | 846, 564-969, 565-778, 566-806, 566-896, 567-654, 567-861, 567-1101, 567-1107, 567-1179, 567-1185, 568-1061, |
| | 573-818, 573-1065, 574-837, 574-847, 574-1061, 576-951, 577-848, 578-821, 579-810, 581-826, 585-951, 588-809, |
| | 588-910, 588-984, 588-1041, 594-725, 598-788, 598-809, 598-822, 598-884, 598-1010, 598-1067, |
| | 603-944, 604-800, 604-814, 606-1059, 606-1061, 610-1067, 612-1082, 613-878, 615-878, 615-1061, 615-1082, 618- |
| | 1050, 618-1078, 618-1081, 619-1080, 620-1082, 621-880, 621-1067, 623-832, 625-926, 626-881, 626-908, 627-960, |
| | 628-1067, 630-1082, 633-882, 633-963, 633-978, 634-833, 634-1060, 635-883, 636-1082, 637-885, 637-1122, 637- |
| | 1191, 640-1067, 641-765, 644-1080, 646-899, 646-1047, 646-1082, 647-1064, 648-1064, 650-853, 653-1059, 653- |
| | 1064, 655-895, 655-1082, 657-865, 657-1061, 658-1081, 660-1082, 661-1075, 661-1082, 662-1076, 664-1183, 666- |
| | 901, 667-882, 670-874, 670-911, 670-1014, 670-1061, 670-1089, 672-878, 674-1082, 679-1058, 685-942, 685-1064, |
| | 686-915, 687-1060, 687-1082, 688-1073, 689-940, 689-1061, 690-891, 690-935, 693-905, 693-906, 693-922, 693- |
| | 1061, 695-823, 695-1078, 696-1079, 700-897, 700-1080, 704-940, 705-1064, 705-1065, 706-1082, 708-877, 708- |
| | 957, 711-1028, 712-979, 713-972, 716-970, 720-1064, 720-1066, 724-941, 725-1060, 725-1062, 726-922, 726-923, |
| | 730-1064, 731-997, 731-1078, 736-1391, 737-938, 737-976, 739-1036, 742-1145, 747-1012, 753-953, 754-1064, |
| | 758-992, 764-1018, 764-1026, 777-1198, 784-930, 788-1061, 789-1067, 796-1061, |
| | 799-1019, 799-1060, 799-1067, 803-1078, 804-1025, 814-1059, 817-1082, 820-1081, 822-1065, 826-1082, 831- |
| | 1082, 832-1042, 836-1039, 839-1057, 839-1061, 839-1082, 852-1061, 860-1021, 869-1082, 871-1082, |
| | 872-1081, 875-1067, 878-1060, 880-982, 880-1081, 880-1082, 886-1071, 900-1053, 902-1082, 917-1036, 919-1035, |
| | 920-1082, 921-1077, 939-1061, 945-1081, 957-1019, 957-1062, 962-1055, 962-1068, 969-1064, 969-1185, 989- |
| | 1060, 1002-1307, 1016-1300, 1041-1198 |
| 114/7503696CB1/ | 1-275, 1-499, 1-3494, 413-678, 413-912, 413-925, 831-1170, 860-1128, 865-1357, 934-1337, 1032-1168, 1050- |
| 3579 | 1398, 1084-1539, 1084-1540, 1084-1541, 1084-1544, 1084-1553, 1084-1559, 1084-1562, 1084-1564, 1084-1566, |
| | 1084-1568, 1084-1572, 1084-1574, 1085-1561, 1087-1559, 1089-1570, 1092-1561, 1092-1574, 1093-1545, 1093- |
| | 1566, 1098-1563, 1099-1544, 1100-1561, 1101-1561, 1102-1544, 1119-1335, 1431-1585, 1445-2061, 1445-2065, |
| | 1445-2067, 1445-2095, 1445-2124, 1445-2231, 1445-2271, 1445-2284, 1446-2061, 1469-1925, 1469-1958, 1537- |
| | 2075, 1537-2248, 1538-2161, 1538-2169, 1538-2187, 1538-2259, 1539-2209, 1669-2434, 1708-2247, 1712-2425, |
| | 1719-2434, 1752-2425, 1781-2367, 1845-2434, 1951-2358, 2070-2425, 2133-2426, 2766-3024, 2766-3281, 2776- |
| | 3436, 2779-3190, 2782-3400, 2786-3316, 2803-3424, 2805-3396, 2825-3399, 2833-3470, 2842-3463, 2846-3321, |
| | 2863-3353, 2866-3380, 2868-3473, 2883-3460, 2888-3384, 2888-3476, 2902-3445, 2904-3500, 2908-3299, 2938- |
| | 3525, 2938-3554, 2946-3445, 2948-3523, 2950-3558, 3018-3579, 3022-3480, 3031-3553, 3086-3538, |
| | 3093-3484, 3146-3497, 3155-3518, 3204-3463 |

TABLE 5

| Polynucleotide SEQ | | |
|---|---|---|
| ID NO: | Incyte Project ID: | Representative Library |
| 58 | 1318936CB1 | OVARDIR01 |
| 59 | 033784CB1 | TESTNOF01 |
| 60 | 4160218CB1 | ADRENOT14 |
| 61 | 7501989CB1 | THP1NOT03 |
| 62 | 7500695CB1 | BLADTUT04 |
| 63 | 7502088CB1 | TLYJTXF01 |
| 64 | 7198825CB1 | MUSLTDR02 |
| 65 | 71863981CB1 | BRAHTDR03 |
| 66 | 7500513CB1 | HEARNOT01 |
| 67 | 7501080CB1 | 293TF4T01 |
| 68 | 3144878CB1 | HNT2AZS07 |
| 69 | 7502112CB1 | SKINDIA01 |
| 70 | 7502788CB1 | TLYJTXN01 |
| 71 | 7500171CB1 | LUNGNON07 |
| 72 | 7500172CB1 | CERVNOT01 |
| 73 | 7500899CB1 | FIBPFEN06 |
| 74 | 7500916CB1 | TLYMNOT08 |
| 75 | 7502479CB1 | PROSTUS23 |
| 76 | 3015057CB1 | BONRFEC01 |
| 77 | 6303570CB1 | NERDTDN03 |
| 78 | 7501579CB1 | ISLTNOT01 |
| 79 | 7503812CB1 | BRSTTUT01 |
| 80 | 7503970CB1 | BRAHNON05 |
| 81 | 2578986CB1 | KIDNTUT13 |
| 82 | 7503786CB1 | BRAXNOA01 |
| 83 | 7505684CB1 | BRSTTUT08 |
| 84 | 302655CB1 | TESTNOC01 |
| 85 | 4757551CB1 | CORPNOT02 |
| 86 | 7503157CB1 | PLACNOB01 |
| 87 | 7503702CB1 | UTRENOT10 |
| 88 | 72096217CB1 | BLADNOT05 |
| 89 | 7503620CB1 | CARGDIT01 |
| 90 | 7503621CB1 | CARGDIT01 |
| 91 | 7503624CB1 | CARGDIT01 |
| 92 | 7503625CB1 | CARGDIT01 |
| 93 | 7503627CB1 | BRAITUT03 |
| 94 | 7505744CB1 | KERANOT02 |
| 95 | 7503712CB1 | BRAHNON05 |
| 96 | 7503713CB1 | BRAHNON05 |
| 97 | 7502225CB1 | BRAHNON05 |
| 98 | 7502778CB1 | BRSTNOT24 |
| 99 | 7503699CB1 | UTRENOT10 |
| 100 | 6026715CB1 | TESTNOT03 |
| 101 | 7503631CB1 | CARGDIT01 |
| 102 | 7503664CB1 | CARGDIT01 |
| 103 | 7504812CB1 | ADRETUR01 |
| 104 | 7504996CB1 | KERANOT02 |
| 105 | 7377279CB1 | ESOGTUE01 |
| 106 | 2060984CB1 | BRAINON01 |
| 107 | 4085993CB1 | SMCCNOS01 |
| 108 | 6756305CB1 | UTRSTMR01 |
| 109 | 7504842CB1 | BSTMNON02 |
| 110 | 7500901CB1 | FIBPFEN06 |
| 111 | 7500898CB1 | FIBPFEN06 |
| 112 | 7500912CB1 | TLYMNOT08 |
| 113 | 7505851CB1 | TLYMNOT08 |
| 114 | 7503696CB1 | TLYMNOT08 |

TABLE 6

| Library | Vector | Library Description |
|---|---|---|
| 293TF4T01 | pINCY | Library was constructed using RNA isolated from a transformed embryonal cell line |
| | | (293-EBNA) derived from kidney epithelial tissue transfected with e2fl and dp1. The |
| | | cells were transformed with adenovirus 5 DNA. |
| ADRENOT14 | pINCY | Library was constructed using RNA isolated from adrenal gland tissue removed from an |
| | | 8-year-old Black male, who died from anoxia. |
| ADRETUR01 | PCDNA2.1 | This random primed library was constructed using RNA isolated from left upper pole, |
| | | adrenal gland tumor tissue removed from a 52-year-old Caucasian male during |
| | | nephroureterectomy and local destruction of renal lesion. Pathology indicated |
| | | grade 3 adrenal cortical carcinoma forming a mass that infiltrated almost the whole |
| | | adrenal parenchyma and extended to adjacent adipose tissue. A metastatic tumor |
| | | nodule was identified in the hilar region. The renal vein was infiltrated by tumor |
| | | and the neoplastic process was present at the resection margin of the renal vein. |
| | | Fragments of adrenal cortical carcinoma and thrombus were found in the inferior |
| | | vena cava. Patient history included abnormal weight loss. Family history included |
| | | skin cancer, type I diabetes, and neurotic depression. |
| BLADNOT05 | pINCY | Library was constructed using RNA isolated from bladder tissue removed from a 60- |
| | | year-old Caucasian male during a radical cystectomy, prostatectomy, and vasectomy. |
| | | Pathology for the associated tumor tissue indicated grade 3 transitional cell |
| | | carcinoma. Carcinoma in-situ was identified in the dome and trigone. Patient history |
| | | included tobacco use. |
| BLADTUT04 | pINCY | Library was constructed using RNA isolated from bladder tumor tissue removed from a |
| | | 60-year-old Caucasian male during a radical cystectomy, prostatectomy, and vasectomy. |
| | | Pathology indicated grade 3 transitional cell carcinoma in the left bladder wall. |
| | | Carcinoma in-situ was identified in the dome and trigone. Patient history included |
| | | tobacco use. Family history included type I diabetes, malignant neoplasm of the |
| | | stomach, atherosclerotic coronary artery disease, and acute myocardial infarction. |
| BONRFEC01 | pINCY | This large size-fractionated library was constructed using RNA isolated from rib |
| | | bone tissue removed from a Caucasian male fetus who died from Patau's syndrome |
| | | (trisomy 13) at 20-weeks' gestation. Serologies were negative. |
| BRAHNON05 | pINCY | This normalized hippocampus tissue library was constructed from 1.6 million |
| | | independent clones from a hippocampus tissue library. Starting RNA was made from |
| | | posterior hippocampus removed from a 35-year-old Caucasian male who died from |
| | | cardiac failure. Pathology indicated moderate leptomeningeal fibrosis and multiple |
| | | microinfarctions of the cerebral neocortex. The cerebral hemisphere revealed |
| | | moderate fibrosis of the leptomeninges with focal calcifications. There was evidence |
| | | of shrunken and slightly eosinophilic pyramidal neurons throughout the cerebral |
| | | hemispheres. There were small microscopic areas of cavitation with gliosis, |
| | | scattered through the cerebral cortex. Patient history included cardiomyopathy, CHF, |
| | | cardiomegaly, an enlarged spleen and liver. Patient medications included simethi- |
| | | cone, Lasix, Digoxin, Colace, Zantac, captopril, and Vasotec. The library was |
| | | normalized in two rounds using conditions adapted from Soares et al., PNAS (1994) |
| | | 91: 9228 and Bonaldo et al., Genome Research 6 (1996): 791, except that a significantly |
| | | longer (48 hours/round) reannealing hybridization was used. |
| BRAHTDR03 | PCDNA2.1 | This random primed library was constructed using RNA isolated from archaecortex, |
| | | anterior hippocampus tissue removed from a 55-year-old Caucasian female who died |
| | | from cholangiocarcinoma. Pathology indicated mild meningeal fibrosis predominately |
| | | over the convexities, scattered axonal spheroids in the white matter of the |
| | | cingulate cortex and the thalamus, and a few scattered neurofibrillary tangles in |
| | | the entorhinal cortex and the periaqueductal gray region. Pathology for the asso- |
| | | ciated tumor tissue indicated well-differentiated cholangiocarcinoma of the liver |
| | | with residual or relapsed tumor. Patient history included cholangiocarcinoma, post- |
| | | operative Budd-Chiari syndrome, biliary ascites, hydrothorax, dehydration, |
| | | malnutrition, oliguria and acute renal failure. Previous surgeries included |
| | | cholecystectomy and resection of 85% of the liver. |
| BRAINON01 | PSPORT1 | Library was constructed and normalized from 4.88 million independent clones from a |
| | | brain tissue library. RNA was made from brain tissue removed from a 26-year-old |
| | | Caucasian male during cranioplasty and excision of a cerebral meningeal lesion. |
| | | Pathology for the associated tumor tissue indicated a grade 4 oligoastrocytoma in |
| | | the right fronto-parietal part of the brain. The normalization and hybridization |
| | | conditions were adapted from Soares et al., PNAS (1994) 91: 9228, except that a |
| | | significantly longer (48-hour) reannealing hybridization was used. |
| BRAITUT03 | PSPORT1 | Library was constructed using RNA isolated from brain tumor tissue removed from the |
| | | left frontal lobe of a 17-year-old Caucasian female during excision of a cerebral |
| | | meningeal lesion. Pathology indicated a grade 4 fibrillary giant and small-cell |
| | | astrocytoma. Family history included benign hypertension and cerebrovascular disease. |
| BRAXNOA01 | PSPORT1 | This amplified library was constructed using RNA isolated from sensory-motor cortex |
| | | tissue removed from a 35-year-old Caucasian male who died from cardiac failure. |
| | | Pathology indicated moderate leptomeningeal fibrosis and multiple microinfarctions |
| | | of the cerebral neocortex. Microscopically, the cerebral hemisphere revealed |
| | | moderate fibrosis of the leptomeninges with focal calcifications. There was |
| | | evidence of shrunken and slightly eosinophilic pyramidal neurons throughout the |
| | | cerebral hemispheres. Scattered throughout the cerebral cortex, there were multiple |
| | | small microscopic areas of cavitation with surrounding gliosis. Patient history |
| | | included dilated cardiomyopathy, congestive heart failure, cardiomegaly and an |
| | | enlarged spleen and liver. |
| BRSTNOT24 | pINCY | Library was constructed using RNA isolated from diseased breast tissue removed from |
| | | a 46-year-old Caucasian female during bilateral subcutaneous mammectomy. Pathology |
| | | indicated nonproliferative fibrocystic disease. Family history included breast |
| | | cancer and cardiovascular disease. |
| BRSTTUT01 | PSPORT1 | Library was constructed using RNA isolated from breast tumor tissue removed from a |
| | | 55-year-old Caucasian female during a unilateral extended simple mastectomy. |
| | | Pathology indicated invasive grade 4 mammary adenocarcinoma of mixed lobular and |
| | | ductal type, extensively involving the left breast. The tumor was identified in the |
| | | deep dermis near the lactiferous ducts with extracapsular extension. Seven mid and |
| | | low and five high axillary lymph nodes were positive for tumor. Proliferative |
| | | fibrocysytic changes were characterized by apocrine metaplasia, sclerosing adenosis, |
| | | cyst formation, and ductal hyperplasia without atypia. Patient history included |
| | | atrial tachycardia, blood in the stool, and a benign breast neoplasm. Family history |
| | | included benign hypertension, atherosclerotic coronary artery disease, |
| | | cerebrovascular disease, and depressive disorder. |
| BRSTTUT08 | pINCY | Library was constructed using RNA isolated from breast tumor tissue removed from a |
| | | 45-year-old Caucasian female during unilateral extended simple mastectomy. Pathology |
| | | indicated invasive nuclear grade 2-3 adenocarcinoma, ductal type, with 3 of 23 lymph |
| | | nodes positive for metastatic disease. Greater than 50% of the tumor volume was in |
| | | situ, both comedo and non-comedo types. Immunostains were positive for estrogen/ |
| | | progesterone receptors, and uninvolved tissue showed proliferative changes. The |
| | | patient concurrently underwent a total abdominal hysterectomy. Patient history |
| | | included valvuloplasty of mitral valve without replacement, rheumatic mitral |
| | | insufficiency, and rheumatic heart disease. Family history included acute myocardial |
| | | infarction, atherosclerotic coronary artery disease, and type II diabetes. |
| BSTMNON02 | PSPORT1 | This normalized brain stem library was constructed from 2.84 million independent |
| | | clones from a brain stem library. Starting RNA was made from the brain stem tissue |
| | | of a 72-year-old Caucasian male who died from myocardial infarction. Patient history |
| | | included coronary artery disease, insulin-dependent diabetes mellitus, and arthritis. |
| | | Normalization and hybridization conditions were adapted from Soares et al. (PNAS |
| | | (1994) 91: 9228). |
| CARGDIT01 | pINCY | Library was constructed using RNA isolated from diseased cartilage tissue. Patient |
| | | history included osteoarthritis. |
| CERVNOT01 | PSPORT1 | Library was constructed using RNA isolated from the uterine cervical tissue of a 35- |
| | | year-old Caucasian female during a vaginal hysterectomy with dilation and curettage. |
| | | Pathology indicated mild chronic cervicitis. Family history included atherosclerotic |
| | | coronary artery disease and type II diabetes. |
| CORPNOT02 | pINCY | Library was constructed using RNA isolated from diseased corpus callosum tissue |
| | | removed from the brain of a 74-year-old Caucasian male who died from Alzheimer's |
| | | disease. |
| ESOGTUE01 | pINCY | This 5′ biased random primed library was constructed using RNA isolated from |
| | | esophageal tumor tissue removed from a 61 year-old Caucasian male during a partial |
| | | esophagectomy, proximal gastrectomy, pyloromyotomy, and regional lymph node excision. |
| | | Pathology indicated an invasive grade 3 adenocarcinoma in the esophagus, extending |
| | | distally to involve the gastroesophageal junction. The tumor extended through the |
| | | muscularis to involve periesophageal and perigastric soft tissues. One perigastric |
| | | and two periesophageal lymph nodes were positive for tumor. There were multiple |
| | | perigastric and periesophageal tumor implants. The patient presented with deficiency |
| | | anemia and myelodysplasia. Patient history included hyperlipidemia, and tobacco and |
| | | alcohol abuse in remission. Previous surgeries included adenotonsillectomy, |
| | | rhinoplasty, vasectomy, and hemorrhoidectomy. A previous bone marrow aspiration |
| | | found the marrow to be hypercellular for age and had a cellularity-to-fat ratio of |
| | | 95:5. The marrow was focally densely fibrotic. Granulocytic precursors were slightly |
| | | increased with normal maturation. |
| | | The estimate of blast cells was greater than 5%. Megakaryocytes were increased and |
| | | appeared atypical in clusters. Storage cells and granulomata were absent. Patient |
| | | medications included Epoetin, Danocrine, Berocca Plus tablets, Selenium, vitamin B6 |
| | | phosphate, vitamins E & C, and beta carotene. Family history included alcohol abuse, |
| | | atherosclerotic coronary artery disease, type II diabetes, chronic liver disease, |
| | | and primary cardiomyopathy in the father; and benign hypertension and cerebrovas- |
| | | cular disease in the mother. |
| FIBPFEN06 | pINCY | The normalized prostate stromal fibroblast tissue libraries were constructed from |
| | | 1.56 million independent clones from a prostate fibroblast library. Starting RNA was |
| | | made from fibroblasts of prostate stroma removed from a male fetus, who died after |
| | | 26 weeks' gestation. The libraries were normalized in two rounds using conditions |
| | | adapted from Soares et al., PNAS (1994) 91: 9228 and Bonaldo et al.. Genome Research |
| | | (1996) 6: 791, except that a significantly longer (48-hours/round)reannealing |
| | | hybridization was used. The library was then linearized and recircularized to select |
| | | for insert containing clones as follows: plasmid DNA was prepped from approximately |
| | | 1 million clones from the normalized prostate stromal fibroblast tissue libraries |
| | | following soft agar transformation. |
| HEARNOT01 | PBLUESCRIPT | Library was constructed using RNA isolated from the whole heart tissue of a 56-year- |
| | | old male, who died from an intracranial bleed. |
| HNT2AZS07 | PSPORT1 | This subtracted library was constructed from RNA isolated from an hNT2 cell line |
| | | (derived from a human teratocarcinoma that exhibited properties characteristic of a |
| | | committed neuronal precursor) treated for three days with 0.35 micromolar AZ. The |
| | | hybridization probe for subtraction was derived from a similarly constructed library |
| | | from untreated hNT2 cells. 3.08 M clones from the AZ-treated library were subjected |
| | | to three rounds of subtractive hybridization with 3.04 M clones from the untreated |
| | | library. Subtractive hybridization conditions were based on the methodologies of |
| | | Swaroop et al. (NAR (1991) 19: 1954) and Bonaldo et al. (Genome Research (1996) |
| | | 6: 791). |
| ISLTNOT01 | pINCY | Library was constructed using RNA isolated from a pooled collection of pancreatic |
| | | islet cells. |
| KERANOT02 | PSPORT1 | Library was constructed using RNA isolated from epidermal breast keratinocytes |
| | | (NHEK). NHEK (Clontech #CC-2501) is a human breast keratinocyte cell line derived |
| | | from a 30-year-old black female during breast-reduction surgery. |
| KIDNTUT13 | pINCY | Library was constructed using RNA isolated from kidney tumor tissue removed from a |
| | | 51-year-old Caucasian female during a nephroureterectomy. Pathology indicated a |
| | | grade 3 renal cell carcinoma. Patient history included depressive disorder, |
| | | hypoglycemia, and uterine endometriosis. Family history included calculus of the |
| | | kidney, colon cancer, and type II diabetes. |
| LUNGNON07 | pINCY | This normalized lung tissue library was constructed from 5.1 million independent |
| | | clones from a lung tissue library. Starting RNA was made from RNA isolated from lung |
| | | tissue. The library was normalized in two rounds using conditions adapted from |
| | | Soares et al., PNAS (1994) 91: 9228-9232 and Bonaldo et al., Genome Research |
| | | (1996) 6: 791, except that a significantly longer (48 hours/round) reannealing |
| | | hybridization was used. |
| MUSLTDR02 | PCDNA2.1 | This random primed library was constructed using RNA isolated from right lower thigh |
| | | muscle tissue removed from a 58-year-old Caucasian male during a wide resection of |
| | | the right posterior thigh. Pathology indicated no residual tumor was identified in |
| | | the right posterior thigh soft tissue. Changes were consistent with a previous |
| | | biopsy site. On section through the soft tissue and muscle there was a smooth cystic |
| | | cavity with hemorrhage around the margin on one side. The wall of the cyst was |
| | | smooth and pale-tan. Pathology for the matched tumor tissue indicated a grade II |
| | | liposarcoma. Patient history included liposarcoma (right thigh), and hypercholes- |
| | | terolemia. Previous surgeries included resection of right thigh mass. Family history |
| | | included myocardial infarction and an unspecified rare blood disease. |
| NERDTDN03 | pINCY | This normalized dorsal root ganglion tissue library was constructed from 1.05 |
| | | million independent clones from a dorsal root ganglion tissue library. Starting RNA |
| | | was made from dorsal root ganglion tissue removed from the cervical spine of a 32- |
| | | year-old Caucasian male who died from acute pulmonary edema, acute bronchopneumonia, |
| | | bilateral pleural effusions, pericardial effusion, and malignant lymphoma (natural |
| | | killer cell type). The patient presented with pyrexia of unknown origin, malaise, |
| | | fatigue, and gastrointestinal bleeding. Patient history included probable |
| | | cytomegalovirus infection, liver congestion, and steatosis, splenomegaly, |
| | | hemorrhagic cystitis, thyroid hemorrhage, respiratory failure, pneumonia of the left |
| | | lung, natural killer cell lymphoma of the pharynx, Bell's palsy, and tobacco and |
| | | alcohol abuse. Previous surgeries included colonoscopy, closed colon biopsy, |
| | | adenotonsillectomy, and nasopharyngeal endoscopy and biopsy. Patient medications |
| | | included Diflucan (fluconazole), Deltasone (prednisone), hydrocodone, Lortab, |
| | | Alprazolam, Reazodone, ProMace-Cytabom, Etoposide, Cisplatin, Cytarabine, and |
| | | dexamethasone. |
| | | The patient received radiation therapy and multiple blood transfusions. The library |
| | | was normalized in 2 rounds using conditions adapted from Soares et al., PNAS (1994) |
| | | 91: 9228-9232 and Bonaldo et al., Genome Research 6 (1996): 791, except that a |
| | | significantly longer (48 hours/round) reannealing hybridization was used. |
| OVARDIR01 | PCDNA2.1 | This random primed library was constructed using RNA isolated from right ovary |
| | | tissue removed from a 45-year-old Caucasian female during total abdominal |
| | | hysterectomy, bilateral salpingo-oophorectomy, vaginal suspension and fixation, and |
| | | incidental appendectomy. Pathology indicated stromal hyperthecosis of the right and |
| | | left ovaries. Pathology for the matched tumor tissue indicated a dermoid cyst |
| | | (benign cystic teratoma) in the left ovary. Multiple (3) intramural leiomyomata were |
| | | identified. The cervix showed squamous metaplasia. Patient history included |
| | | metrorrhagia, female stress incontinence, alopecia, depressive disorder, pneumonia, |
| | | normal delivery, and deficiency anemia. Family history included benign hypertension, |
| | | atherosclerotic coronary artery disease, hyperlipidemia, and primary tuberculous |
| | | complex. |
| PLACNOB01 | PBLUESCRIPT | Library was constructed using RNA isolated from placenta. |
| PROSTUS23 | pINCY | This subtracted prostate tumor library was constructed using 10 million clones from |
| | | a pooled prostate tumor library that was subjected to 2 rounds of subtractive |
| | | hybridization with 10 million clones from a pooled prostate tissue library. The |
| | | starting library for subtraction was constructed by pooling equal numbers of clones |
| | | from 4 prostate tumor libraries using mRNA isolated from prostate tumor removed from |
| | | Caucasian males at ages 58 (A), 61 (B), 66 (C), and 68 (D) during prostatectomy with |
| | | lymph node excision. Pathology indicated adenocarcinoma in all donors. History |
| | | included elevated PSA, induration and tobacco abuse in donor A; elevated PSA, |
| | | induration, prostate hyperplasia, renal failure, osteoarthritis, renal artery |
| | | stenosis, benign HTN, thrombocytopenia, hyperlipidemia, tobacco/alcohol abuse and |
| | | hepatitis C (carrier) in donor B; elevated PSA, induration, and tobacco abuse in |
| | | donor C; and elevated PSA, induration, hypercholesterolemia, and kidney calculus in |
| | | donor D. |
| | | The hybridization probe for subtraction was constructed by pooling equal numbers of |
| | | cDNA clones from 3 prostate tissue libraries derived from prostate tissue, prostate |
| | | epithelial cells, and fibroblasts from prostate stroma from 3 different donors. |
| | | Subtractive hybridization conditions were based on the methodologies of Swaroop et |
| | | al., NAR 19 (1991): 1954 and Bonaldo, et al. Genome Research 6 (1996): 791. |
| SKINDIA01 | PSPORT1 | This amplified library was constructed using RNA isolated from diseased skin tissue |
| | | removed from 1 female and 4 males during skin biopsies. Pathologies indicated |
| | | tuberculoid and lepromatious leprosy. |
| SMCCNOS01 | pINCY | This subtracted coronary artery smooth muscle cell library was constructed using |
| | | 7.56 × 10e6 clones from a coronary artery smooth muscle cell library and was |
| | | subjected to two rounds of subtraction hybridization for 48 hours with 6.12 × 10e6 |
| | | clones from a second coronary artery smooth muscle cell library. The starting |
| | | library for subtraction was constructed using RNA isolated from coronary artery |
| | | smooth muscle cells removed from a 3-year-old Caucasian male. The cells were treated |
| | | with TNF alpha & IL-1 beta 10 ng/ml each for 20 hours. The hybridization probe for |
| | | subtraction was derived from a similarly constructed library from RNA isolated from |
| | | untreated coronary artery smooth muscle cells from the same donor. Subtractive |
| | | hybridization conditions were based on the methodologies of Swaroop et al., (NAR |
| | | (1991) 19: 1954) and Bonaldo, et al. (Genome Research (1996) 6: 791-806). |
| TESTNOC01 | PBLUESCRIPT | This large size fractionated library was constructed using RNA isolated from |
| | | testicular tissue removed from a pool of eleven, 10 to 61-year-old Caucasian males. |
| TESTNOF01 | PSPORT1 | This 5′ cap isolated full-length library was constructed using RNA isolated from |
| | | testis tissue removed from a 26-year-old Caucasian male who died from head trauma |
| | | due to a motor vehicle accident. Serologies were negative. Patient history included |
| | | a hernia at birth, tobacco use (1½ ppd), marijuana use, and daily |
| | | alcohol use (beer and hard liquor). |
| TESTNOT03 | PBLUESCRIPT | Library was constructed using RNA isolated from testicular tissue removed from a 37- |
| | | year-old Caucasian male, who died from liver disease. Patient history included |
| | | cirrhosis, jaundice, and liver failure. |
| THP1NOT03 | pINCY | Library was constructed using RNA isolated from untreated THP-1 cells. THP-1 is a |
| | | human promonocyte line derived from the peripheral blood of a 1-year-old Caucasian |
| | | male with acute monocytic leukemia (ref: Int. J. Cancer (1980) 26: 171). |
| TLYJTXF01 | pRARE | This 5′ cap isolated full-length library was constructed using RNA isolated from |
| | | a treated Jurkat cell line derived from the T cells of a male. The cells were |
| | | treated with 5 nM of PMA and 50 ng/mL of Ionomycin for 1 hour. Patient history |
| | | included acute T-cell leukemia. |
| TLYJTXN01 | pRARE | This normalized Jurkat cell line library was constructed from independent clones |
| | | from a treated Jurkat cell line library. Starting RNA was made from a treated Jurkat |
| | | cell line derived from the T cells of a male. The cells were treated with 0.1 uM of |
| | | PMA for 30 minutes. Patient history included acute T-cell leukemia. The library was |
| | | normalized in one round using conditions adapted from Soares et al., PNAS (1994) |
| | | 91: 9228-9232 and Bonaldo et al., Genome Research 6 (1996): 791, except that a |
| | | significantly longer (48 hours/round) reannealing hybridization was used. |
| TLYMNOT08 | pINCY | The library was constructed using RNA isolated from anergicallogenic T-lymphocyte |
| | | tissue removed from an adult (40-50-year-old) Caucasian male. The cells were |
| | | incubated for 3 days in the presence of 1 microgram/ml OKT3 mAb and 5% human serum. |
| UTRENOT10 | pINCY | Library was constructed using RNA isolated from pooled uterine endometiral tissue |
| | | removed from three adult females during endometrial biopsy. Pathology indicated |
| | | normal endometrium. All three patients were positive for Beta-3 integrin. |
| UTRSTMR01 | pINCY | Library was constructed using RNA isolated from uterine myometrial tissue removed |
| | | from a 41-year-old Caucasian female during a vaginal hysterectomy. The endometrium |
| | | was secretory and contained fragments of endometrial polyps. Pathology for |
| | | associated tumor tissue indicated uterine leiomyoma. Patient history included |
| | | ventral hernia and a benign ovarian neoplasm. |

TABLE 7

| Program | Description | Reference | Parameter Threshold |
|---|---|---|---|
| ABI | A program that removes vector | Applied Biosystems, | |
| FACTURA | sequences and masks ambiguous | Foster City, CA. | |
| | bases in nucleic acid sequences. | | |
| ABI/ | A Fast Data Finder useful in | Applied Biosystems, | Mismatch <50% |
| PARACEL | comparing and annotating amino | Foster City, CA; | |
| PDF | acid or nucleic acid sequences. | Paracel Inc., | |
| | | Pasadena, CA. | |
| ABI | A program that assembles | Applied Biosystems, | |
| AutoAssembler | nucleic acid sequences. | Foster City, CA. | |
| BLAST | A Basic Local Alignment Search | Altschul, S. F. et al. (1990) | ESTs: Probability value = |
| | Tool useful in sequence | J. Mol. Biol. 215: 403-410; | 1.0E−8 or less; Full Length |
| | similarity search for amino acid | Altschul, S. F. et al. (1997) | sequences: Probability |
| | and nucleic acid sequences. | Nucleic Acids Res. 25: 3389-3402. | value = 1.0E−10 or less |
| | BLAST includes five functions: | | |
| | blastp, blastn, blastx, tblastn, | | |
| | and tblastx. | | |
| FASTA | A Pearson and Lipman algorithm | Pearson, W. R. and D. J. Lipman | ESTs: fasta E value = |
| | that searches for similarity | (1988) Proc. Natl. Acad Sci. USA | 1.06E−6; Assembled ESTs: |
| | between a query sequence and | 85: 2444-2448; Pearson, | fasta Identity = 95% or |
| | a group of sequences of the | W. R. (1990) Methods Enzymol. | greater and Match length = |
| | same type. FASTA comprises as | 183: 63-98; and Smith, T. F. | 200 bases or greater; |
| | least five functions: fasta, | and M. S. Waterman (1981) | fastx E value = 1.0E−8 |
| | tfasta, fastx, tfastx, and | Adv. Appl. Math. 2: 482-489. | or less; Full Length sequences: |
| | ssearch. | | fastx score = 100 or greater |
| BLIMPS | A BLocks IMProved Searcher | Henikoff, S. and J. G. Henikoff | Probability value = |
| | that matches a sequence against | (1991) Nucleic Acids Res. 19: | 1.0E−3 or less |
| | those in BLOCKS, PRINTS, DOMO, | 6565-6572; Henikoff, J. G. and | |
| | PRODOM, and PFAM databases to | S. Henikoff (1996) Methods Enzymol. | |
| | search for gene families, | 266:88-105; and Attwood, T. K. | |
| | sequence homology, and | et al. (1997) J. Chem. Inf. Comput. | |
| | structural fingerprint regions. | Sci. 37: 417-424. | |
| HMMER | An algorithm for searching a | Krogh, A. et al. (1994) J. Mol. Biol. | PFAM, INCY, SMART or |
| | query sequence against hidden | 235: 1501-1531; Sonnhammer, | TIGRFAM hits: Probability |
| | Markov model (HMM)-based data- | E. L. L. et al. (1988) Nucleic Acids | value = 1.0E−3 or less; |
| | bases of protein family consensus | Res. 26: 320-322; Durbin, R. et al. | Signal peptide hits: |
| | sequences, such as PFAM, INCY, | (1998) Our World View, in a Nutshell, | Score = 0 or greater |
| | SMART and TIGRFAM. | Cambridge Univ. Press, pp. 1-350. | |
| ProfileScan | An algorithm that searches for | Gribskov, M. et al. (1988) CABIOS | Normalized quality score ≧ GCG |
| | structural and sequence motifs | 4: 61-66; Gribskov, M. et al. | specified “HIGH” value for |
| | in protein sequences that match | (1989) Methods Enzymol. 183: 146-159; | that particular Prosite motif. |
| | sequence patterns defined in | Bairoch, A. et al. (1997) Nucleic Acids | Generally, score = 1.4-2.1. |
| | Prosite. | Res. 25: 217-221. | |
| Phred | A base-calling algorithm that | Ewing, B. et al. (1998) Genome Res. | |
| | examines automated sequencer | 8: 175-185; Ewing, B. and P. Green | |
| | traces with high sensitivity | (1998) Genome Res. 8: 186-194. | |
| | and probability. | | |
| Phrap | A Phils Revised Assembly Program | Smith, T. F. and M. S. Waterman | Score = 120 or greater; |
| | including SWAT and CrossMatch, | (1981) Adv. Appl. Math. 2: 482-489; | Match length = 56 or |
| | programs based on efficient | Smith, T. F. and M. S. Waterman | greater |
| | implementation of the Smith- | (1981) J. Mol. Biol. 147: 195-197; | |
| | Waterman algorithm, useful in | and Green, P., University of | |
| | searching sequence homology and | Washington, Seattle, WA. | |
| | assembling DNA sequences. | | |
| Consed | A graphical tool for viewing | Gordon, D. et al. (1998) Genome | |
| | and editing Phrap assemblies. | Res. 8: 195-202. | |
| SPScan | A weight matrix analysis program | Nielson, H. et al. (1997) Protein | Score = 3.5 or greater |
| | that scans protein sequences for | Engineering 10: 1-6; Claverie, | |
| | the presence of secretory signal | J. M. and S. Audic (1997) CABIOS | |
| | peptides. | 12: 431-439. | |
| TMAP | A program that uses weight | Persson, B. and P. Argos (1994) | |
| | matrices to delineate transmembrane | J. Mol. Bio 237: 182-192; | |
| | segments on protein sequences | Persson, B. and P. Argos (1996) | |
| | and determine orientation. | Protein Sci. 5: 363-371. | |
| TMHMMER | A program that uses a hidden | Sonnhammer, E. L. et al. (1998) | |
| | Markov model (HMM) to delineate | Proc. Sixth Intl. Conf. On | |
| | transmembrane segments on | Intelligent Systems for Mol. | |
| | protein sequences and determine | Biol., Glasgow et al., eds., | |
| | orientation. | The Am. Assoc. for Artificial | |
| | | Intelligence (AAAI) Press, | |
| | | Menlo Park, CA, and MIT Press, | |
| | | Cambridge, MA, pp. 175-182. | |
| Motifs | A program that searches amino | Bairoch, A. et al. (1997) Nucleic | |
| | acid sequences for patterns | Acids Res. 25: 217-221; Wisconsin | |
| | that matched those defined in | Package Program Manual, version 9, | |
| | Prosite. | page M51-59, Genetics Computer | |
| | | Group, Madison, WI. | |

1. An isolated polypeptide selected from the group consisting of:
a) a polypeptide comprising an amino acid sequence selected from the group consisting of SEQ ID NO:1-33, SEQ ID NO:35-43, SEQ ID NO:45, SEQ ID NO:48-51, and SEQ ID NO:53-57,
b) a polypeptide consisting essentially of the amino acid sequence selected from the group consisting of SEQ ID NO: NO:34, SEQ ID NO:44, SEQ ID NO:46-47, SEQ ID NO:52,
c) a polypeptide comprising a naturally occurring amino acid sequence at least 90% identical to an amino acid sequence selected from the group consisting of SEQ ID NO:1, SEQ ID NO:3, SEQ ID NO:7, SEQ ID MB-10-11, SEQ ID MB-14, SEQ ID NO:16, SEQ ID NO:20, SEQ ID NO:26-27, SEQ ID NO:32-33, SEQ ID NO:35-36, SEQ ID NO:39-41, SEQ ID NO:43, SEQ ID NO:48-51, SEQ ID NO:53-54, and SEQ ID NO:57,
d) a polypeptide comprising a naturally occurring amino acid sequence at least 92% identical to an amino acid sequence selected from the group consisting of SEQ ID NO:2, SEQ ID NO:9, and SEQ ID NO:38,
e) a polypeptide comprising a naturally occurring amino acid sequence at least 93% identical to the amino acid sequence of SEQ ID NO:19,
f) a polypeptide comprising a naturally occurring amino acid sequence at least 94% identical to the amino acid sequence of SEQ ID NO:24,
g) a polypeptide comprising a naturally occurring amino acid sequence at least 95% identical to an amino acid sequence selected from the group consisting of SEQ ID NO:4, SEQ ID NO:6, and SEQ ID NO:42,
h) a polypeptide comprising a naturally occurring amino acid sequence at least 96% identical to an amino acid sequence selected from the group consisting of SEQ ID NO:21, and SEQ ID NO:45,
i) a polypeptide comprising a naturally occurring amino acid sequence at least 97% identical to the amino acid sequence of SEQ ID MB-15,
j) a polypeptide comprising a naturally occurring amino acid sequence at least 98% identical to the amino acid sequence of SEQ ID NO:30,
k) a polypeptide comprising a naturally occurring amino acid sequence at least 99% identical to the amino acid sequence of SEQ ID NO:8,
l) a polypeptide consisting essentially of a naturally occurring amino acid sequence at least 90% identical to an amino acid sequence selected from the group consisting of SEQ ID MB-17, SEQ ID NO:25, SEQ ID NO:28, SEQ ID NO:31, SEQ ID NO:34, SEQ ID NO:37, SEQ ID NO:44, SEQ ID NO:46, SEQ ID NO:47, SEQ ID NO:52, and SEQ ID NO:56,
m) a biologically active fragment of a polypeptide having an amino acid sequence selected from the group consisting of SEQ ID MB-1-57, and
n) an immunogenic fragment of a polypeptide having an amino acid sequence selected from the group consisting of SEQ ID MB-1-57.

2. An isolated polypeptide of claim 1 selected from the group consisting of:
a) a polypeptide comprising an amino acid sequence selected from the group consisting of SEQ ID MB-1-33, SEQ ID NO:35-43, SEQ ID NO:45, SEQ ID NO:48-51, and SEQ ID NO:53-57, and
b) a polypeptide consisting essentially of an amino acid sequence selected from the group consisting of SEQ ID NO: NO:34, SEQ ID NO:44, SEQ ID NO:46-47, SEQ ID NO:52.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:58-114.

6. A recombinant polynucleotide comprising a promoter sequence operably linked to a polynucleotide of claim 3.

7. A cell transformed with a recombinant polynucleotide of claim 6.

8. (CANCELLED)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID MB-1-57.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:58-114,
b) a polynucleotide comprising a naturally occurring polynucleotide sequence at least 90% identical to a polynucleotide sequence selected from the group consisting of SEQ ID NO:58-77, SEQ ID NO:79-85, SEQ ID NO:87, SEQ ID NO:89-94,and and SEQ ID NO:96-114,
c) a polynucleotide comprising a naturally occurring polynucleotide sequence at least 92% identical to a polynucleotide sequence selected from the group consisting of SEQ ID NO:78 and SEQ ID NO:88,
d) a polynucleotide comprising a naturally occurring polynucleotide sequence at least 94% identical to the polynucleotide sequence of SEQ ID NO:95,
e) a polynucleotide comprising a naturally occurring polynucleotide sequence at least 99% identical to the polynucleotide sequence of SEQ ID NO:86,
f) a polynucleotide complementary to a polynucleotide of a),
g) a polynucleotide complementary to a polynucleotide of b),
h) a polynucleotide complementary to a polynucleotide of c),
i) a polynucleotide complementary to a polynucleotide of d),
j) a polynucleotide complementary to a polynucleotide of e), and
k) an RNA equivalent of a)-j).

13. (CANCELLED)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (CANCELLED)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide is selected from the group consisting of:
a) a polypeptide comprising an amino acid sequence selected from the group consisting of SEQ ID MB-1-33, SEQ ID NO:35-43, SEQ ID NO:45, SEQ ID NO:48-51, and SEQ ID NO:53-57, and
b) a polypeptide consisting essentially of an amino acid sequence selected from the group consisting of SEQ ID NO: NO:34, SEQ ID NO:44, SEQ ID NO:46-47, SEQ ID NO:52.

19. (CANCELLED)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21-22. (CANCELLED).

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24-25. (CANCELLED).

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (CANCELLED)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-169. (CANCELLED).
